(19) United States
(12) Patent Application Publication
(10) Pub. No.: US 2016/0340704 A1
(43) Pub. Date: Nov. 24, 2016

(54) METHOD OF MAKING A BENZYLISOQUINOLINE ALKALOID (BIA) METABOLITE, ENZYMES THEREFORE

(71) Applicant: VALORBEC SOCIÉTÉ EN COMMANDITE, Montreal (CA)

(72) Inventors: VINCENT MARTIN, MONTREAL (CA); LAUREN NARCROSS, MONTREAL (CA); ANDREW EKINS, VERDUN (CA); ELENA FOSSATI, BLAINVILLE (CA); YUN ZHU, SAINT-LAZARE (CA)

(21) Appl. No.: 15/111,041

(22) PCT Filed: Jan. 13, 2015
(86) PCT No.: PCT/CA2015/050021

§ 371 Date: Jul. 12, 2016

Related U.S. Application Data
(60) Provisional application No. 61/926,648, filed on Jan. 13, 2014.

(51) Int. Cl. C12P 17/12; C12N 9/10; C12N 15/52; C07D 217/04; C12N 15/81

(52) U.S. Cl. CPC C12P 17/12; C07D 217/04; C12N 15/81; C12N 15/52; C12N 9/1007; C12Y 201/01075; C12Y 201/0114; C07B 2200/07

(57) ABSTRACT

There is provided a method of preparing a benzylisoquinoline alkaloid (BIA) metabolite comprising: a. culturing a host cell under conditions suitable for protein production, including a pH of between about 7 and about 10 said host cell comprising: b. a first heterologous coding sequence encoding a first enzyme involved in a metabolite pathway that converts (R,S)-norlaudanosoline into the metabolite; c. a second heterologous coding sequence encoding a second enzyme involved in a metabolite pathway that converts (R,S)-norlaudanosoline into the metabolite; d. a third heterologous coding sequence encoding a second enzyme involved in a metabolite pathway that converts (R,S)-norlaudanosoline into the metabolite; (d) adding (R,S)-norlaudanosoline to the cell culture; and recovering the metabolite from the cell culture

### CROSS REFERENCE TO RELATED APPLICATIONS

This application is a PCT application Serial No. PCT/CA2015/0* filed on Jan. 13, 2015 and published in English under PCT Article 21(2), which itself claims benefit of U.S. provisional application Ser. No. 61/926,648, filed on Jan. 13, 2014. All documents above are incorporated herein in their entirety by reference.

### STATEMENT REGARDING FEDERALLY SPONSORED RESEARCH OR DEVELOPMENT

N.A.

### FIELD OF THE INVENTION

The present invention relates to a method of making a benzylisoquinoline alkaloid (BIA) metabolite. More specifically, the present invention is concerned with an improved method of making a (BIA) metabolite (e.g., reticuline) in a recombinant host cell.

### BACKGROUND OF THE INVENTION

Plant secondary metabolites are a rich source of bioactive molecules. The chemical diversity of these compounds derives from enzymes that have diversified to perform an array of stereo- and enantioselective modifications. Coupling these reactions by chemical synthesis to reach high yields can be difficult if not impossible. While plants remain the main source of these valuable natural compounds, the use of microbial platform has emerged as an attractive alternative.

Many pharmaceutical drugs are isolated directly from plants or are semisynthetic derivatives of natural products^{1,2}. Information from New Drug Applications and clinical trials is evidence that the pharmaceutical industry continues to use natural products as a source of new drug leads³. However, the pipeline of drug discovery is difficult to sustain, due to technical challenges in isolating new compounds with diverse structures and complex chemistries in sufficient quantities for screening⁴.

Next generation DNA sequencing technology has provided rapid access to the genetic diversity underpinning the immense biosynthetic capacity of plants and microbes^{5,6}. Although Saccharomyces cerevisiae has traditionally been used for the biosynthesis of simple molecules derived from central metabolism⁷, advances in recombinant DNA technology streamlining the cloning of large DNA sequences makes this microbe an attractive platform for functional characterization of enzymes as well as the reconstitution of complex metabolic pathways^{8,9}. When combined with genetic information on an ever-increasing number of species, microbial hosts provide new opportunities for the discovery and production of diverse and complex natural products. A recent example demonstrating the power of these combined technologies is the high-level production of the artemisinin antimalarial drug precursor artemisinic acid in yeast¹⁰.

Benzylisoquinoline alkaloids (BIAs) are a diverse class of plant secondary metabolites including such pharmaceuticals as the antitussive codeine and its derivatives, the analgesic morphine and its derivatives, the antitussive and anticancer drug noscapine¹¹ and the antibacterial and potential antineoplastic drugs berberine and sanguinarine¹². Their complex molecular backbone and the presence of multiple stereocenters make the complete chemical synthesis of most BIAs commercially unfeasible¹³⁻¹⁵. Consequentially, plant extraction is the only commercial source of BIAs, which limits the diversity of BIA structures available for drug discovery due to their low abundance¹⁶. The pharmaceutical value of BIAs and advances made in the elucidation of their biosynthesis in plants have made these compounds high-value candidates for production using microbial hosts¹⁷.

Despite their structural diversity, BIAs share many common biosynthetic steps and intermediates (FIG. 1). Escherichia coli was recently engineered to produce the key BIA intermediate (S)-reticuline from glucose or glycerol^{18,19} and co-cultured with S. cerevisiae expressing heterologous enzymes to synthesize (S)-magnoflorine and (S)-scoulerine²⁰. While production of the key intermediate (S)-reticuline from simple carbon sources in E. coli is an undeniable success, S. cerevisiae is a more attractive host for BIA synthesis, due to its superior ability to express the cytochrome P450s common in downstream alkaloid synthesis²¹. Strains of S. cerevisiae were also engineered for the production of the BIA intermediates reticuline and (S)-scoulerine, and the protoberberine intermediates (S)-tetrahydrocolumbamine and (S)-canadine using the fed substrate (R,S)-norlaudanosoline²². However, gaps in biosynthetic pathways and the complexity of multi-gene co-expression in microbial hosts have prevented the production of a more diverse set of BIAs thus far.

Sanguinarine is a BIA with recognized antimicrobial activities and potential as an antineoplastic drug^{23,24}. The last steps in sanguinarine biosynthesis were recently elucidated, laying the groundwork for complete synthesis of this molecule in a heterologous host²⁵⁻²⁷. In the present invention, the applicants combine gene discovery with multi-gene heterologous expression in S. cerevisiae to reconstitute a 10-gene BIA pathway for the biosynthesis of dihydrosanguinarine and sanguinarine from the commercial precursor (R,S)-norlaudanosoline. The applicants also demonstrate the activity of tetrahydroprotoberberine cis-N-methyltransferase (TNMT) towards scoulerine and cheilanthifoline and synthesize N-methylscoulerine and N-methylcheilanthifoline in yeast and show that the pathway for reticuline synthesis from norlaudanosoline is enantioselective for (S)-reticuline. The applicants also identify novel Ring A and Ring B closers able to convert scoulerine, nandinine and/or cheilanthifoline into BIA metabolites. The reconstitution of a complex pathway for BIA synthesis in S. cerevisiae represents an important advance towards the production of a broader class of alkaloids in a microbial host.

The present description refers to a number of documents, the content of which is herein incorporated by reference in their entirety.

### SUMMARY OF THE INVENTION

Next-generation sequencing technology and the accelerated discovery of genes combined with advances in synthetic biology are opening up new opportunities for the reconstitution of plant natural product biosynthetic pathways in microbes⁴⁶. In the last five years, there has been a growing trend in the complexity and diversity of the chemical structures achieved by these pathways. For example microbes have been engineered for the synthesis of terpenoids such as artemisinin acid¹⁰ and taxa-di-ene⁴⁷, BIAs such as magnoflorine²⁰ and canadine²² and glucosinates such as indolylglucosinolate⁴⁸. Cytochrome P450s are required for the synthesis of a wide range of plant natural products and the efficient recombinant expression of this class of enzyme can be difficult. Of all pathways reconstituted in microbes thus far, only those of the mammalian hydrocortisone and plant dihydrosanguinarine pathways require the heterologous expression of four cytochrome P450s. The dihydrosanguinarine pathway described herein represents the most complex plant alkaloid biosynthetic pathway ever reconstituted in yeast and provides a glimpse into the potential of engineering microbes for the synthesis of ever more complex plant natural products.

In an aspect, the applicants reconstituted a multiple-gene plant pathway in Saccharomyces cerevisiae that allows for the production of various metabolites (e.g., reticuline, stylopine and dihydrosanguinarine and its oxidized derivative sanguinarine) from (R,S)-norlaudanosoline. Synthesis of dihydrosanguinarine also yields the side-products N-methylscoulerine and N-methylcheilanthifoline, the latter of which had not been detected in plants before then. The present invention provides the longest reconstituted alkaloid pathway ever assembled in yeast and demonstrates the feasibility of the production of high-value alkaloids in microbial systems.

More specifically, in accordance with an aspect of the present invention, there is provided a method of preparing a benzylisoquinoline alkaloid (BIA) metabolite comprising: (a) culturing a host cell under conditions suitable for protein production, including a pH of between about 7 and about 10 said host cell comprising: a. a first heterologous coding sequence encoding a first enzyme involved in a metabolite pathway that converts (R,S)-norlaudanosoline into the metabolite; b. a second heterologous coding sequence encoding a second enzyme involved in a metabolite pathway that converts (R,S)-norlaudanosoline into the metabolite; c. a third heterologous coding sequence encoding a second enzyme involved in a metabolite pathway that converts (R,S)-norlaudanosoline into the metabolite; (b) adding (R,S)-norlaudanosoline to the cell culture; and (c) recovering the metabolite from the cell culture.

More specifically, in accordance with an aspect of the present invention, there is provided a method of preparing a benzylisoquinoline alkaloid (BIA) metabolite comprising: (a) culturing a host cell under conditions suitable for protein production, said host cell comprising: a. a first heterologous coding sequence encoding a first enzyme involved in a metabolite pathway that converts (R,S)-norlaudanosoline into the metabolite; b. a second heterologous coding sequence encoding a second enzyme involved in a metabolite pathway that converts (R,S)-norlaudanosoline into the metabolite; c. a third heterologous coding sequence encoding a second enzyme involved in a metabolite pathway that converts (R,S)-norlaudanosoline into the metabolite; (b) adding (R,S)-norlaudanosoline to the cell culture; and (c) recovering the metabolite from the cell culture. In a specific embodiment, the conditions include a first pH (i.e. first fermentation at a pH of) between about 7 and about 10. This may be useful for a synthesis comprising the full or a part of the sequence of enzymes of blocks 1 and 2 (see e.g., FIG. 2), wherein CFs and/or SPS are preferably replaced by a relevant Ring B and Ring A closers, respectively (see FIG. 10-12)

in accordance with another aspect of the present invention, there is provided a method of preparing a benzylisoquinoline alkaloid (BIA) metabolite comprising: (a) culturing a host cell under conditions suitable for protein production, including a first fermentation at a pH of between about 7 and about 10, and, optionnaly followed by a second fermentation at a pH between about 3 and about 6, said host cell comprising: a. a first heterologous coding sequence encoding a first enzyme involved in a metabolite pathway that converts (R,S)-norlaudanosoline into the metabolite; b. a second heterologous coding sequence encoding a second enzyme involved in a metabolite pathway that converts (R,S)-norlaudanosoline into the metabolite; c. a third heterologous coding sequence encoding a second enzyme involved in a metabolite pathway that converts (R,S)-norlaudanosoline into the metabolite; (b) adding (R,S)-norlaudanosoline to the cell culture; and (c) recovering the metabolite from the cell culture.

In a specific embodiment of these methods, the host cell is a yeast cell. In another specific embodiment, the yeast is Saccharomyces. In another specific embodiment, the Sacharomyces is Sacharomyces cerevisiae.

In a specific embodiment, the metabolite is (S)-reticuline. In another specific embodiment, the first enzyme is 6-O-methyltransferase (6OMT); the second enzyme is coclaurine N-methyltransferase (CNMT); and/or the third enzyme is 4′-O-methyltransferase 2 (4′OMT2).

In another specific embodiment, the 6OMT is as set forth in any one of the sequences as depicted in FIG. 14A or 15A; the CNMT is as set forth in any one of the sequences as depicted in FIG. 14B or 15B; and/or the 4′OMT2 is as set forth in any one of the sequences as depicted in FIG. 14C or 15C.

In another specific embodiment, 6OMT is from Papaver somniferum; CNMT is from Papaver somniferum; and/or 4′OMT2 is from Papaver somniferum.

In another specific embodiment, Ps6OMT is as set forth in SEQ ID NO: 34 (FIG. 13); PsCNMT is as set forth in SEQ ID NO: 38 (FIG. 13); and/or Ps4′OMT2 is as set forth in SEQ ID NO: 42 (FIG. 13).

In another specific embodiment, the cell further comprises a fourth heterologous coding sequence encoding a fourth enzyme involved in a metabolite pathway that converts (R,S)-norlaudanosoline into the metabolite. In another specific embodiment, the metabolite is (S)-scoulerine. In another specific embodiment, the fourth enzyme is berberine bridge enzyme (BBE). In another specific embodiment, the BBE is as set forth in any one of the sequences as depicted in FIG. 14D or 15D. In another specific embodiment, BBE is from Papaver somniferum (Ps). In another specific embodiment, the amino acid N-terminal membrane-spanning domain from PsBBE was truncated (PsBBEΔN). In another specific embodiment, PsBBEΔN is as set forth in SEQ ID NO: 46 (FIG. 13).

In another specific embodiment, the cell further comprises a fifth heterologous coding sequence encoding a fifth enzyme involved in a metabolite pathway that converts (R,S)-norlaudanosoline into the metabolite. In another specific embodiment, the metabolite is nandinine or (S)-cheilanthifoline. In another specific embodiment, the fifth enzyme is a Ring B closer able to transform scoulerine into cheilanthifoline. In another specific embodiment, the Ring B closer is further able to transform nandinine in stylopine. In another specific embodiment, the Ring B closer is as set forth in any one of the sequences depicted in FIG. 17A-C. In another specific embodiment, the Ring B closer is as set forth in any one of the sequences depicted in FIG. 17B-C. In another specific embodiment, the Ring B closer is as set forth in any one of the sequences depicted in FIG. 17C. In another specific embodiment, the fifth enzyme is cheilanthifoline synthase (CFS). In another specific embodiment, the CFS is as set forth in any one of the sequences as depicted in FIG. 14E or 15E. In another specific embodiment, CFS is from Papaver somniferum (Ps). In another specific embodiment, PsCFS is as set forth in FIG. 13 (SEQ ID NO: 50 or 52).

In another specific embodiment, the cell further comprises a sixth heterologous coding sequence encoding a sixth enzyme involved in a metabolite pathway that converts (R,S)-norlaudanosoline into the metabolite. In another specific embodiment, the metabolite is (S)-stylopine. In another specific embodiment, the sixth enzyme is a Ring A closer able to transform cheilanthifoline in (S)-stylopine. In another specific embodiment, the Ring A closer is further able to transform scoulerine in nandinine. In another specific embodiment, the Ring A closer is as set forth in any one of the sequences depicted in FIG. 17D-E. In another specific embodiment, the Ring A closer is as set forth in any one of the sequences depicted in FIG. 17E. In another specific embodiment, the Ring B closer is as set forth in SEQ ID NO: 485 and the Ring A closer is as set forth in SEQ ID NO: 487. In another specific embodiment, the Ring B closer is as set forth in SEQ ID NO: 333; or SEQ ID NO: 377 and the Ring A closer is as set forth in SEQ ID NO: 321, SEQ ID NO: 335, SEQ ID NO: 346, SEQ ID NO: 355, SEQ ID NO: or SEQ ID NO: 380. In another specific embodiment, the sixth enzyme is stylopine syntase (SPS). In another specific embodiment, the SPS is as set forth in any one of the sequences as depicted in FIG. 14F or 15F. In another specific embodiment, SPS is from Papaver somniferum (Ps).

In another specific embodiment, the method comprises the second fermentation and wherein the cell further comprises a seventh heterologous coding sequence encoding a seventh enzyme involved in a metabolite pathway that converts (R,S)-norlaudanosoline into the metabolite. In another specific embodiment, the metabolite is (S)—N-cis-methylstylopine. In another specific embodiment, the seventh enzyme is tetrahydroprotoberberine cis-N-methyltransferase (TNMT). In another specific embodiment, the TNMT is as set forth in any one of the sequences as depicted in FIG. 14G or 14G. In another specific embodiment, TNMT is from Papaver somniferum (Ps). In another specific embodiment, PsTNMT is as set forth in SEQ ID NO: 58 (FIG. 13).

In another specific embodiment, the cell further comprises a eight heterologous coding sequence encoding a eight enzyme involved in a metabolite pathway that converts (R,S)-norlaudanosoline into the metabolite. In another specific embodiment, the metabolite is protopine. In another specific embodiment, the eighth enzyme is (S)-cis-N-methylstylopine 14-hydroxylase (MSH). In another specific embodiment, the MSH is as set forth in any one of the sequences as depicted in FIG. 14H or 14H. In another specific embodiment, MSH is from Papaver somniferum (Ps).

In another specific embodiment, wherein the cell further comprises a ninth heterologous coding sequence encoding a ninth enzyme involved in a metabolite pathway that converts (R,S)-norlaudanosoline into the metabolite. In another specific embodiment, In another specific embodiment, the metabolite is 6-hydroxyprotopine. In another specific embodiment, the ninth enzyme is protopine 6-hydroxylase (P6H). In another specific embodiment, the P6H is as set forth in any one of the sequences as depicted in FIG. 14I or 14I. In another specific embodiment, P6H is from Eschscholzia californica (Ec). In another specific embodiment, EcP6H is as set forth in SEQ ID NO: 62 (FIG. 13).

In another specific embodiment, the cell further comprises a tenth heterologous coding sequence encoding a tenth enzyme involved in a metabolite pathway that converts (R,S)-norlaudanosoline into the metabolite. In another specific embodiment, the tenth enzyme is cytochrome P450 reductase (CPR). In another specific embodiment, the CPR is as set forth in any one of the sequences as depicted in FIG. 14J or 14J. In another specific embodiment, CPR is from Papaver somniferum (Ps). In another specific embodiment, PsCPR is as set forth in SEQ ID NO: 66 (FIG. 13).

In another specific embodiment, 6OMT, CNMT and 4′OMT2 are expressed from a plasmid. In another specific embodiment, BBE and CPR are expressed from a plasmid and CFS, SPS, TNMT, MSH and P6H are expressed from a chromosome.

In another specific embodiment, the metabolite is (S)-stylopine. In another specific embodiment, the first enzyme is berberine bridge enzyme (BBE); the second enzyme is cheilanthifoline synthase (CFS) or a Ring B closer able to transform scoulerine into cheilanthifoline; the third enzyme is stylopine syntase (SPS) or a Ring A closer able to transform cheilanthifoline in (S)-stylopine; and/or the fourth enzyme is cytochrome P450 reductase (CPR).

In another specific embodiment, the BBE is as set forth in any one of the sequences as depicted in FIG. 14D or 15D; the CFS is as set forth in any one of the sequences as depicted in FIG. 14E or 15E or the Ring B closer is as set forth in any one of the sequences depiced in 17A-C; the SPS is as set forth in any one of the sequences as depicted in FIG. 14F or 15F or the Ring A closer is as set forth in any one of the sequences depiced in 17D-E; and/or the CPR is as set forth in any one of the sequences as depicted in FIG. 14J or 15J.

In another specific embodiment, BBE is from Papaver somniferum; CFS is from Papaver somniferum; SPS is from Papaver somniferum; and/or CPR is from Papaver somniferum.

In another specific embodiment, PsBBE is as set forth in SEQ ID NO: 48 (FIG. 13); PsCFS is as set forth in SEQ ID NO: 50 or 52 (FIG. 13) or the Ring B closer is as set forth in SEQ ID NO: 485 (FIG. 17); PsSPS is as set forth in SEQ ID NO: 56 (FIG. 13) or the Ring A closer is as set forth in SEQ ID NO: 487 (FIG. 17); and/or PsCPR is as set forth in SEQ ID NO: 66 (FIG. 13).

In another specific embodiment, the method comprises the second fermentation and the cell further comprises a fifth heterologous coding sequence encoding a fifth enzyme involved in a metabolite pathway that converts (R,S)-norlaudanosoline into the metabolite. In another specific embodiment, the metabolite is (S)—N-cis-methylstylopine. In another specific embodiment, the fifth enzyme is tetrahydroprotoberberine cis-N-methyltransferase (TNMT). In another specific embodiment, the TNMT is as set forth in any one of the sequences as depicted in FIG. 14G or 15G. In another specific embodiment, TNMT is from Papaver somniferum (Ps). In another specific embodiment, PsTNMT is as set forth in SEQ ID NO: 58 (FIG. 13).

In another specific embodiment, the cell further comprises a sixth heterologous coding sequence encoding a sixth enzyme involved in a metabolite pathway that converts (R,S)-norlaudanosoline into the metabolite. In another specific embodiment, the metabolite is protopine. In another specific embodiment, the sixth enzyme is (S)-cis-N-methylstylopine 14-hydroxylase (MSH). In another specific embodiment, the MSH is as set forth in any one of the sequences as depicted in FIG. 14H or 15H. In another specific embodiment, MSH is from Papaver somniferum (Ps).

In another specific embodiment, the cell further comprises a seventh heterologous coding sequence encoding a seventh enzyme involved in a metabolite pathway that converts (R,S)-norlaudanosoline into the metabolite. In another specific embodiment, the metabolite is 6-hydroxyprotopine. In another specific embodiment, the seventh enzyme is protopine 6-hydroxylase (P6H). In another specific embodiment, the P6H is as set forth in any one of the sequences as depicted in FIG. 14I or 15I. In another specific embodiment, P6H is from Eschscholzia californica (Ec). In another specific embodiment, EcP6H is as set forth in SEQ ID NO: 62 (FIG. 13).

In another specific embodiment, the BBE, CFS, SPS and CPR are expressed from plasmid(s). In another specific embodiment, the TNMT, MSH and P6H are are expressed from plasmid(s). In another specific embodiment, the BBE, CFS and SPS are expressed from chromosome.

In another specific embodiment, the method comprises the second fermentation and wherein the metabolite is (S)-dihydrosanguinarine.

In another specific embodiment, the first enzyme is tetrahydroprotoberberine cis-N-methyltransferase (TNMT); the second enzyme is (S)-cis-N-methylstylopine 14-hydroxylase (MSH); the third enzyme is protopine 6-hydroxylase (P6H); and/or the fourth enzyme is cytochrome P450 reductase (CPR).

In another specific embodiment, the TNMT is as set forth in any one of the sequences as depicted in FIG. 14G or 15G; the MSH is as set forth in any one of the sequences as depicted in FIG. 14H or 15H; the P6H is as set forth in any one of the sequences as depicted in FIG. 14I or 15I; and/or the CPR is as set forth in any one of the sequences as depicted in FIG. 14J or 15J.

In another specific embodiment, TNMT is from Papaver somniferum; MSH is from Papaver somniferum; P6H is from Eschscholzia californica; and/or CPR is from Papaver somniferum.

In another specific embodiment, PsTNMT is as set forth in SEQ ID NO: 58 (FIG. 13); PsMSH is as set forth in SEQ ID NO: 268 (FIG. 13); EcP6H is as set forth in SEQ ID NO: 62 (FIG. 13); and/or PsCPR is as set forth in SEQ ID NO: 66 (FIG. 13).

In another specific embodiment, the TNMT, MSH and P6H are expressed from a plasmid.

In another specific embodiment, the host cell further expresses a cytochrome b5 (Cytb5). In another specific embodiment, the Cytb5 is as set forth in any one of the sequences as depicted in FIG. 14K.

In accordance with another aspect of the present invention, there is provided a plasmid comprising nucleic acid encoding: (a) the 6OMT, CNMT and 4′OMT2 enzymes as defined in the present invention; (b) the (i) BBE, (ii) (a) CFS or (b) Ring B closer, and (iii) (a) SPS or (b) Ring A closer enzymes as defined in the present invention; (c) the TNMT, MSH and P6H enzymes as defined in the present invention; (c) the CPR enzyme as defined in the present invention; or (d) the BBE enzyme as defined in the present invention.

In a specific embodiment, the plasmid further comprises a terminator and/or a promoter. In another specific embodiment, the plasmid is as set forth in: SEQ ID NO: 7 (FIG. 13, pGC1062); SEQ ID NO: 8 (FIG. 13, pGC994); or SEQ ID NO: 9 (FIG. 13, pGC997).

In accordance with another aspect of the present invention, there is provided a host cell expressing (a) the 6OMT, CNMT and 4′OMT2 enzymes as defined in the present invention; (b) the (i) BBE, (ii) (a) CFS or (b) Ring B closer, and (iii) (a) SPS or (b) Ring A closer enzymes as defined in the present invention; (c) the TNMT, MSH and P6H enzymes as defined in the present invention, and the CPR enzyme as defined in the present invention; (d) the enzymes of (a) and (b); or (b) and (c); (e) the enzymes of (a), (b) and (c); or (f) one or more of the plasmids as defined in the present invention.

In a specific embodiment, the host cell expresses the enzymes of (a) in a plasmid. In another specific embodiment, the host cell expresses the enzymes of (b) in a plasmid. In another specific embodiment, the host cell expresses the enzymes of (b) in a chromosome. In another specific embodiment, the host cell expresses the enzymes of (c) in a plasmid. In another specific embodiment, the host cell expresses the enzymes of (b) and (c) in a chromosome. In another specific embodiment, the host cell expresses in a plasmid the enzymes of (a) and BBE; and in a chromosome, the enzymes of (b) and (c).

In another specific embodiment, the host cell further expresses cytochrome b5.

In accordance with another aspect of the present invention, there is provided a CYP719 polypeptide that is any one of EX45-48 (SEQ ID NOs: 324-327), EX53-58 (SEQ ID NOs: 332-337), EX65-76 (SEQ ID NOs: 344-355), EX78-80 (SEQ ID NOs: 357-359), EX82 (SEQ ID NO: 361), EX86-93 (SEQ ID NOs: 365-372), EX95-101 (SEQ ID NOs: 374-380) and EX104-105 (SEQ ID NOs: 383-384).

In accordance with another aspect of the present invention, there is provided a method of preparing a benzylisoquinoline alkaloid (BIA) metabolite comprising contacting (a) a CYP719 polypeptide of the present invention; or (b) A CYP719 polypeptide that is any one of EX43-44 (SEQ ID NOs: 322-323), EX49 (SEQ ID NO:328), EX51-52 (SEQ ID NOs: 330-331), EX63-64 (SEQ ID NOs: 342-343), EX77 (SEQ ID NO: 356) or EX103 (SEQ ID NO: 382), with scoulerine, nandinine and/or cheilanthifoline.

In accordance with another aspect of the present invention, there is provided a method of producing (i) N-methylcheilanthifoline; or (ii) N-methylcoulerine, comprising contacting cheilanthifoline or scoulerine, respectively, with tetrahydroprotoberberine cis-N-methyltransferase (TNMT), whereby (i) N-methylcheilanthifoline; or (ii) N-methylcoulerine are produced.

In accordance with another aspect of the present invention, there is provided a method of producing nandinine comprising contacting scoulerine with a Ring B closer as set forth in SEQ ID NO: 483, SEQ ID NO: 484, SEQ ID NO: 485, SEQ ID NO: 324, SEQ ID NO: 353, SEQ ID NO: 320, SEQ ID NO: 363, SEQ ID NO: 338, SEQ ID NO: 378, SEQ ID NO: 333, SEQ ID NO: 377, SEQ ID NO: 344, or SEQ ID NO: 374.

In another specific embodiment, the Ring B closer as set forth in SEQ ID NO: 484, SEQ ID NO: 485, SEQ ID NO: 324, SEQ ID NO: 333 or SEQ ID NO: 377

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1. Common steps in the native biosynthetic pathways of structurally diverse BIAs. The L-tyrosine derivatives dopamine and 4-hydroxyphenylacetaldehyde condense to generate (S)-norcoclaurine in a reaction catalysed by the enantioselective enzyme norcoclaurine synthase (NCS). (S)-Norcoclaurine is O-methylated at positions 6 and 4′ by the enzymes 6-O-methyltransferase (6OMT) and 4′-O-methyltransferase 2 (4′OMT2) respectively, N-methylated by coclaurine-N-methyltransferase (CNMT) and hydroxylated at 3′ by (S)—N-methylcoclaurine-3′-hydroxylase (NMCH) to give (S)-reticuline. (S)-reticuline is a common precursor in the synthesis of morphinan, protoberberine and benzophenanthridine alkaloids and it is converted to (S)-scoulerine by the berberine bridge enzyme (BBE). (S)-scoulerine is also a common precursor involved in the synthesis of protoberberine and benzophenanthridine alkaloids. The common branch point intermediates (S)-reticuline and (S)-scoulerine are highlighted. Dashed arrows indicate more than one enzymatic step. Full arrows indicate single enzymatic step.

FIG. 2. Description of the enzyme block strategy used in the reconstitution of the sanguinarine biosynthetic pathway in S. cerevisiae. Enzymes were tested for functional activity by blocks of sequential enzymes and on the basis of availability of feeding substrates. Boxed text indicates alkaloids used as feeding substrates. Abbreviations are as follows: 6OMT, 6-O-methyltransferase; 4′OMT2, 4′-O-methyltransferase 2; CNMT, coclaurine-N-methyltransferase; BBE, berberine bridge enzyme; CFS, cheilanthifoline synthase (CYP719A25); SPS, stylopine synthase (CYP719A20); TNMT, tetrahydroprotoberberine cis-N-methyltransferase; MSH, (S)-cis-N-methylstylopine 14-hydroxylases (CYP82N4); P6H, protopine 6-hydroxylase (CYP82N2v2); and DBOX, dihydrobenzophenanthridine oxidase. P6H is from Eschscholzia californica, all other enzymes used in the examples presented herein are from P. somniferum. The P. somniferum cytochrome P450 reductase (CPR) was also expressed for functional expression of cytochromes P450s together with Block 2 and/or 3.

FIG. 3. Characterization of PsCFS and PsSPS. (A) Relative transcript abundance of CYP719A25 (PsCFS) and CYP719A20 (PsSPS) in opium poppy roots and stems presented as Fragments Per Kilobase of exons model per Million mapped reads (FPKM). Data presented derive from a single experiment. (B) Immunoblot analysis of microsomal proteins from S. cerevisiae strains (i) GCY1192 (expressing PsCPR); (ii) GCY1193 (expressing PsCPR and PsCFS); or (iii) a mixture of GCY1193 (expressing PsCPR and PsCFS) and GCY1194 (expressing PsCPR and PsSPSΔN). α-c-Myc and α-FLAG antibodies were used to detect CPR, and PsCFS and PsSPSΔN (arrowheads), respectively. (C) LC-MS profiles of enzyme assays of microsomal proteins from S. cerevisiae strains (i) GCY1192; (ii) GCY1193; or (iii) a mixture of GCY1193 and GCY1194 incubated with (S)-scoulerine. Abbreviation: nd—not detected.

FIG. 4. BIA yields from engineered strains incubated with pathway intermediates. (A) BIA yields from cell feeding assays of strains expressing Block 1, 2 and 3 individually or in combinations. (B) Cell feeding assays of strain GCY1125 with one of the four different substrates, namely (R,S)-norlaudanosoline (nor), (S)-reticuline (ret), (S)-scoulerine (scou), and (S)-stylopine (sty). Percent yield of end product is indicated in the appropriate column. Percent conversion was calculated as the ratio of total moles of end product recovered (sum of both cell extract and supernatant) to moles of supplemented substrate. Enzyme expressed from plasmid (dark grey); enzyme expressed from chromosome (light grey); enzymes expressed but not necessary for substrate conversion (no shading). BBE_{2μ} signifies BBE expression from a 2μ vector. Data presented represents the mean±s.d. of at least three biological replicates.

FIG. 5. LC-FT-MS profiles of BIAs from cell feeding assays. (A) Cell feeding assay of strain GCY1086 harbouring Block 1 enzymes and incubated with (R,S)-norlaudanosoline (*). (B) Cell feeding assay of strain GCY1090 harbouring Block 2 enzymes and incubated with (S)-scoulerine (*). (C) Cell feeding assay of strain GCY1094 harbouring Block 3 enzymes and incubated with (S)-stylopine (*). Chromatograms shown are the summed ion counts of supernatant and cell extract.

FIG. 6. Relative abundance of BIAs in culture supernatants and cell extracts. (A) Fractionation of BIAs in the absence of heterologous enzymes. Control strain S. cerevisiae CEN.PK2-1 D incubated with either (R,S)-norlaudanosoline (nor), (S)-reticuline (ret), (S)-scoulerine (scou), or (S)-stylopine (sty) for 0 or 16 hours. (B) Fractionation of BIAs in the presence of heterologous enzymes. Cell feeding assays of strain GCY1086 incubated with (R,S)-norlaudanosoline, strain GCY1090 incubated with (S)-scoulerine, and strain GCY1094 incubated with (S)-stylopine were extracted after 16 hours. BIAs in supernatant and cell extract fractions were analysed using LC-FT-MS. Percent BIA recovery in supernatant and cell extract relative to total BIA recovery is shown for each individual alkaloid. Nor: norlaudanosoline; 316: intermediates with m/z 316; ret: reticuline; scou: scoulerine; che: cheilanthifoline; sty: stylopine; N-st: N-methylstylopine; DHS: dihydrosanguinarine. Data presented represents the mean±s.d. of at least two biological replicates.

FIG. 7 Chiral analysis of reticuline produced from (R,S)-norlaudanosoline by engineered S. cerevisiae. HPLC-MS chromatographic profile of authentic standards of a (R)-reticuline, b (S)-reticuline and c a mixture of (S)- and (R)-reticuline. d Chiral analysis of reticuline produced from (R,S)-norlaudanosoline in cell feeding assays of strain GCY1125 expressing the opium poppy Ps6OMT, PsCNMT, Ps4′OMT2 and or a complete dihydrosanguinarine pathway. e Chiral analysis of reticuline produced from (R,S)-norlaudanosoline in cell feeding assays of strain GCY1086 expressing the opium poppy Ps6OMT, PsCNMT and Ps4′OMT2. f Methylation pathway for conversion of (R,S)-norlaudanosoline to (S)-reticuline.

FIG. 8. LC-MS analysis of alkaloids N-methylated by TNMT. (A) Cell feeding assay of strain GCY1104 harbouring Block 2 and 3 enzymes and incubated with (S)-scoulerine (*) demonstrates the accumulation of the side-products N-methylscoulerine and N-cheilanthifoline. (B) LC-FT-MS chromatographic profiles and MS spectra of BIAs from cell feeding assays of strains GCY1101 and GCY1127 incubated with (S)-scoulerine. In strain GCY1127 but not GCY1101, the products N-methylscoulerine, N-methylcheilanthifoline, and N-methylstylopine were detected. Chromatograms shown are the summed ion counts of supernatant and cell extract. Parent ion:

FIG. 9. Testing Block 2+Block 3 at different pH conditions. Abbreviations not mentioned in the text above: Scou is scoulerine, Chei is cheilanthifoline; Sty is stylopine; N-sty is N-methyl-stylopine; DHS is dihydrosanguinarine. YNB indicates YNB-DO-GLU.

FIG. 10A-B: Presents conversion of (S)-scoulerine to downstream BIAs PsSPSΔN and PsCFS used as controls. G. conversion of (S)-scoulerine into cheilanthifoline or nandinine by GC1333 strains containing an integrated PsCPR and one of CYP719s EX41 to EX105. H. conversion of (S)-scoulerine into cheilanthifoline, nandinine or stylopine by GC1316 strains containing PsCPR and PsCFS integrated into the genome and a CYP719s shown to effectively convert (S)-scoulerine into cheilanthifoline or nandinine in A.

FIG. 10C-H (C) phylogenetic tree of CYP719s generated with MEGA6; (D) Predicted activities of CYP719s based on alignments of CYP719s with published Ring A-closure, Ring B-closure, or salutaridine synthase activities (grey arrows). (E) Activity (either Ring A- or Ring B-closure) of CYP719s on (S)-scoulerine: enzymes with more (black) or less (dark grey) than 5% conversion of scoulerine are indicated. Enzymes not assayed for activity are in light grey. (F) Activity (either Ring A- or Ring B-closure) of CYP719s on (S)-scoulerine: enzymes with more (black) or less (dark grey) less than 50% conversion of scoulerine are indicated. Enzymes not assayed for activity are in light grey. (G) Activity (either Ring A- or Ring B-closure) of CYP719s on (S)-scoulerine: enzymes with more (black) or less (dark grey) less than 95% conversion of scoulerine are indicated. Enzymes not assayed for activity are in light grey. (H) Stylopine synthase activity (either Ring A closure of cheilanthifoline or Ring B closure of nandinine) of selected CYP719s: enzymes with more than 95% conversion of supplemented BIA are indicated in black.

FIG. 11A-B: showing conversion of (S)-cheilanthifoline (FIG. 11A) or (S)-nandinine (FIG. 11B) to (S)-stylopine by purchased CYP719s. Plasmids harboring CYP719s were transformed into GC1333 containing an integrated PsCPR. Strains were incubated with the appropriate BIA overnight, and then total BIAs were extracted and total molar ratios were compared. PsSPSΔN was used as a control.

FIG. 12A-B: showing conversion of (S)-scoulerine to downstream BIAs by CYP719s. More particularly, FIG. 12A it shows the ability of various combinations of two CYP719 enzymes to transform scoulerine into nandinine or cheilanthifoline, and nandinine and cheilanthifoline into stylopine and FIG. 12B compares the efficacy of various combinations of two CYP719 enzymes in terms of avoiding build-up of unwanted N-methyl side products. PsCFS, PsSPSΔN of FIG. 13 as set forth in SEQ ID NO: 50 and 54 were used as controls.

FIG. 13A-E. A) nucleotide sequences of vectors pGREG503 (SEQ ID NO: 1); pGREG504 (SEQ ID NO: 2); pGREG505 (SEQ ID NO: 3); pGREG506 (SEQ ID NO: 4); 2μ vector pYES2 (SEQ ID NO: 5); 2μ vector pESC-Leu (SEQ ID NO: 6); B) nucleotide sequences of plasmids: pGC1062 (block 1 plasmid) (SEQ ID NO: 7); pGC994 (block 2 plasmid) (SEQ ID NO: 8); pGC997 (block 3 plasmid) (SEQ ID NO: 9); pGC557 (CPR plasmid) (SEQ ID NO: 10); pGC655 (BBEΔN plasmid) (SEQ ID NO: 11); C) nucleotide sequences of promoters: TDH3 promoter (SEQ ID NO: 12); FBA1 promoter (SEQ ID NO: 13); PDC1 promoter (SEQ ID NO: 14); PMA1 promoter (SEQ ID NO: 15); GAL1 promoter (SEQ ID NO: 16); GAL10 promoter (SEQ ID NO: 17); TEF1 promoter (SEQ ID NO: 18); TEF2 promoter (SEQ ID NO: 19); PGK1 promoter (SEQ ID NO: 20); PYK1 promoter (SEQ ID NO: 21); TPI1 promoter (SEQ ID NO: 22); TDH2 promoter (SEQ ID NO: 23); ENO2 promoter (SEQ ID NO: 24); HXT9 promoter (SEQ ID NO: 25); D) nucleotide sequences of terminators: CYC1 terminator (SEQ ID NO: 26); ADH1 terminator (SEQ ID NO: 27); PGI1 terminator (SEQ ID NO: 28); ADH2 terminator (SEQ ID NO: 29); ENO2 terminator (SEQ ID NO: 30); FBA1 terminator (SEQ ID NO: 31); TDH2 terminator (SEQ ID NO: 32); TPI1 terminator (SEQ ID NO: 33); E) Amino acid and nucleotide sequences of enzymes: 6OMT (KF544154 Synthetic construct for Papaver somniferum (R,S)-norcoclaurine 6-O-methyltransferase gene, complete cds amino acid sequence (SEQ ID NO: 34) and nucleotide sequence encoding same (SEQ ID NO: 35); AY217335 Ps6OMT Papaver somniferum S-adenosyl-L-methionine:norcoclaurine 6-O-methyltransferase (SEQ ID NO: 36) and nucleotide sequence encoding same (SEQ ID NO: 37)); CNMT (KF661326 Synthetic construct S-adenosyl-L-methionine:coclaurine N-methyltransferase gene, complete cds amino acid sequence (SEQ ID NO: 38) and nucleotide sequence encoding same (SEQ ID NO: 39); AY217336 Papaver somniferum S-adenosyl-L-methionine:coclaurine N-methyltransferase mRNA, complete cds amino acid sequence (SEQ ID NO: 40) and nucleotide sequence encoding same (SEQ ID NO: 41)); 4′OMT2 (KF661327 Synthetic construct S-adenosyl-L-methionine:3′-hydroxy-N-methylcoclaurine 4′-O-methyltransferase 2 gene amino acid sequence (SEQ ID NO: 42) and nucleotide sequence encoding same (SEQ ID NO: 43); AY217334 S-adenosyl-L-methionine:3′-hydroxy-N-methylcoclaurine 4′-O-methyltransferase 2 Papaver somniferum amino acid sequence (SEQ ID NO: 44) and nucleotide sequence encoding same (SEQ ID NO: 45); BBE (Truncated Papaver somniferum BBE (PsBBEΔN) amino acid sequence (SEQ ID NO: 46) and nucleotide sequence encoding same (SEQ ID NO: 47); AF025430 Papaver somniferum berberine bridge enzyme (bbe1) gene, complete cds amino acid sequence (SEQ ID NO: 48) and nucleotide sequence encoding same (SEQ ID NO: 49); CFS (ADB89213 Papaver somniferum Cheilanthifoline synthase amino acid sequence (SEQ ID NO: 50) and nucleotide sequence encoding same (SEQ ID NO: 51); Papaver somniferum Cheilanthifoline synthase cloned into pGC994 amino acid sequence (SEQ ID NO: 52) and nucleotide sequence encoding same (SEQ ID NO: 53); SPS (Synthetic SPS gene with the N-terminus LsGAO spanning domain -KF481962 N-terminus Lactuca sativa germacrene A oxidase fused to 29 amino acids N-truncated Papaver somniferum stylopine synthase (psSPSΔN) amino acid sequence (SEQ ID NO: 54) and nucleotide sequence encoding same (SEQ ID NO: 55); PsSPS native sequence (SEQ ID NO: 56) and nucleotide sequence encoding same (SEQ ID NO: 57); TNMT (DQ028579 Papaver somniferum S-adenosyl-L-methionine:(S)-tetrahydroprotoberberine-cis-N-methyltransferase mRNA, complete cds amino acid sequence (SEQ ID NO: 58) and nucleotide sequence encoding same (SEQ ID NO: 59)); MSH (KC154003 Papaver somniferum (S)-cis-N-methylstylopine 14-hydroxylase mRNA, complete cds amino acid sequence (SEQ ID NO: 60) and nucleotide sequence encoding same (SEQ ID NO: 61)); P6H (AB598834 Eschscholzia californica P6H mRNA for protopine 6-hydroxylase, complete cds amino acid sequence (SEQ ID NO: 62) and nucleotide sequence encoding same (SEQ ID NO: 63)); CPR (KF661328 Synthetic construct NADPH:ferrihemoprotein oxidoreductase gene, complete cds amino acid sequence (SEQ ID NO: 64) and nucleotide sequence encoding same (SEQ ID NO: 65); U67185 Papaver somniferum NADPH:ferrihemoprotein oxidoreductase mRNA, complete cds amino acid sequence (SEQ ID NO: 66) and nucleotide sequence encoding same (SEQ ID NO: 67)); and cytochrome b5 (Papaver somniferum cytochrome b5 amino acid sequence (SEQ ID NO: 68) and nucleotide sequence encoding same (SEQ ID NO: 69); JQ582841 Synthetic construct cytochrome b5 gene, partial cds (derived from Artemisia annua) amino acid sequence (SEQ ID NO: 70) and nucleotide sequence encoding same (SEQ ID NO: 71)) are provided.

FIG. 14A to 14K. Present alignments of various enzymes established by Clustal™ Omega multiple sequence alignment, namely amino acid alignments of orthologs for each of enzymes 6OMT; CNMT; 4′OMT2; BBE; CFS; SPS; TNMT; MSH; P6H; CPR and Cytb5; and consensus sequences derived therefrom. In these alignments, “*” denotes that the residues in that column are identical in all sequences of the alignment, “:” denotes that conserved substitutions have been observed, and “.” denotes that semi-conserved substitutions have been observed. Consensus sequences derived from these alignments are also presented wherein X is any amino acid. Sequences corresponding to the N-terminal membrane-spanning domains of the enzymes are shaded. Sequences (those of orthologs and consensuses) devoid of these transmembrane domains are encompassed by the present invention (e.g., BBEΔN, SPSΔN, CFSΔN; MSHΔN; and P6HΔN).

FIG. 14A. 6OMT from Papaver somniferum (SEQ ID NO: 36), Papaver bracteatum (SEQ ID NO: 72), Sanguinaria canadensis (SEQ ID NO: 73), Chelidonium majus (SEQ ID NO: 74), Stylophorum diphyllum (SEQ ID NO: 75), Eschscholzia californica (SEQ ID NO: 76); Glaucium flavum (SEQ ID NO: 77), Argemone Mexicana (SEQ ID NO: 78), Thalictrum flavum (SEQ ID NO: 79), Hydrastis Canadensis (SEQ ID NO: 80), Nigella sativa (SEQ ID NO: 81), Xanthorhiza simplicissima (SEQ ID NO: 82), Berberis thunbergii (SEQ ID NO: 83), Mahonia aquifolium (SEQ ID NO: 84), Jeffersonia diphylla (SEQ ID NO: 85), Menispermum canadense (SEQ ID NO: 86), Corydalis cheilanthifolia (SEQ ID NO: 87), Nandina domestica (SEQ ID NO: 88), Cissampelos mucronata (SEQ ID NO: 89), Tinospora cordifolia (SEQ ID NO: 90), Cocculus trilobus (SEQ ID NO: 91), Coptis japonica (SEQ ID NO: 92) and consensus sequence (SEQ ID NO: 93);

FIG. 14B. CNMT from Papaver somniferum (SEQ ID NO: 40), Papaver bracteatum (SEQ ID NO: 94), Sanguinaria canadensis (SEQ ID NO: 95), Chelidonium majus candidate 1 (SEQ ID NO: 96), Chelidonium majus candidate 2 (SEQ ID NO: 97), Stylophorum diphyllum candidate 1 (SEQ ID NO: 98), Stylophorum diphyllum candidate 2 (SEQ ID NO: 99), Eschscholzia californica (SEQ ID NO: 100); Glaucium flavum (SEQ ID NO: 101), Argemone Mexicana (SEQ ID NO: 102), Corydalis cheilanthifolia (SEQ ID NO: 103), Thalictrum flavum (SEQ ID NO: 104), Hydrastis Canadensis (SEQ ID NO: 105), Nigella sativa (SEQ ID NO: 106), Xanthorhiza simplicissima (SEQ ID NO: 107), Berberis thunbergii (SEQ ID NO: 108), Jeffersonia diphylla (SEQ ID NO: 109), Nandina domestica (SEQ ID NO: 110), Menispermum canadense (SEQ ID NO: 111), Cissampelos mucronata (SEQ ID NO: 112), Tinospora cordifolia (SEQ ID NO: 113), Cocculus trilobus (SEQ ID NO: 114) and consensus sequence (SEQ ID NO: 115);

FIG. 14C. 4′OMT2 from Papaver somniferum (SEQ ID NO: 44), Papaver bracteatum (SEQ ID NO: 116), Sanguinaria canadensis (SEQ ID NO: 117), Chelidonium majus (SEQ ID NO: 118), Stylophorum diphyllum (SEQ ID NO: 119), Eschscholzia californica (SEQ ID NO: 120); Glaucium flavum (SEQ ID NO: 121), Argemone Mexicana (SEQ ID NO: 122), Corydalis cheilanthifolia (SEQ ID NO: 123), Thalictrum flavum (SEQ ID NO: 124), Hydrastis Canadensis (SEQ ID NO: 125), Nigella sativa (SEQ ID NO: 126), Xanthorhiza simplicissima (SEQ ID NO: 127), Berberis thunbergii (SEQ ID NO: 128), Mahonia aquifolium (SEQ ID NO: 129), Jeffersonia diphylla (SEQ ID NO: 130), Nandina domestica (SEQ ID NO: 131), Menispermum canadense (SEQ ID NO: 132), Cissampelos mucronata (SEQ ID NO: 133), Cocculus trilobus (SEQ ID NO: 134), Coptis japonica (SEQ ID NO: 135) and consensus sequence (SEQ ID NO: 136);

FIG. 14D. BBE from Papaver somniferum (SEQ ID NO:48), Papaver bracteatum candidate 1 (SEQ ID NO: 137), Papaver bracteatum candidate 2 (SEQ ID NO: 138), Sanguinaria canadensis (SEQ ID NO: 139), Chelidonium majus (SEQ ID NO: 140), Stylophorum diphyllum (SEQ ID NO: 141), Eschscholzia californica (SEQ ID NO: 142); Glaucium flavum (SEQ ID NO: 143), Argemone Mexicana (SEQ ID NO: 144), Corydalis cheilanthifolia (SEQ ID NO: 145), Thalictrum flavum (SEQ ID NO: 146), Hydrastis Canadensis (SEQ ID NO: 147), Xanthorhiza simplicissima (SEQ ID NO: 148), Berberis thunbergii (SEQ ID NO: 149), Jeffersonia diphylla (SEQ ID NO: 150), Nandina domestica (SEQ ID NO: 151), Cissampelos mucronata (SEQ ID NO: 152), Cocculus trilobus (SEQ ID NO: 153); and consensus sequences: full (SEQ ID NO: 154), and truncated (e.g., devoid of shaded domain) (SEQ ID NO: 155). Truncated versions of each specific species sequence is also shown (i.e., devoid of shaded domain);

FIG. 14E. CFS from Papaver somniferum (SEQ ID NO: 50), Papaver bracteatum candidate 1 (SEQ ID NO: 156), Papaver bracteatum candidate 2 (SEQ ID NO: 157), Sanguinaria canadensis candidate 1 (SEQ ID NO: 158), Sanguinaria canadensis candidate 2 (SEQ ID NO: 159), Sanguinaria canadensis candidate 3 (SEQ ID NO: 160), Sanguinaria canadensis candidate 4 (SEQ ID NO: 161), Sanguinaria canadensis candidate 5 (SEQ ID NO: 162), Sanguinaria canadensis candidate 6 (SEQ ID NO: 163), Sanguinaria canadensis candidate 7 (SEQ ID NO: 164), Sanguinaria canadensis candidate 8 (SEQ ID NO: 165), Chelidonium majus candidate 1 (SEQ ID NO: 166), Chelidonium majus candidate 2 (SEQ ID NO: 167), Chelidonium majus candidate 3 (SEQ ID NO: 168), Chelidonium majus candidate 4 (SEQ ID NO: 169), Stylophorum diphyllum candidate 1 (SEQ ID NO: 170), Stylophorum diphyllum candidate 2 (SEQ ID NO: 171), Stylophorum diphyllum candidate 3 (SEQ ID NO: 172), Eschscholzia californica candidate 1 (SEQ ID NO: 173); Eschscholzia californica candidate 2 (SEQ ID NO: 174); Eschscholzia californica candidate 3 (SEQ ID NO: 175); Eschscholzia californica candidate 4 (SEQ ID NO: 176); Eschscholzia californica candidate 5 (SEQ ID NO: 177); Glaucium flavum candidate 1 (SEQ ID NO: 178), Glaucium flavum candidate 2 (SEQ ID NO: 179), Glaucium flavum candidate 3 (SEQ ID NO: 180), Glaucium flavum candidate 4 (SEQ ID NO: 181), Argemone Mexicana candidate 1 (SEQ ID NO: 182), Corydalis cheilanthifolia candidate 1 (SEQ ID NO: 183), Coridalys cheilanthifolia candidate 2 (SEQ ID NO: 184), Corydalis cheilanthifolia candidate 3 (SEQ ID NO: 185, Thalictrum flavum candidate 1 (SEQ ID NO: 186), Thalictrum flavum candidate 2 (SEQ ID NO: 187), Thalictrum flavum candidate 3 (SEQ ID NO: 188), Hydrastis canadensis candidate 1 (SEQ ID NO: 189), Xanthorhiza simplicissima candidate 1 (SEQ ID NO: 190), Berberis thunbergii candidate 1 (SEQ ID NO: 191), Berberis thunbergii candidate 2 (SEQ ID NO: 192), Jeffersonia diphylla candidate 1 (SEQ ID NO: 193), Nandina domestica candidate 1 (SEQ ID NO: 194), Nandina domestica candidate 2 (SEQ ID NO: 195), Nandina domestica candidate 3 (SEQ ID NO: 196), Nandina domestica candidate 4 (SEQ ID NO: 197), Nandina domestica candidate 5 (SEQ ID NO: 198), Nandina domestica candidate 6 (SEQ ID NO: 199), Menispermum canadense candidate 1 (SEQ ID NO: 200); and consensus sequence (SEQ ID NO: 201). Truncated versions of each specific species and consensus sequence is also shown (i.e., devoid of shaded domain);

FIG. 14F. SPS from Papaver somniferum (SEQ ID NO:56), Papaver bracteatum candidate 1 (SEQ ID NO: 202), Sanguinaria canadensis candidate 1 (SEQ ID NO: 203), Sanguinaria canadensis candidate 2 (SEQ ID NO: 204), Sanguinaria canadensis candidate 3 (SEQ ID NO: 205), Sanguinaria canadensis candidate 4 (SEQ ID NO: 206), Chelidonium majus candidate 1 (SEQ ID NO: 207), Chelidonium majus candidate 2 (SEQ ID NO: 208), Chelidonium majus candidate 3 (SEQ ID NO: 209), Chelidonium majus candidate 4 (SEQ ID NO: 210), Chelidonium majus candidate 5 (SEQ ID NO: 211), Stylophorum diphyllum candidate 1 (SEQ ID NO: 212), Stylophorum diphyllum candidate 2 (SEQ ID NO: 213), Eschscholzia californica candidate 1 (SEQ ID NO: 214); Eschscholzia californica candidate 2 (SEQ ID NO: 215); Eschscholzia californica candidate 3 (SEQ ID NO: 216); Glaucium flavum candidate 1 (SEQ ID NO: 217), Glaucium flavum candidate 2 (SEQ ID NO: 218), Argemone Mexicana candidate 1 (SEQ ID NO: 219), Corydalis cheilanthifolia candidate 1 (SEQ ID NO: 220), Corydalis cheilanthifolia candidate 2 (SEQ ID NO: 221), Corydalis cheilanthifolia candidate 3 (SEQ ID NO: 222), Corydalis cheilanthifolia candidate 4 (SEQ ID NO: 223), Thalictrum flavum candidate 1 (SEQ ID NO: 224), Thalictrum flavum candidate 2 (SEQ ID NO: 225), Thalictrum flavum candidate 3 (SEQ ID NO: 226), Hydrastis canadensis candidate 1 (SEQ ID NO: 227), Xanthorhiza simplicissima candidate 1 (SEQ ID NO: 228), Berberis thunbergii candidate 1 (SEQ ID NO: 229), Berberis thunbergii candidate 2 (SEQ ID NO: 230), Berberis thunbergii candidate 3 (SEQ ID NO: 231), Jeffersonia diphylla candidate 1 (SEQ ID NO: 232), Nandina domestica candidate 1 (SEQ ID NO: 233), Menispermum canadense candidate 1 (SEQ ID NO: 234); and consensus sequence (SEQ ID NO: 235). Truncated versions of each specific species and consensus sequence is also shown (i.e., devoid of shaded domain);

FIG. 14G. TNMT from Papaver somniferum (SEQ ID NO:58), Papaver bracteatum (SEQ ID NO: 236), Sanguinaria canadensis (SEQ ID NO: 237), Chelidonium majus candidate 1 (SEQ ID NO: 238), Chelidonium majus candidate 2 (SEQ ID NO: 239), Stylophorum diphyllum (SEQ ID NO: 240), Eschscholzia californica (SEQ ID NO: 241); Glaucium flavum (SEQ ID NO: 242), Argemone Mexicana (SEQ ID NO: 243), Corydalis cheilanthifolia (SEQ ID NO: 244), Thalictrum flavum (SEQ ID NO: 245), Hydrastis Canadensis (SEQ ID NO: 246), Nigella sativa (SEQ ID NO: 247), Xanthorhiza simplicissima (SEQ ID NO: 248), Berberis thunbergii (SEQ ID NO: 249), Jeffersonia diphylla (SEQ ID NO: 250), Nandina domestica (SEQ ID NO: 251), Menispermum canadense (SEQ ID NO: 252), Cissampelos mucronata (SEQ ID NO: 253), Tinospora cordifolia (SEQ ID NO: 254), Cocculus trilobus (SEQ ID NO: 255), and consensus sequence (SEQ ID NO: 256);

FIG. 14H. MSH from Papaver somniferum (SEQ ID NO:60), Papaver bracteatum (SEQ ID NO: 257), Sanguinaria canadensis (SEQ ID NO: 258), Chelidonium majus (SEQ ID NO: 259), Stylophorum diphyllum (SEQ ID NO: 260), Eschscholzia californica (SEQ ID NO: 261); Glaucium flavum (SEQ ID NO: 262), Argemone Mexicana (SEQ ID NO: 263), Corydalis cheilanthifolia (SEQ ID NO: 264), Thalictrum flavum (SEQ ID NO: 265), Xanthorhiza simplicissima (SEQ ID NO: 266), Nandina domestica (SEQ ID NO: 267); and consensus sequence (SEQ ID NO: 268). Truncated versions of each specific species and consensus sequence is also shown (i.e., devoid of shaded domain);

FIG. 14I. P6H from Eschscholzia californica (SEQ ID NO: 62); Papaver somniferum (SEQ ID NO:269), Papaver bracteatum (SEQ ID NO: 270), Sanguinaria canadensis (SEQ ID NO: 271), Chelidonium majus (SEQ ID NO: 272), Stylophorum diphyllum (SEQ ID NO: 273), Glaucium flavum (SEQ ID NO: 274), Argemone Mexicana (SEQ ID NO: 275), Corydalis cheilanthifolia (SEQ ID NO: 276), Thalictrum flavum (SEQ ID NO: 277), Nandina domestica (SEQ ID NO: 278); and consensus sequence (SEQ ID NO: 279). Truncated versions of each specific species and consensus sequence is also shown (i.e., devoid of shaded domain);

FIG. 14J. CPR from Papaver somniferum (SEQ ID NO:66), Papaver bracteatum candidate 1 (SEQ ID NO: 280), Papaver bracteatum candidate 2 (SEQ ID NO: 281), Sanguinaria canadensis candidate 1 (SEQ ID NO: 282), Sanguinaria canadensis candidate 2 (SEQ ID NO: 283), Chelidonium majus candidate 1 (SEQ ID NO: 284), Chelidonium majus candidate 2 (SEQ ID NO: 285), Chelidonium majus candidate 3 (SEQ ID NO: 286), Stylophorum diphyllum candidate 1 (SEQ ID NO: 287), Stylophorum diphyllum candidate 2 (SEQ ID NO: 288), Eschscholzia californica candidate 1 (SEQ ID NO: 289); Glaucium flavum candidate 1 (SEQ ID NO: 290), Glaucium flavum candidate 2 (SEQ ID NO: 291), Argemone Mexicana candidate 1 (SEQ ID NO: 292), Argemone Mexicana candidate 2 (SEQ ID NO: 293), Corydalis cheilanthifolia candidate 1 (SEQ ID NO: 294), Corydalis cheilanthifolia candidate 2 (SEQ ID NO: 295), Thalictrum flavum candidate 1 (SEQ ID NO: 296), Thalictrum flavum candidate 2 (SEQ ID NO: 297), Hydrastis canadensis candidate 1 (SEQ ID NO: 298), Nigella sativa candidate 1 (SEQ ID NO: 299), Xanthorhiza simplicissima candidate 1 (SEQ ID NO: 300), Xanthorhiza simplicissima candidate 2 (SEQ ID NO: 301), Berberis thunbergii candidate 1 (SEQ ID NO: 302), Mahonia aquifolium candidate 1 (SEQ ID NO: 303), Mahonia aquifolium candidate 2 (SEQ ID NO: 304), Jeffersonia diphylla candidate 1 (SEQ ID NO: 305), Nandina domestica candidate 1 (SEQ ID NO: 306), Nandina domestica candidate 2 (SEQ ID NO: 307), Menispermum canadense candidate 1 (SEQ ID NO: 308), Menispermum canadense candidate 2 (SEQ ID NO: 309), Cissampelos mucronata candidate 1 (SEQ ID NO: 310), Cissampelos mucronata candidate 2 (SEQ ID NO: 311), Cissampelos mucronata candidate 3 (SEQ ID NO: 312), Tinospora cordifolia candidate 1 (SEQ ID NO: 313), Tinospora cordifolia candidate 2 (SEQ ID NO: 314), Tinospora cordifolia candidate 3 (SEQ ID NO: 315), and consensus sequence (SEQ ID NO: 316);

FIG. 14K. Cytochrome 85 from Papaver somniferum (SEQ ID NO: 68) and Artemisia annua (SEQ ID NO: 318), and consensus sequence (SEQ ID NO: 319).

FIG. 15 presents alignments of the enzymes of FIG. 13 established by Clustal™ Omega multiple sequence alignment, namely amino acid alignments of orthologs also shown in FIG. 14 for each of enzymes 6OMT; CNMT; 4′OMT2; BBE; CFS; SPS; TNMT; MSH; P6H; and CPR; and consensuses derived therefrom. Consensus sequences identified as 60%, 70%, 75%, 80%, 85%, 90% and 95% are presented for each alignments. In these consensuses, small “o” denotes alcohol and refers to S or T; small “I” denotes aliphatic and refers to I, L or V; period “.” denotes any amino acid; small “a” denotes aromatic and refers to F, H, W or Y; small “c” denotes charged and refers to D, E, H, K or R; small “h” denotes hydrophobic and refers to A, C, F, G, H, I, K, L, M, R, T, V, W or Y; minus sign “−” denotes negative and refers to D or E; small “p” denotes polar and refers to C, D, E, H, K, N, Q, R, S or T; plus sign “+” denotes positive and refers to H, K or R; small “s” denotes small and refers to A, C, D, G, N, P, S, T or V; small “u” denotes tiny and refers to A, G or S; small “t” denotes turnlike and refers to A, C, D, E, G, H, K, N, Q, R, S and T.

FIG. 16 presents the amino acid sequences of CYP719s EX41 to EX105 (SEQ ID NOs: 320-384).

FIG. 17A presents an alignment of Ring B closers (scoulerine to cheilanthifoline and/or nandinine to stylopine) identified herein (EX45 (SEQ ID NO: 324); EX74 (SEQ ID NO: 353); EX41 (SEQ ID NO: 320); EX84 (SEQ ID NO: 363); EX59 (SEQ ID NO: 338); EX99 (SEQ ID NO: 378); EX54 (SEQ ID NO: 333); EX98 (SEQ ID NO: 377); EX65 (SEQ ID NO: 344); and EX95 (SEQ ID NO: 374)) and an alignment derived therefrom (SEQ ID NO: 483). FIG. 17B presents an alignment of three Ring B closers of FIG. 17A able to convert scoulerine to nandinine and cheilanthifoline to stylopine (EX45 (SEQ ID NO: 324); EX54 (SEQ ID NO: 333); and EX98 (SEQ ID NO: 377)) and an alignment derived therefrom (SEQ ID NO: 484). FIG. 17C presents an alignment of two Ring B closers of FIG. 17A able to convert scoulerine to nandinine and cheilanthifoline to stylopine (EX54 (SEQ ID NO: 333); and EX98 (SEQ ID NO: 377)) and an alignment derived therefrom (SEQ ID NO: 485). FIG. 17D presents an aligment of Ring A closers (scoulerine to nandinine and/or cheilanthifoline to stylopine) identified herein (EX76 (SEQ ID NO: 355); EX48 (SEQ ID NO: 327); EX46 (SEQ ID NO: 325); EX47 (SEQ ID NO: 326); EX66 (SEQ ID NO: 345); EX60 (SEQ ID NO: 339); EX42 (SEQ ID NO: 321); EX61 (SEQ ID NO: 340); EX96 (SEQ ID NO: 375); EX67 (SEQ ID NO: 346); EX56 (SEQ ID NO: 335); EX101 (SEQ ID NO: 380); EX44 (SEQ ID NO: 323); EX103 (SEQ ID NO: 382); EX50 (SEQ ID NO: 329); EX105 (SEQ ID NO: 384); EX69 (SEQ ID NO: 348); and EX72 (SEQ ID NO: 351)) and an alignment derived therefrom (SEQ ID NO: 486). FIG. 17E presents an alignment of Ring A closers of FIG. 17D able to convert scoulerine to cheilanthifoline (EX50 (SEQ ID NO: 329); EX76 (SEQ ID NO: 355); EX42 (SEQ ID NO: 321); EX96 (SEQ ID NO: 375); EX67 (SEQ ID NO: 346); EX56 (SEQ ID NO: 335); and EX101 (SEQ ID NO: 380)) and nandinine to stylopine and an alignment derived therefrom (SEQ ID NO: 487).

FIG. 18 presents percent identity between each pair of CYP719 EX41 to EX105.

FIG. 19. (S)-scoulerine and selected derivatives in the sanguinarine and noscapine pathways. In the sanguinarine pathway, two CYP719s act on scoulerine (boxed), converting it first to either nandinine or cheilanthifoline, and then to stylopine. TNMT (tetrahydroberberine cis-N-methyltransferase) can N-methylate scoulerine, nandinine, cheilanthifoline, and stylopine. For sanguinarine synthesis, N-methylstylopine is the desired product. In the noscapine pathway, scoulerine is O-methylated at the 9′ position by SOMT (scoulerine-9-O-methyltransferase), yielding tetrahydrocolumbamine. A CYP719 then converts tetrahydrocolumbamine to canadine. The enzyme TNMT can N-methylate scoulerine, tetrahydrocolumbamine, and canadine. For noscapine synthesis, N-methylcanadine is the desired product. All indicated BIAs are (S)-enantiomers.

FIG. 20A-B. Role of CYP719 and TNMT in the noscapine pathway. A. Synthesis of the noscapine precursor N-methylcanadine and synthesis of the side-products N-methylscoulerine and N-methytetrahydrocolumbamine (N-methylTHC) from (S)-scoulerine. B. Alkaloid profile obtained when yeast strains CEN.PK2-1D expressing PsTNMT alone or PsTNMT together with PsSOMT, PsCAS (CYP719A21) and PsCPR are supplemented with 100 μM of scoulerine at pH 8.

FIG. 21. Total recovery of supplemented BIAs. Strains expressing no heterologous enzymes were incubated overnight with 5 uM (R,S)-norlaudanosoline (A), (S)-scoulerine (B), or (S)-stylopine (C) in YNB or media buffered to pH 6, 7, 8, or 9. After 16 hours, supplemented BIAs were extracted and analyzed by HPLC-FT-MS.

FIG. 22. Relative recovery of supplemented BIAs in supernatant and cell extract fractions of yeast cultures. Strains expressing no heterologous enzymes were incubated overnight with 5 uM (R,S)-norlaudanosoline (A), (S)-scoulerine (B), or (S)-stylopine (C) in YNB or media buffered to pH 6, 7, 8, or 9. After 16 hours, supplemented BIAs were extracted and analyzed by HPLC-FT-MS.

FIG. 23 Turnover of supplemented BIAs to downstream end products. Strains expressing Block 1 enzymes (A: GCY1086), Block 2 enzymes and CPR (B: GCY1090), or Block 3 enzymes and CPR (C: GCY1094) were incubated overnight with 5 uM (R,S)-norlaudanosoline, (S)-scoulerine, or (S)-stylopine, respectively, in YNB or media buffered to pH 6, 7, 8, or 9. After 16 hours, total BIAs were extracted and analyzed by HPLC-FT-MS.

FIG. 24A-B: Description of the pBOT vector system. (A) Schematized description of vector. The four pBOT versions available contain a different auxotrophy (LEU, URA, HIS or TRP) and different promoter-terminator pairs associated with each auxotrophy. (B) Plasmid (SEQ ID NOs: 568-569), gene (SEQ ID NOs: 570-571), and ligation product (SEQ ID NOs: 572-573). Any gene of interest can be cloned by SapI restriction digestion and ligation.

### DESCRIPTION OF ILLUSTRATIVE EMBODIMENTS

### Definitions

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

In the present description, a number of terms are extensively utilized. In order to provide a clear and consistent understanding of the specification and claims, including the scope to be given such terms, the following definitions are provided.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the term “consists of” or “consisting of” means including only the elements, steps, or ingredients specifically recited in the particular claimed embodiment or claim.

### Enzymes

The present invention relates to enzymes involved in a BIA synthetic pathway encoded by plasmids or chromosomes in a host cell and improved methods of use thereof to produce various BIA metabolites.

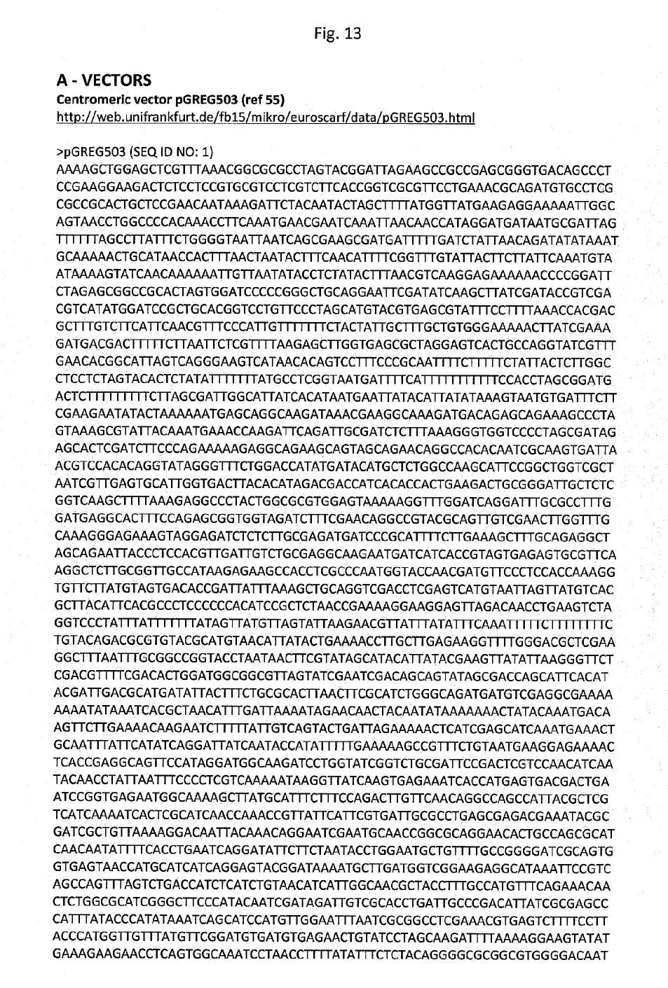

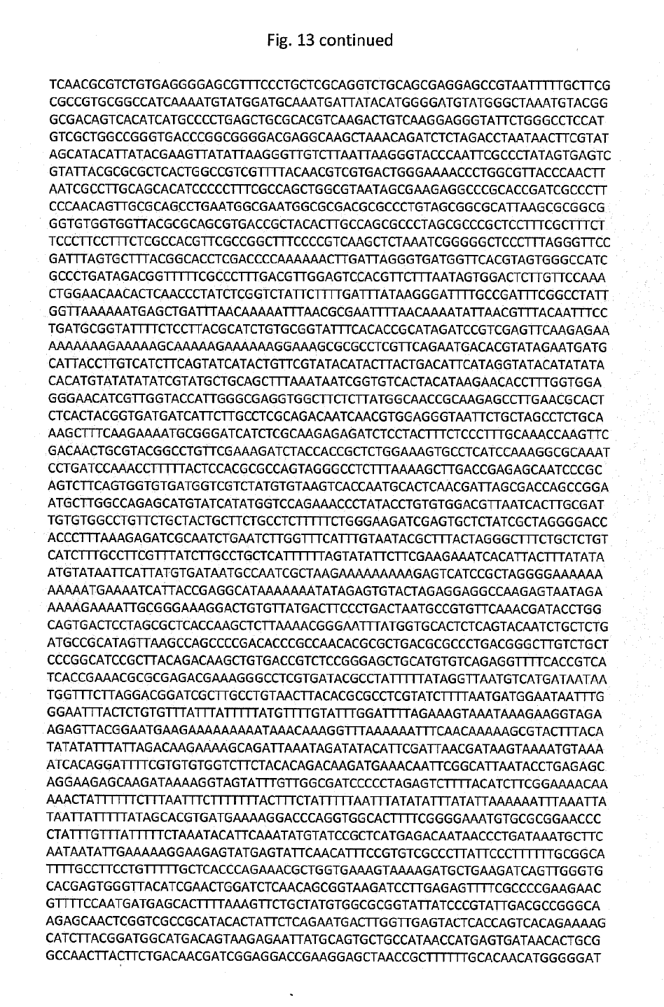

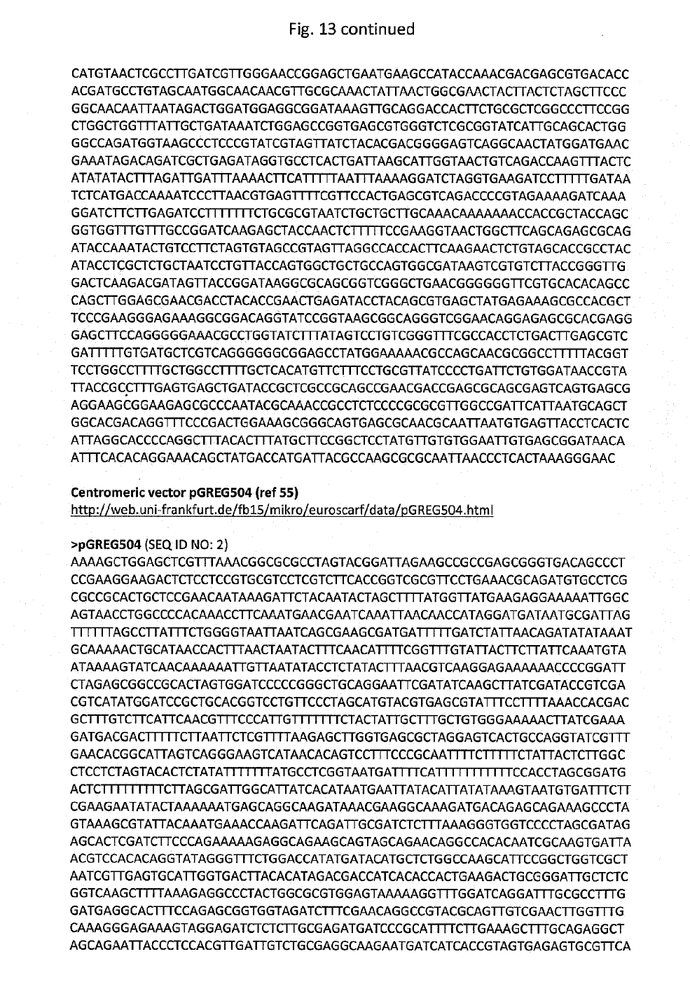

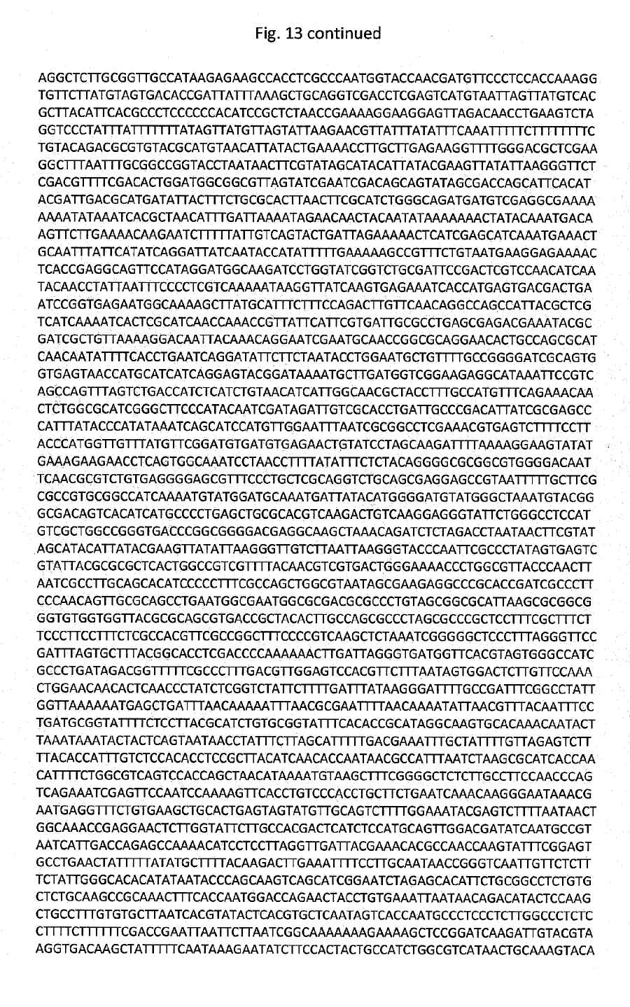

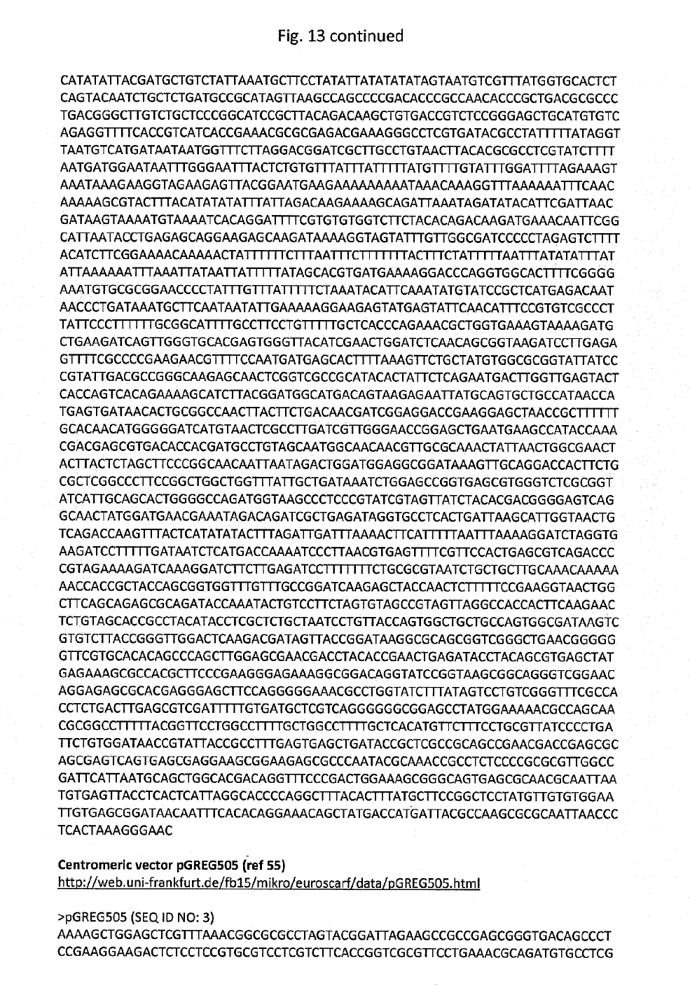

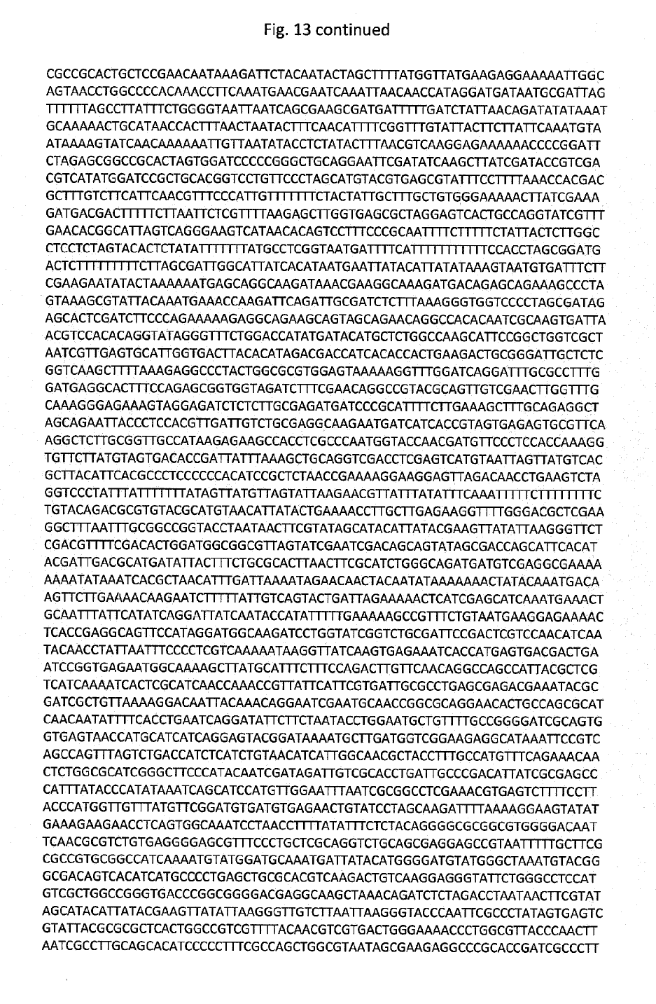

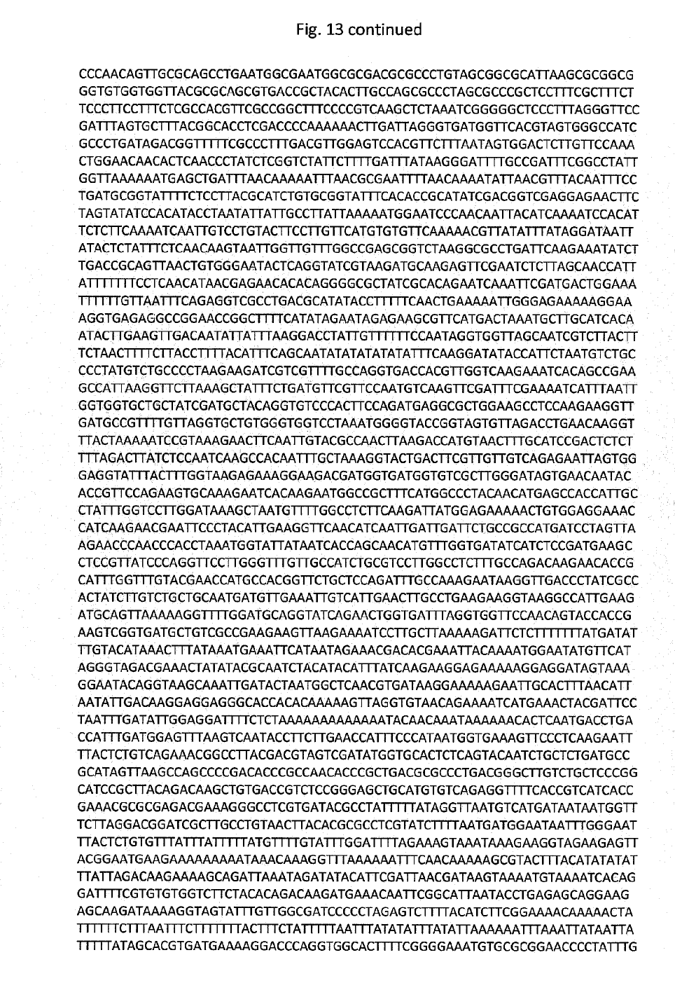

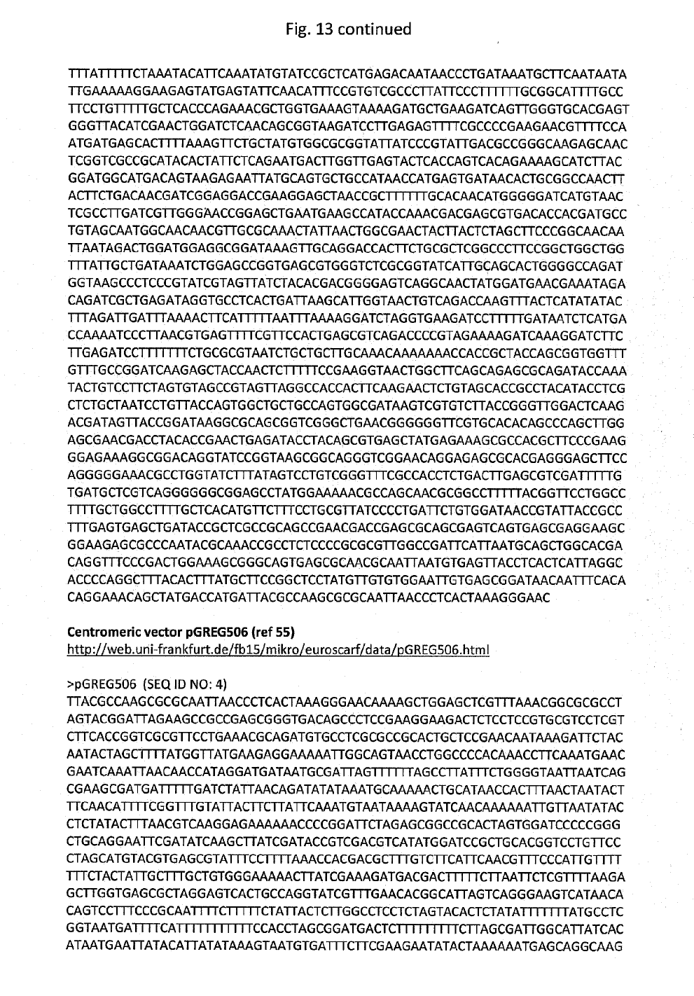

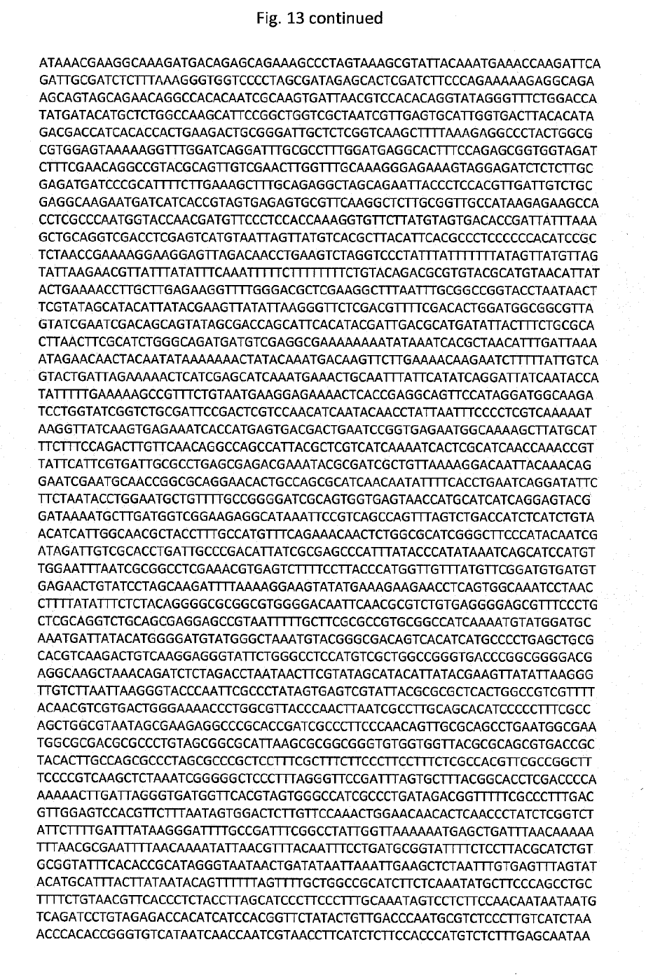

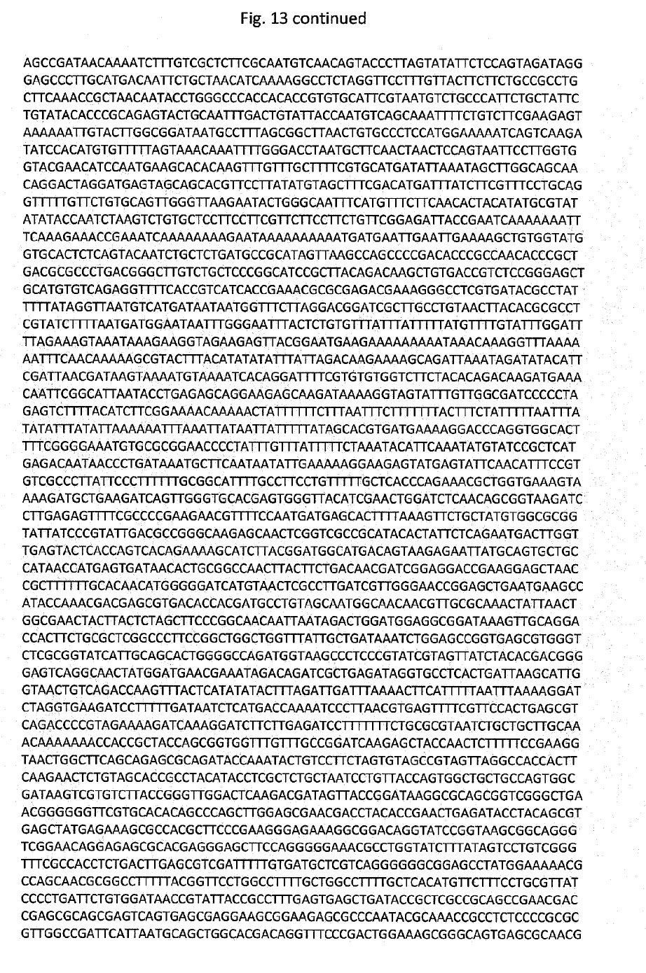

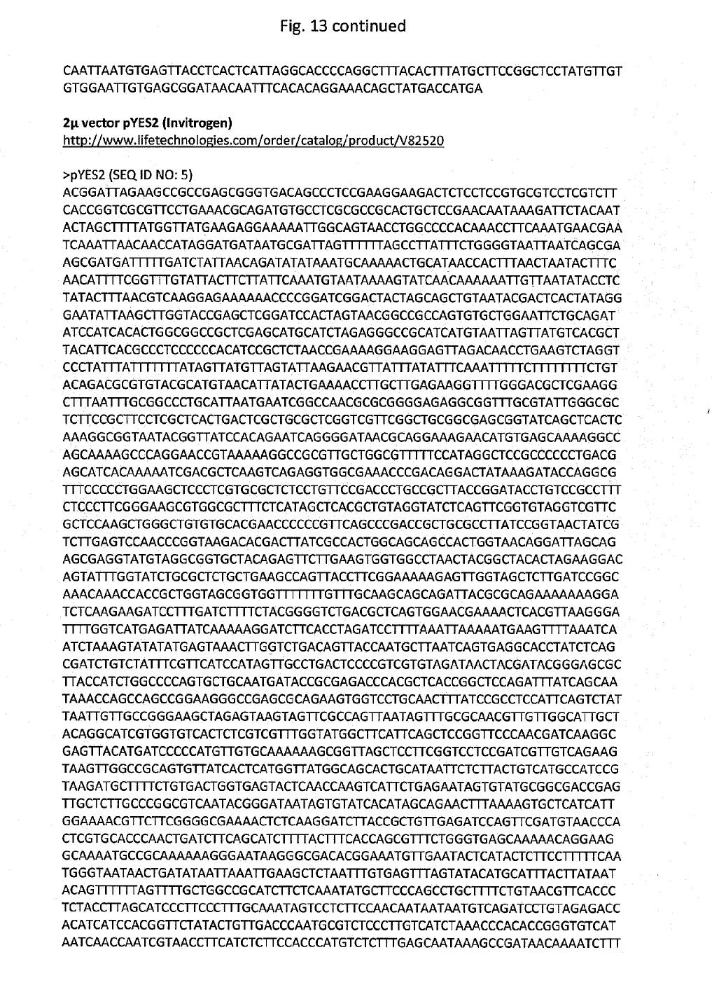

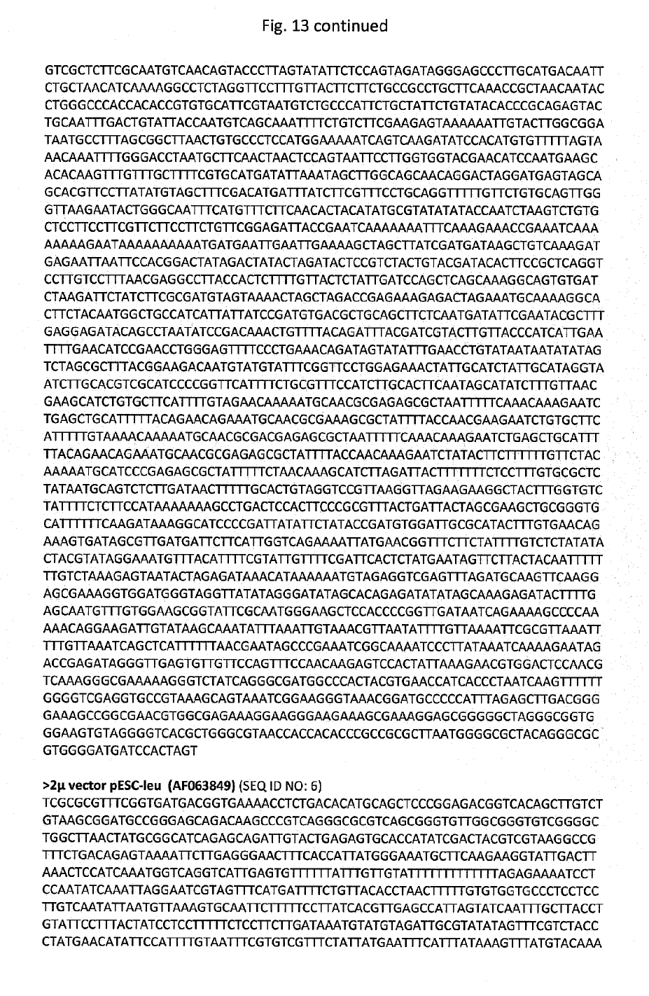

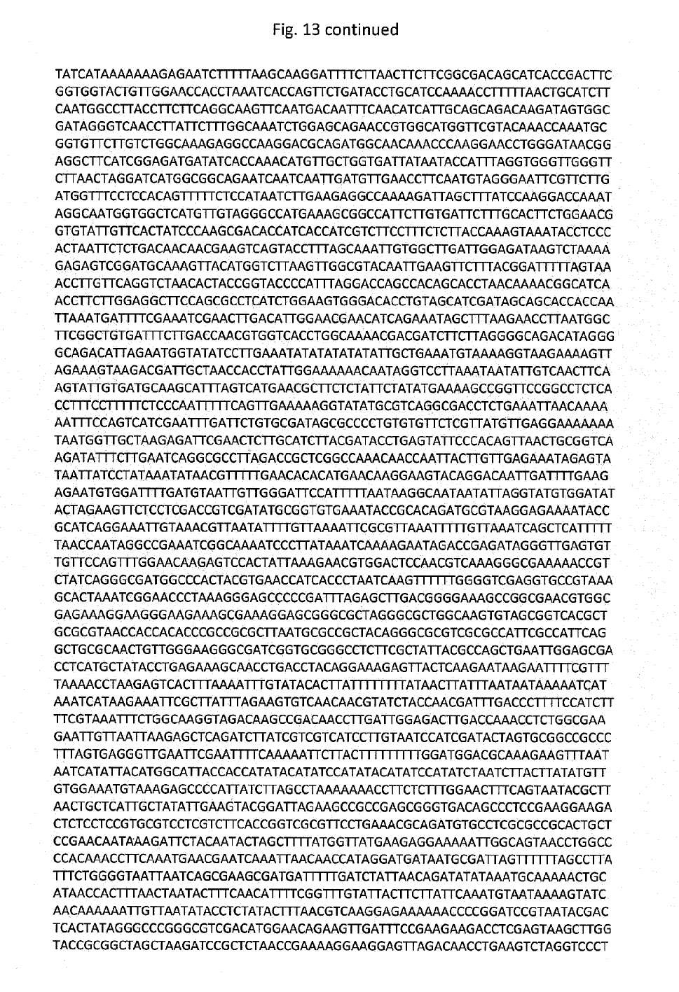

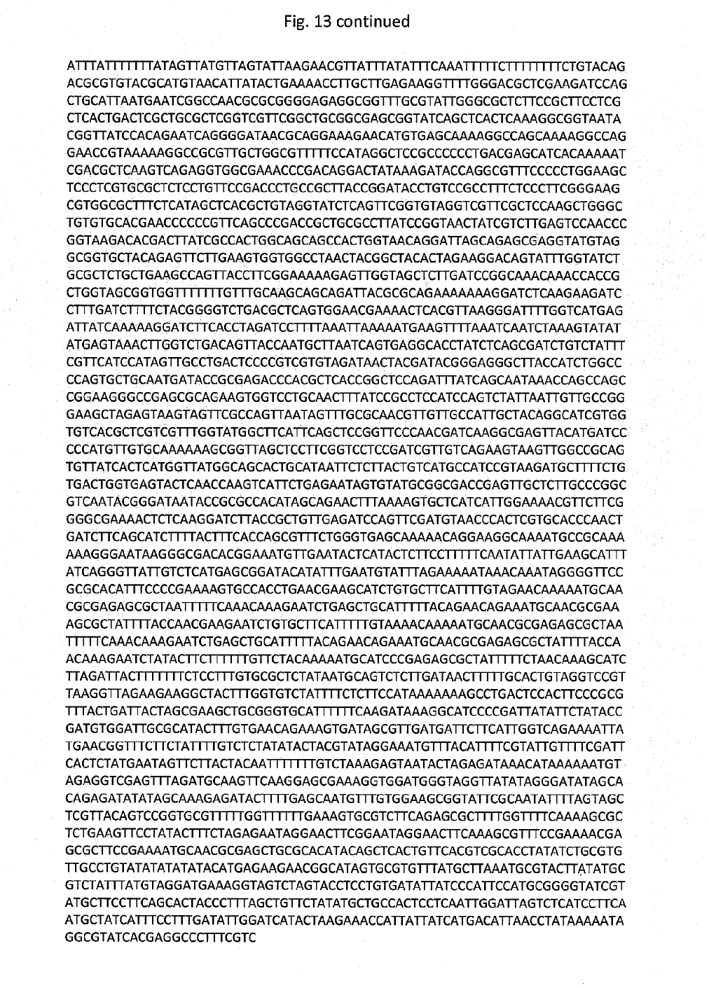

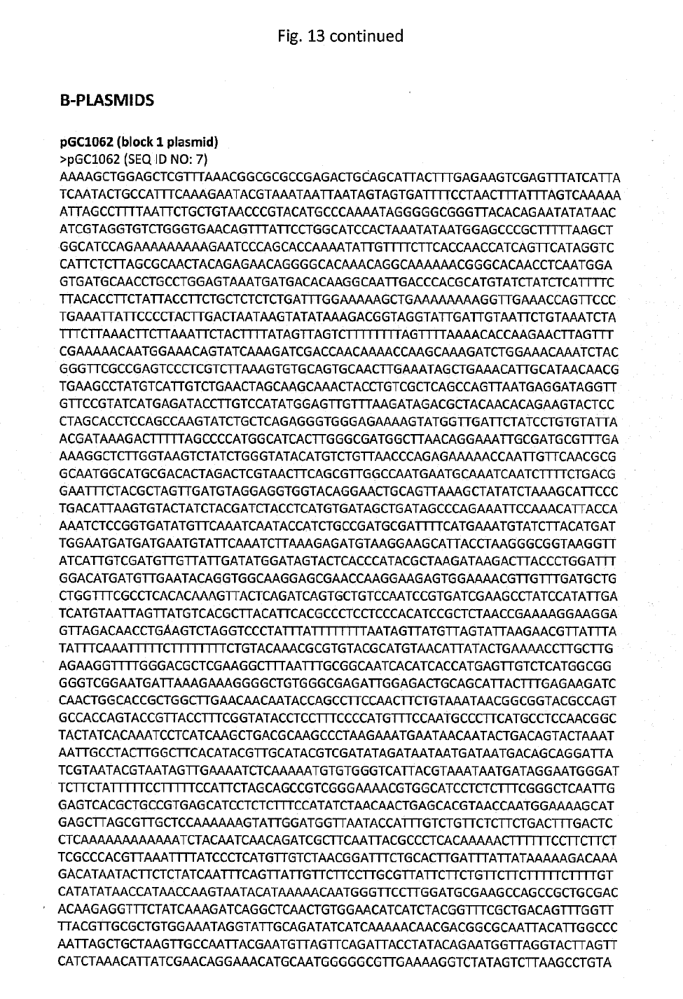

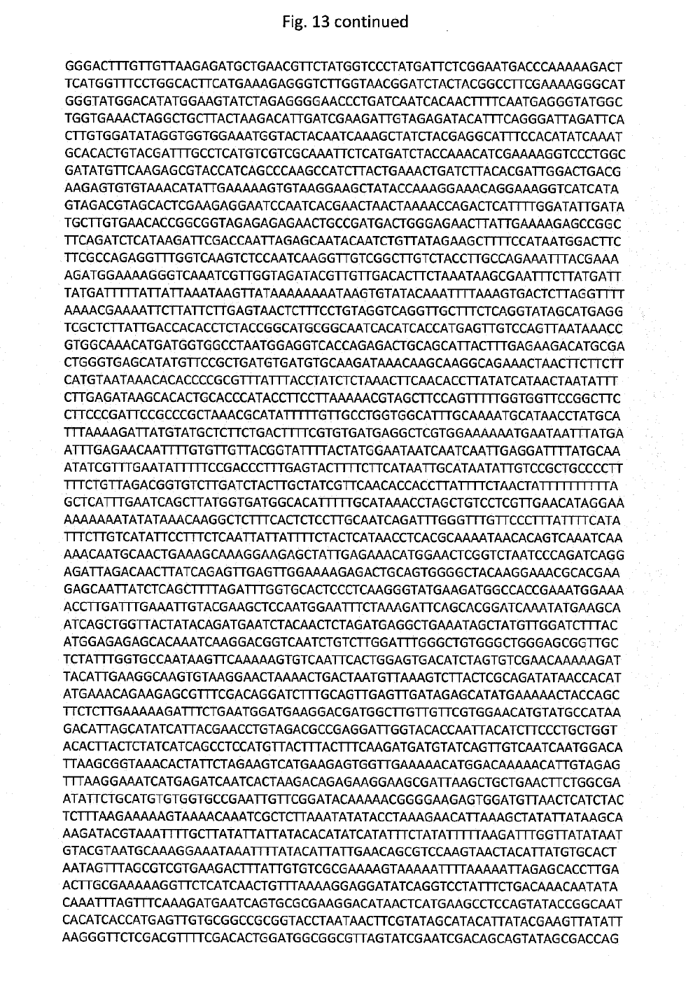

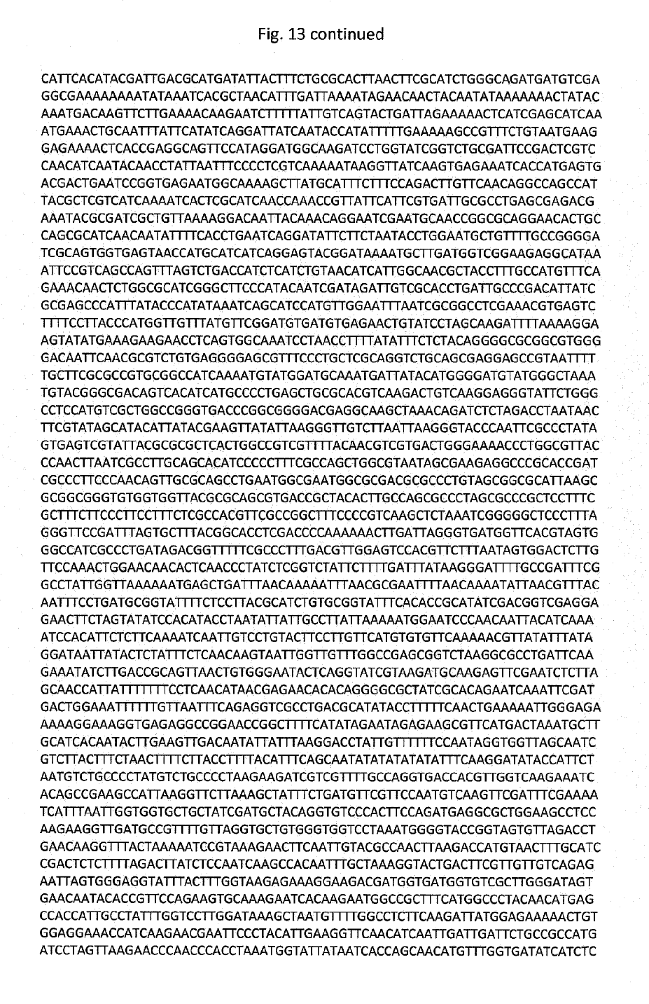

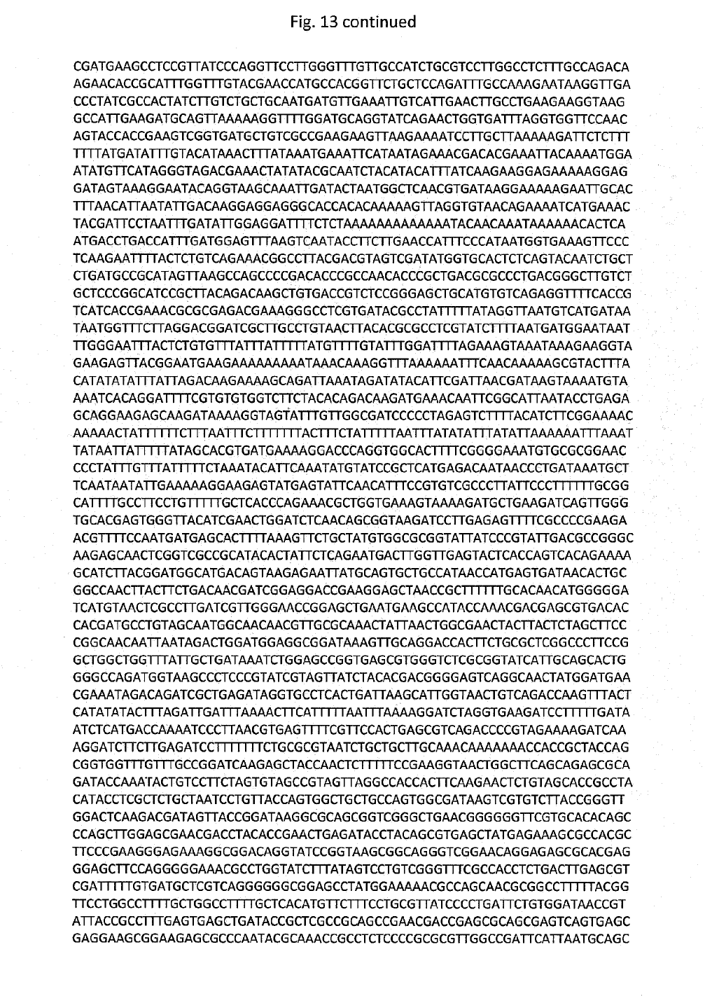

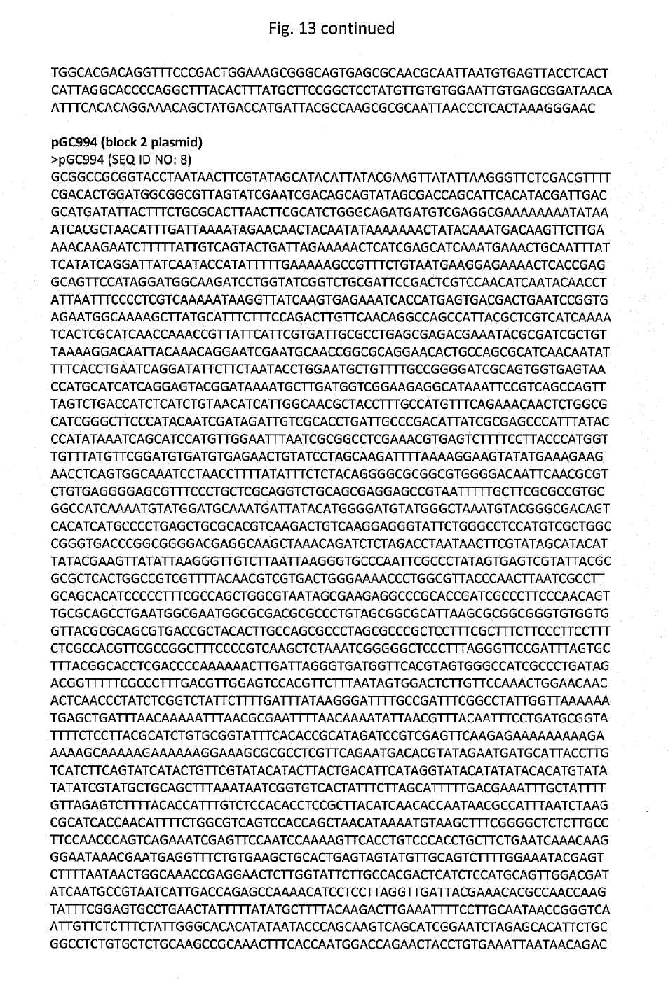

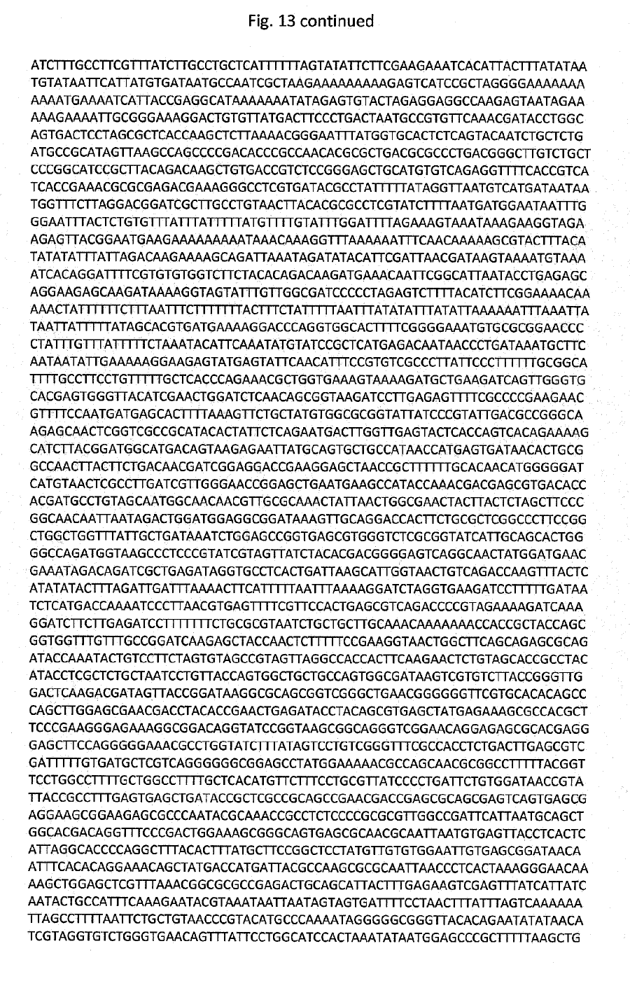

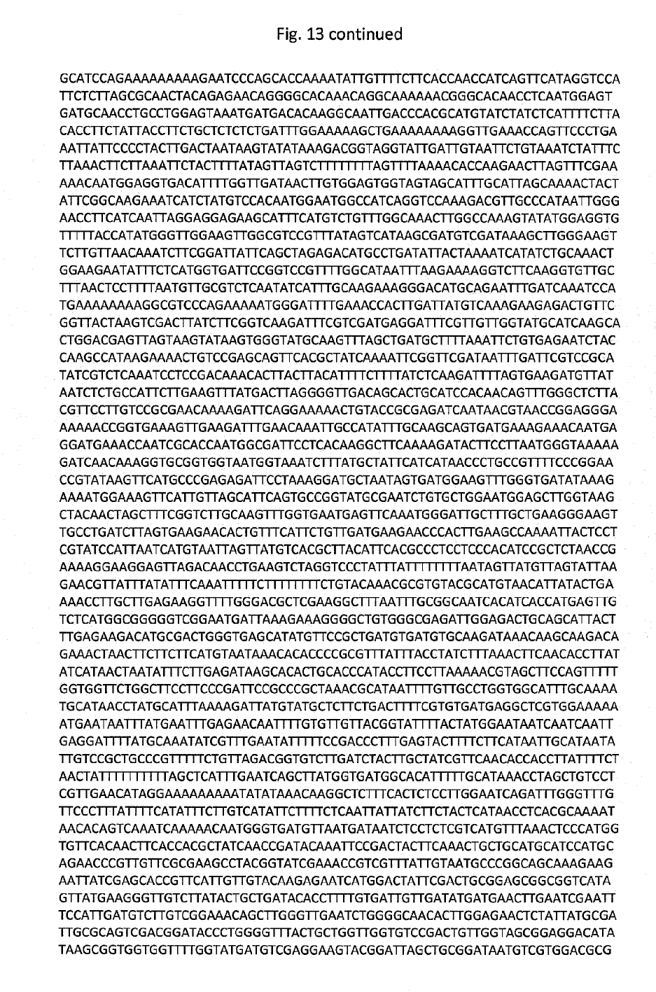

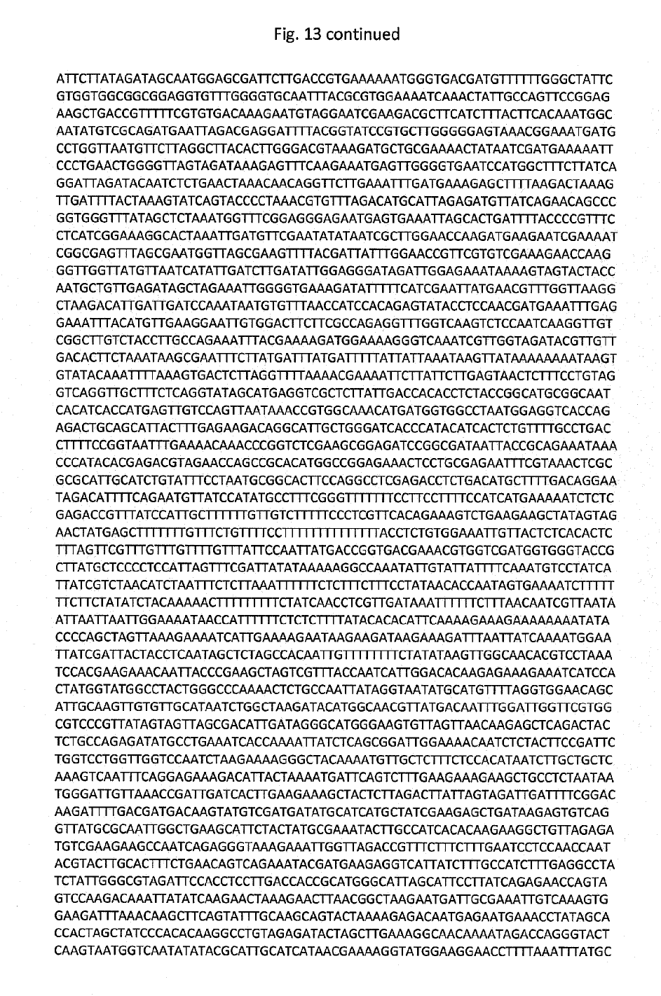

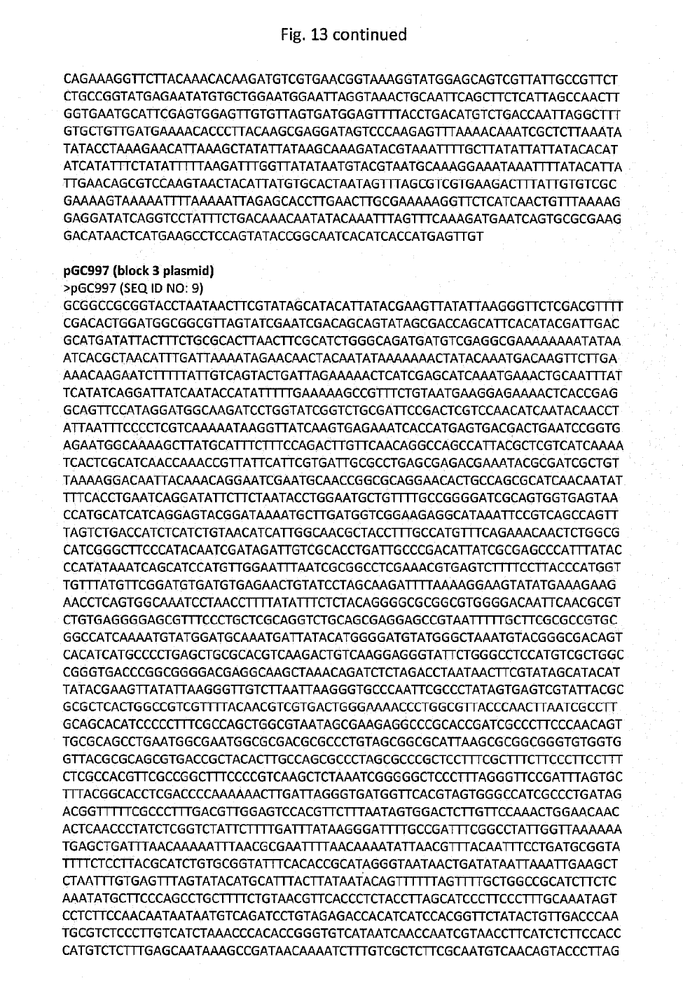

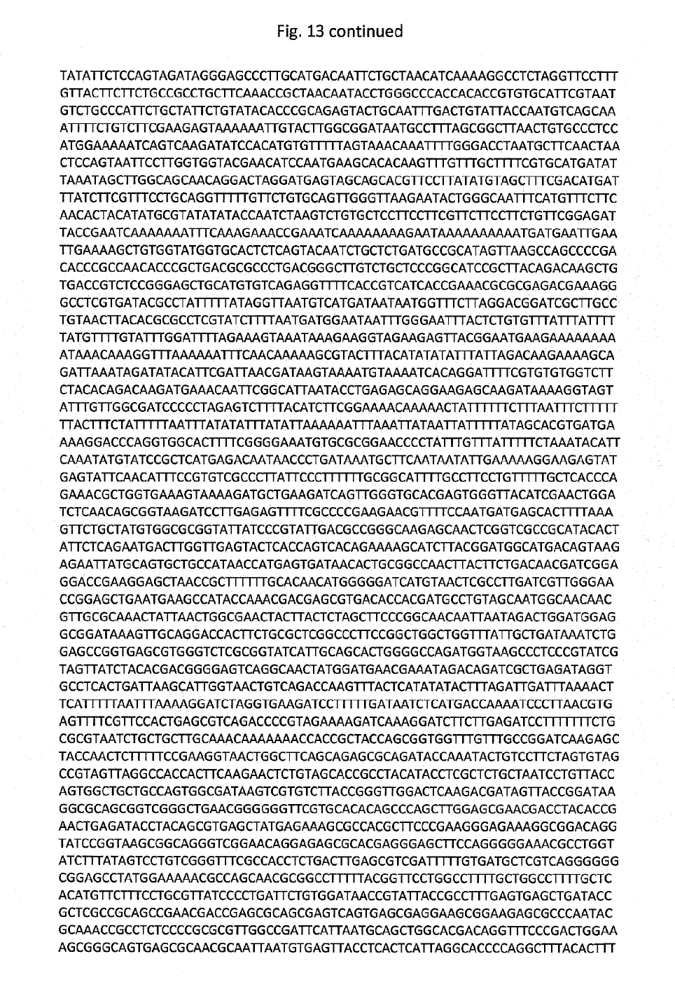

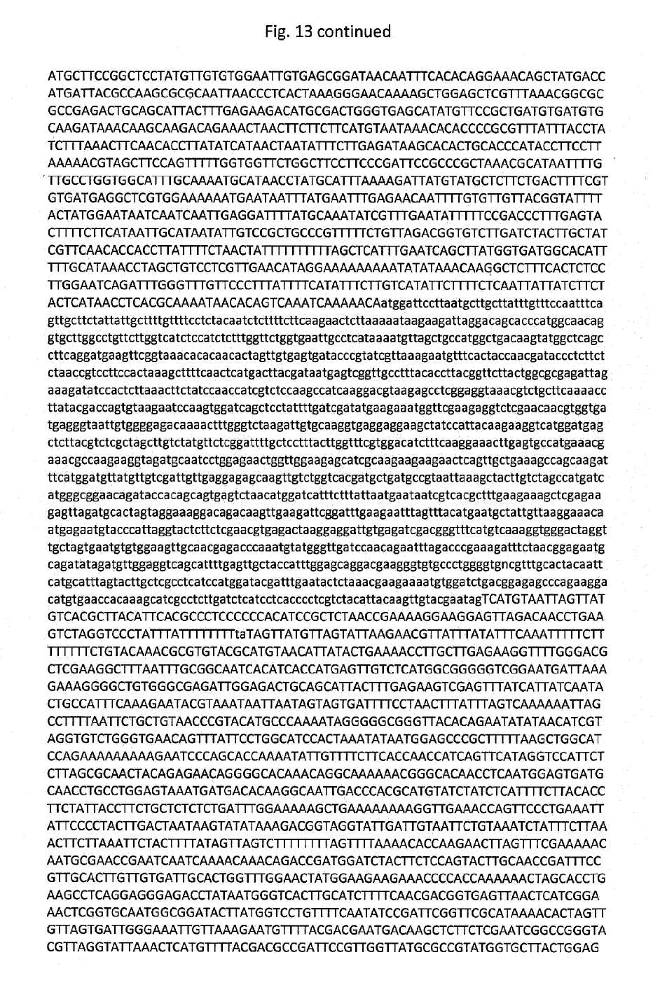

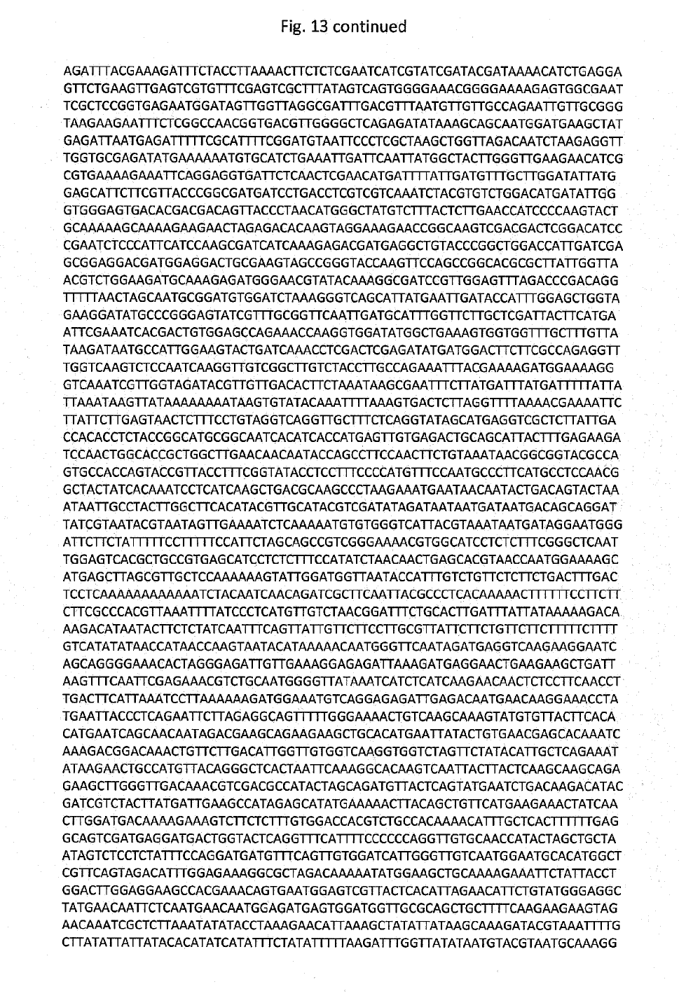

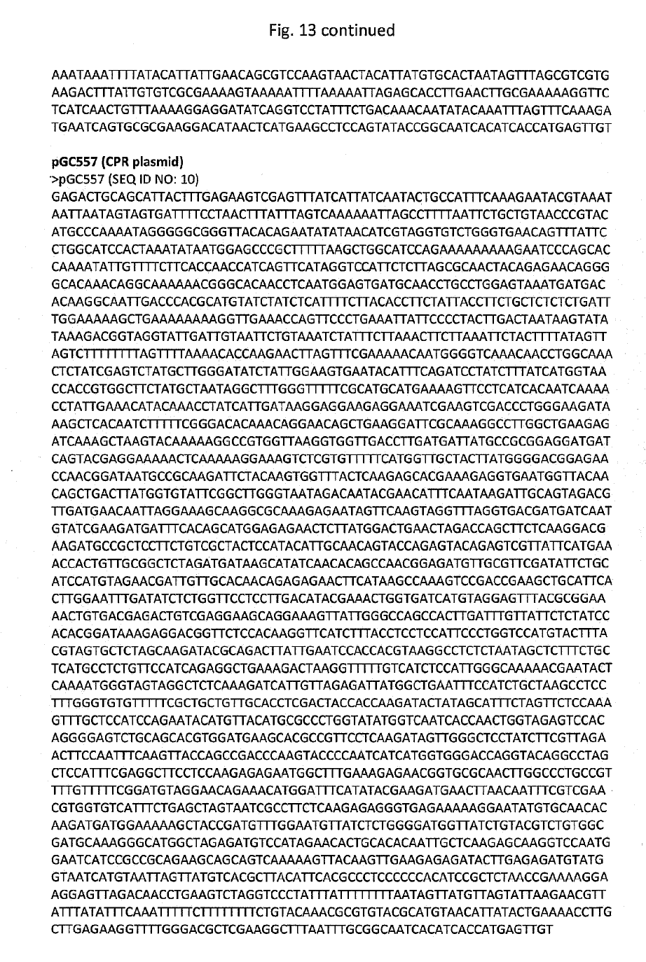

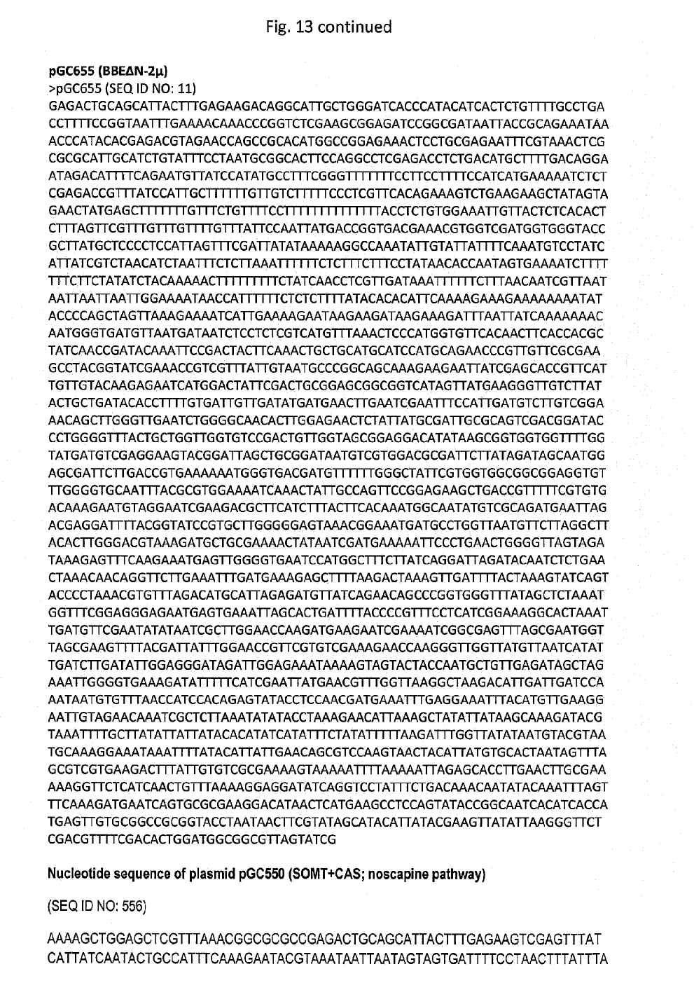

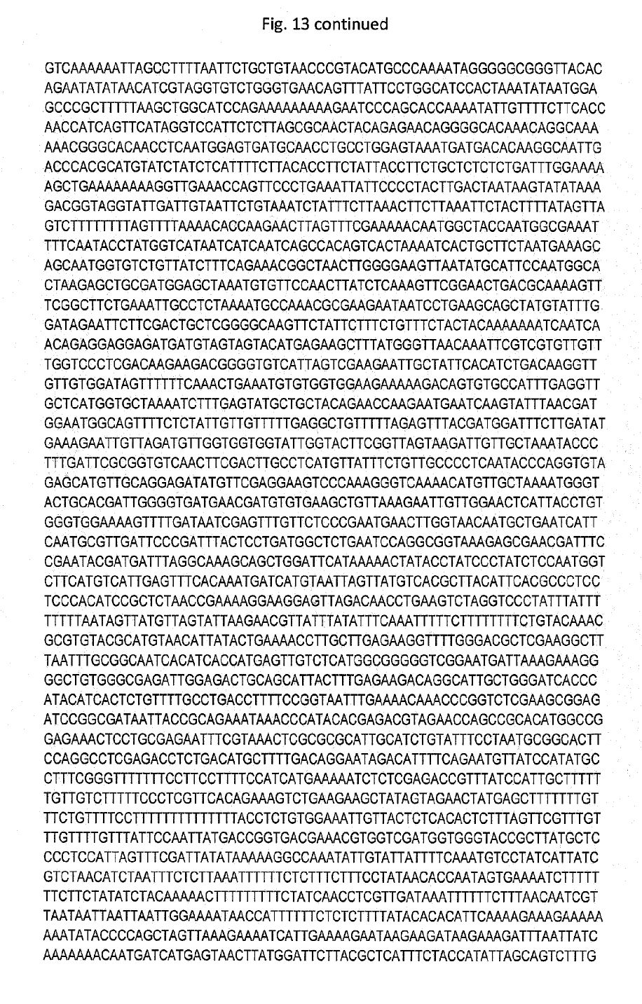

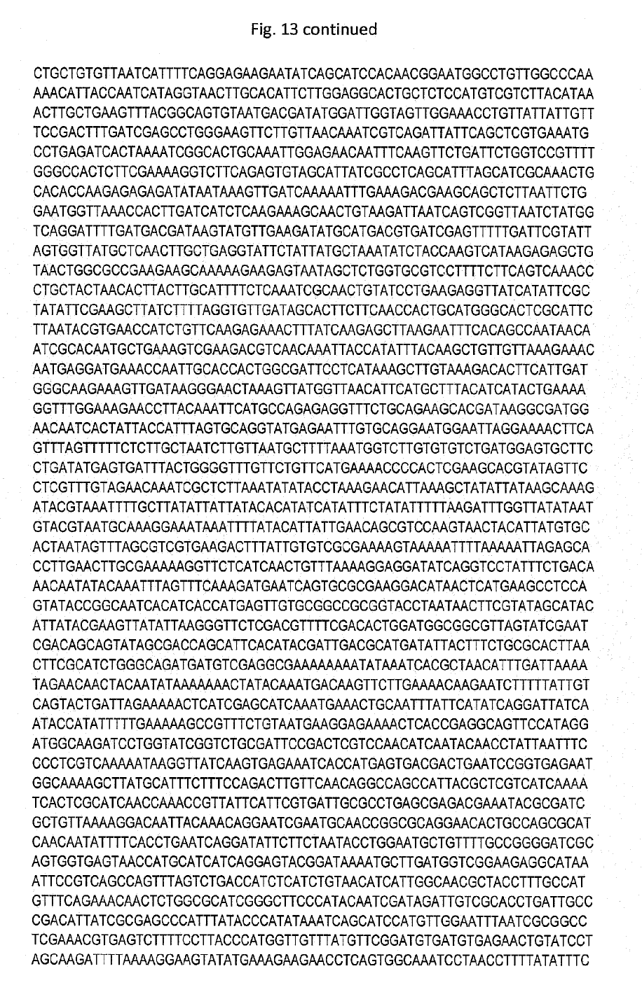

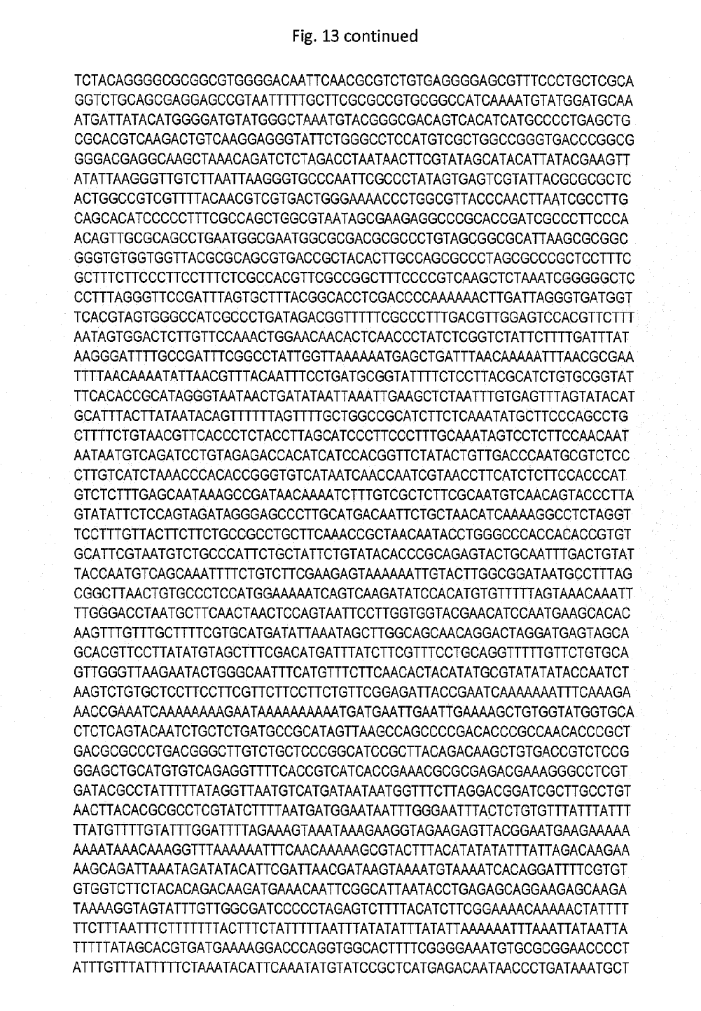

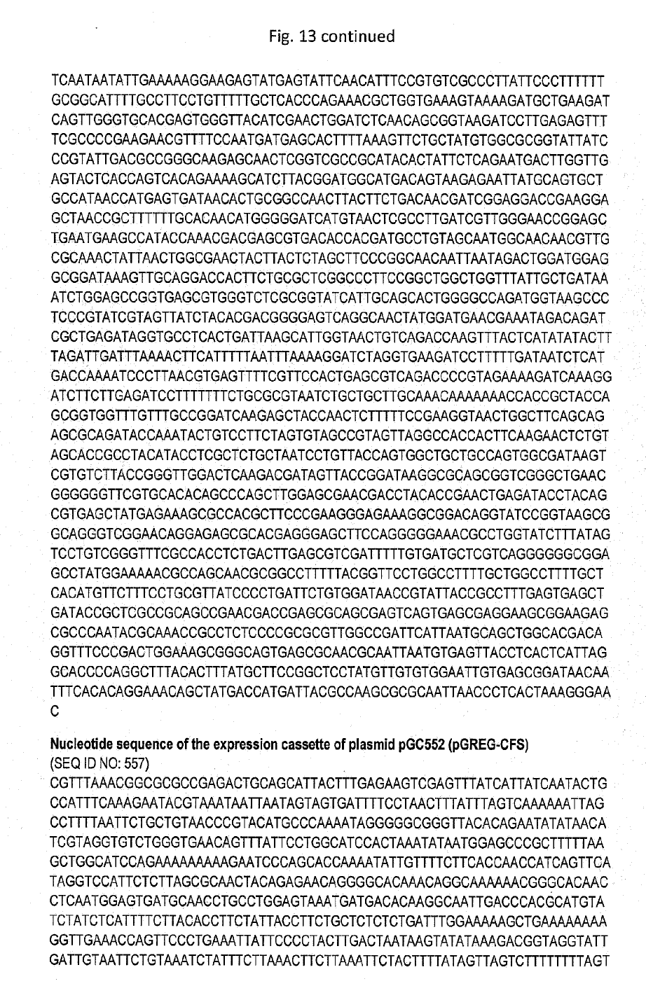

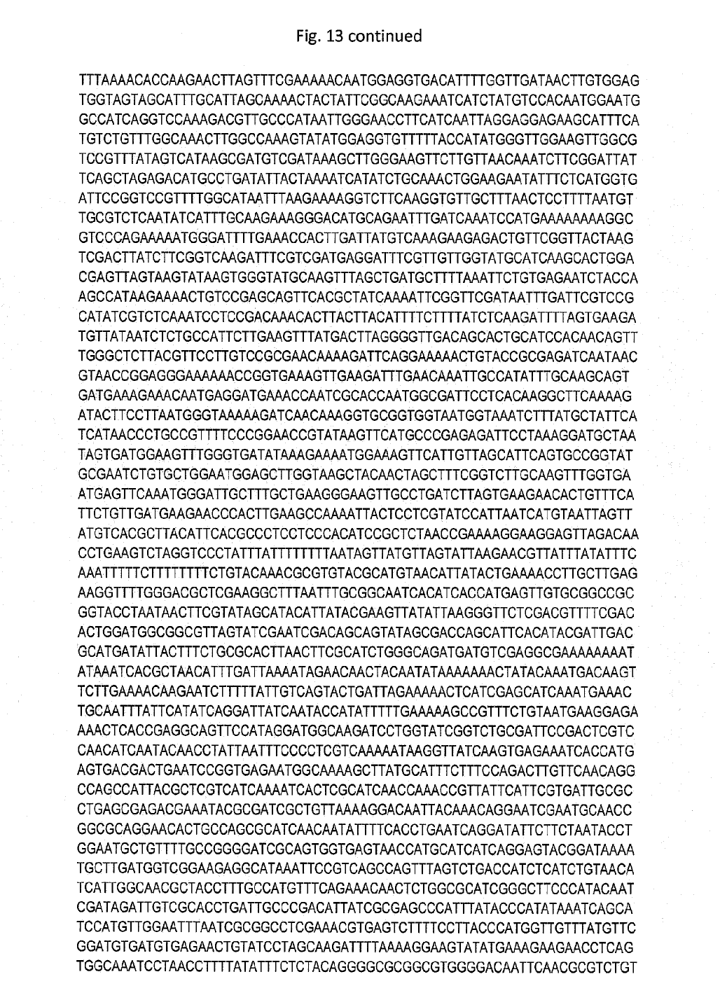

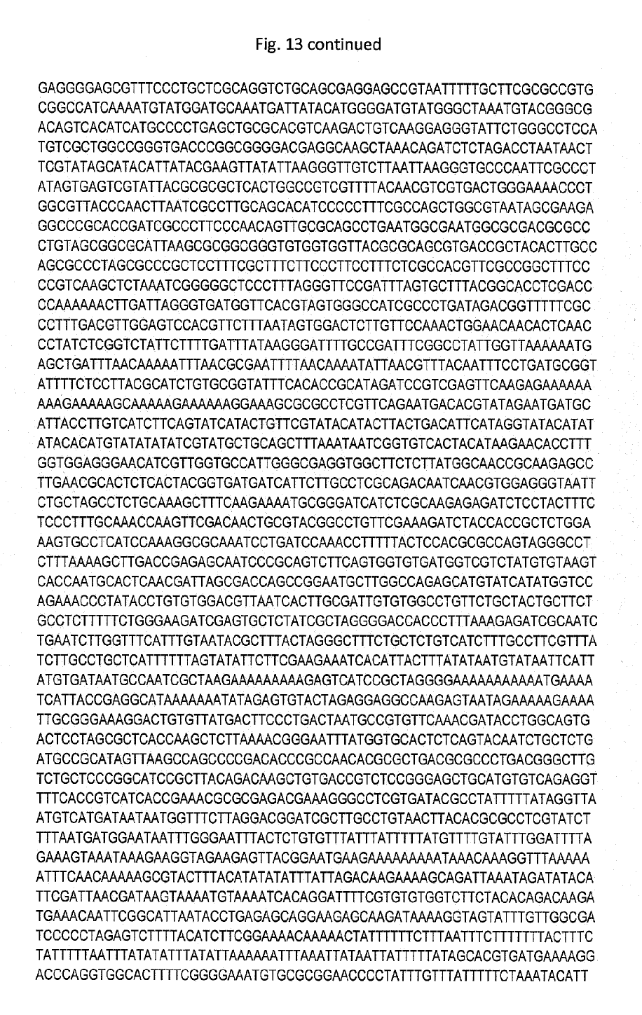

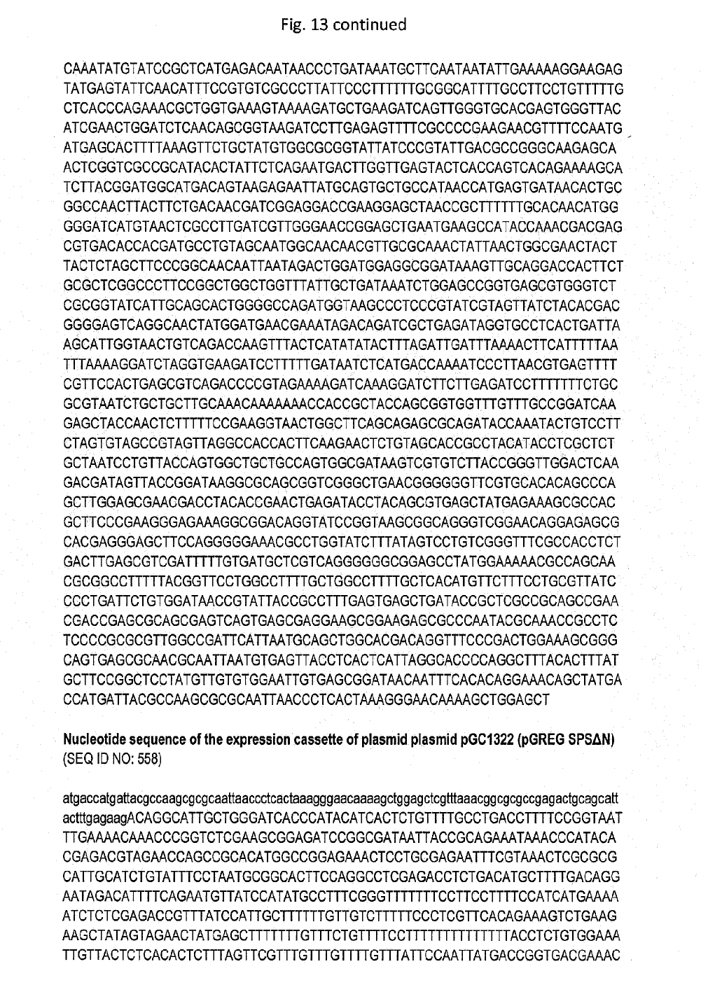

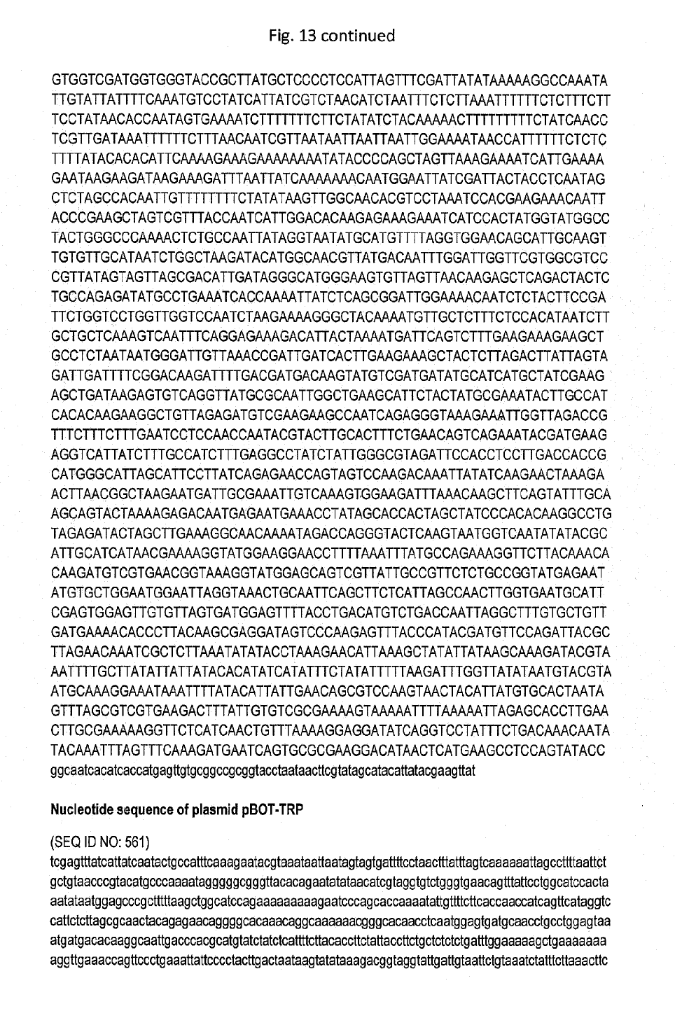

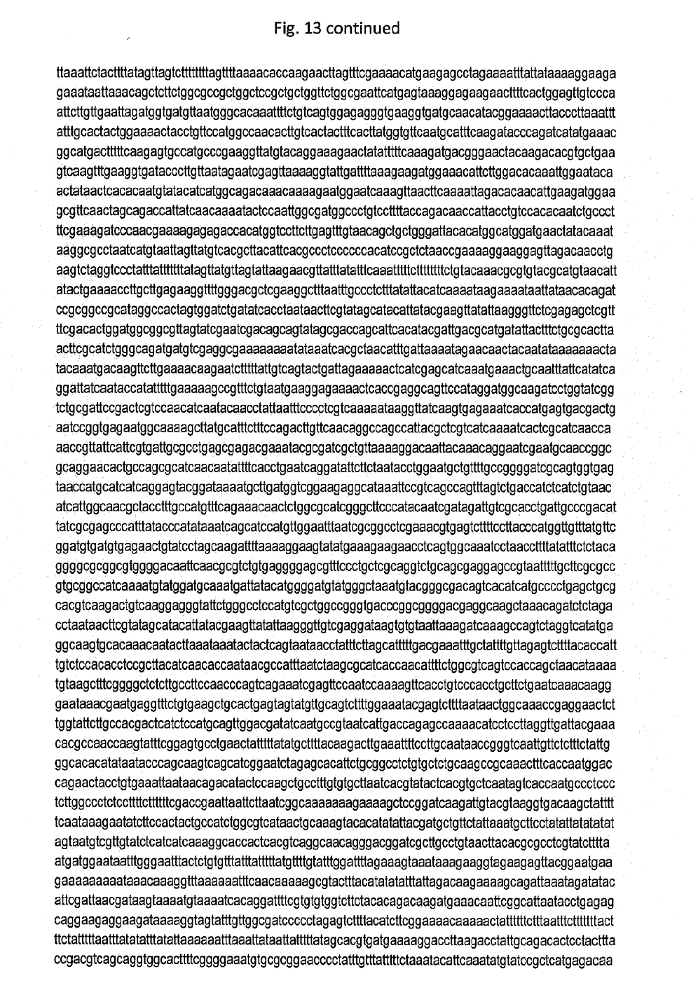

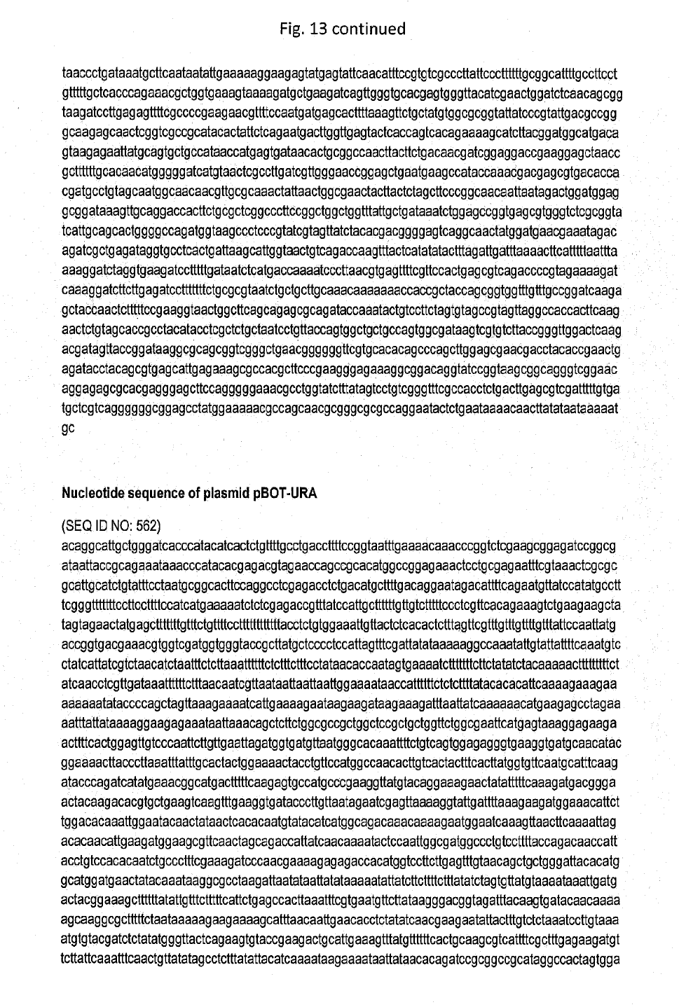

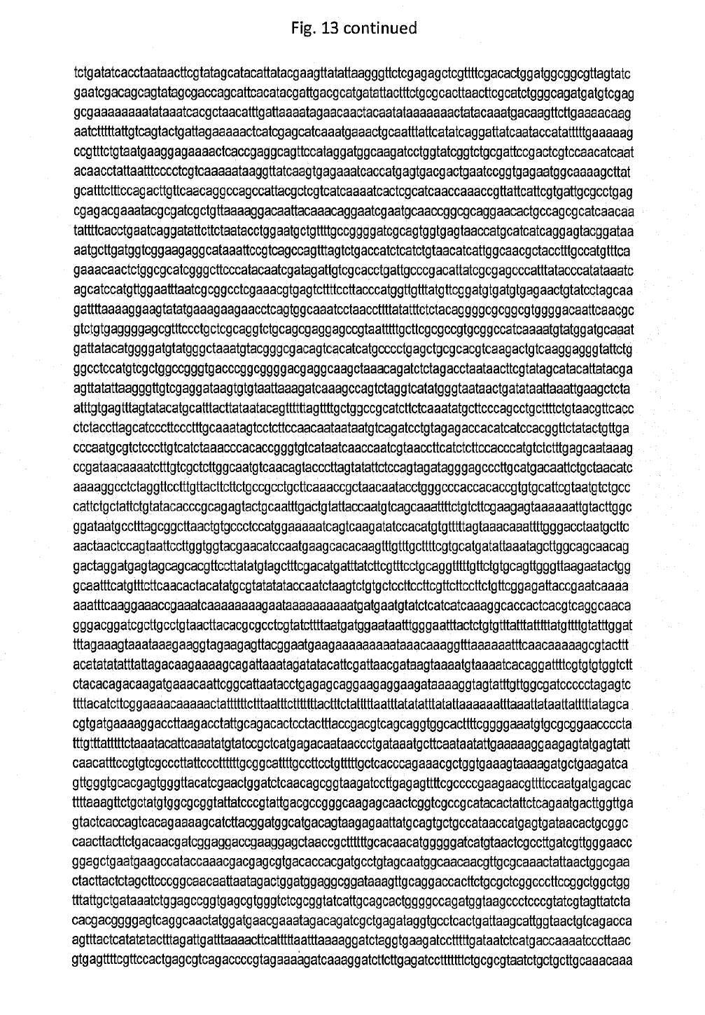

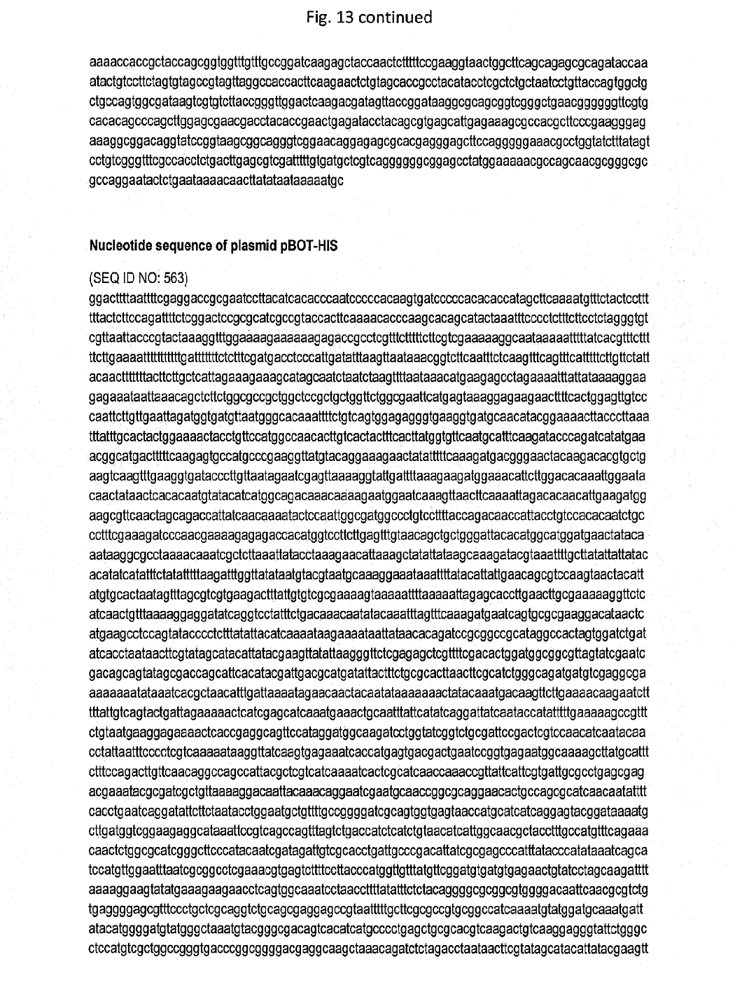

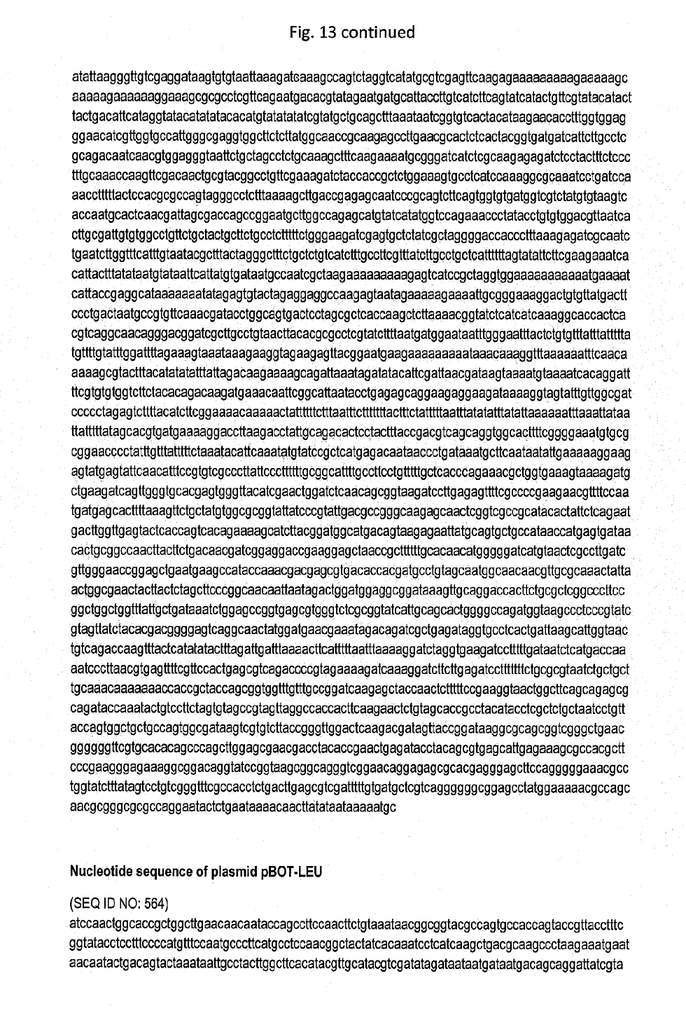

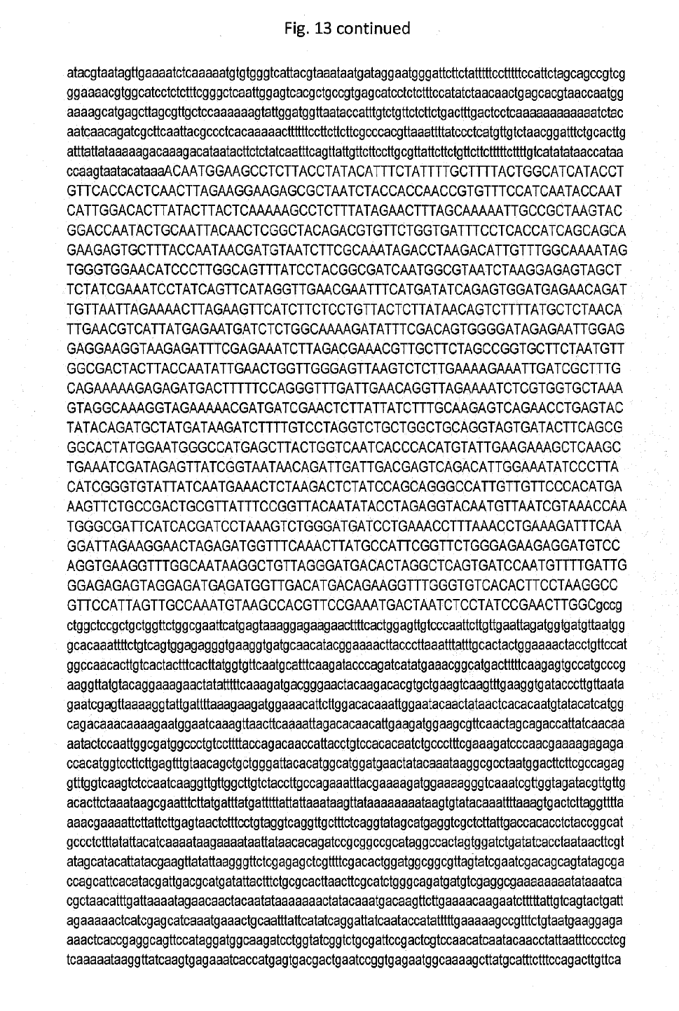

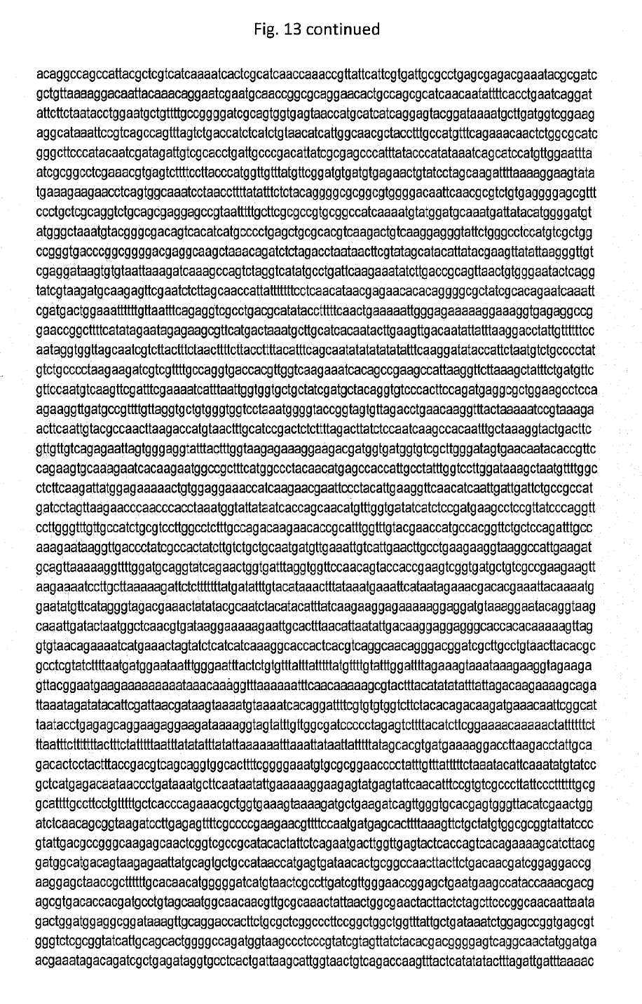

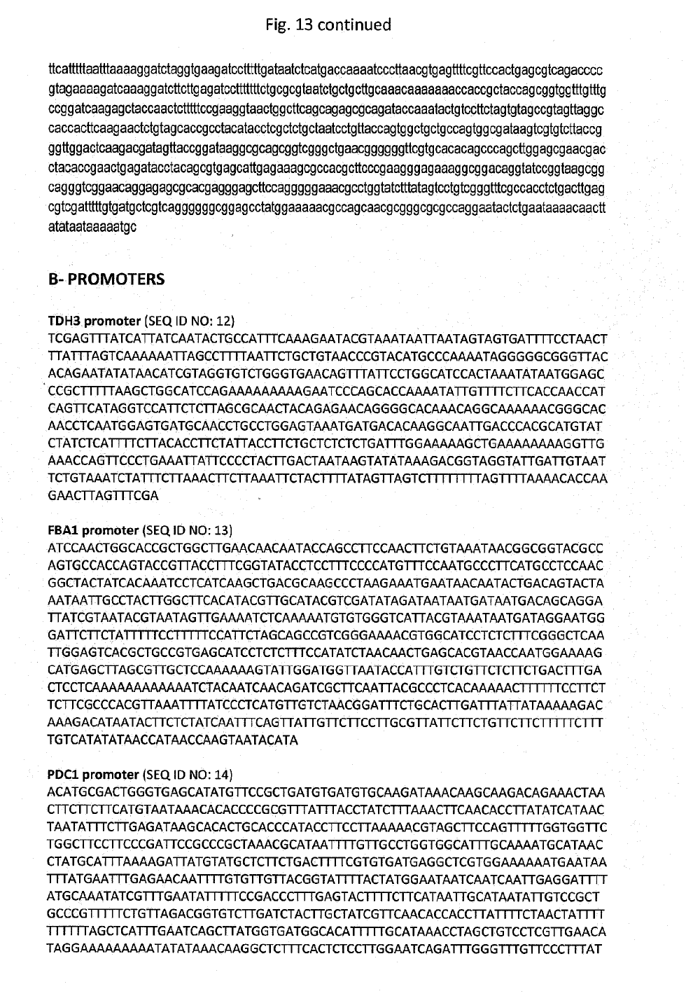

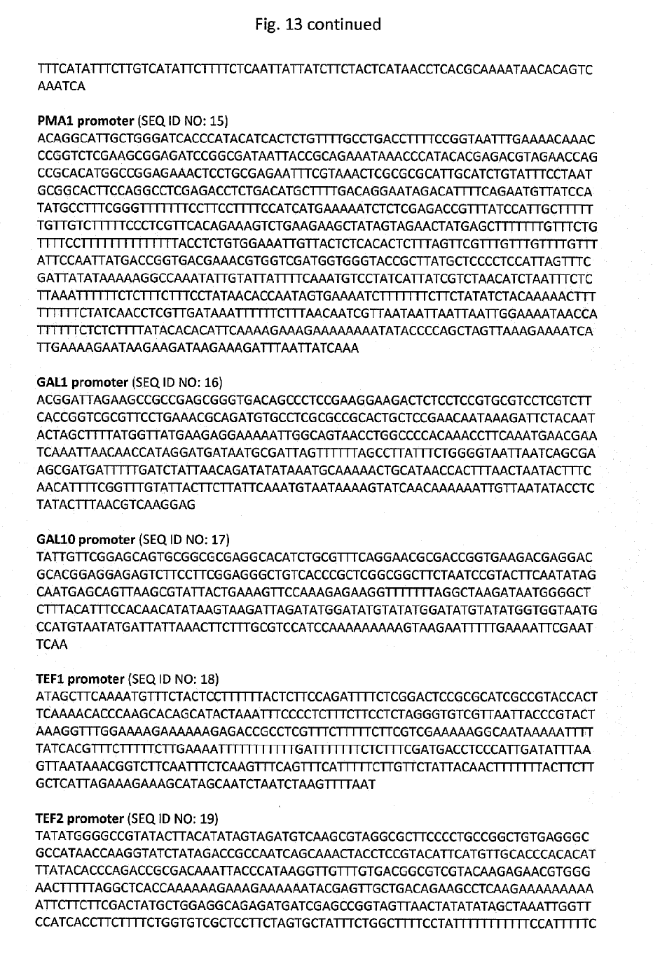

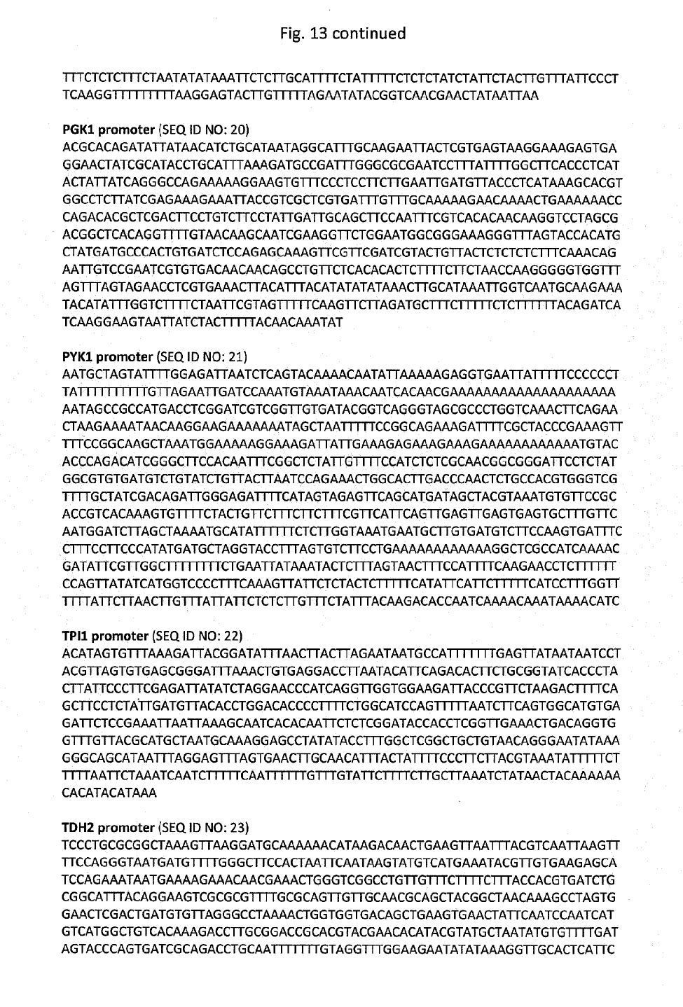

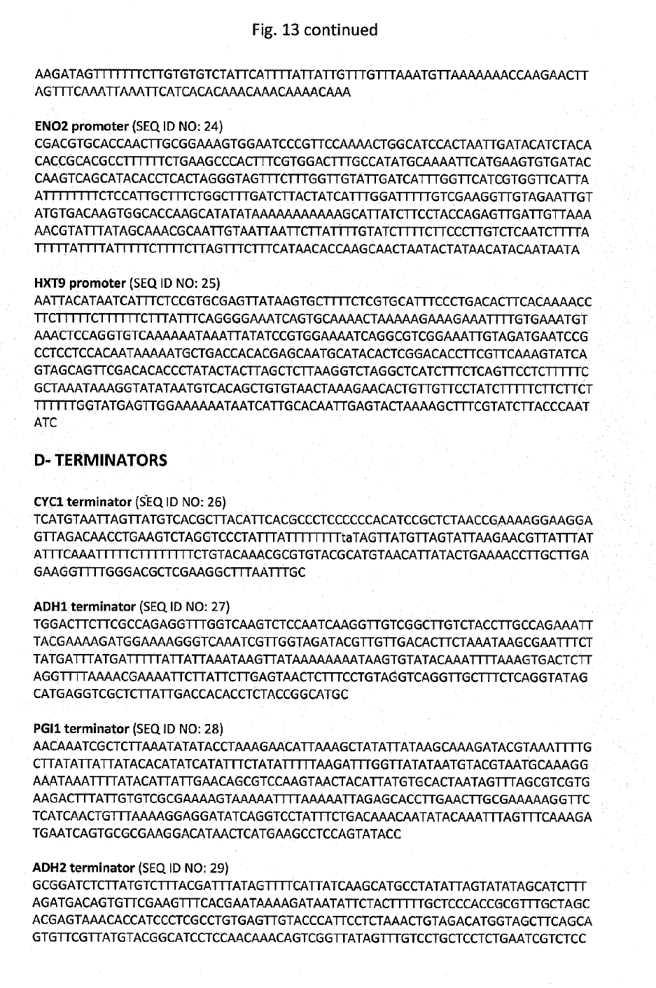

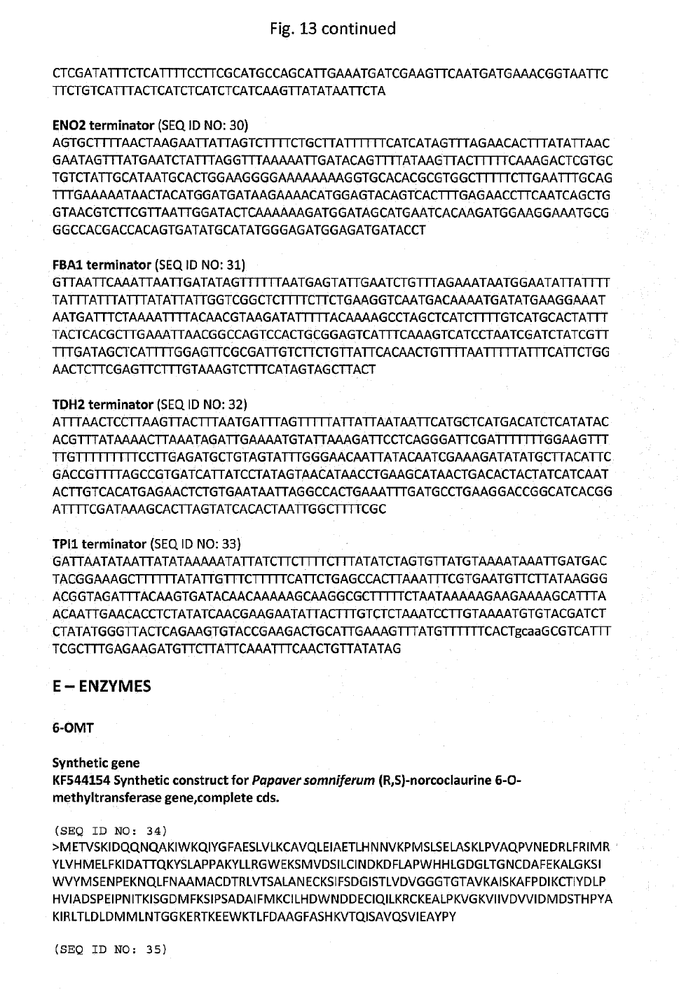

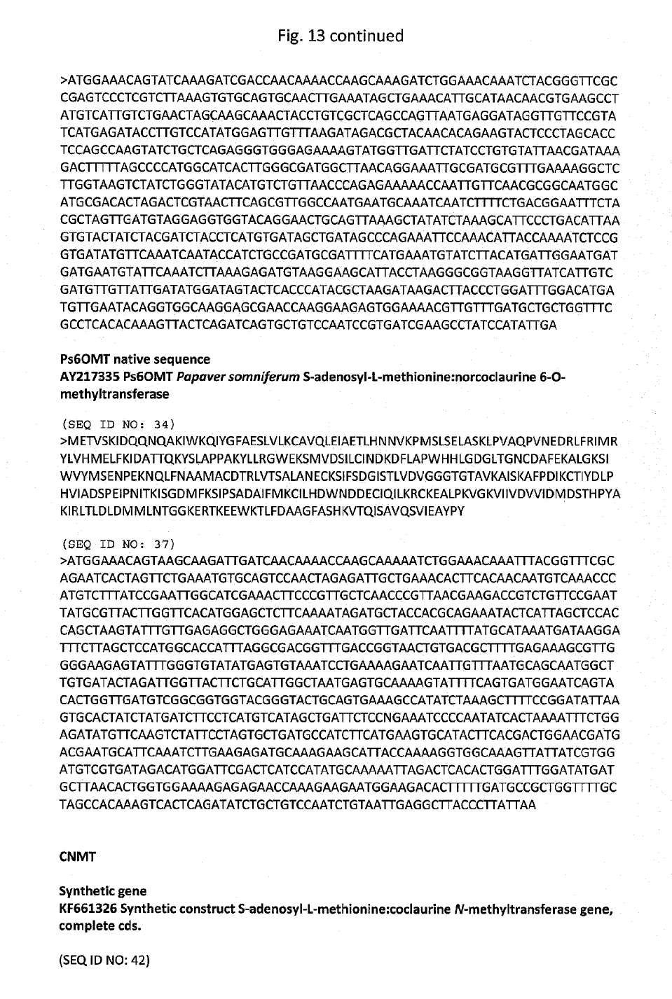

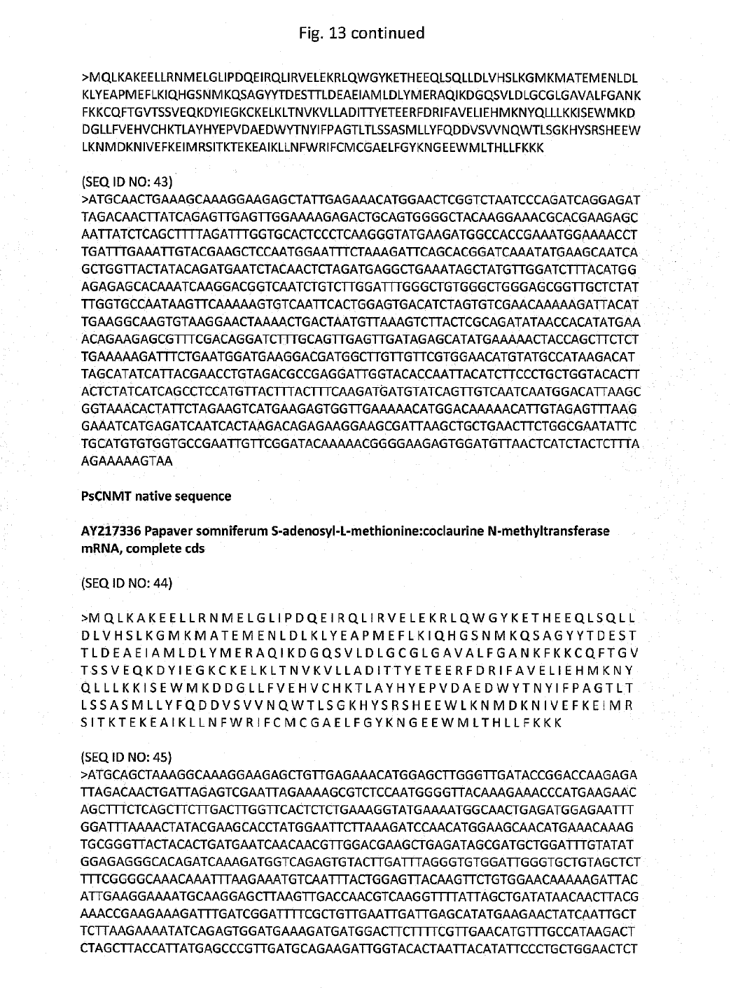

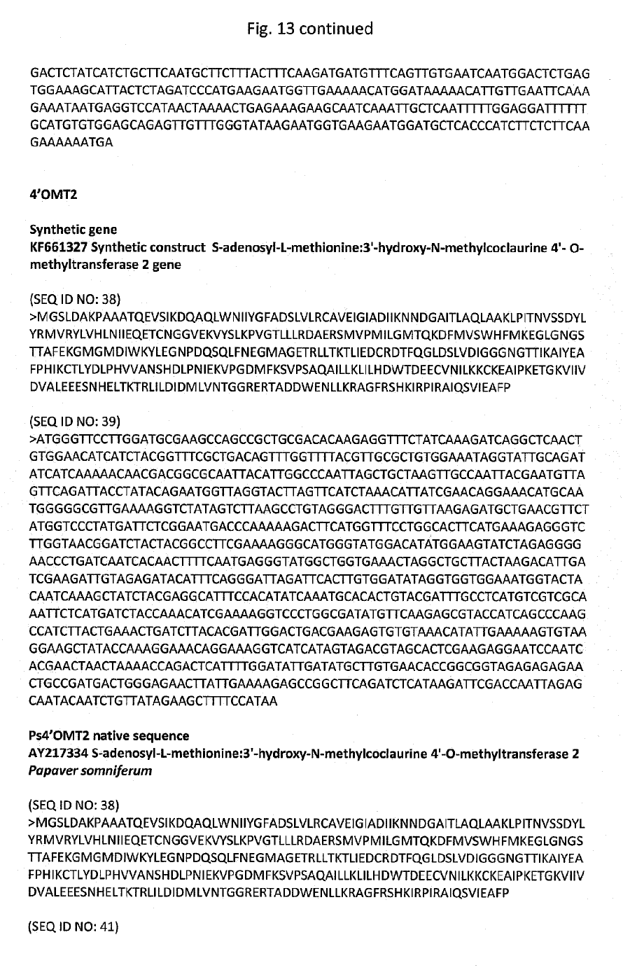

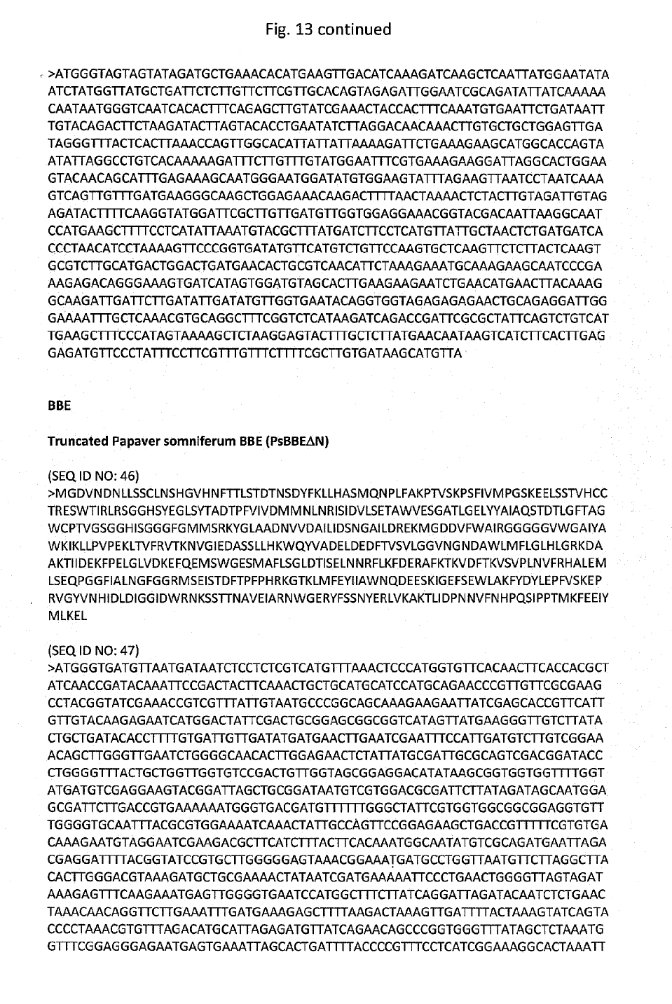

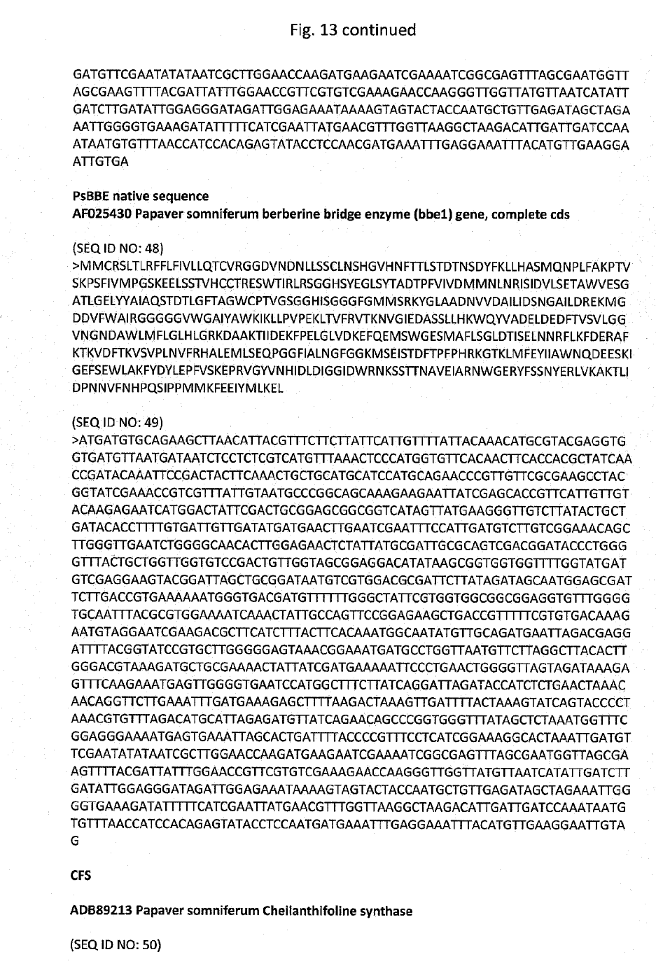

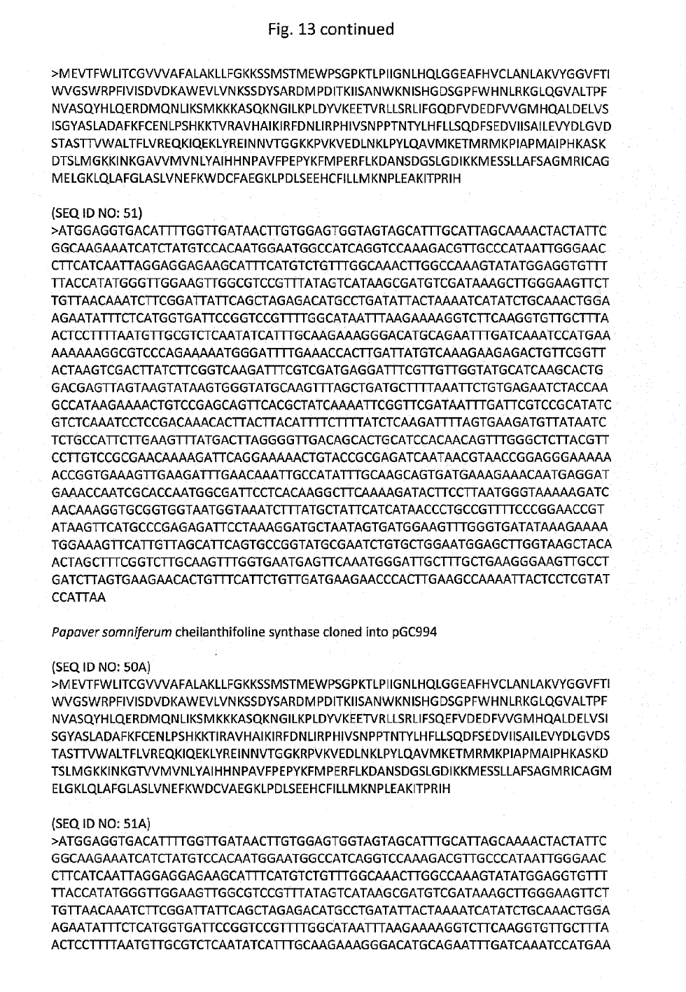

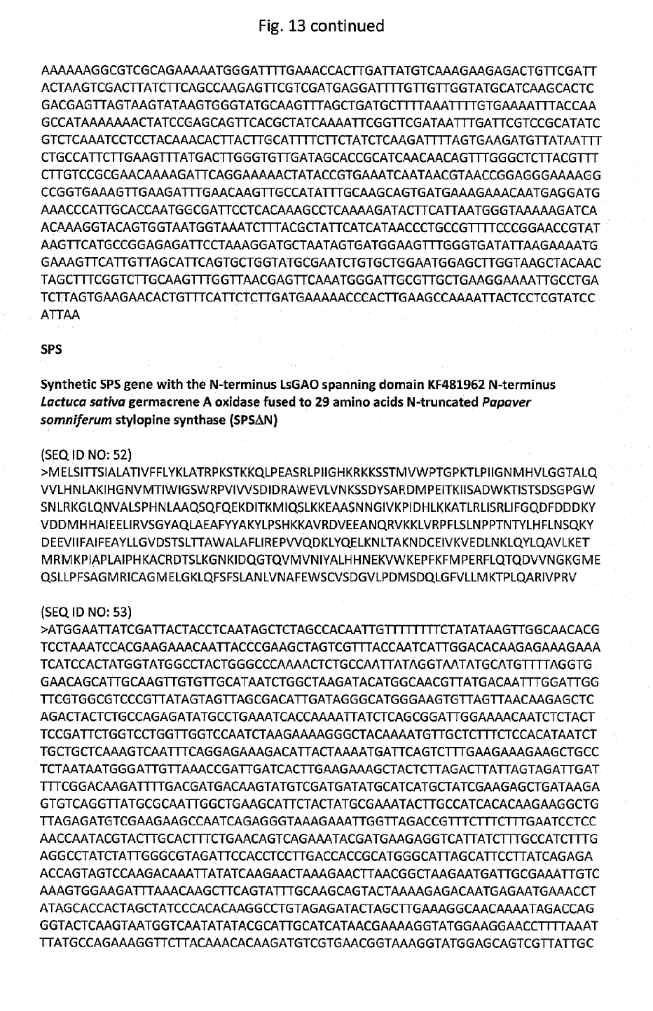

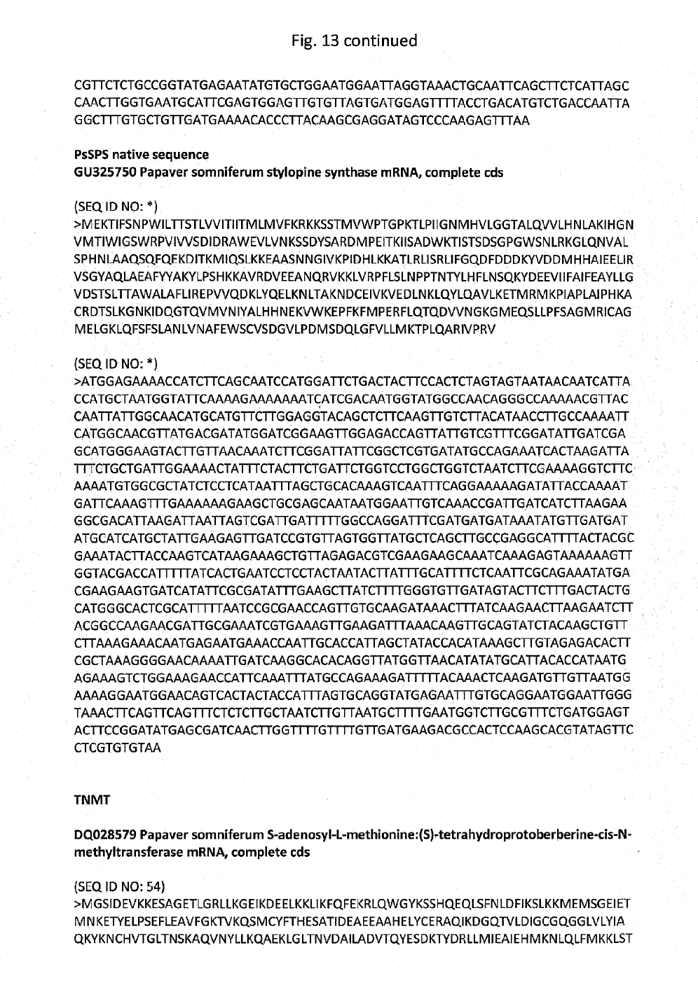

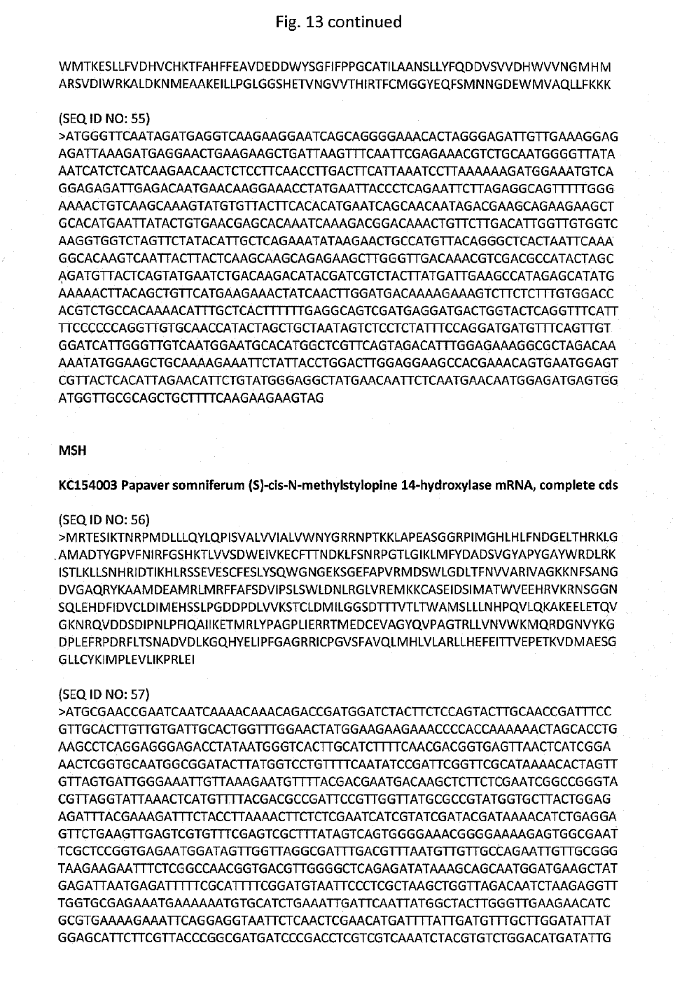

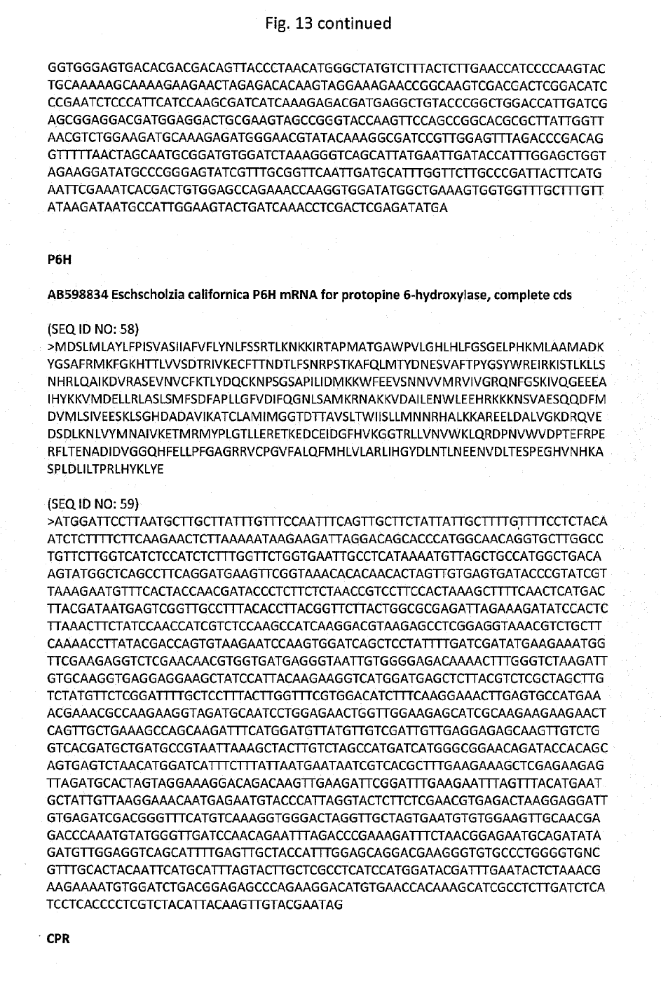

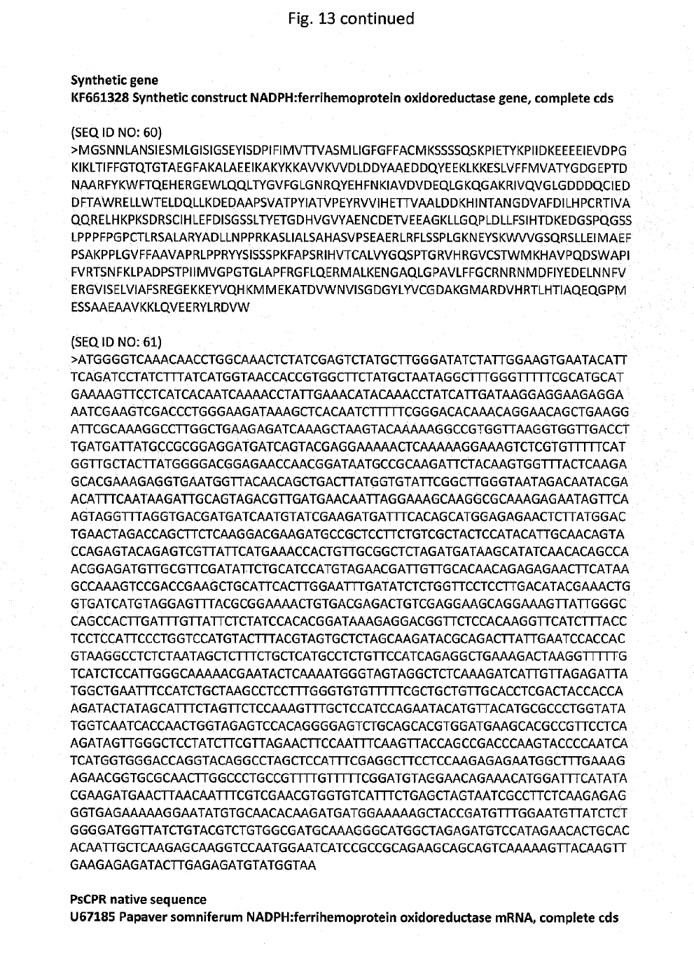

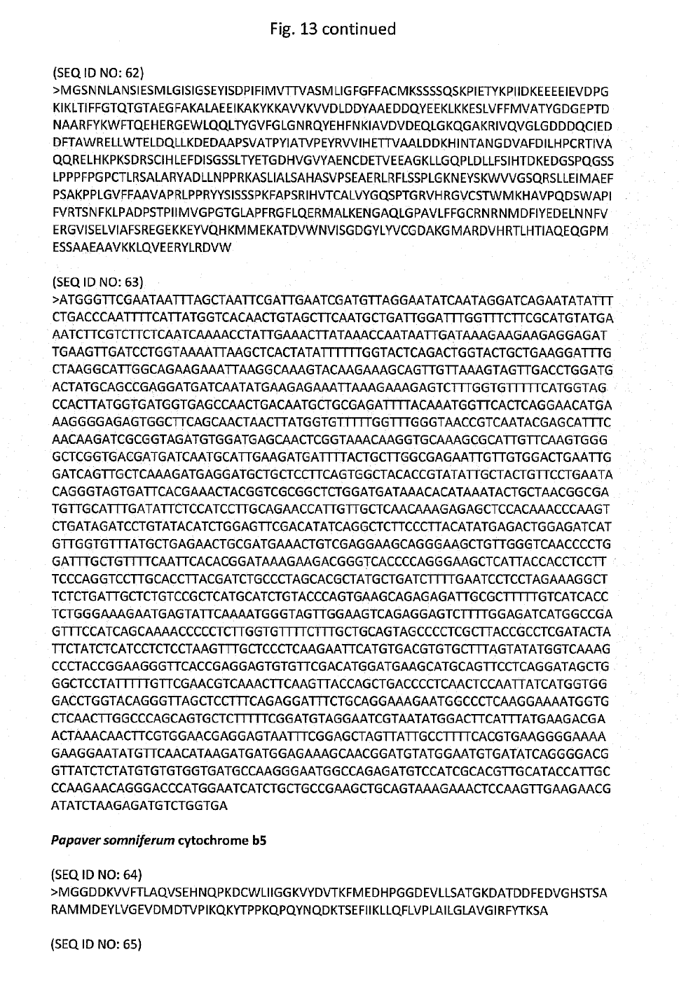

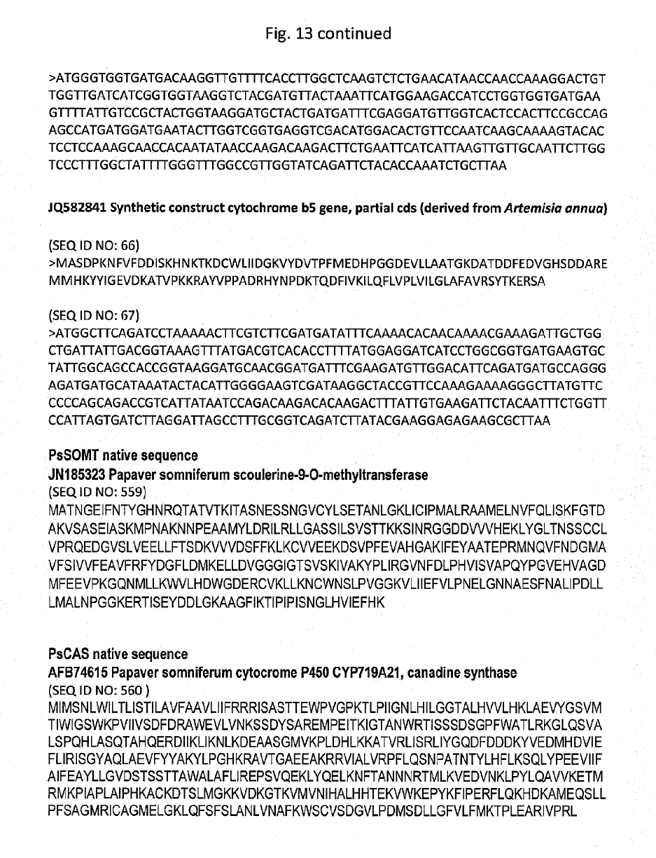

Without being so limited, enzymes encompassed by the present invention include: native or synthetic 6-O-methyltransferase (6OMT); coclaurine-N-methyltransferase (CNMT); 4′-O-methyltransferase 2 (4′OMT2); berberine bridge enzyme (BBE); cheilanthifoline synthase (CFS); stylopine synthase (SPS); protoberberine Ring A closer (e.g., able to convert scoulerine into nandinine and/or cheilanthifoline into stylopine); Ring A closer able to promote production of N-methylcanadine by a high affinity to tetrahydrocolumbamine (e.g., noscapine pathway), protoberberine Ring B closer (e.g., able to convert scoulerine into cheilanthifoline and/or nandinine into stylopine); tetrahydroprotoberberine cis-N-methyltransferase (TNMT); (S)-cis-N-methylstylopine 14-hydroxylases (MSH); protopine 6-hydroxylase (P6H); cytochrome P450 reductase (CPR); cytochrome b5 and dihydrobenzophenanthridine oxidase (DBOX). Useful enzymes for the present invention may be isolated from Papaver somniferum, Eschscholzia californica, other Papaveraceae (e.g., Papaver bracteatum, Sanguinaria canadensis, Chelidonium majus, Stylophorum diphyllum, Glaucium flavum, Argemone mexicana and Corydalis cheilanthifolia), Ranunculaceae (e.g., Thalictrum flavum, Aquilegia Formosa, Hydrastis canadensis, Nigella sativa, Xanthorhiza simplicissima and Coptis japonica), Berberidaceae (e.g., Berberis thunbergii, Mahonia aquifolium, Jeffersonia diphylla, and Nandina domestica), or Menispermaceae (e.g., Menispermum canadense, Cissampelos mucronata, Tinospora cordifolia, and Cocculus trilobus), etc. The truncated (e.g., devoid of transmembrane domains) and full amino acid sequences of illustrative examples of these enzymes are presented in Figs. herein (e.g., FIGS. 13 to 17).

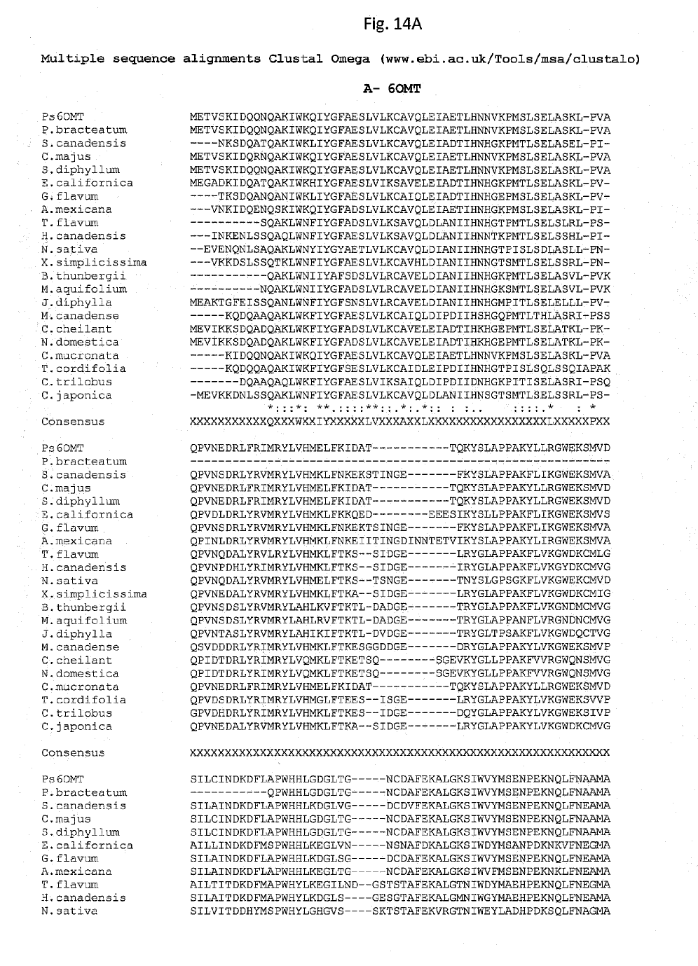

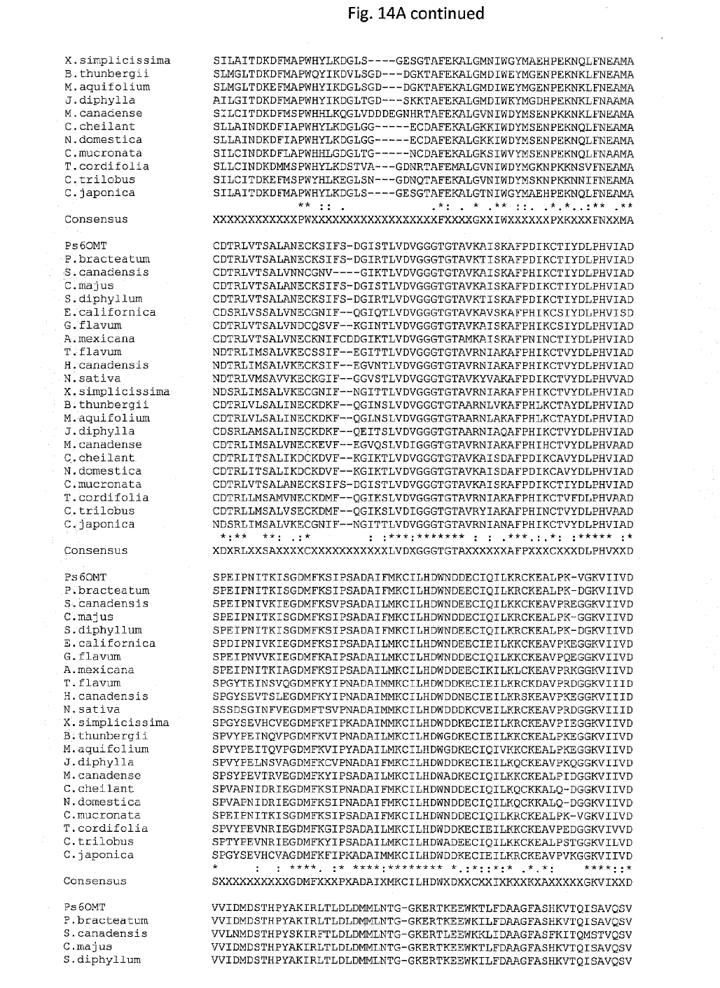

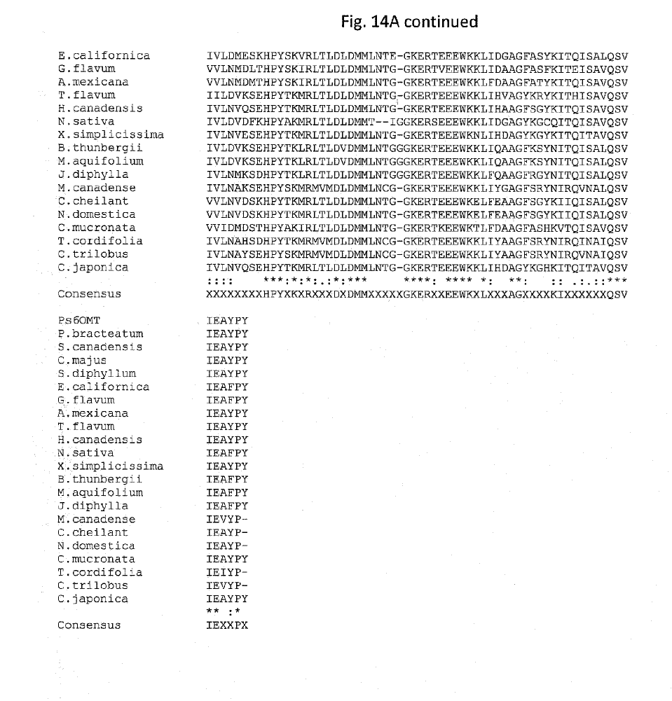

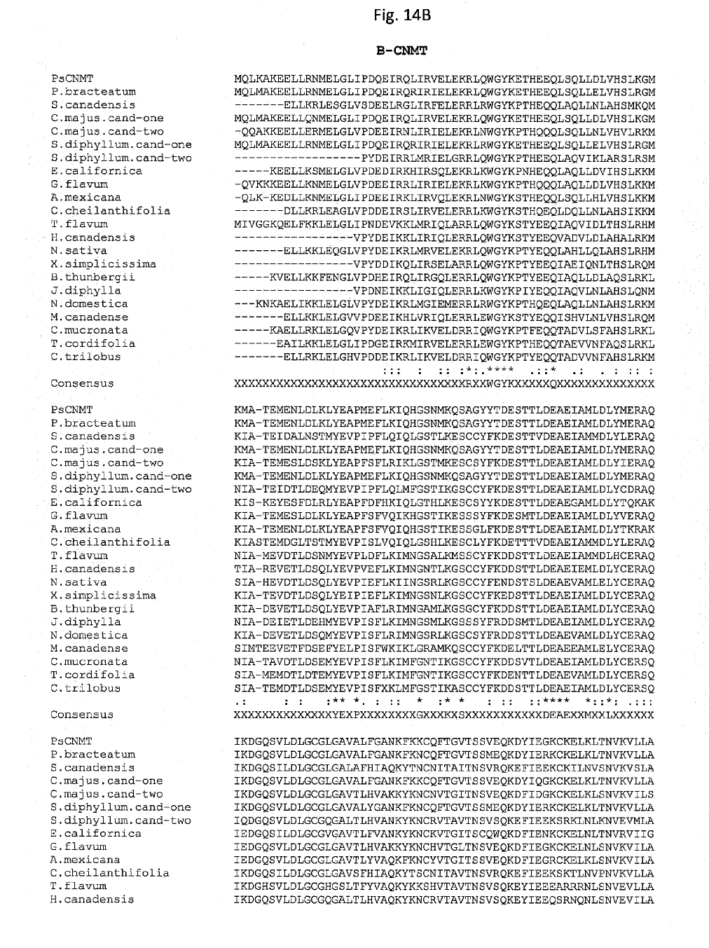

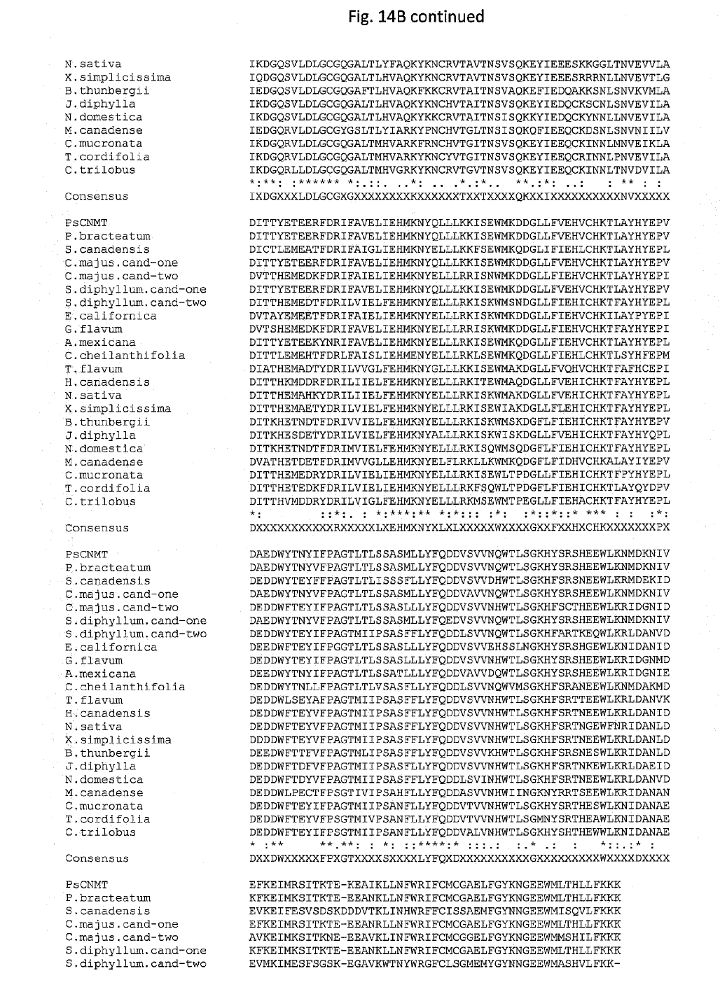

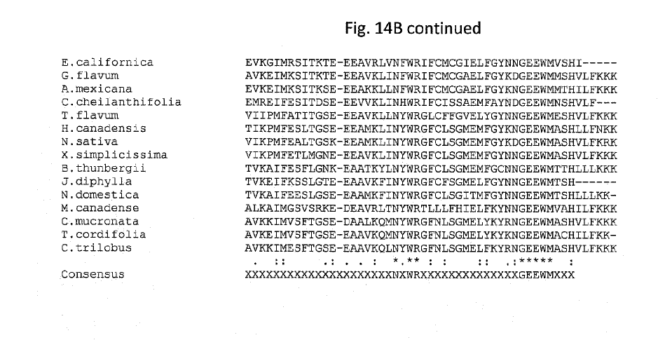

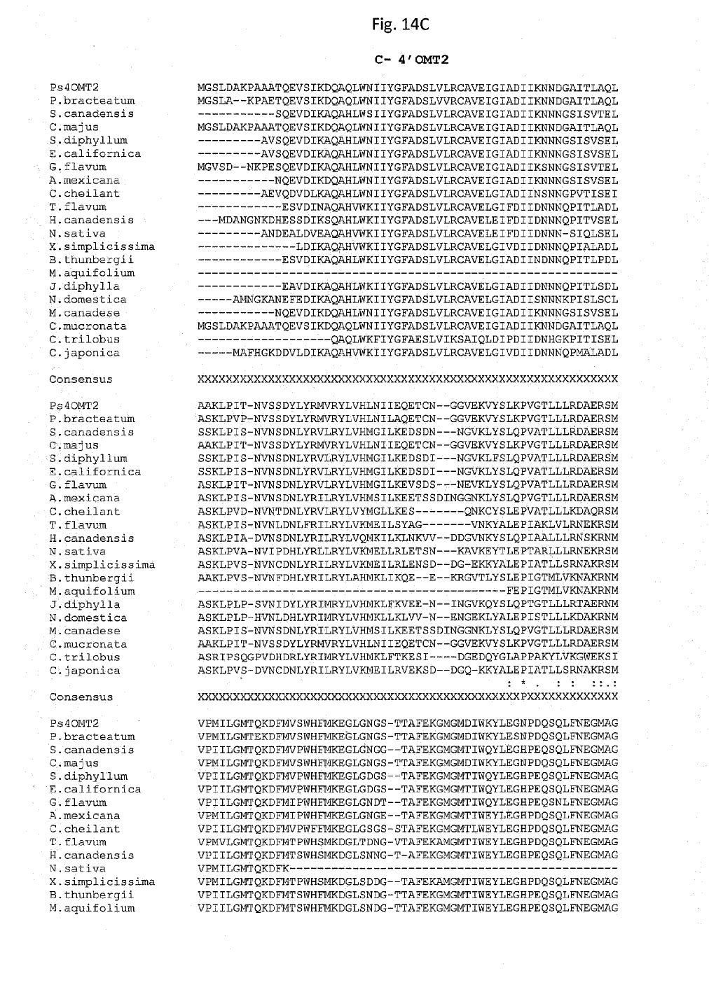

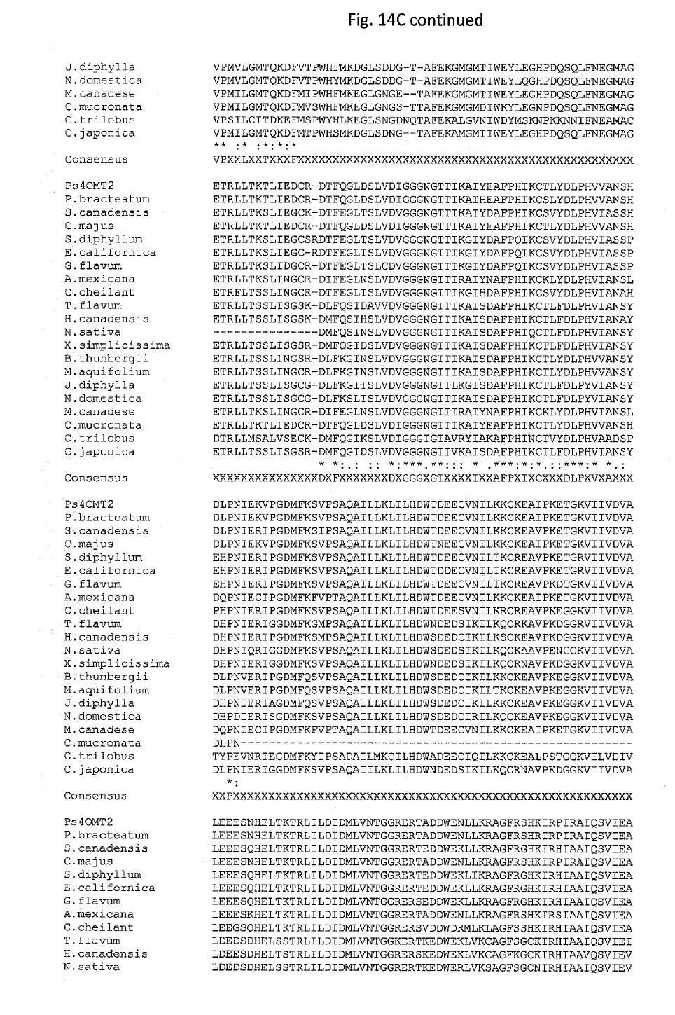

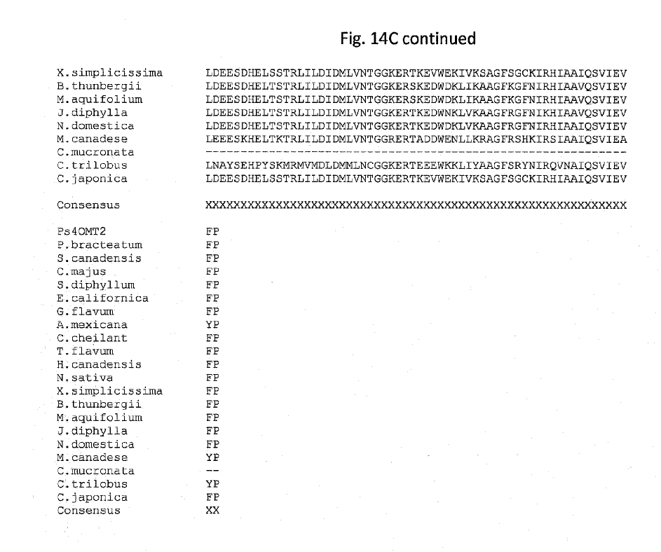

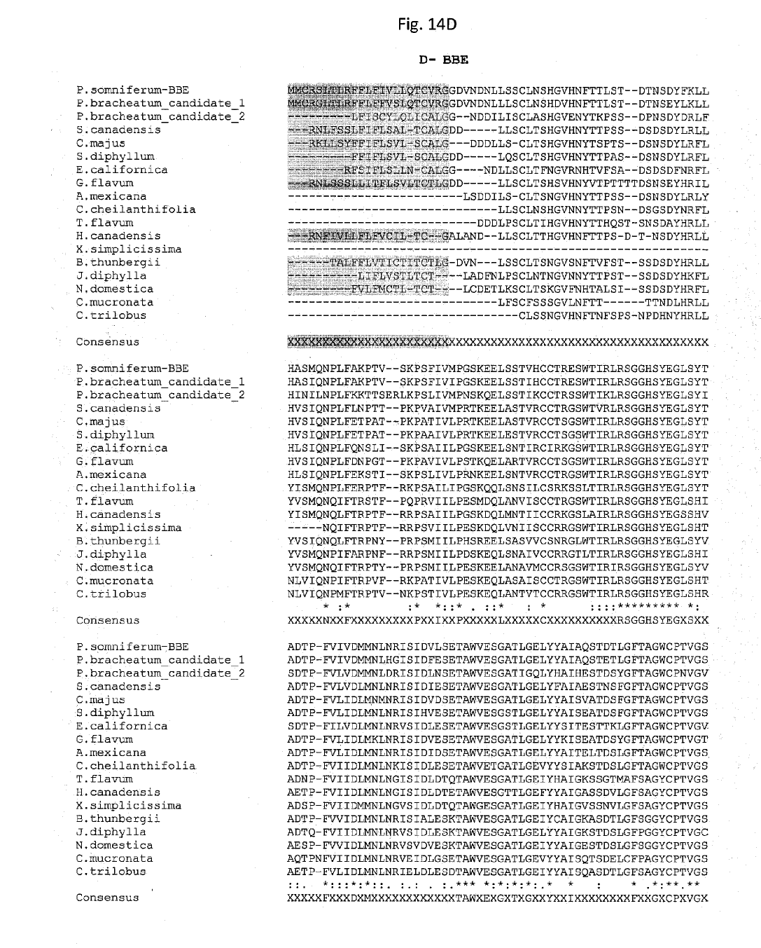

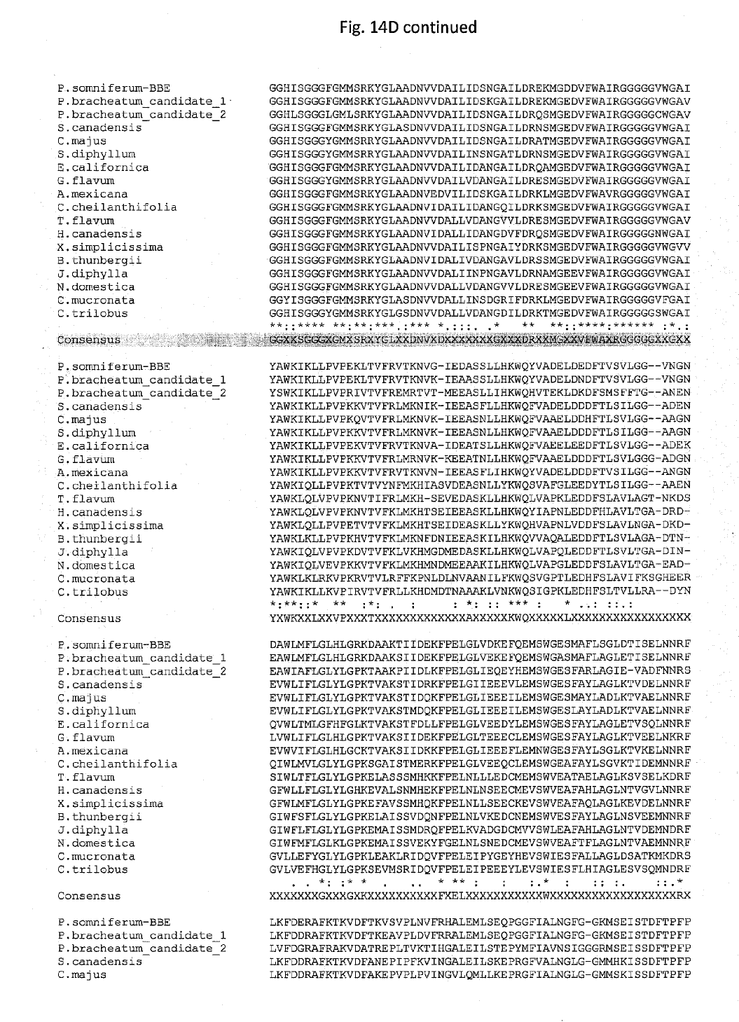

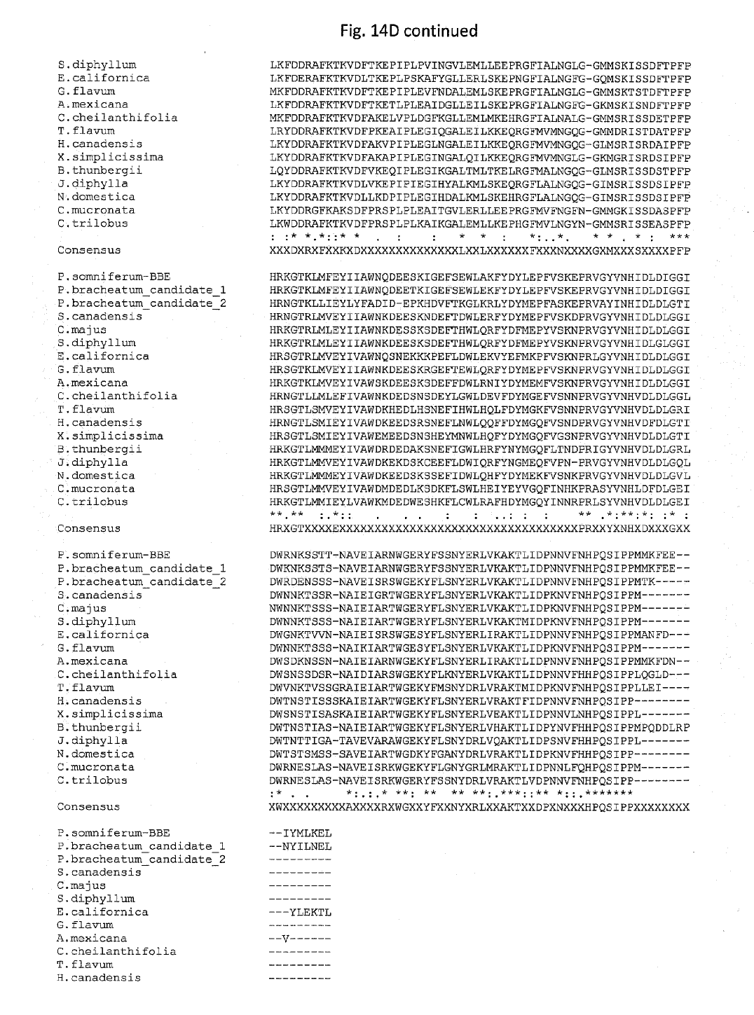

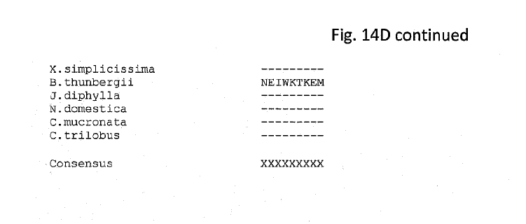

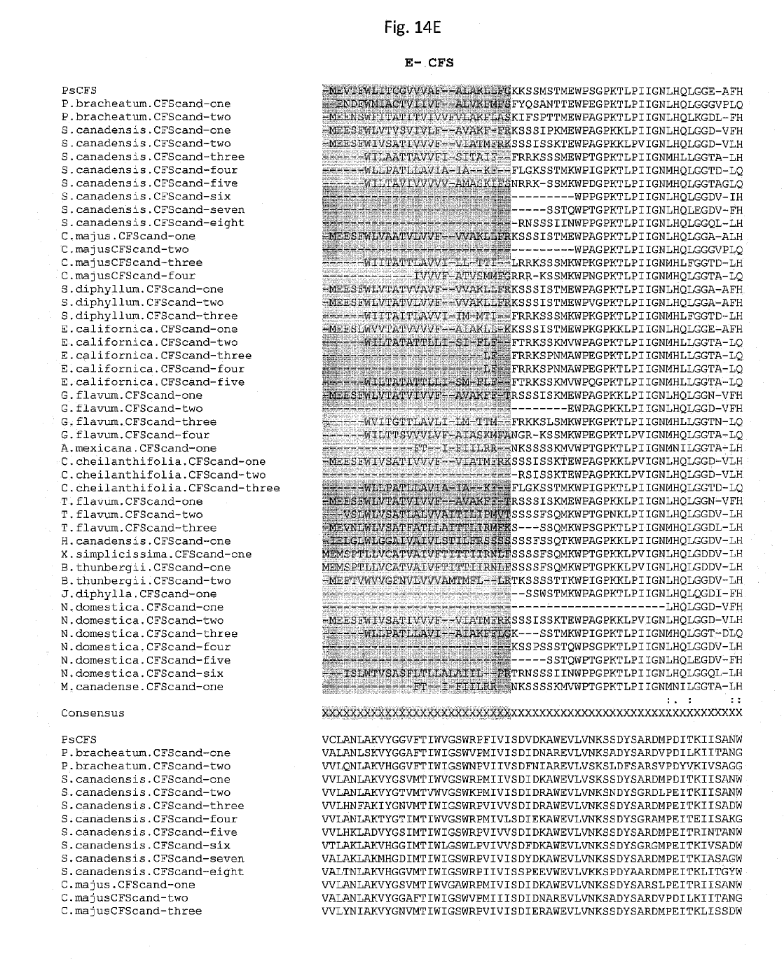

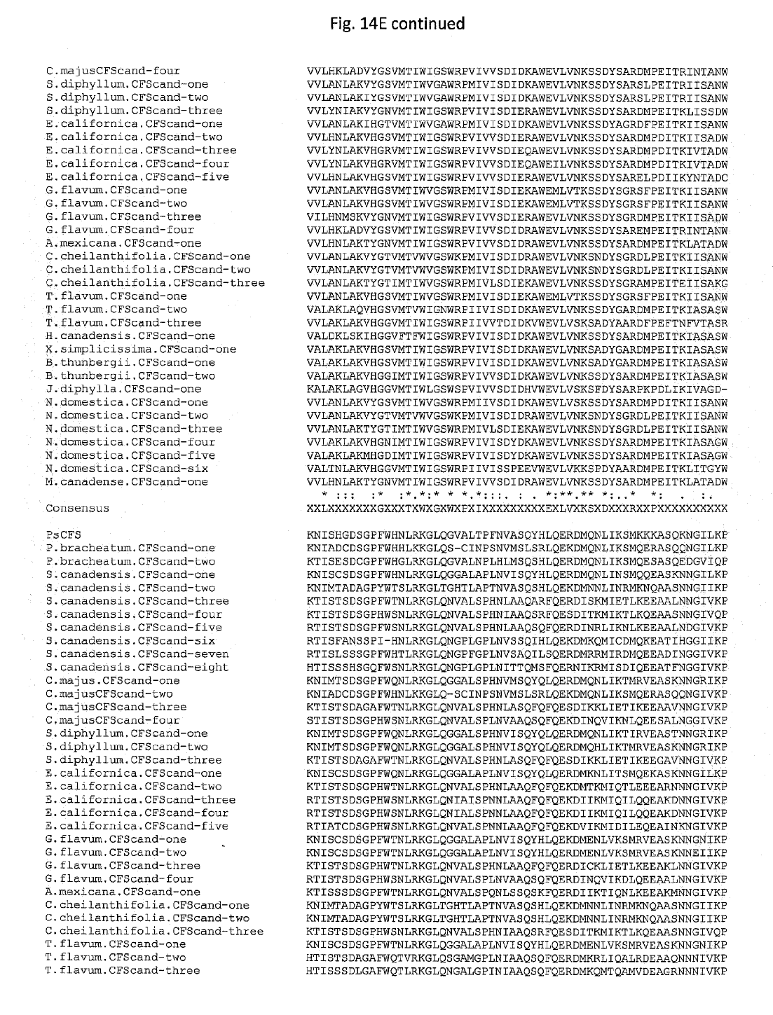

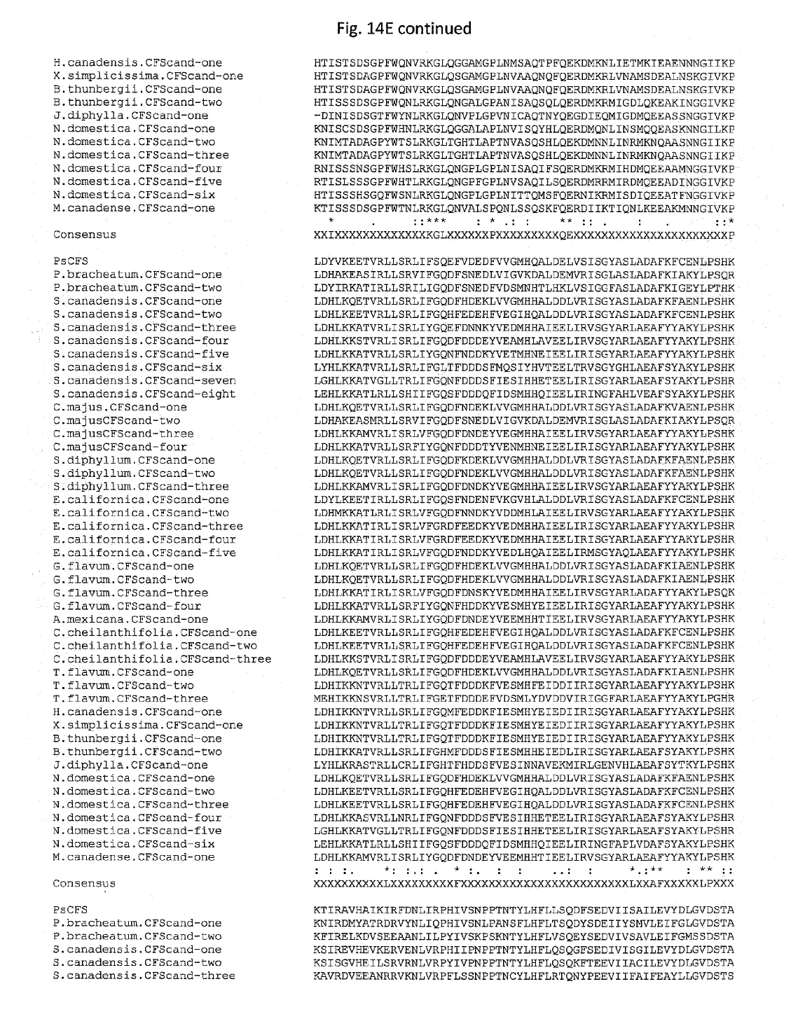

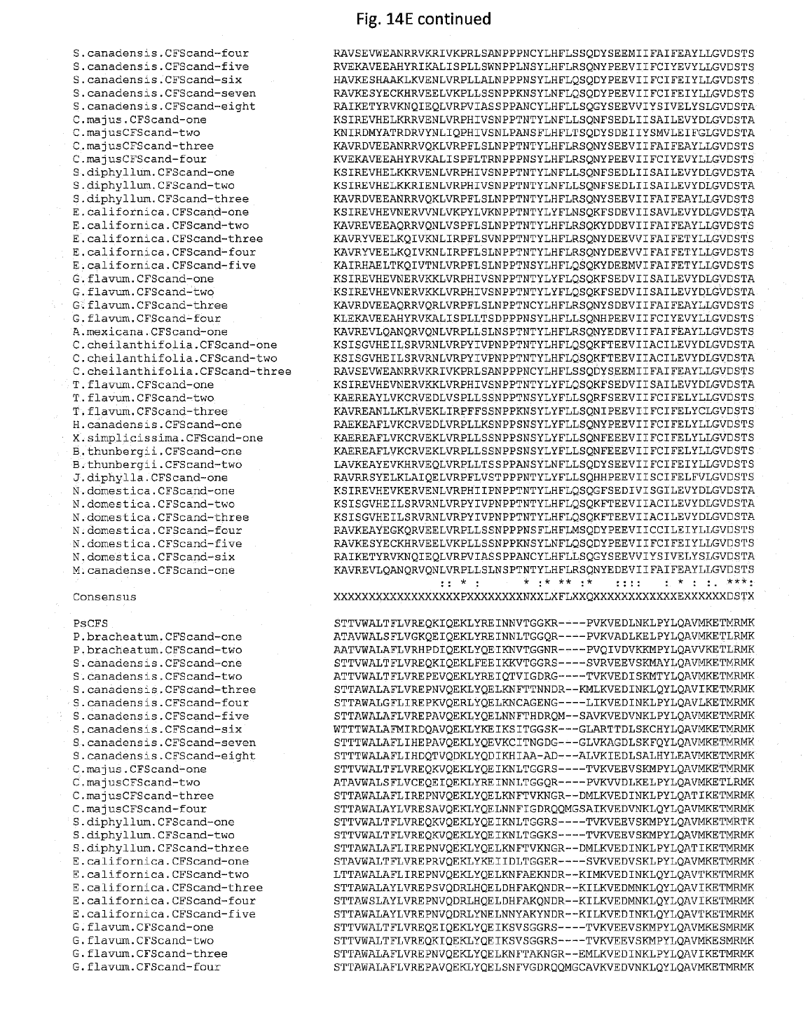

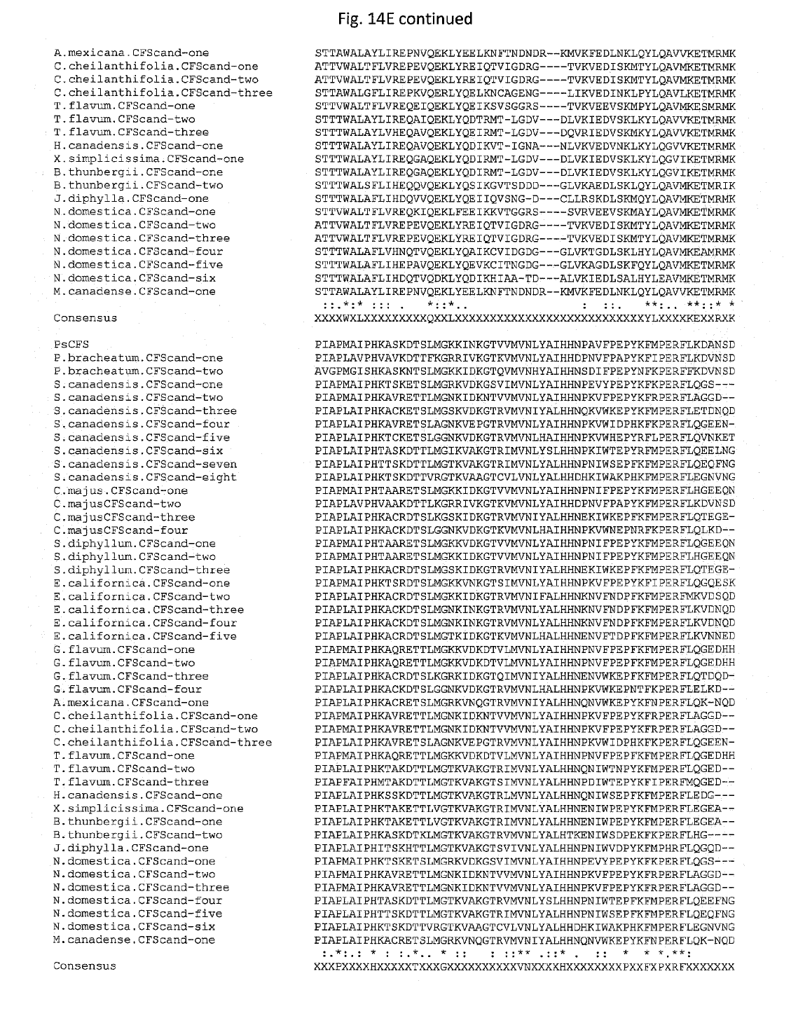

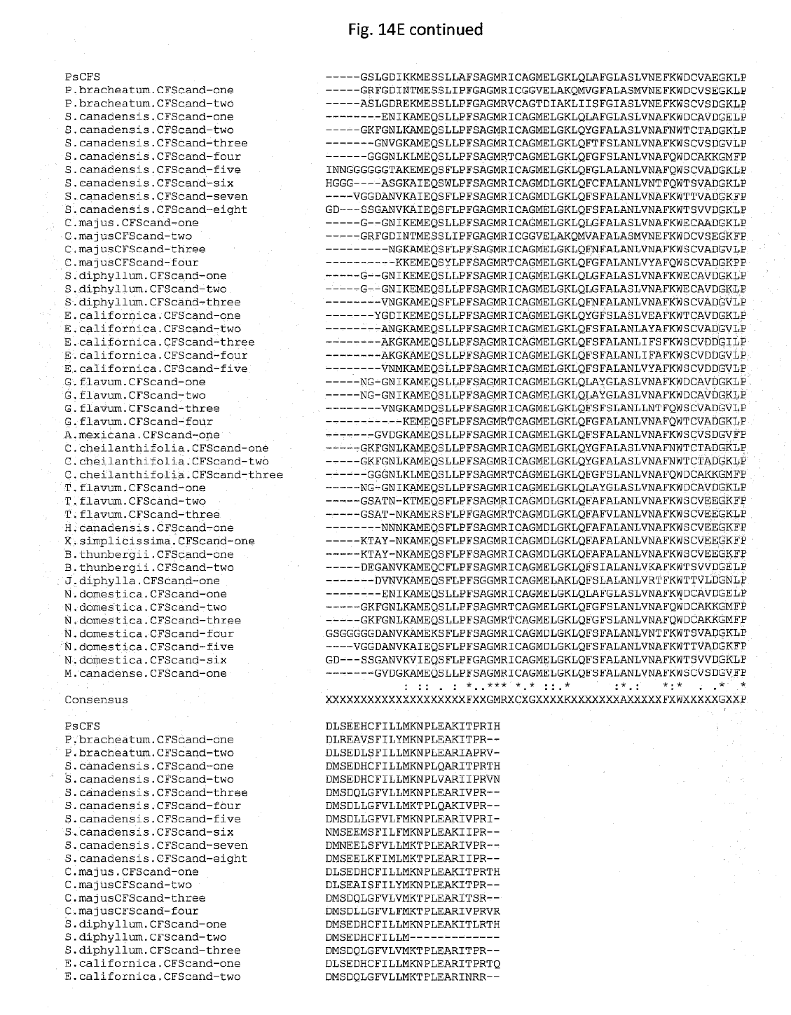

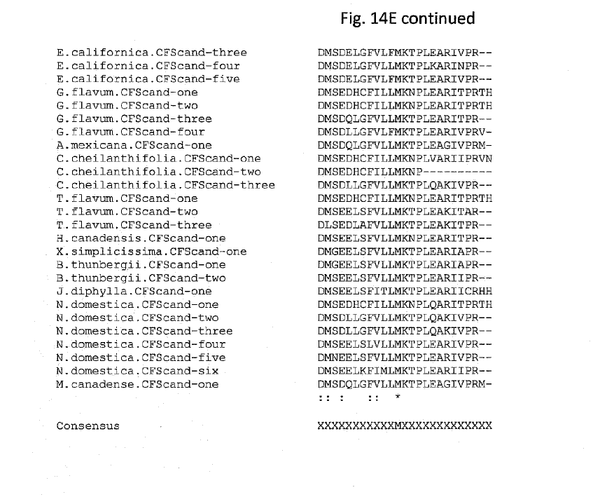

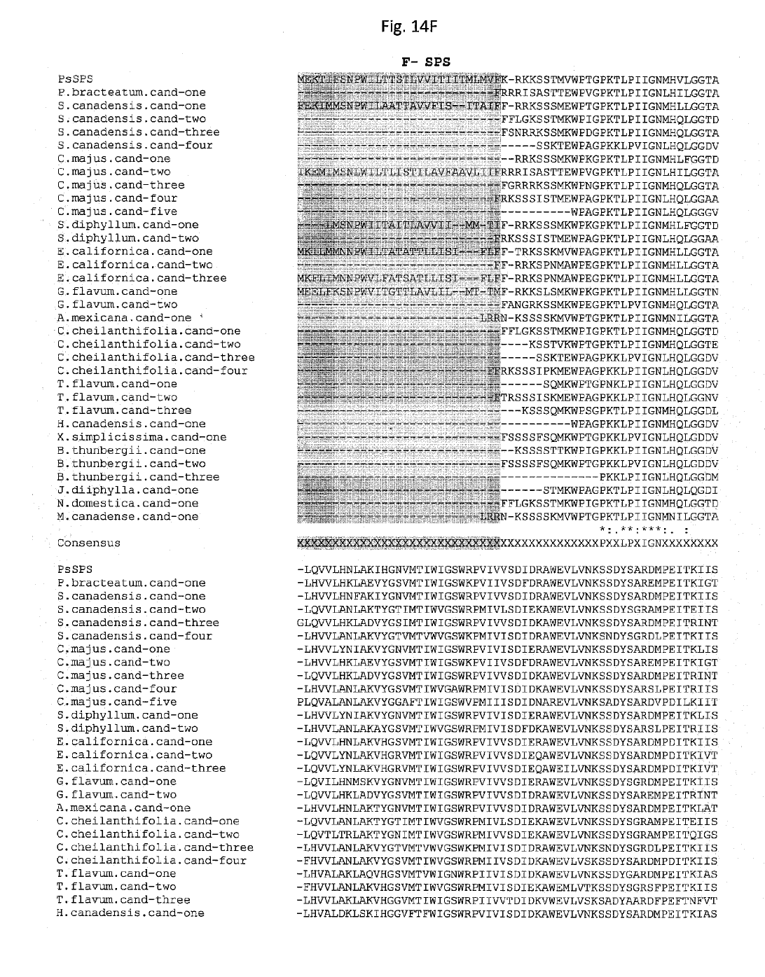

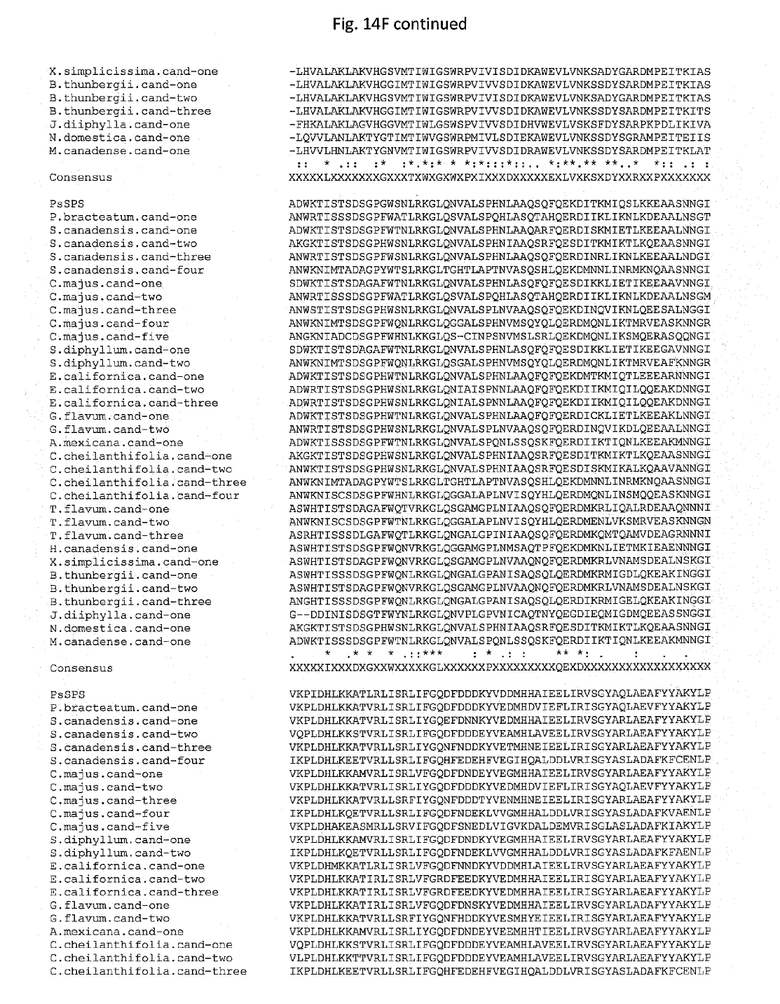

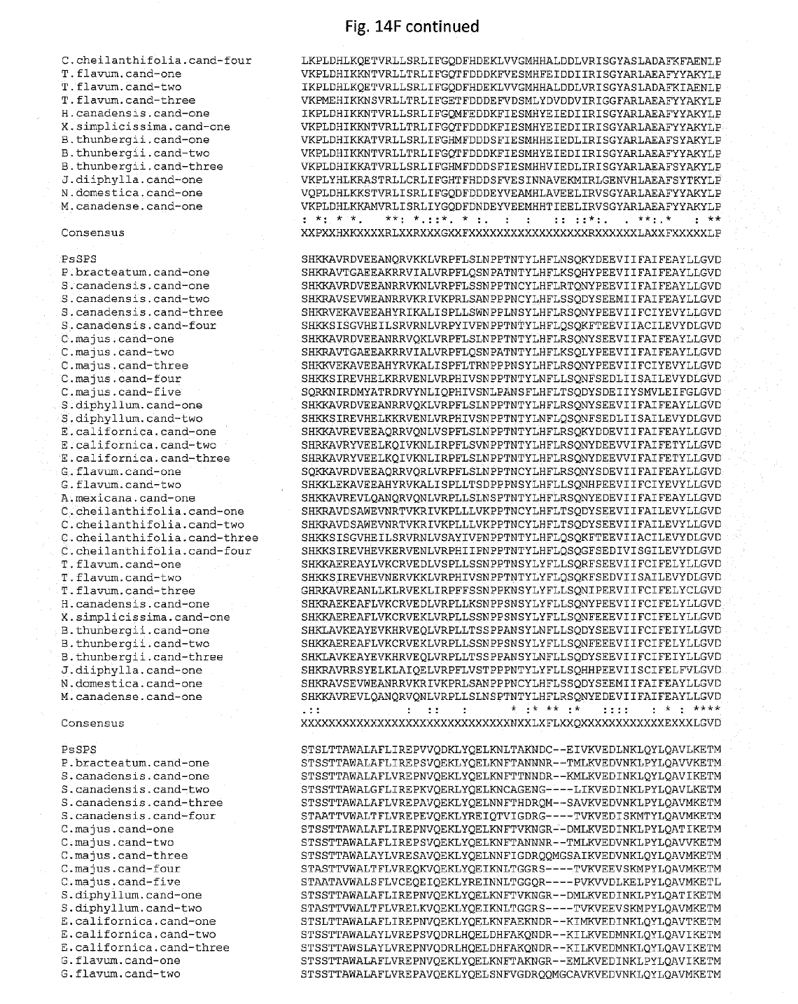

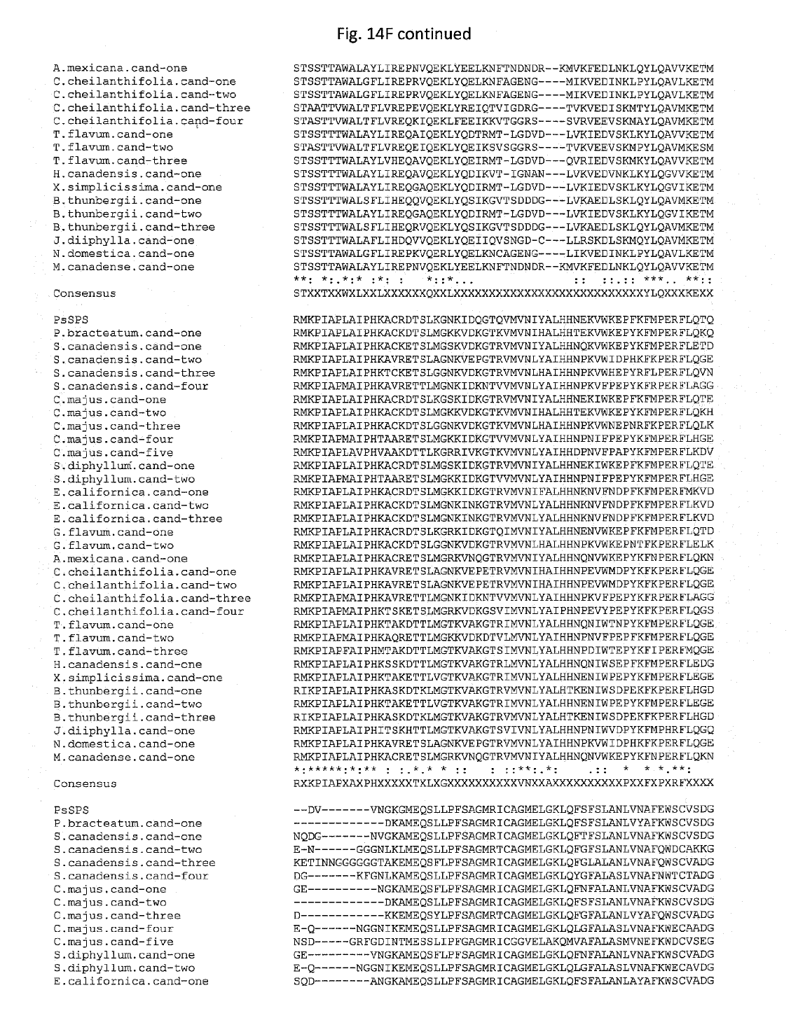

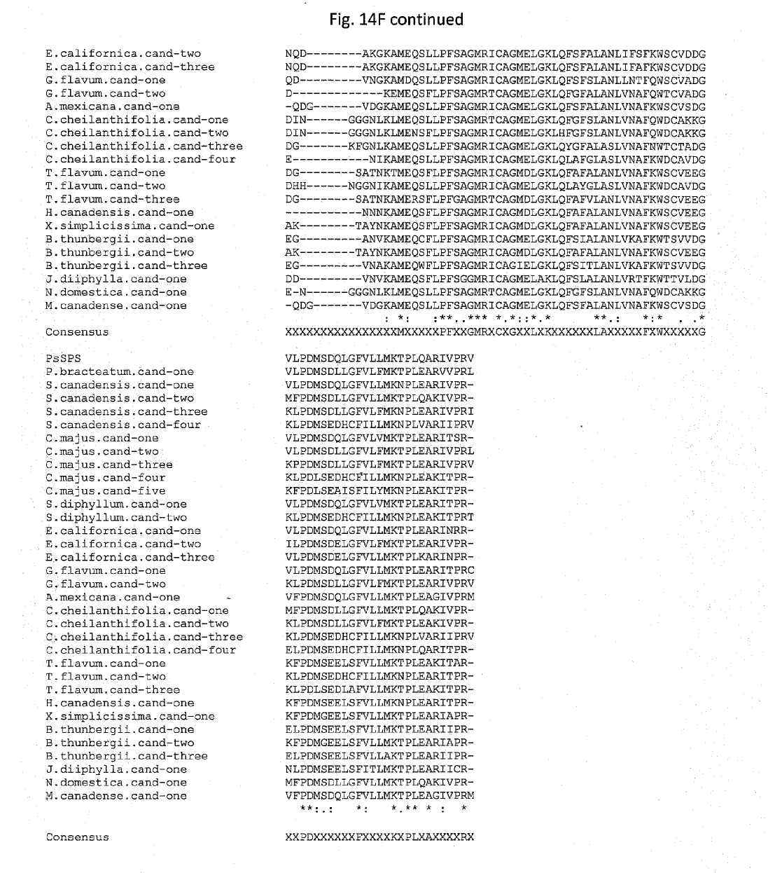

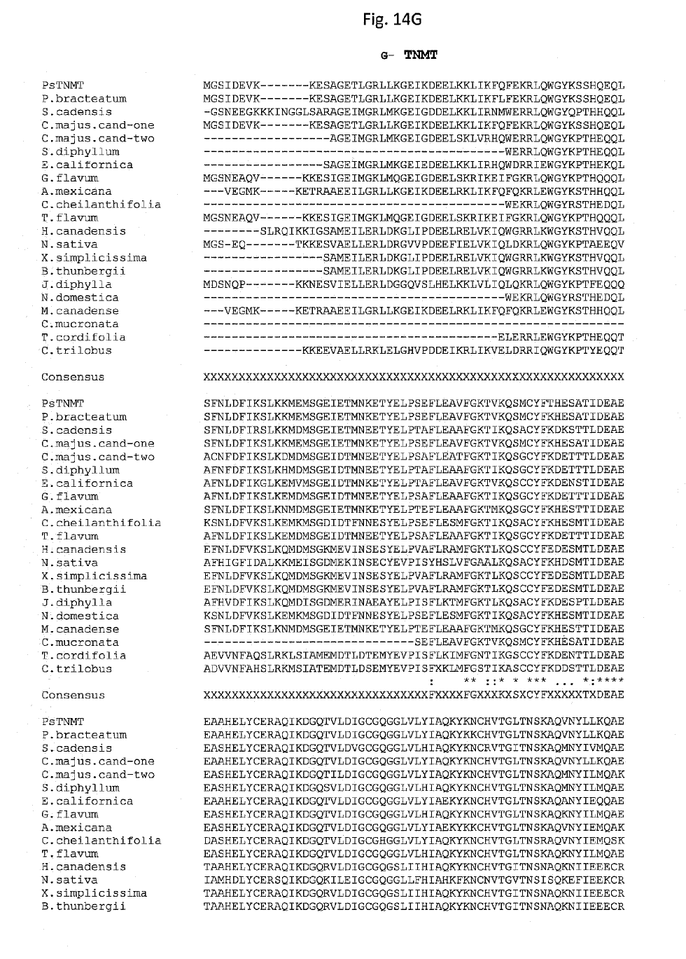

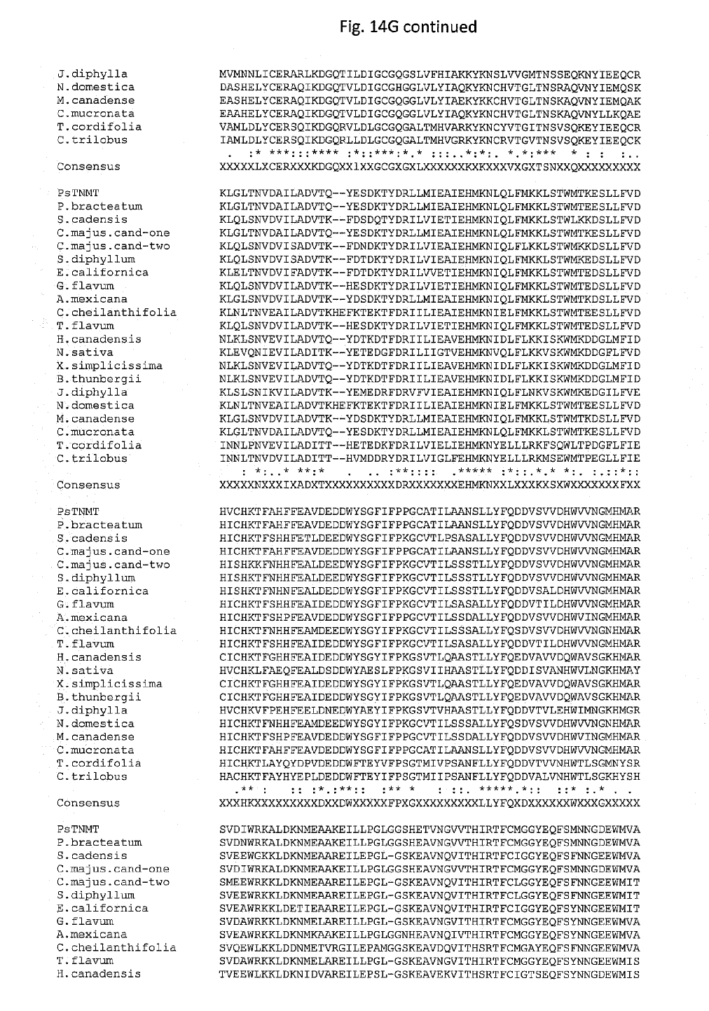

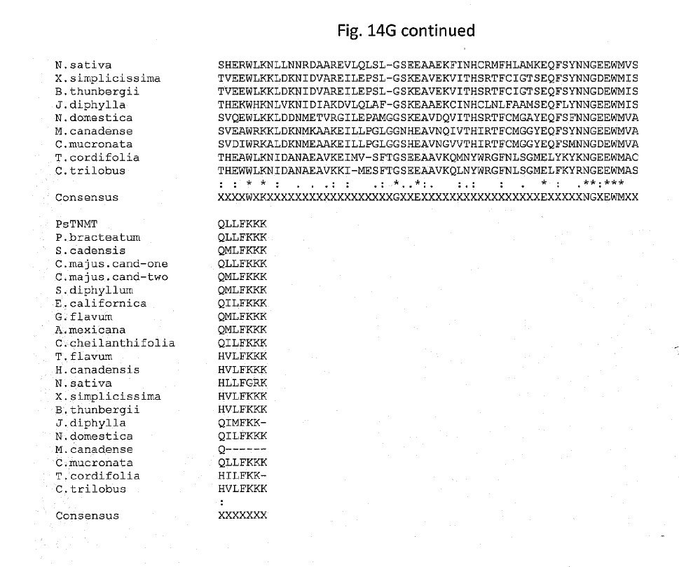

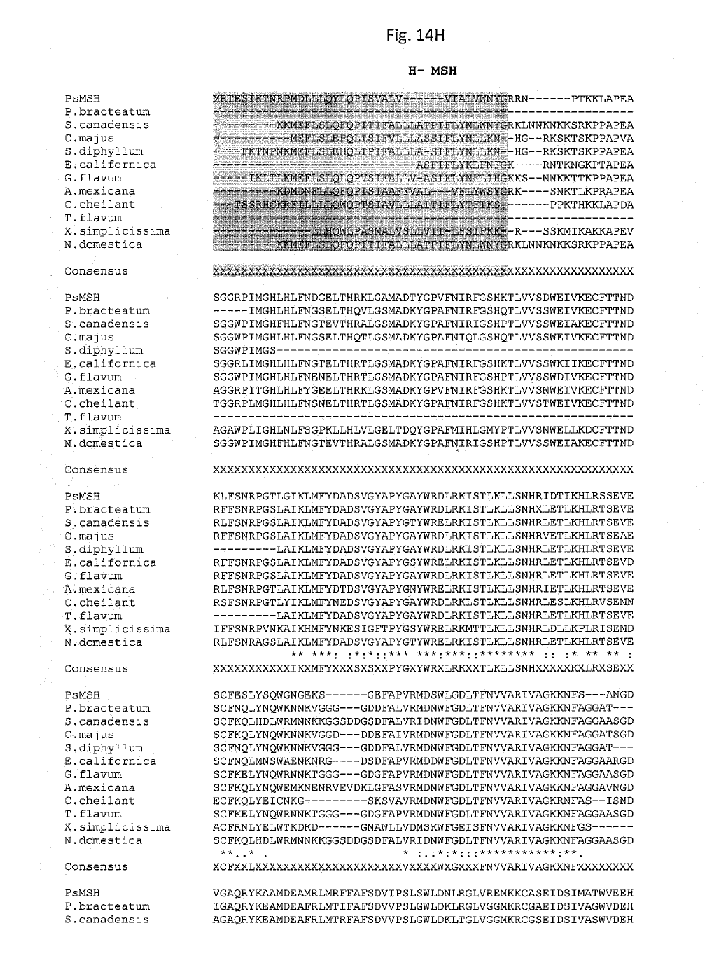

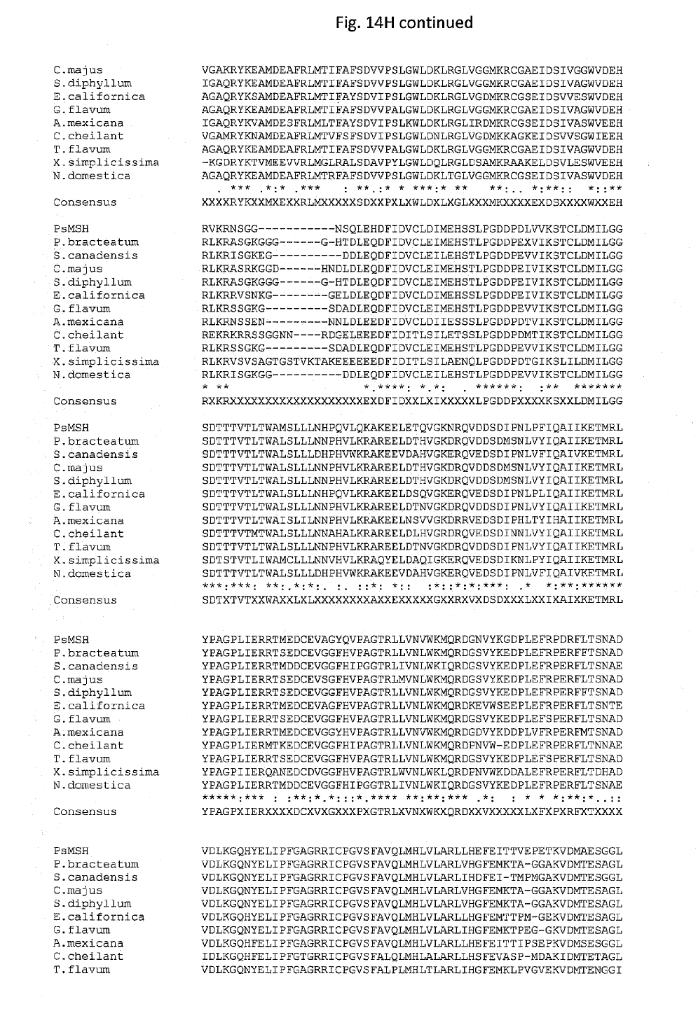

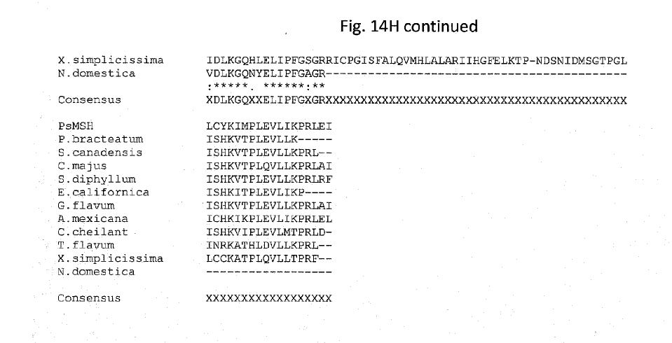

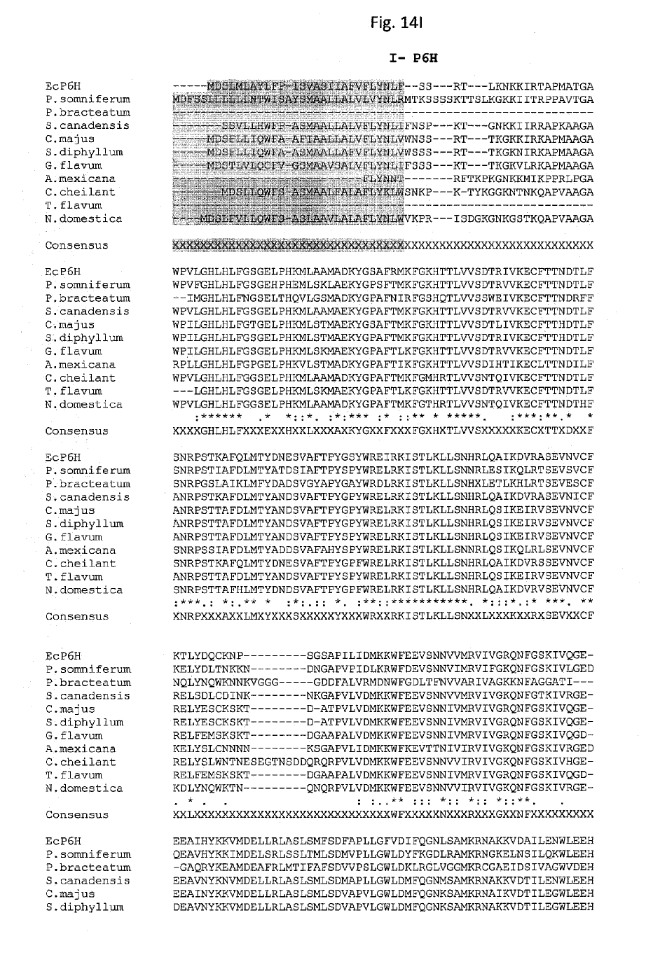

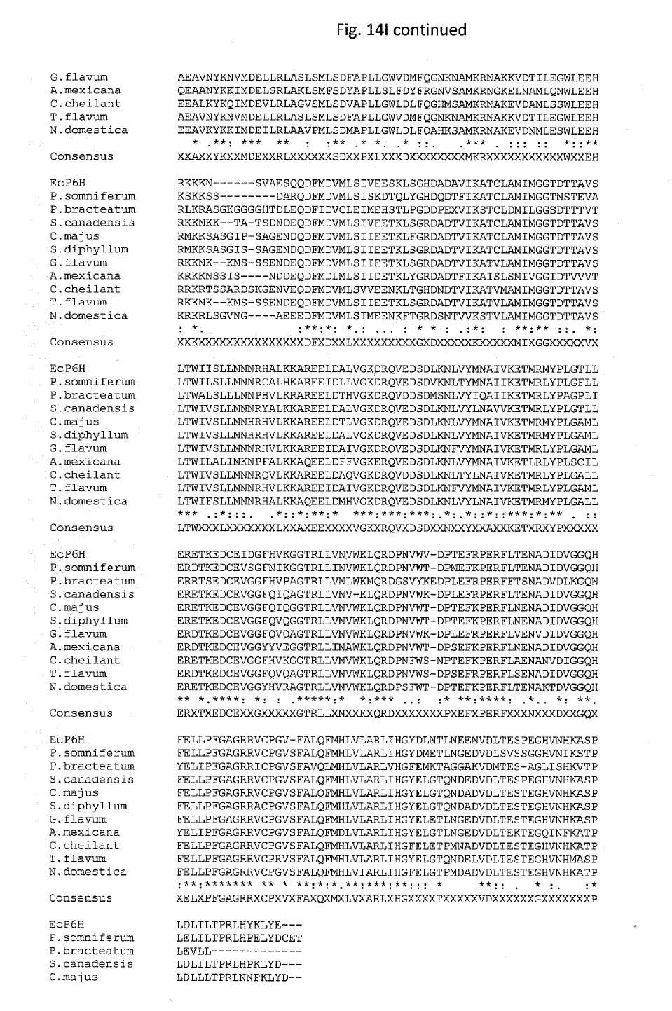

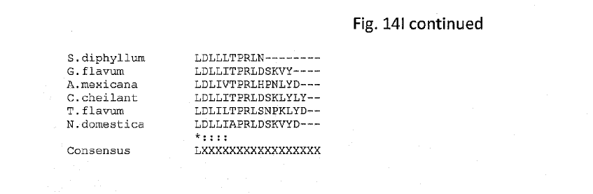

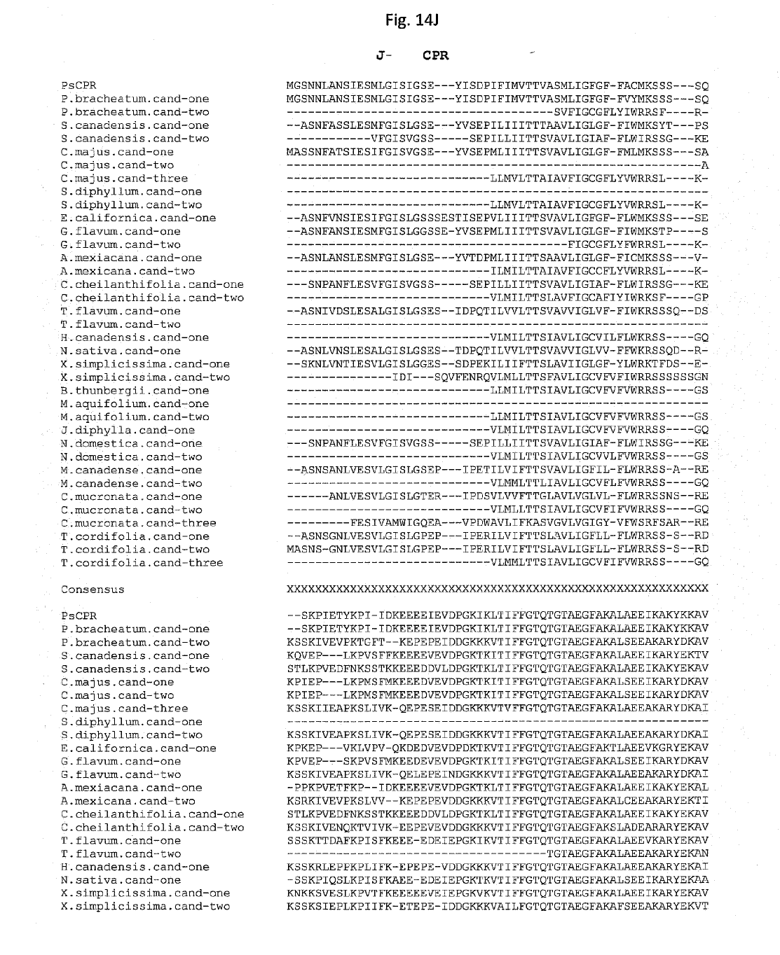

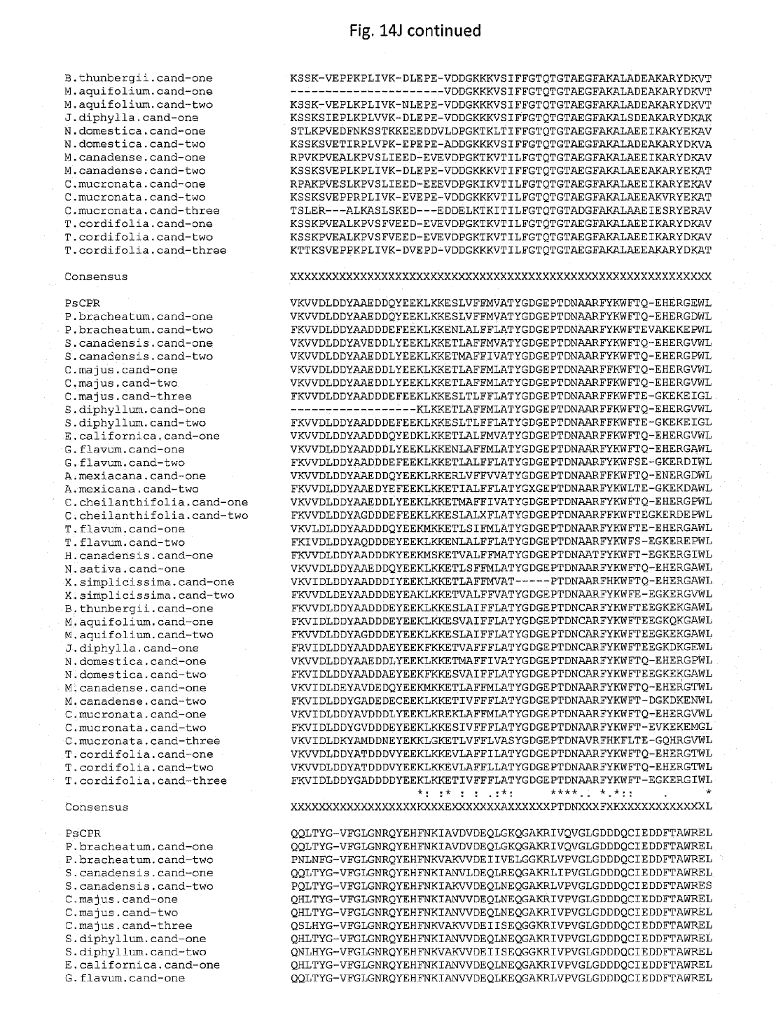

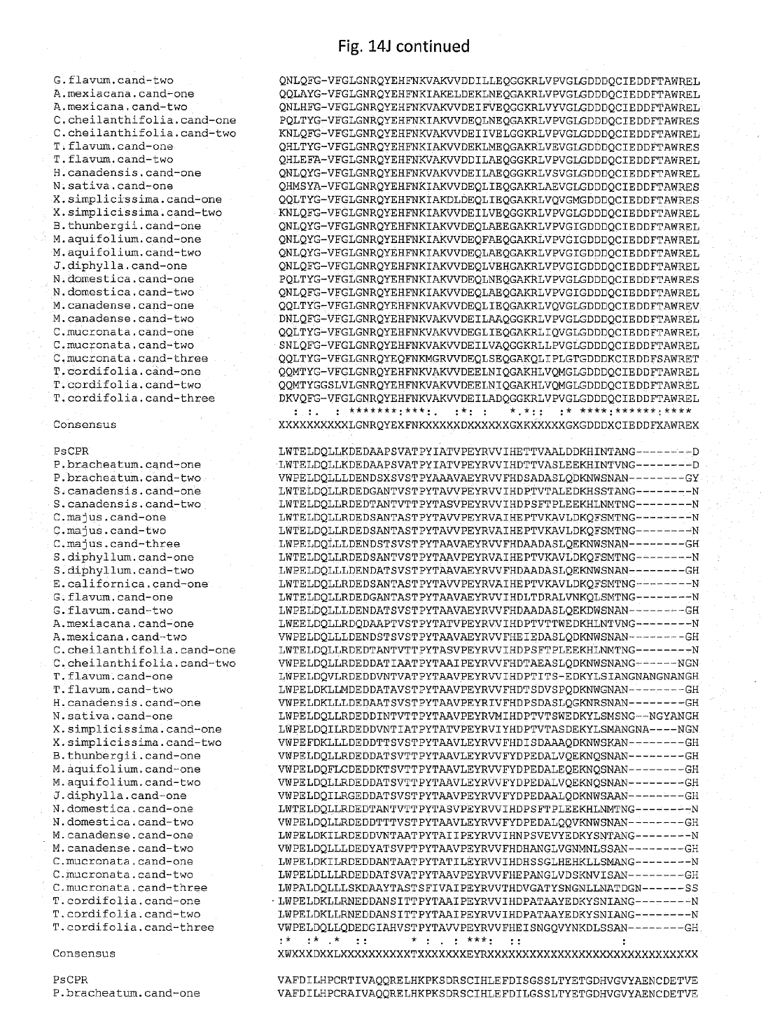

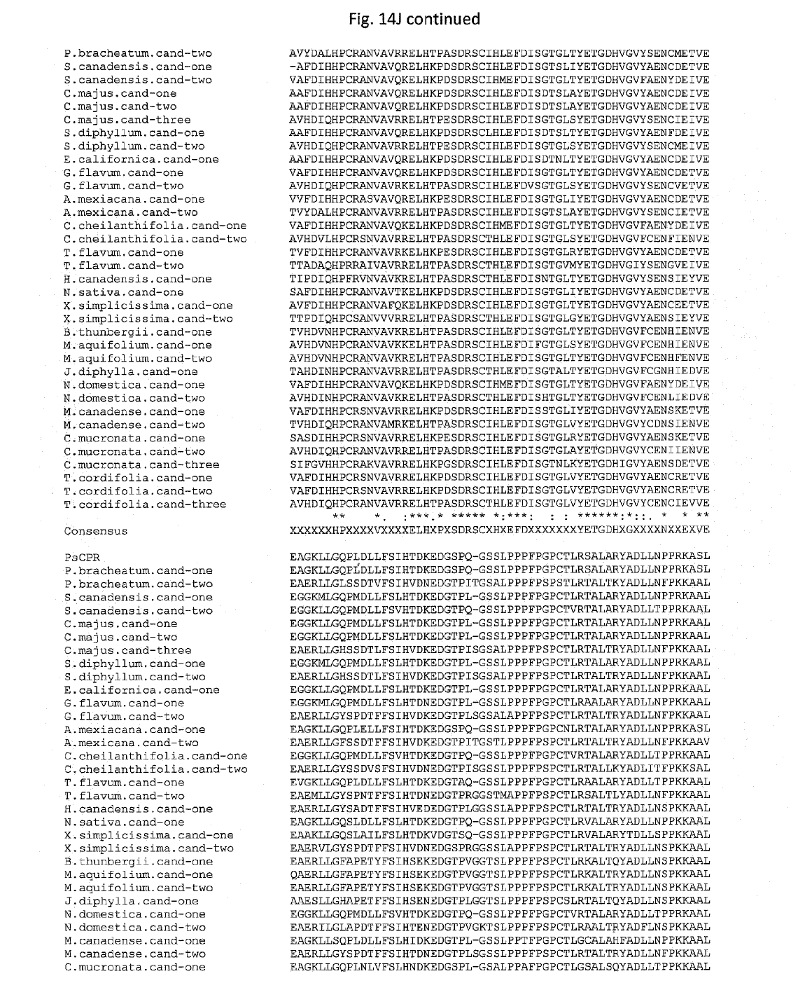

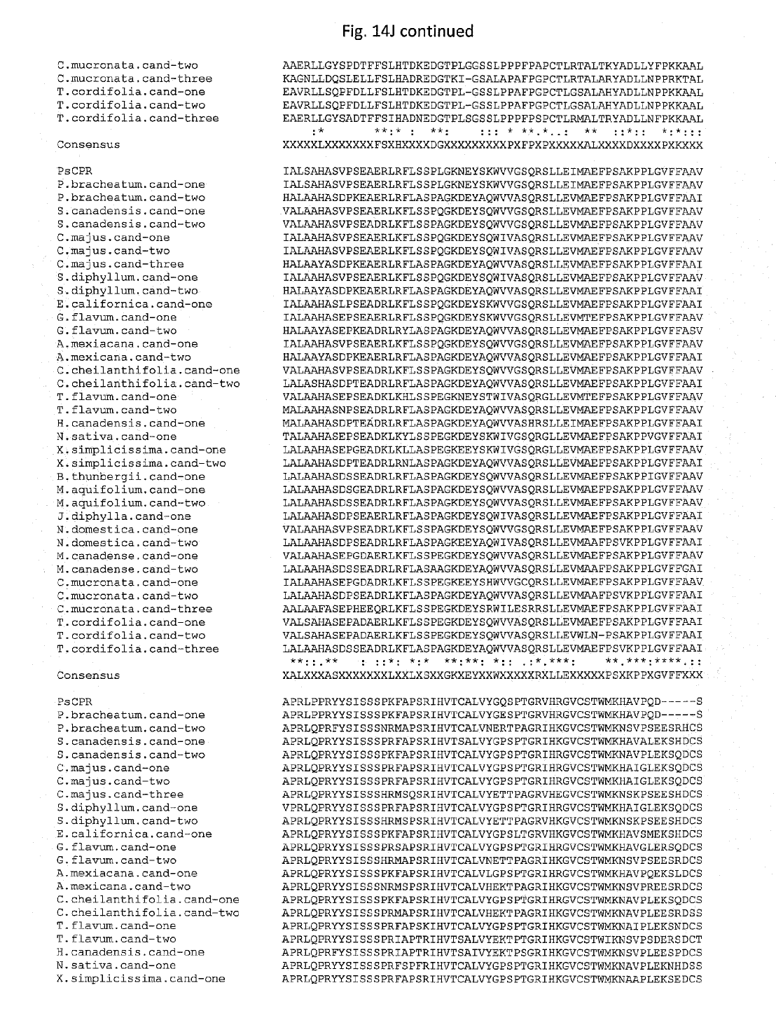

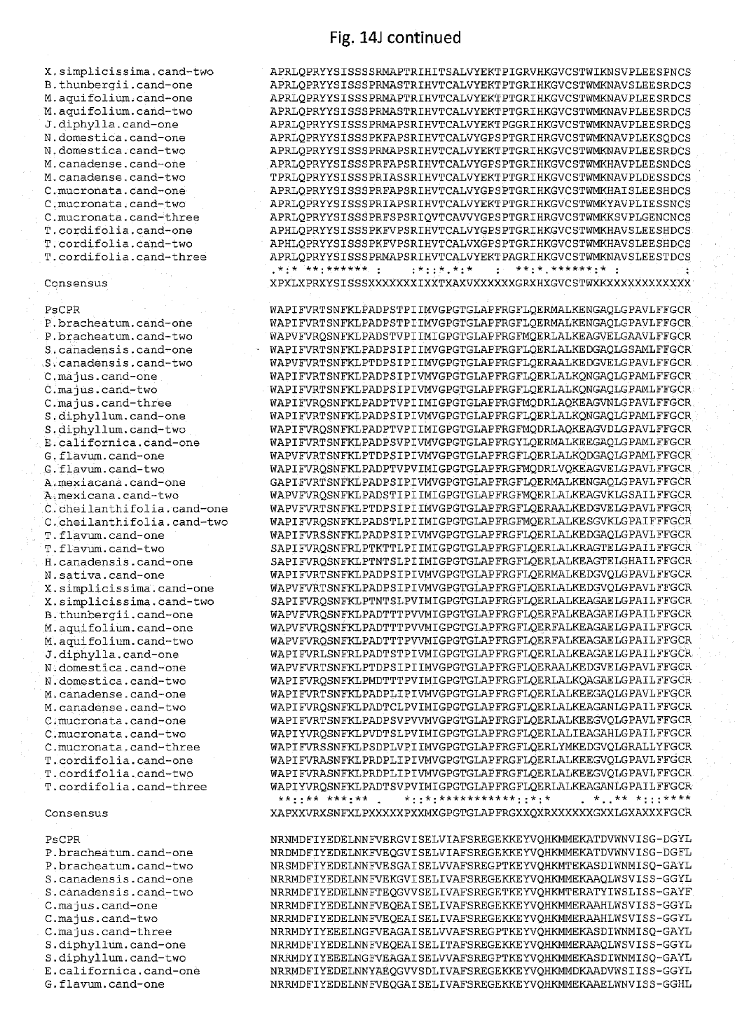

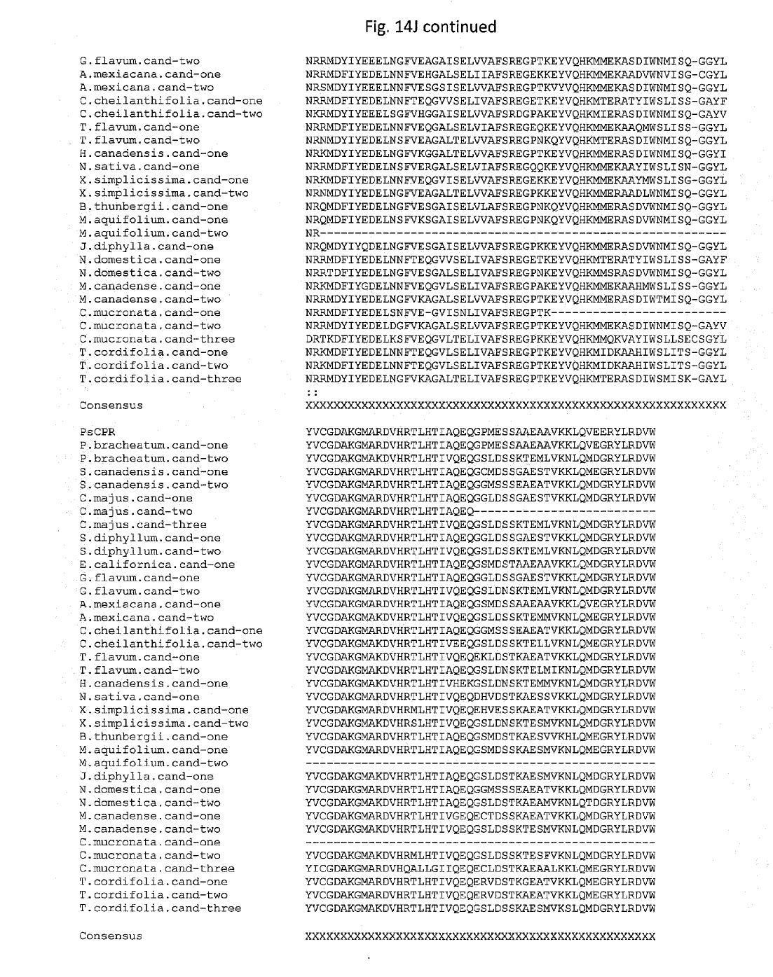

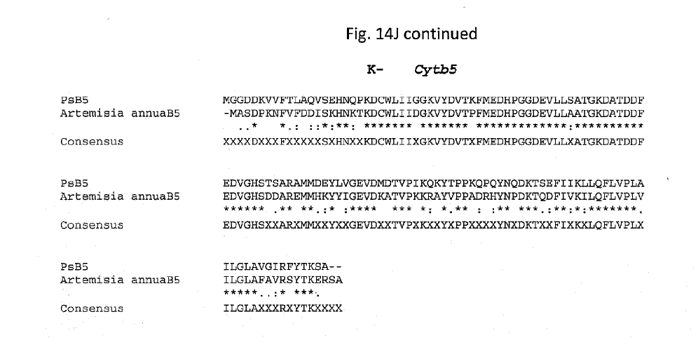

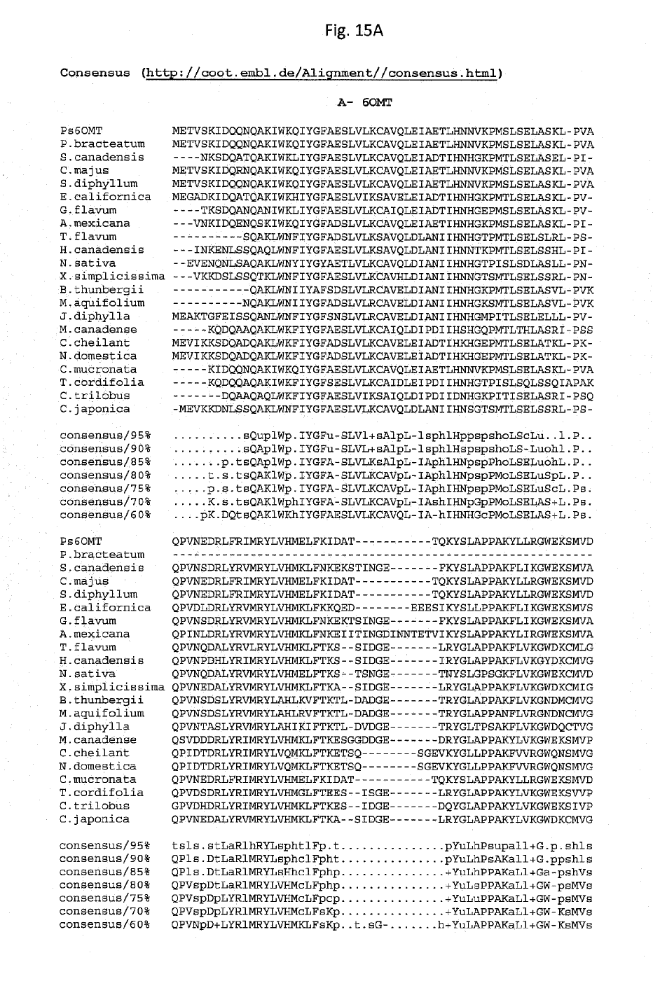

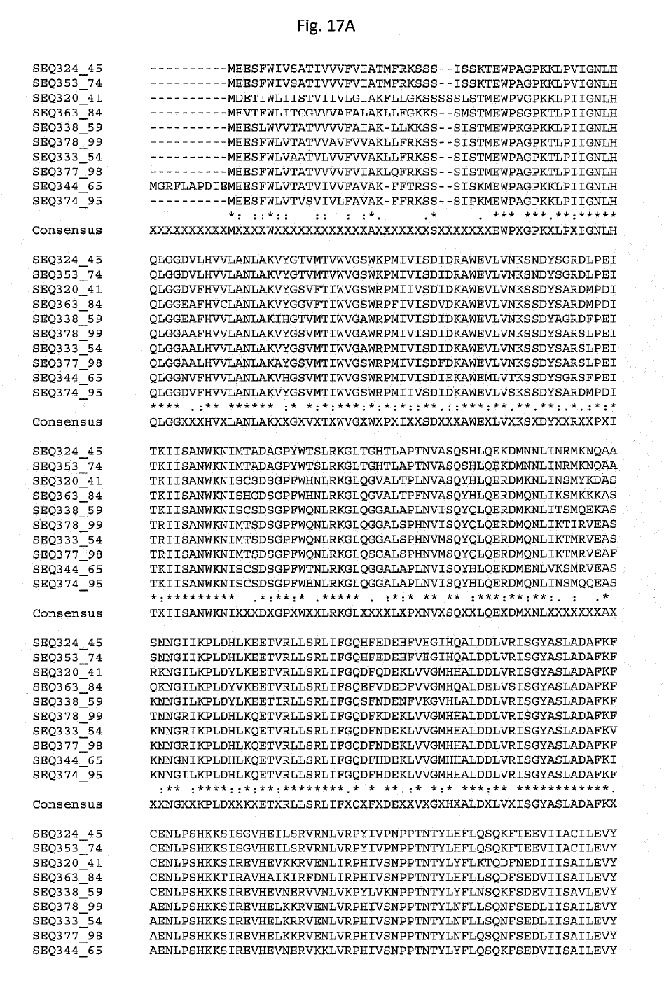

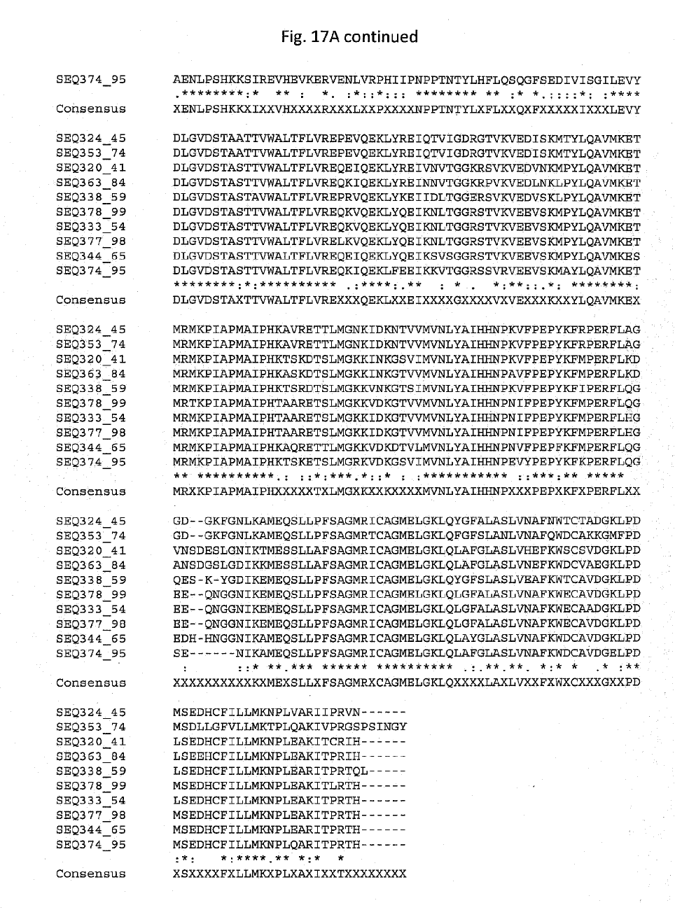

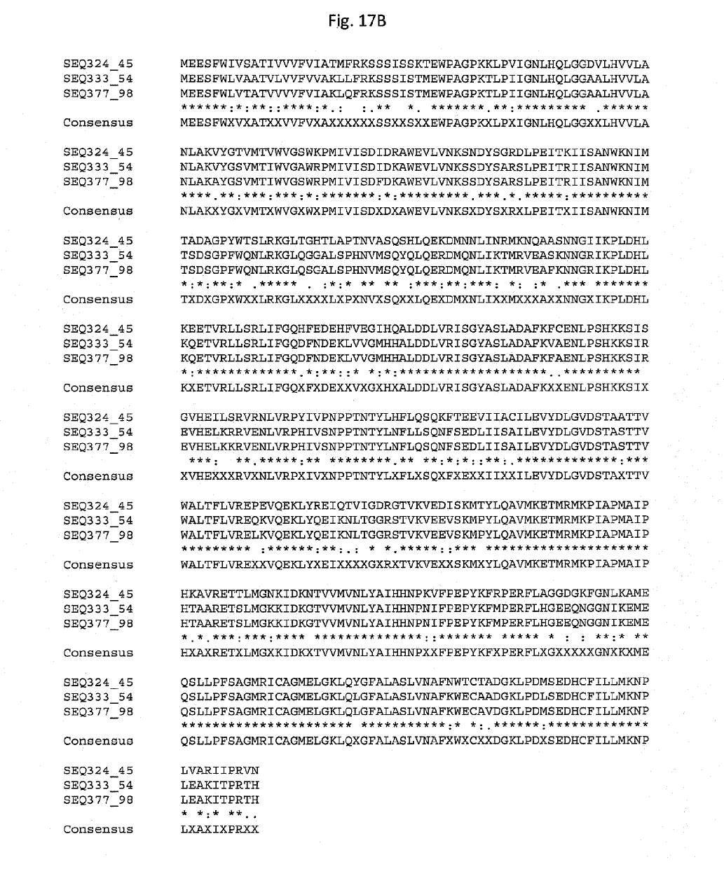

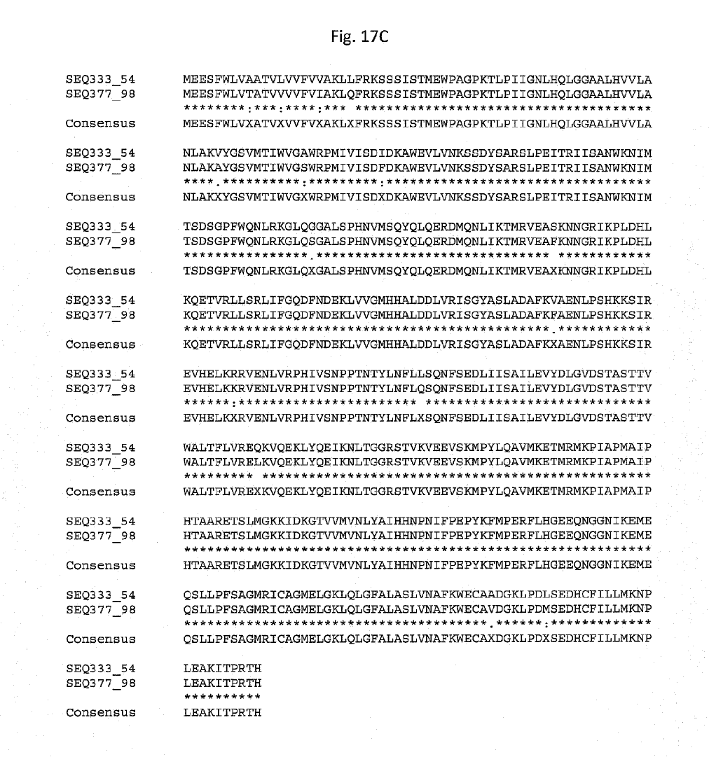

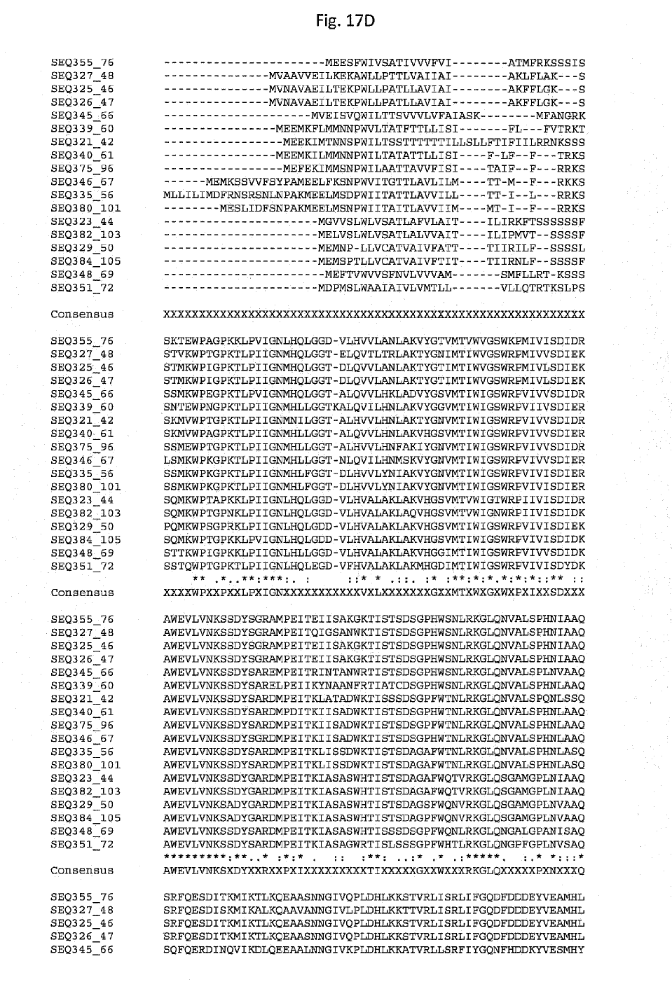

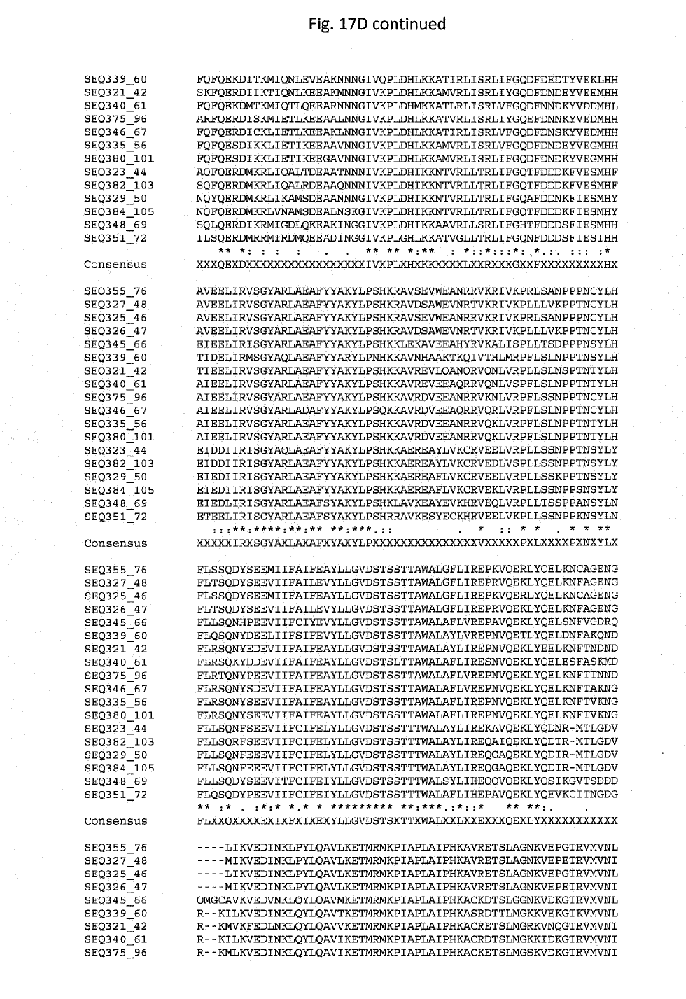

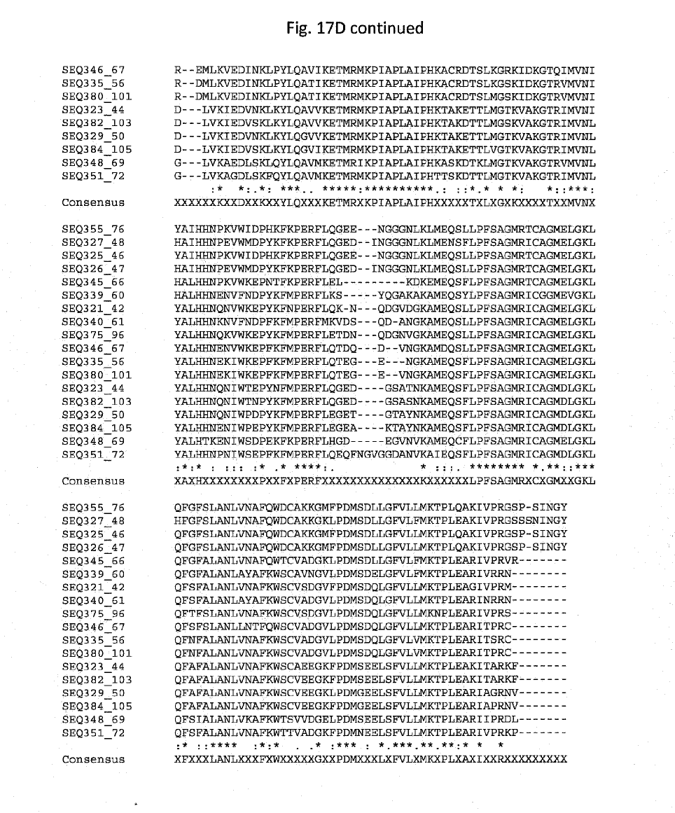

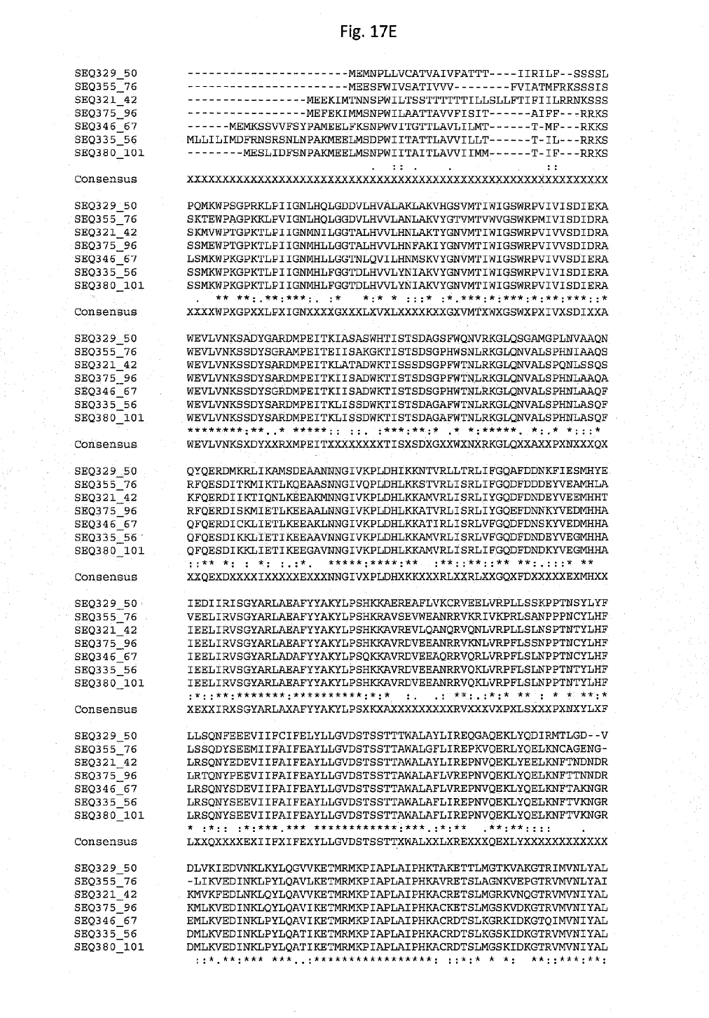

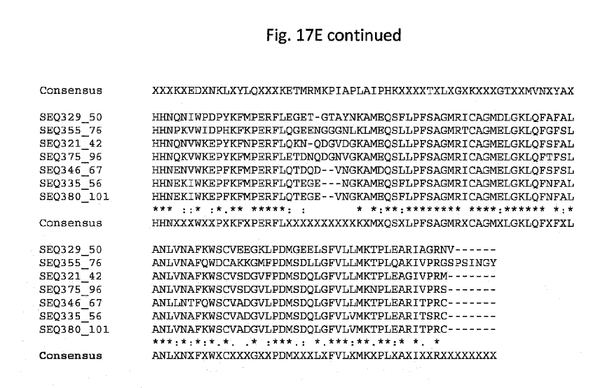

Consensuses derived from the alignments of certain of these orthologues are also presented in FIGS. 14 to 17. In specific embodiment of these consensuses, each X in the consensus sequences (e.g., consensuses in FIGS. 14 and 17) is defined as being any amino acid, or absent when this position is absent in one or more of the orthologues presented in the alignment. In specific embodiment of these consensuses, each X in the consensus sequences is defined as being any amino acid that constitutes a conserved or semi-conserved substitution of any of the amino acid in the corresponding position in the orthologues presented in the alignment, or absent when this position is absent in one or more of the orthologues presented in the alignment. In FIGS. 14 and 17, conservative substitutions are denoted by the symbol “:” and semi-conservative substitutions are denoted by the symbol “.”. In another embodiment, each X refers to any amino acid belonging to the same class as any of the amino acid residues in the corresponding position in the orthologues presented in the alignment, or absent when this position is absent in one or more of the orthologues presented in the alignment. In another embodiment, each X refers to any amino acid in the corresponding position of the orthologues presented in the alignment, or absent when this position is absent in one or more of the orthologues presented in the alignment. The Table below indicates which amino acid belongs to each amino acid class.

| Class | Name of the amino acids |
|---|---|
| Aliphatic | Glycine, Alanine, Valine, Leucine, Isoleucine |
| Hydroxyl or Sulfur/ | Serine, Cysteine, Selenocysteine, |
| Selenium-containing | Threonine, Methionine |
| Cyclic | Proline |
| Aromatic | Phenylalanine, Tyrosine, Tryptophan |
| Basic | Histidine, Lysine, Arginine |
| Acidic and their Amide | Aspartate, Glutamate, Asparagine, Glutamine |

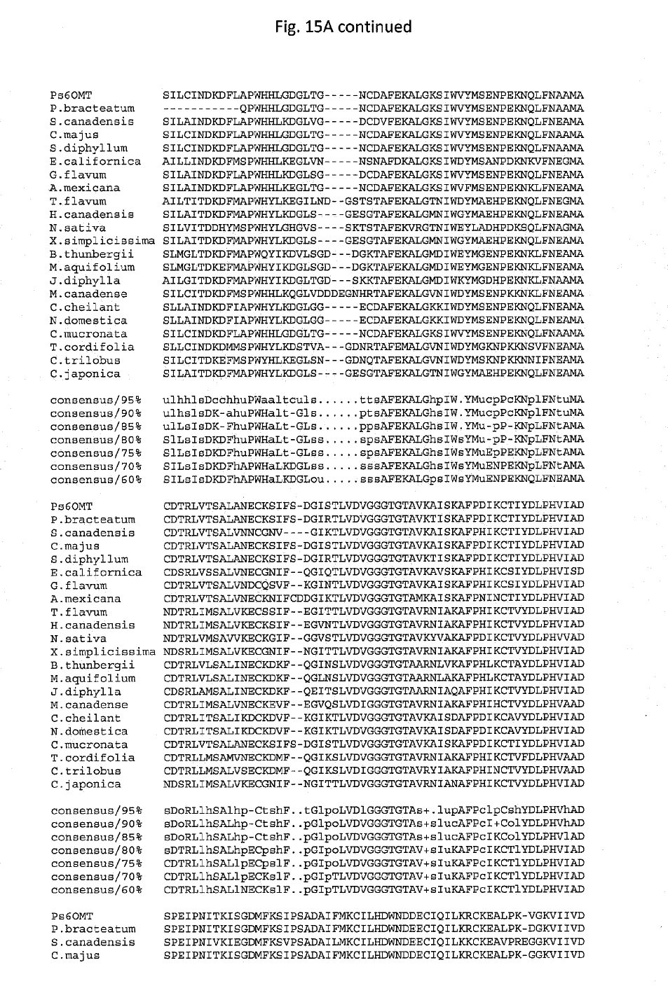

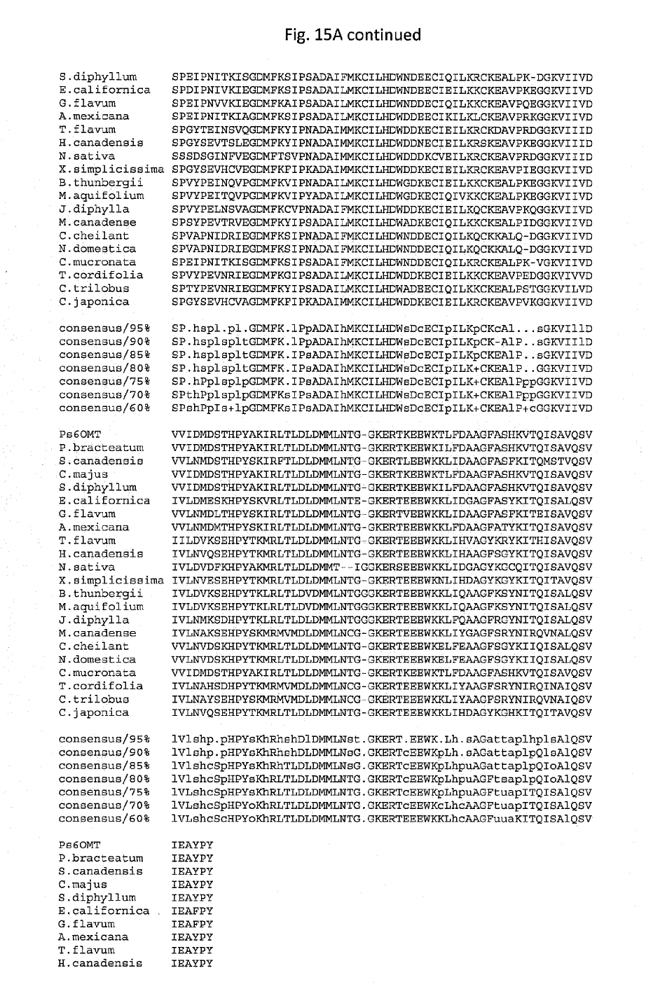

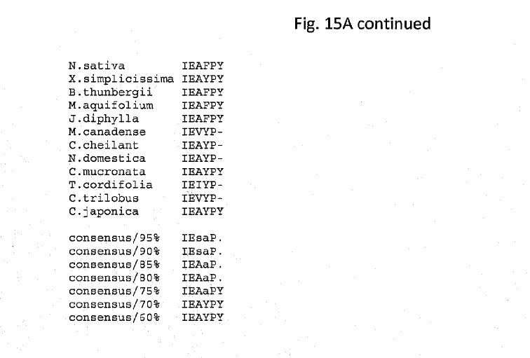

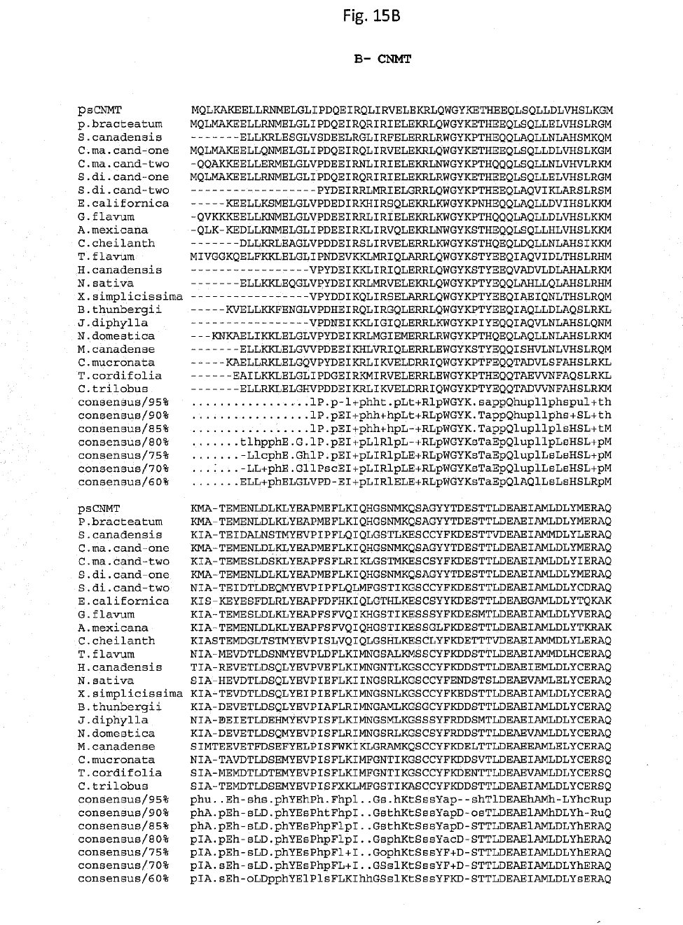

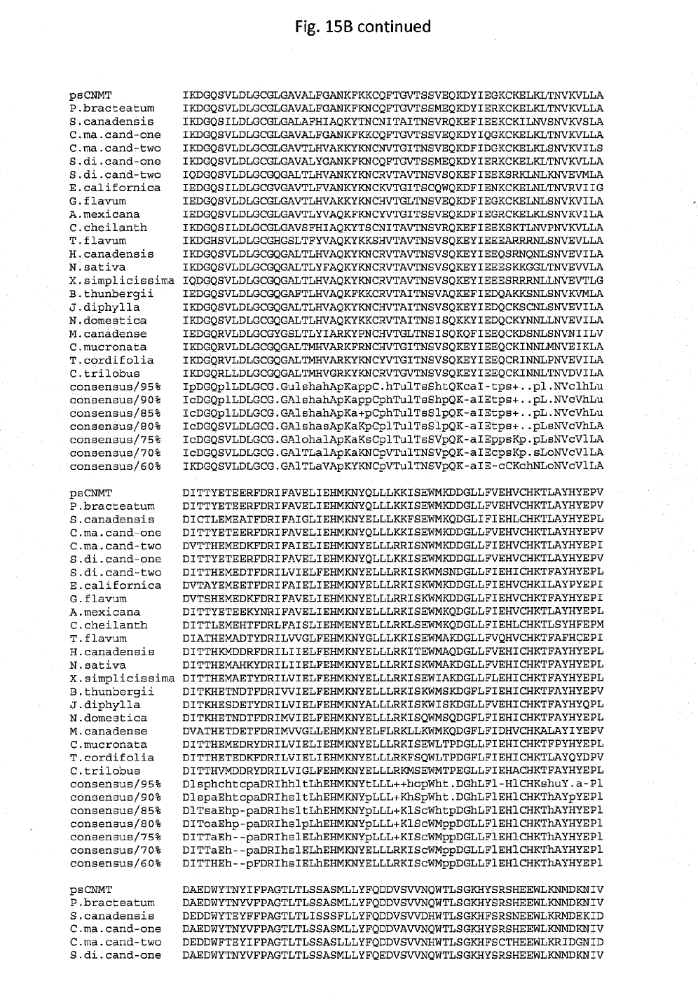

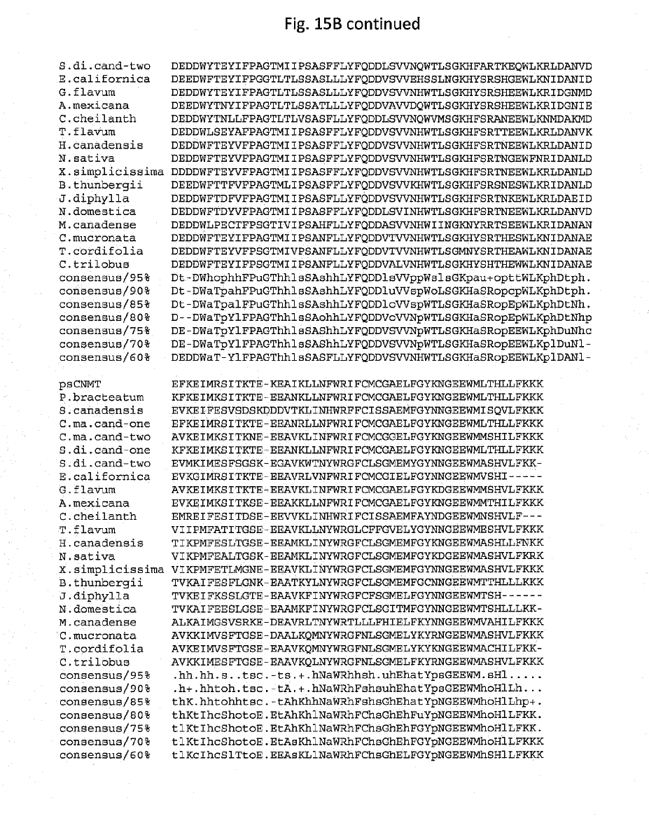

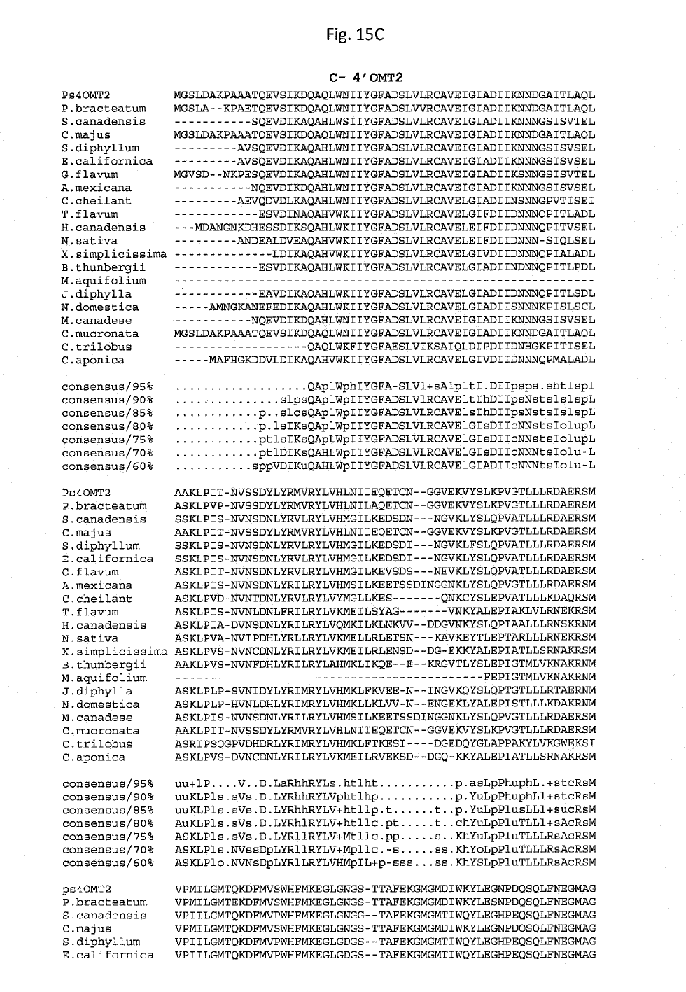

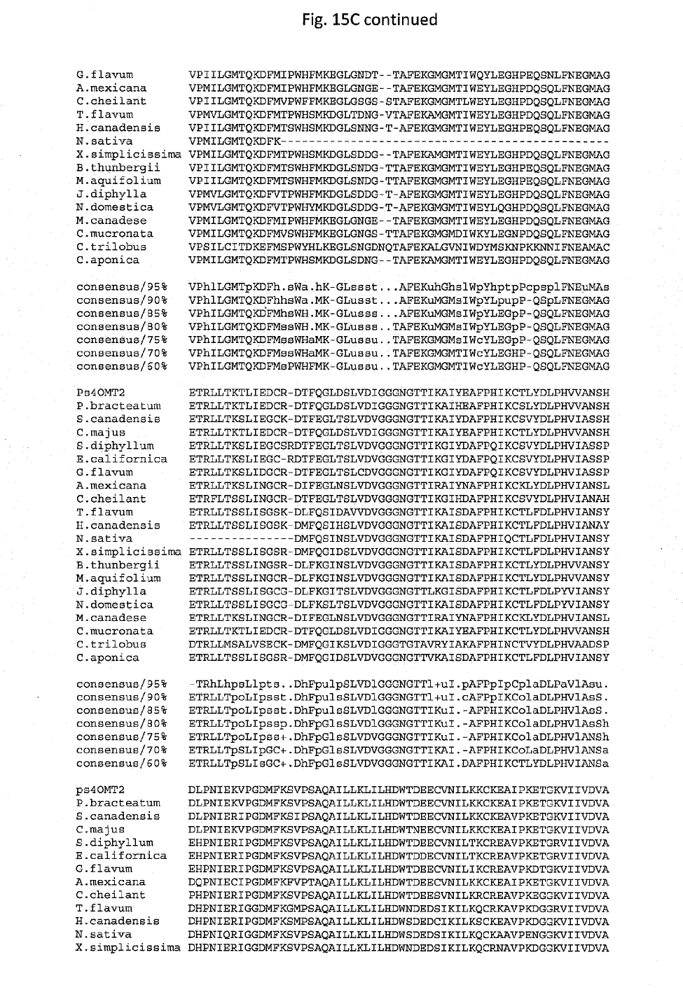

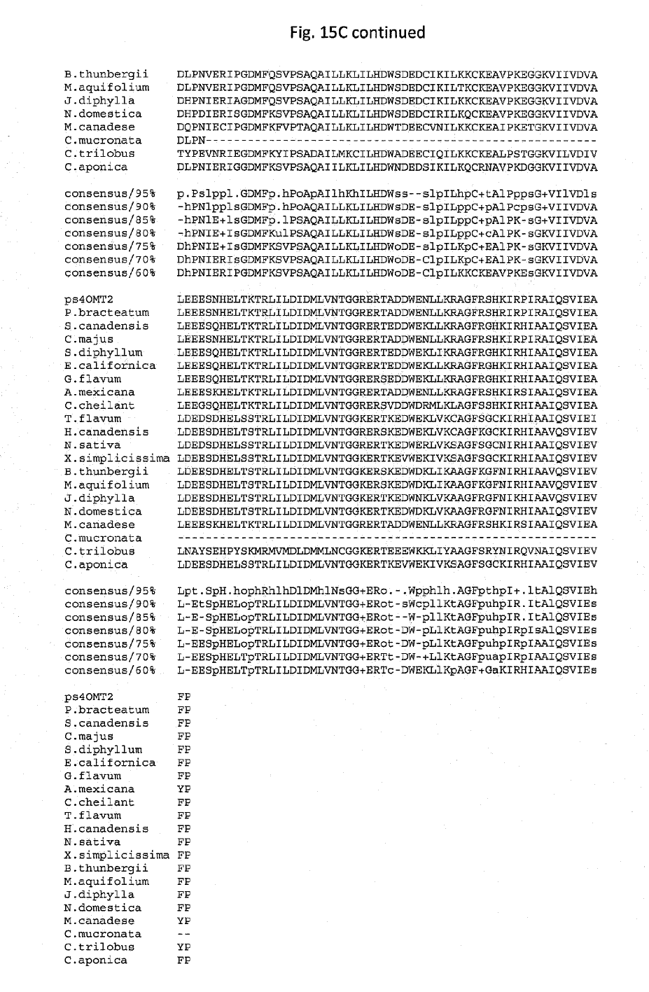

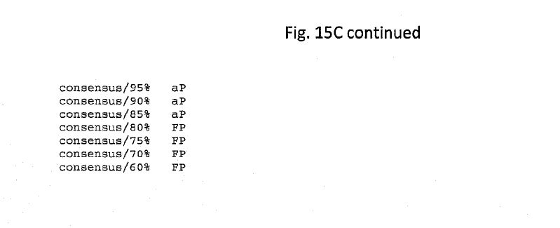

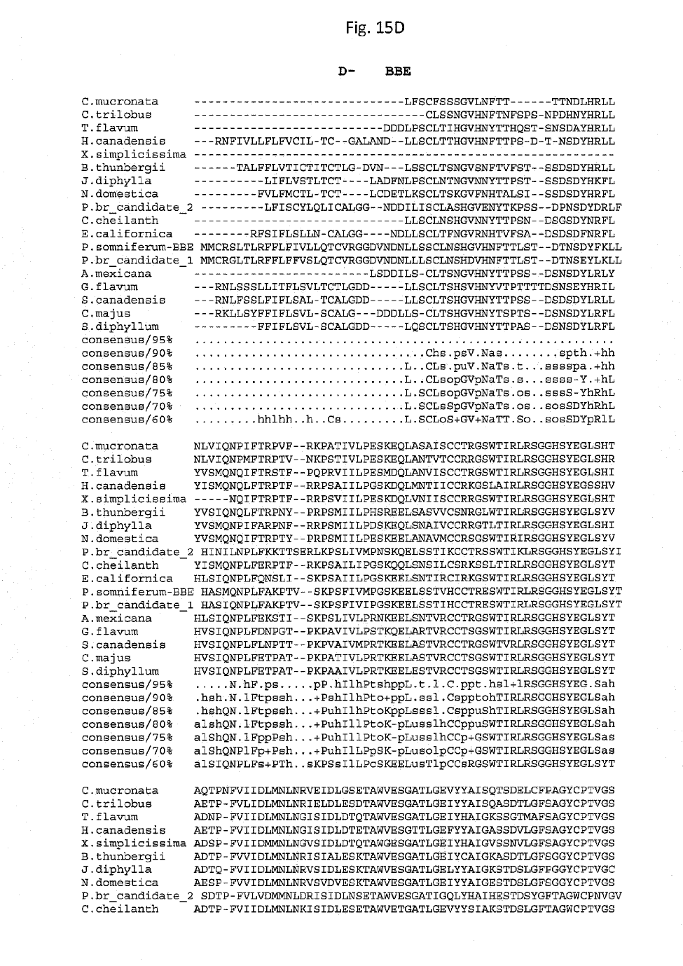

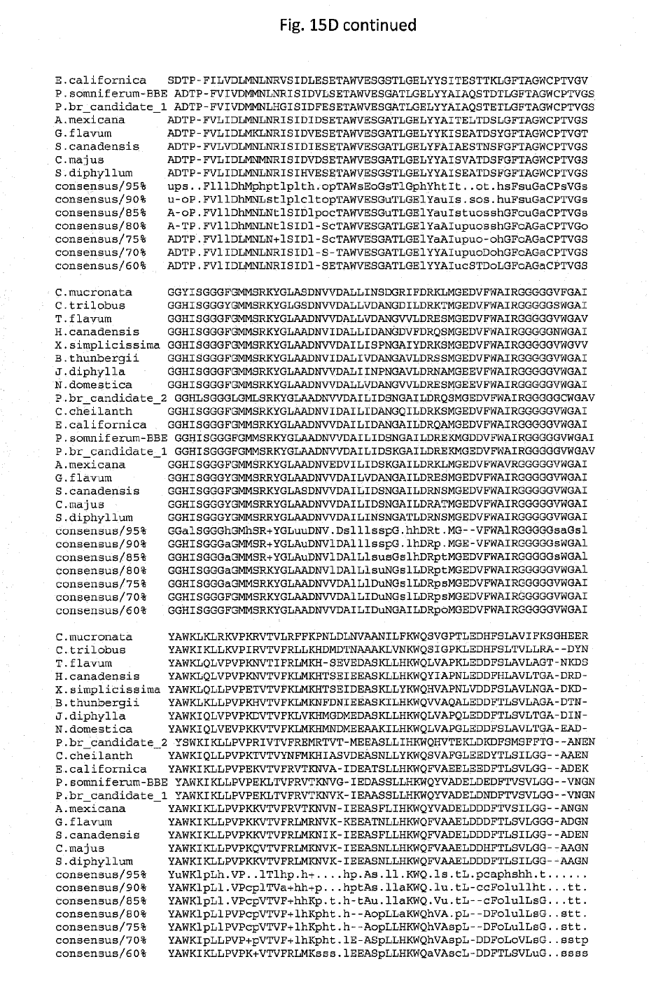

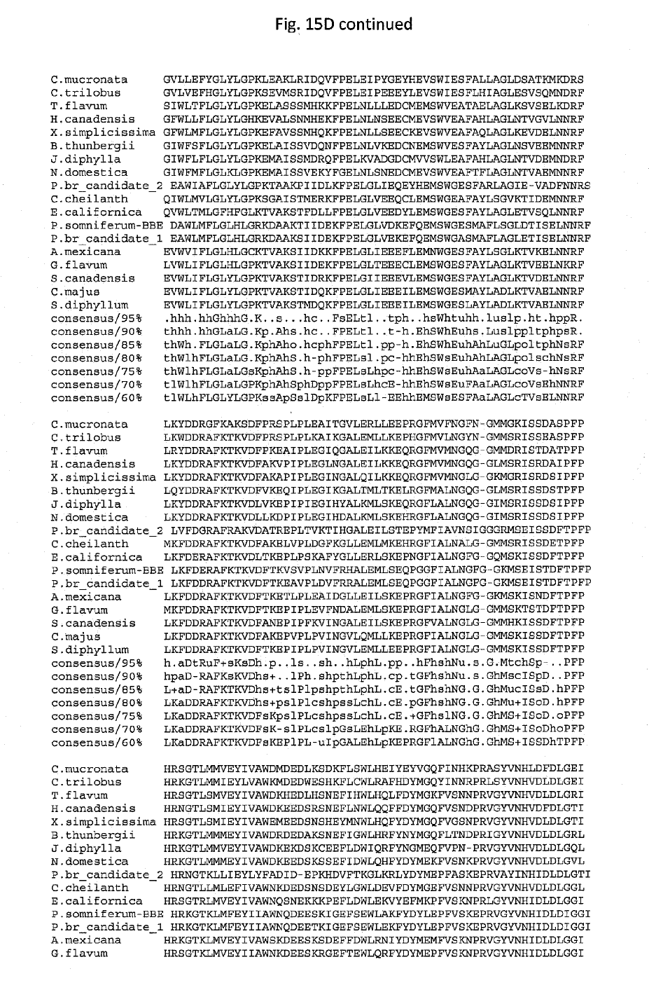

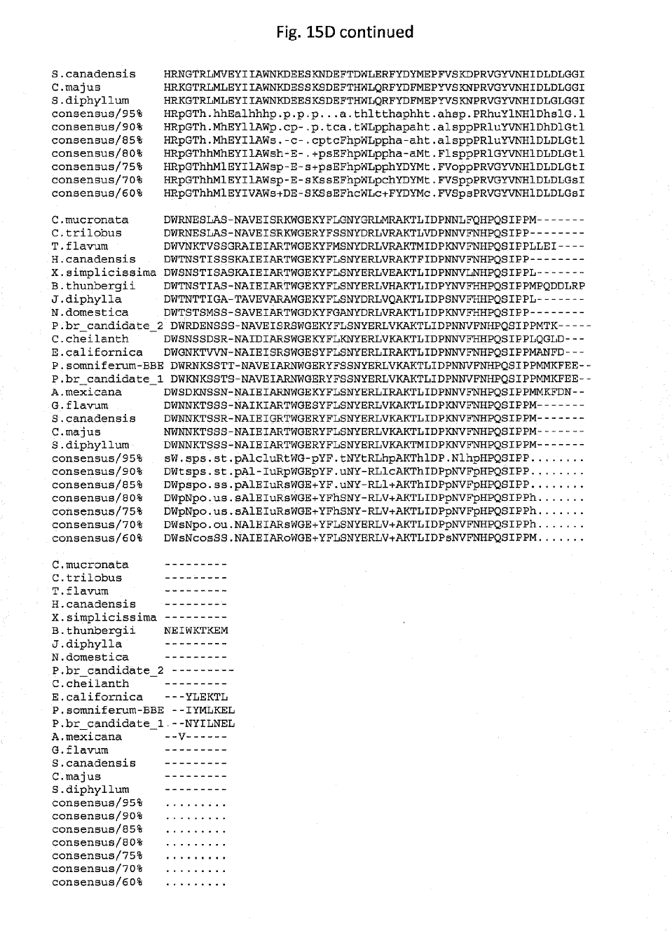

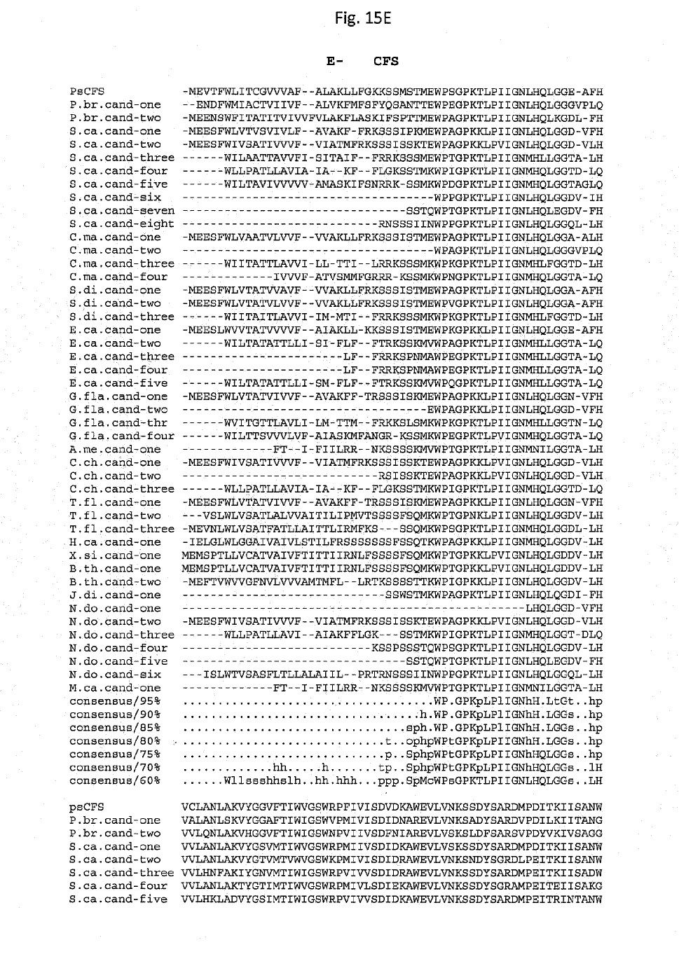

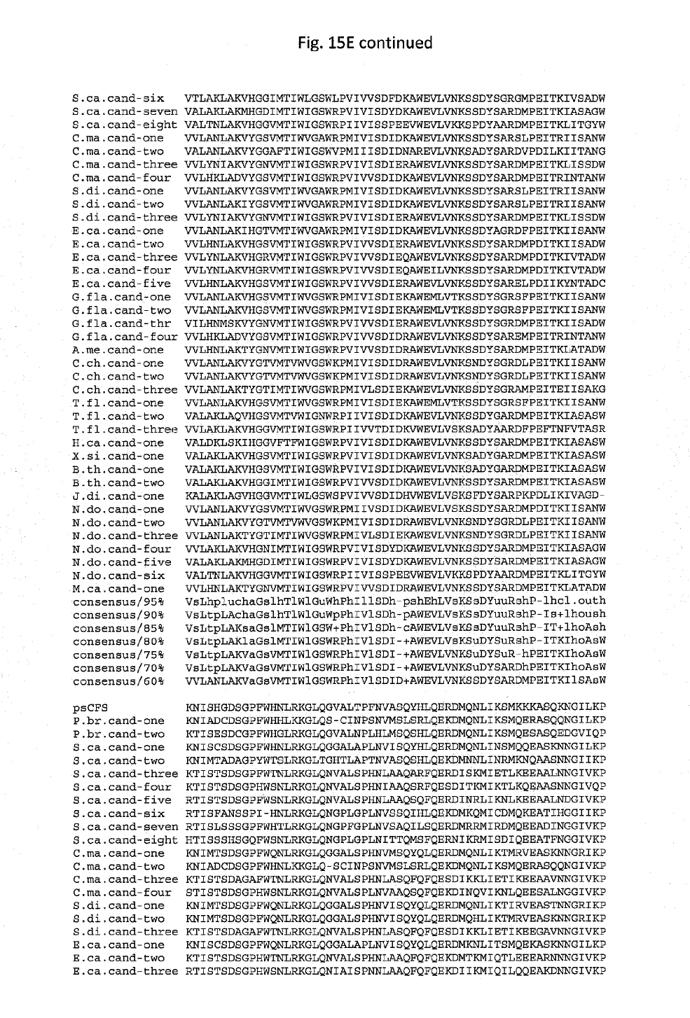

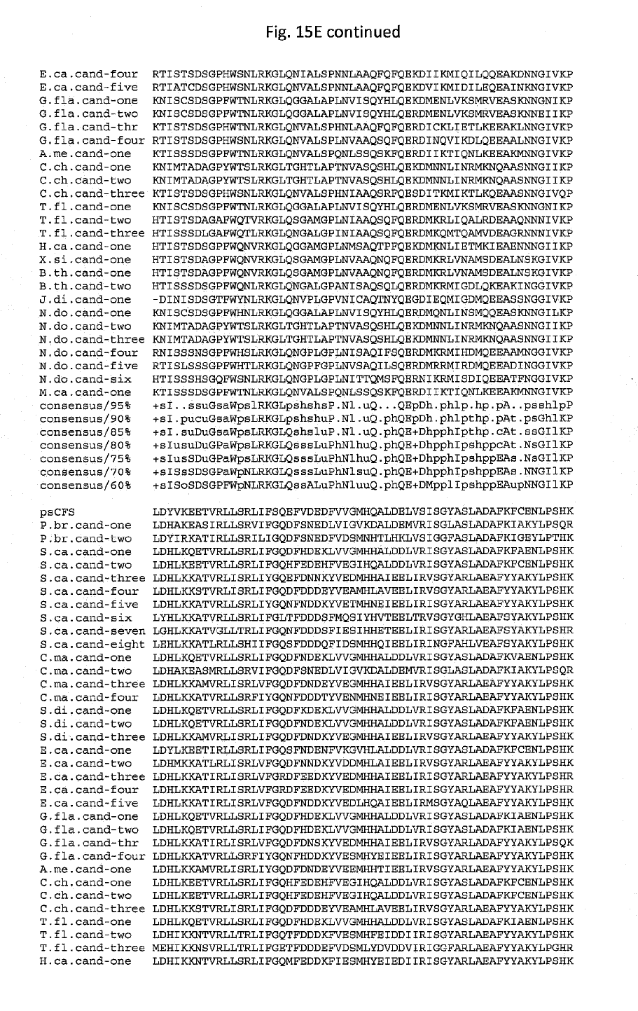

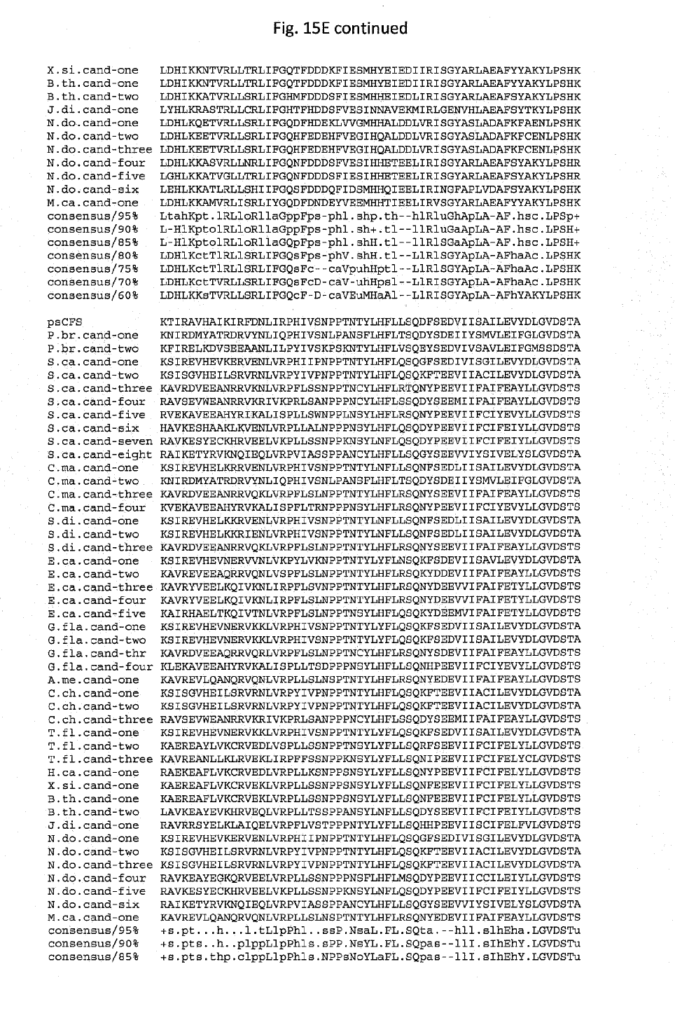

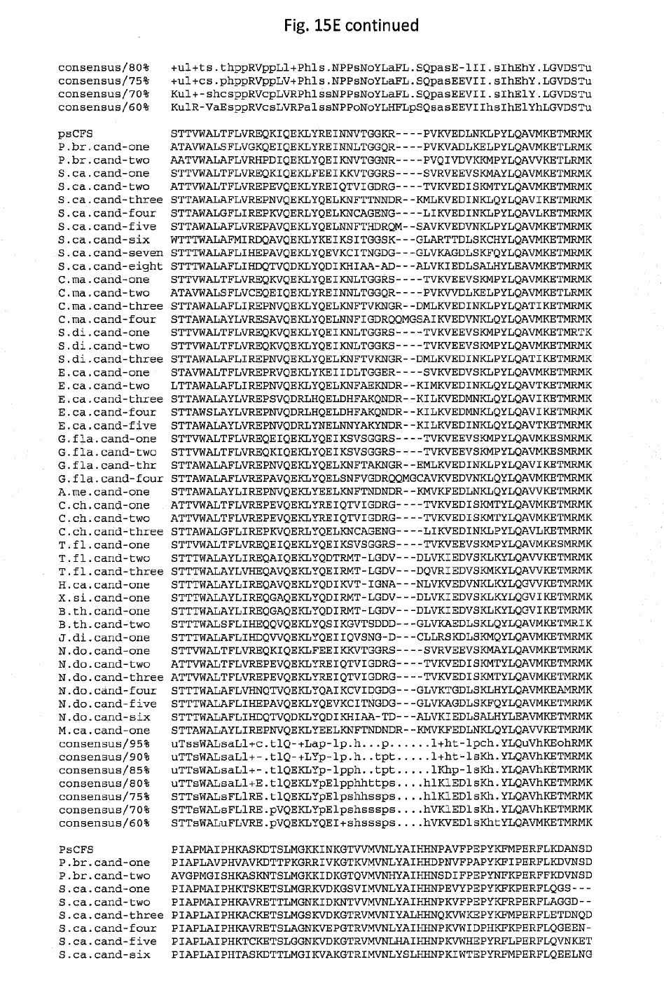

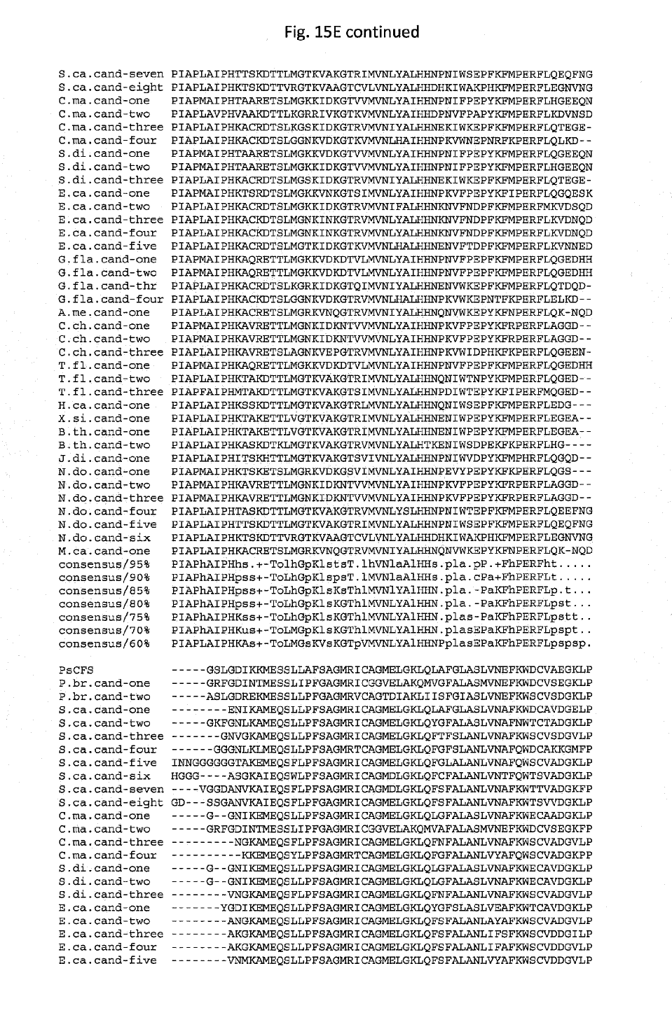

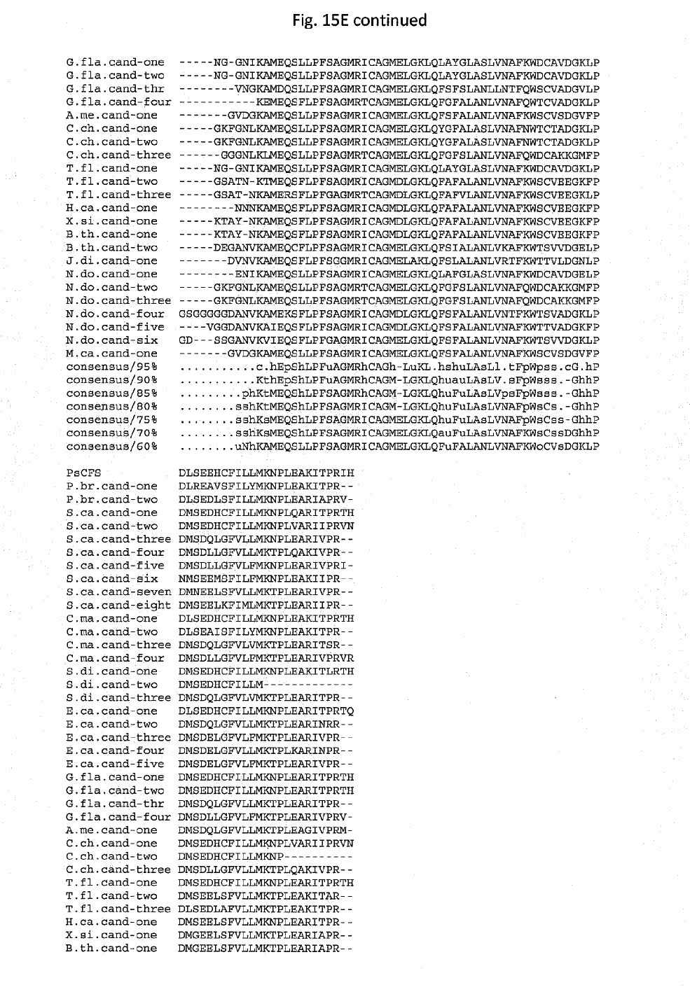

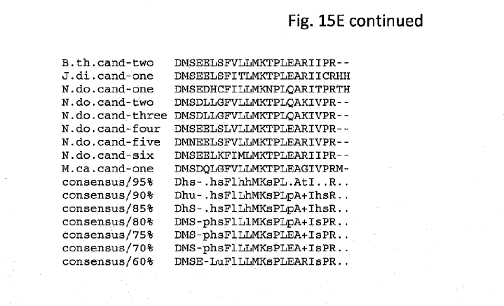

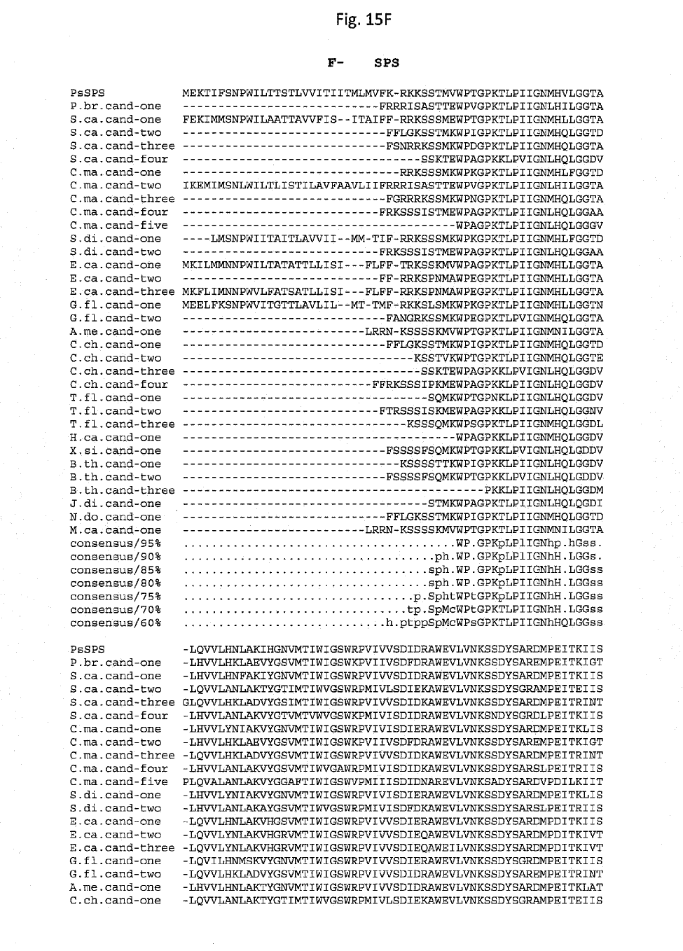

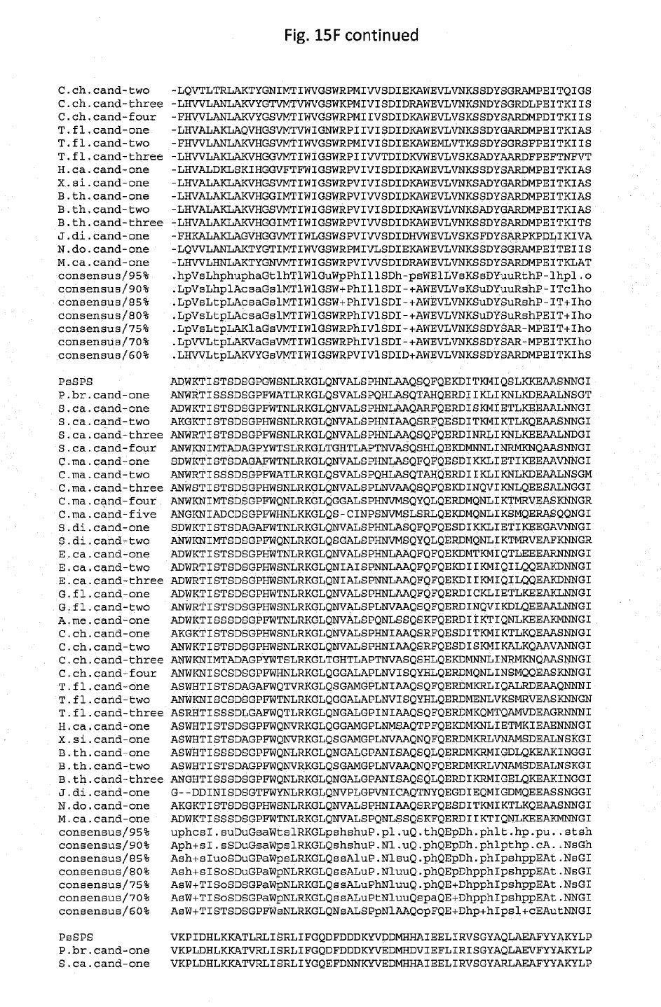

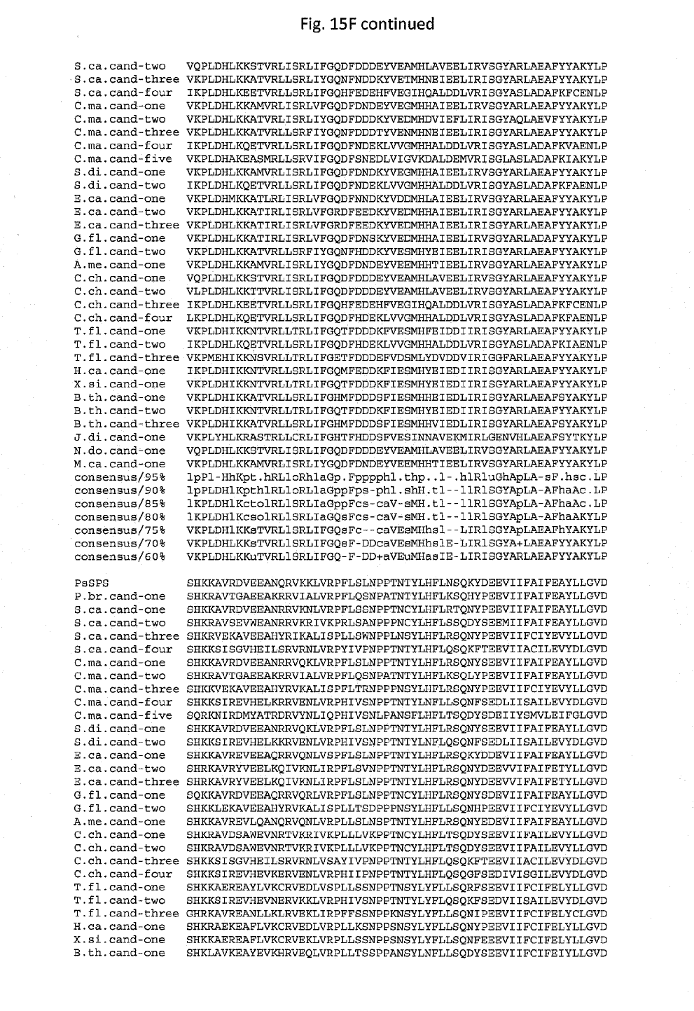

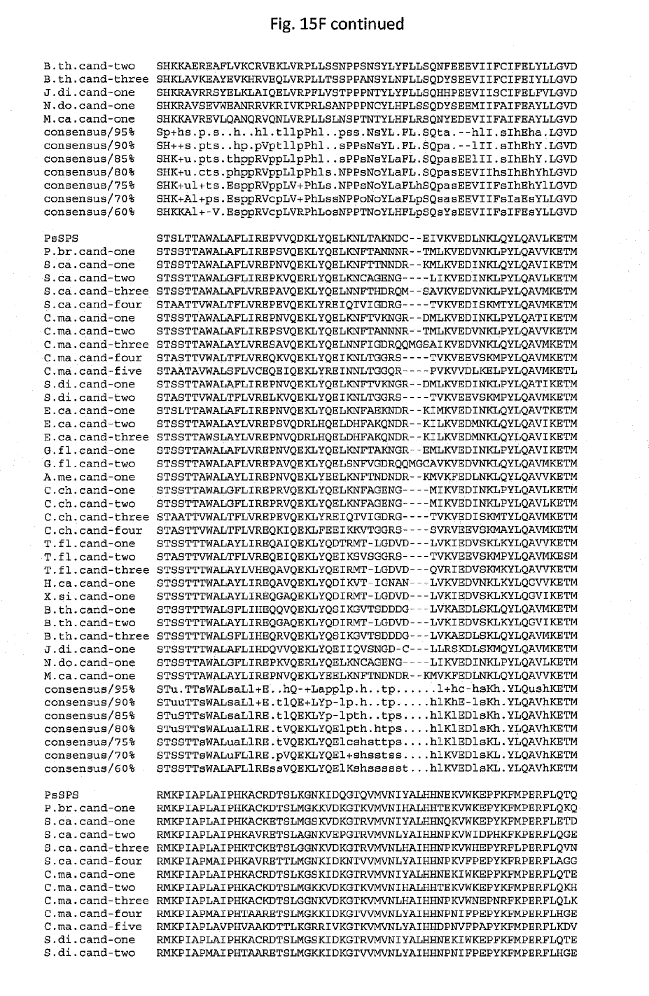

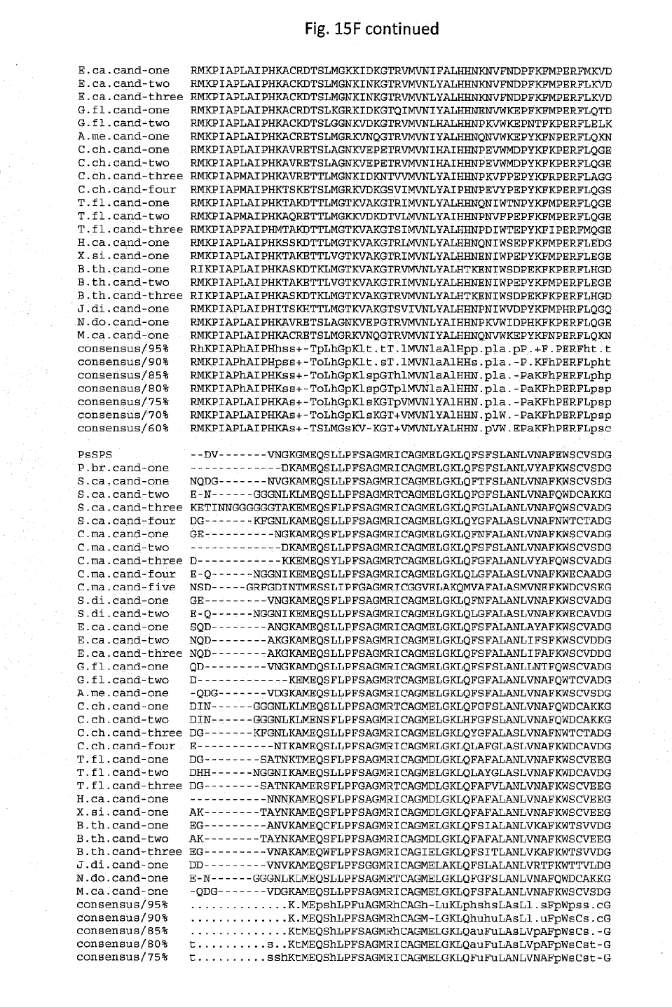

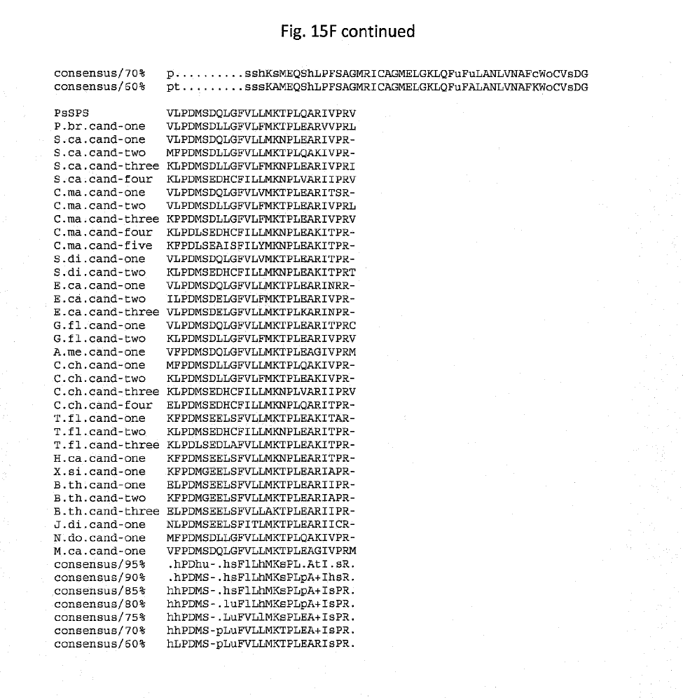

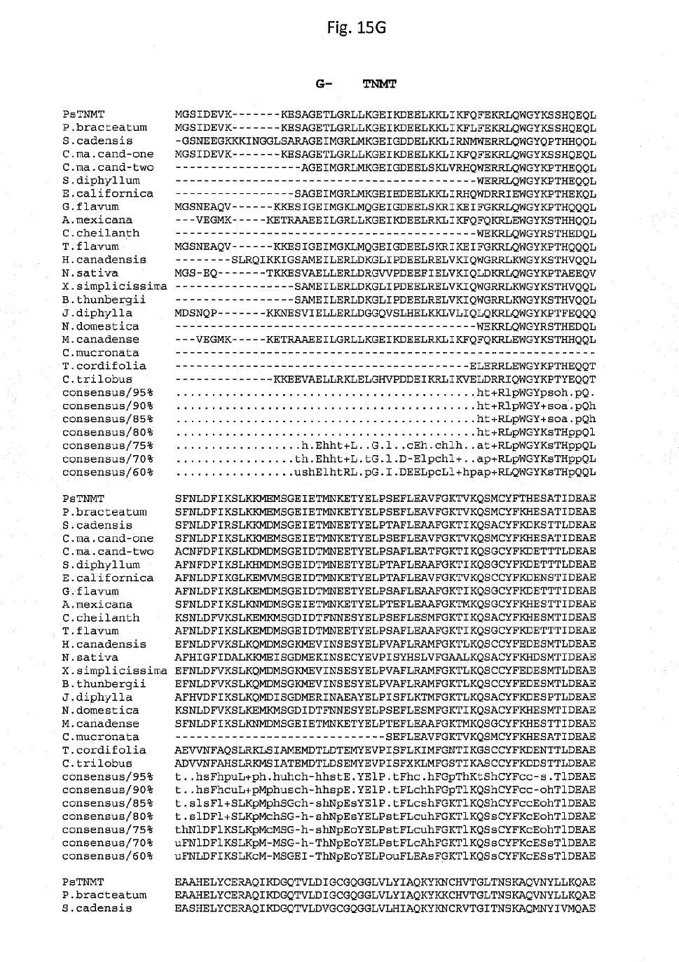

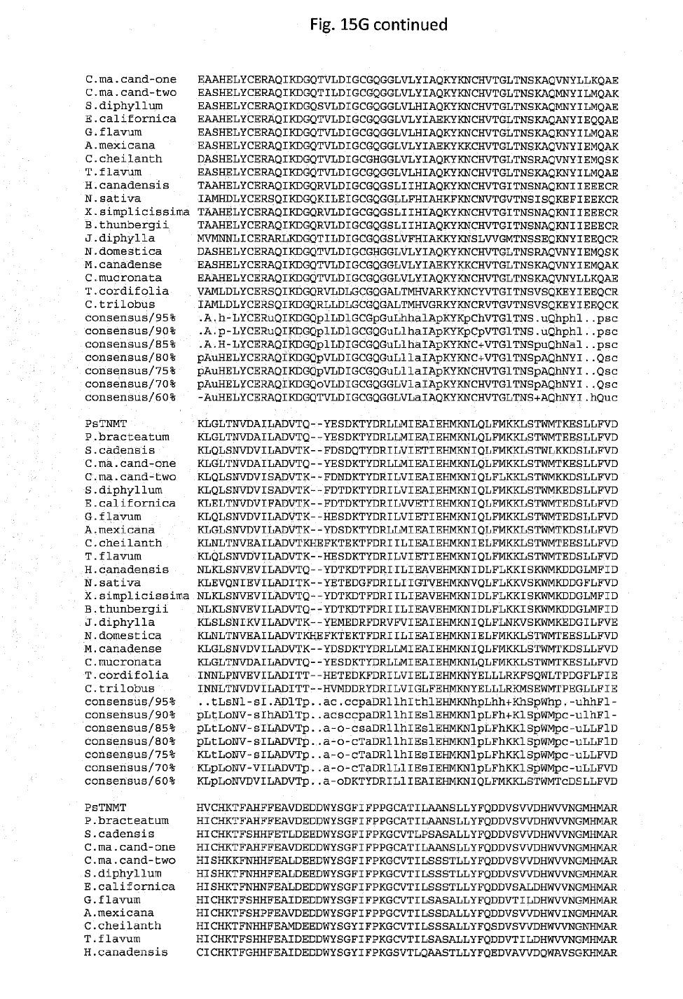

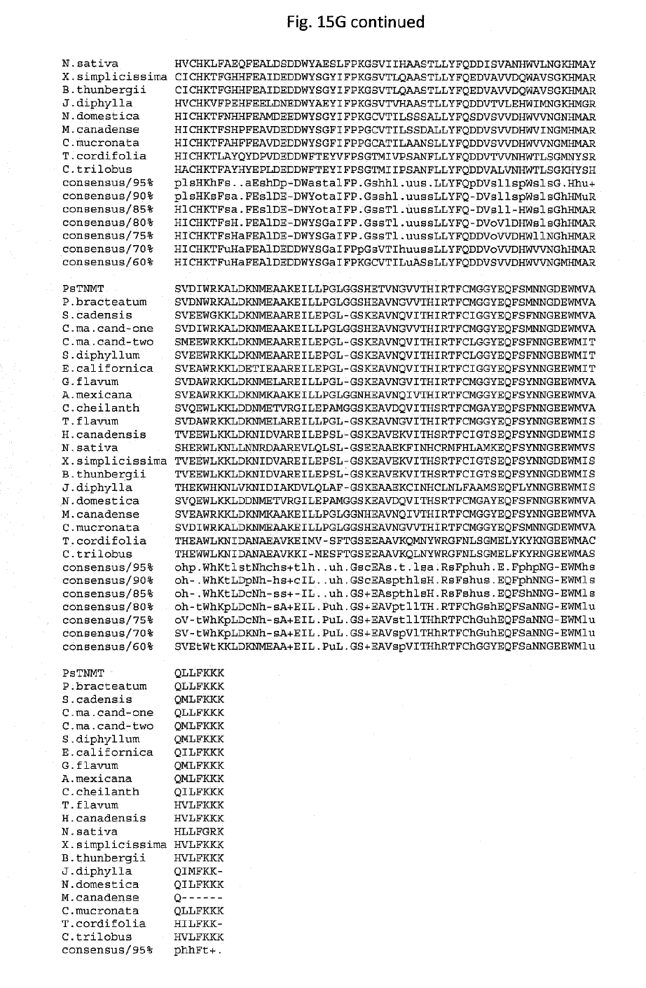

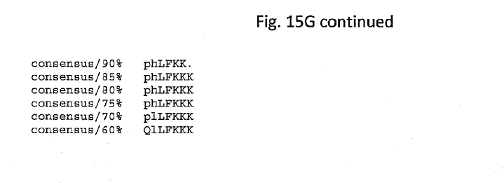

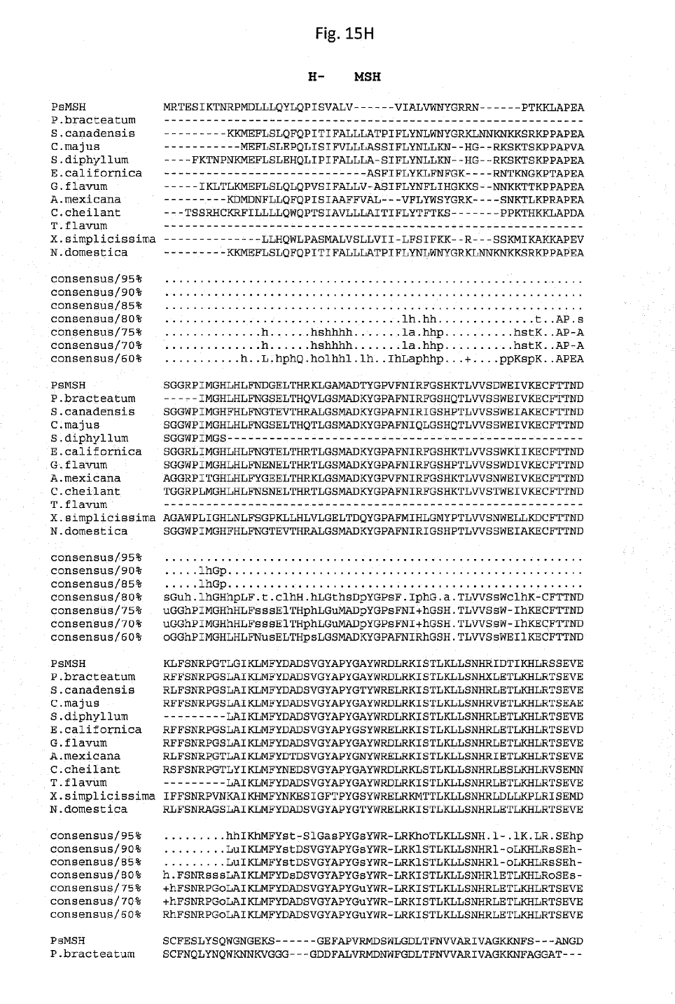

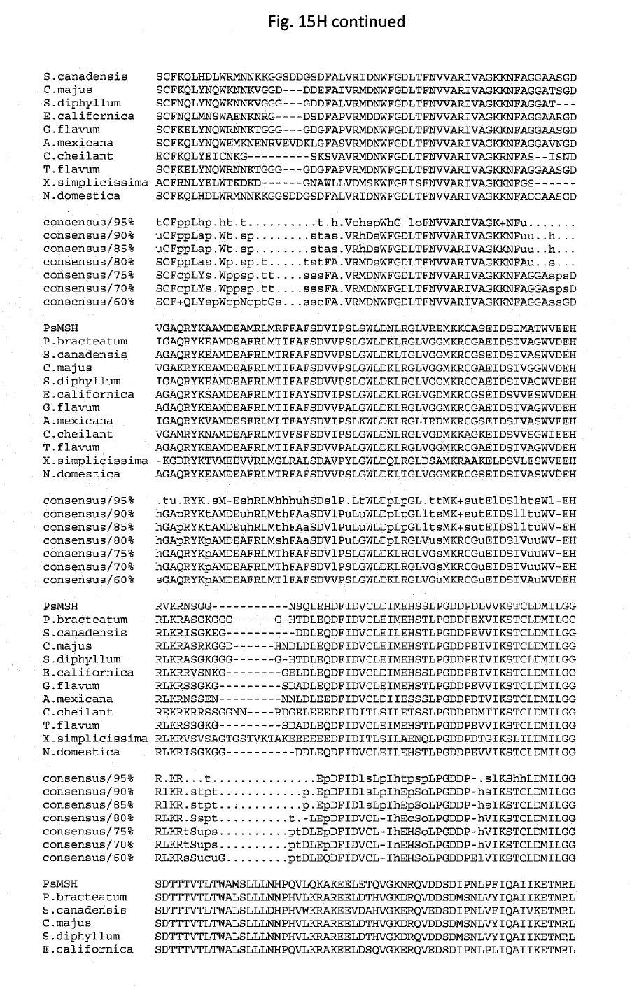

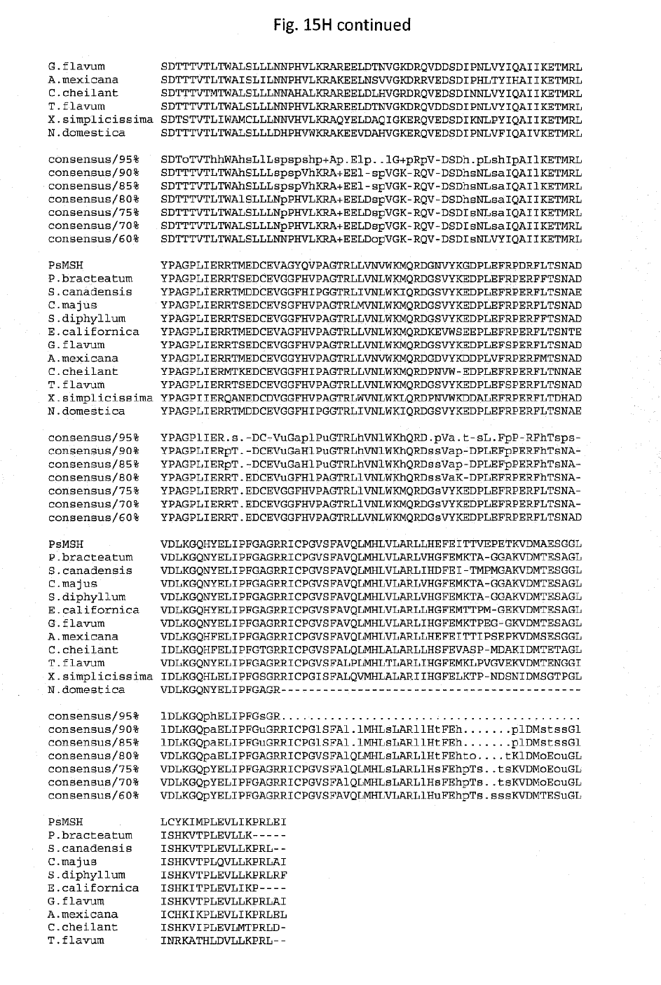

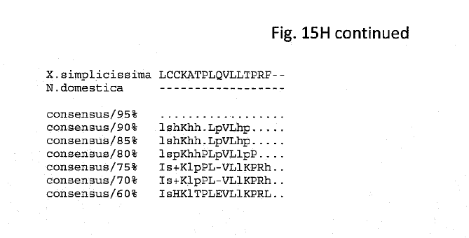

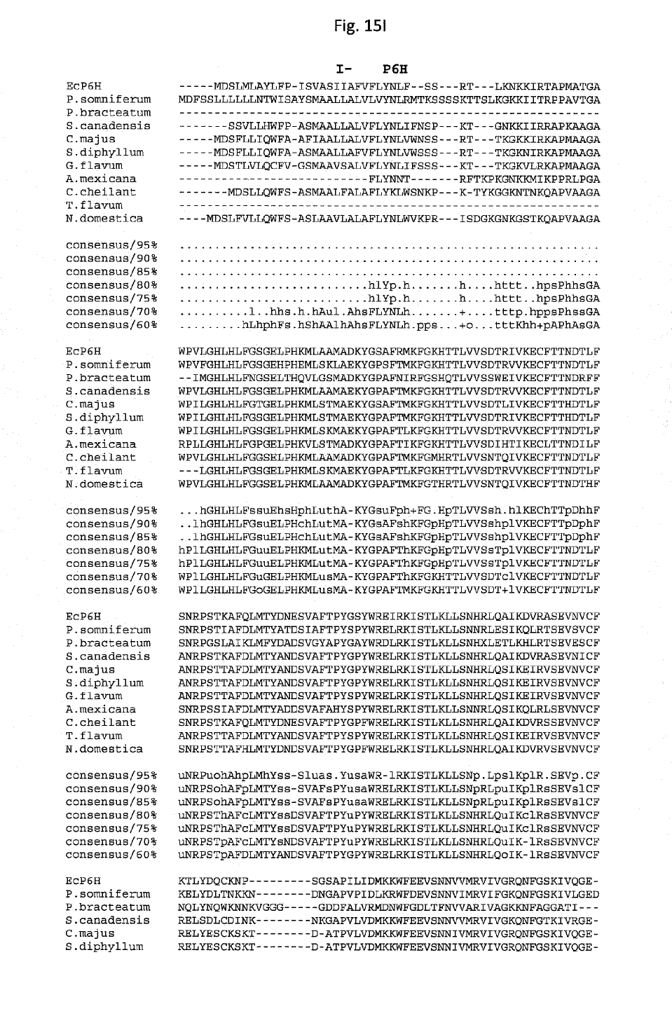

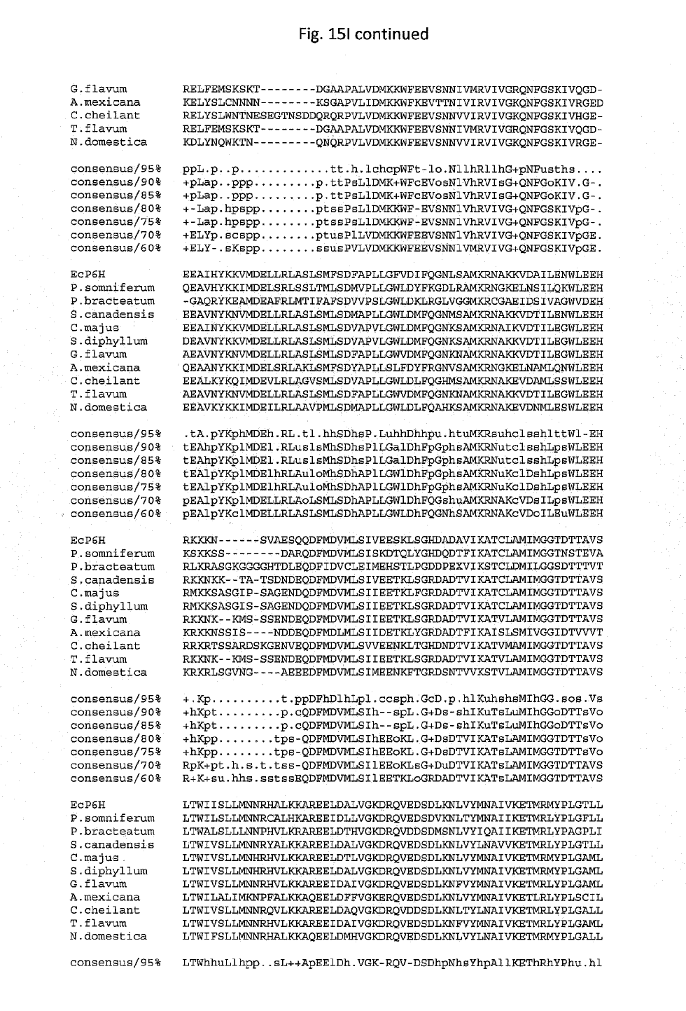

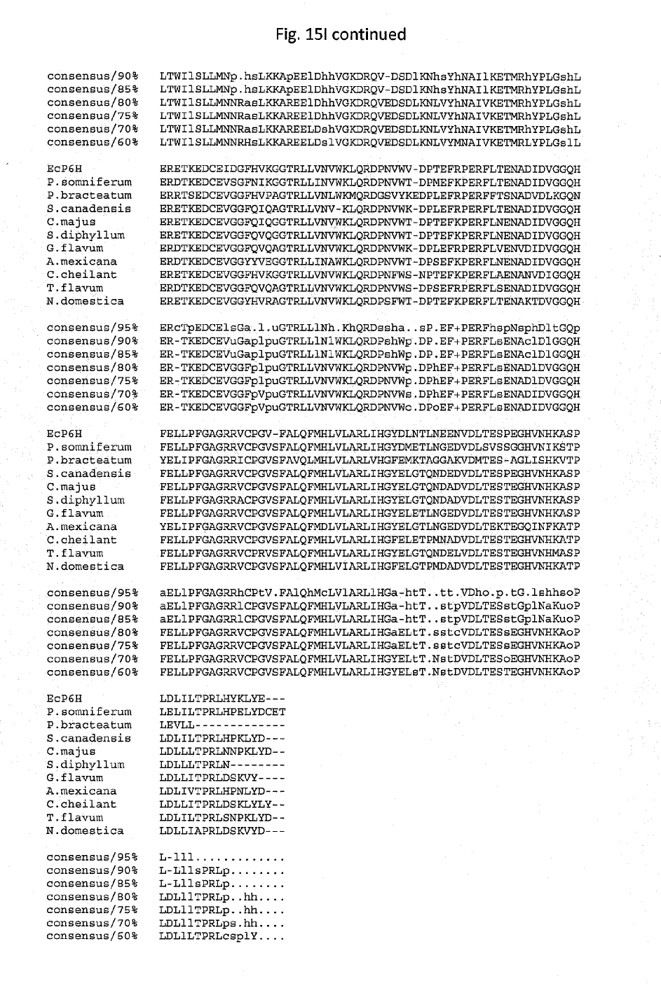

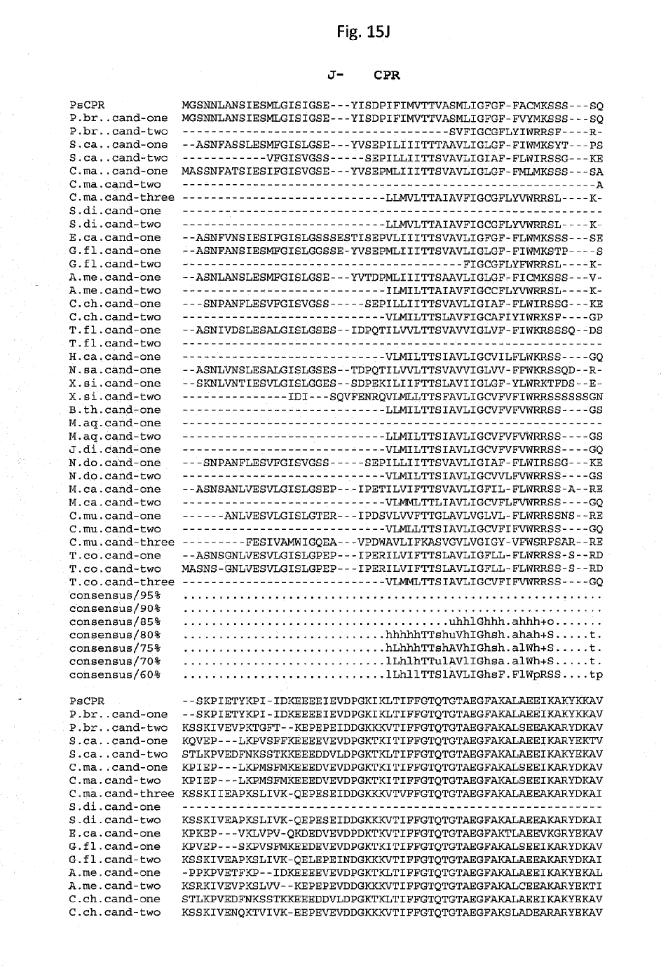

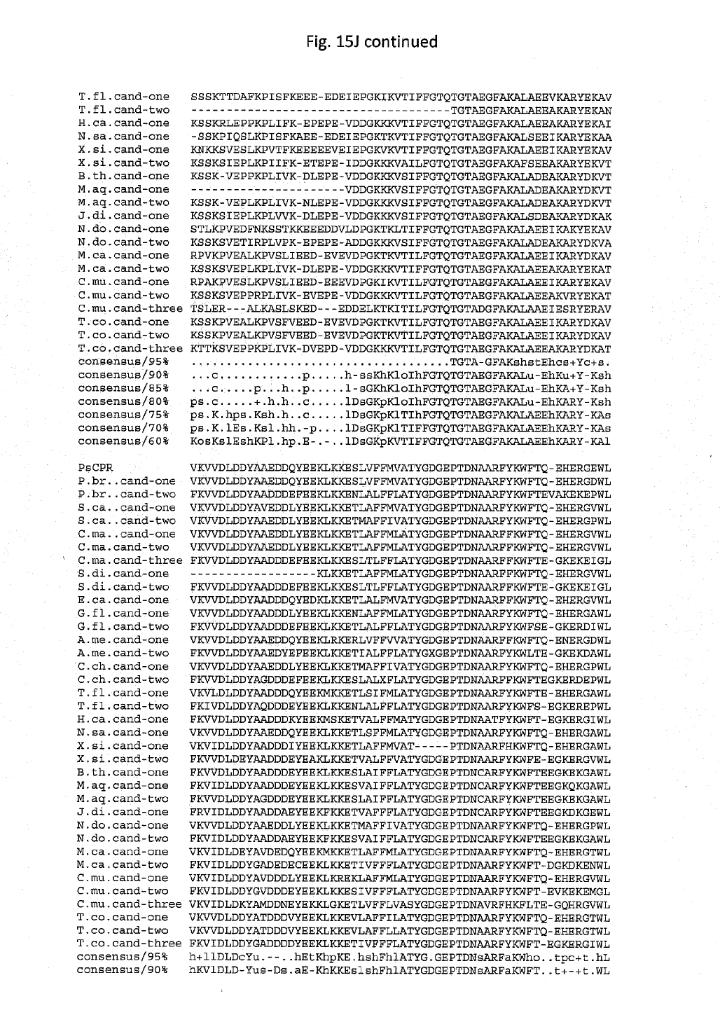

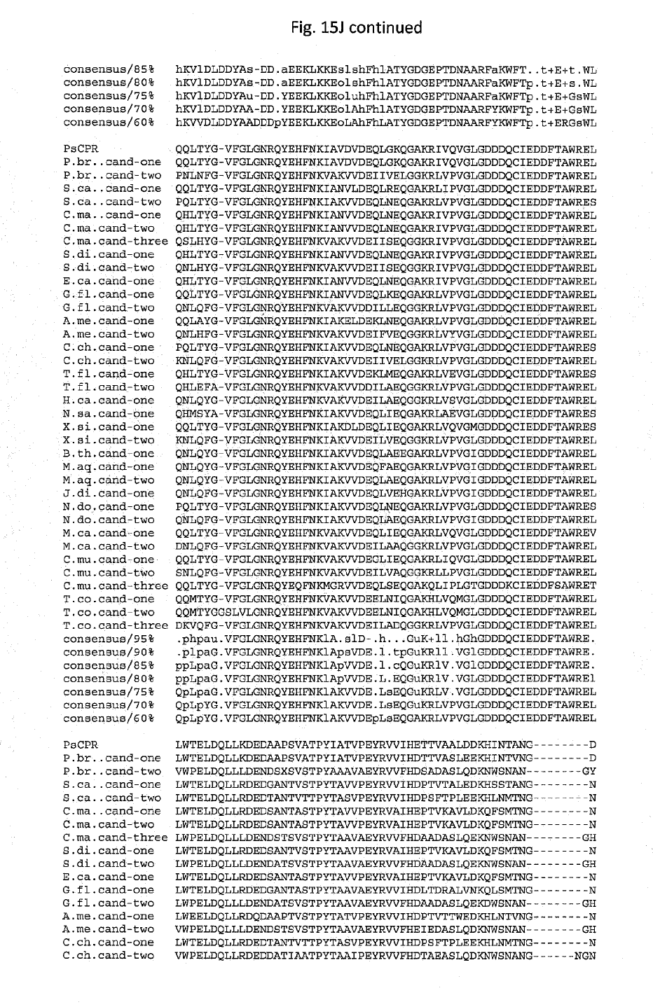

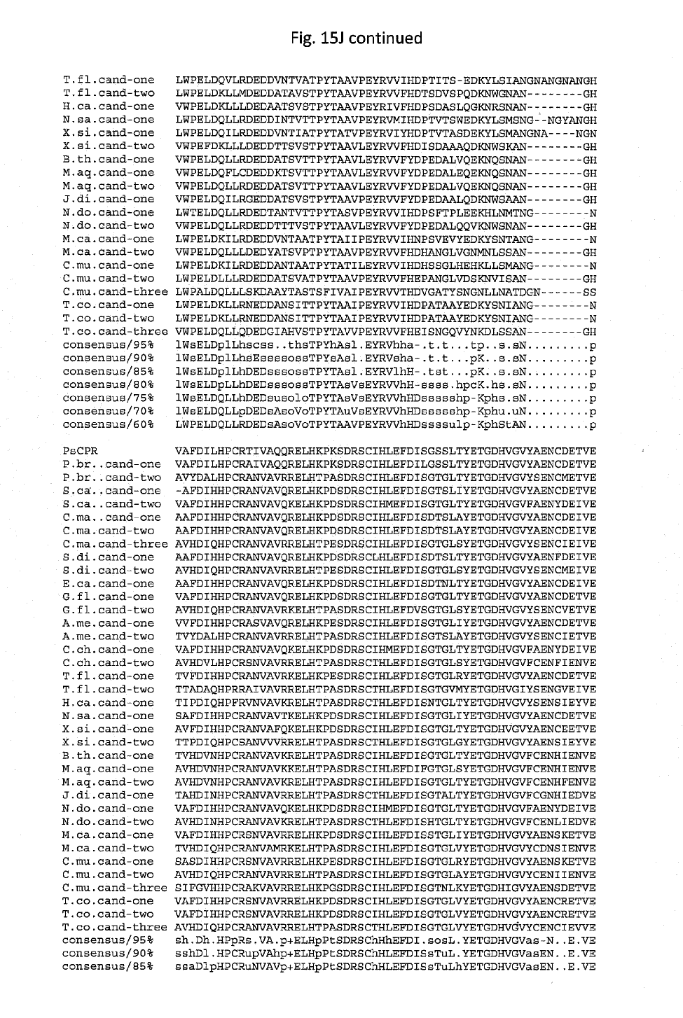

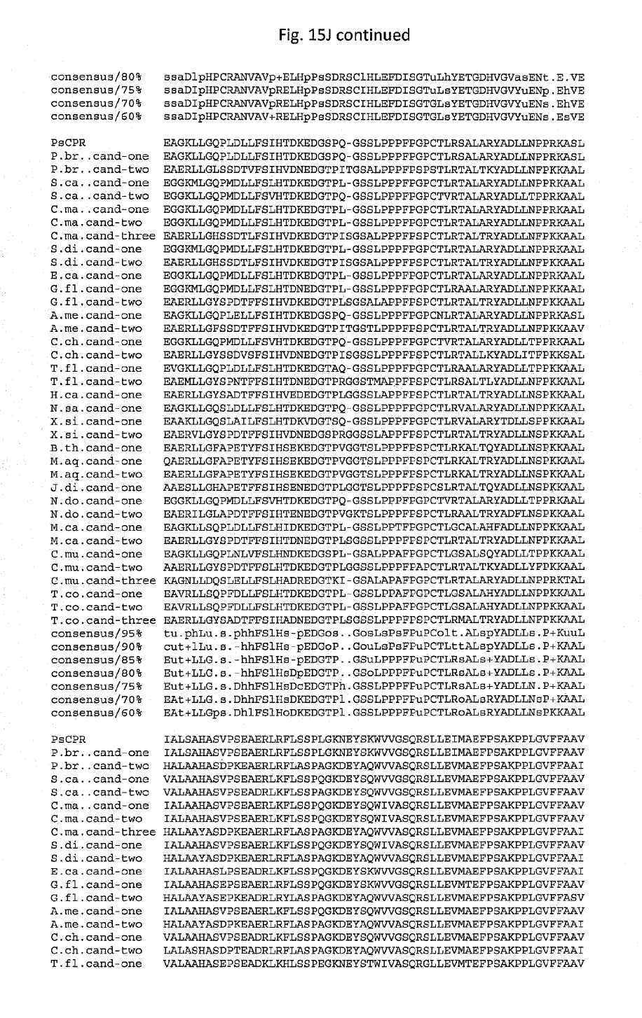

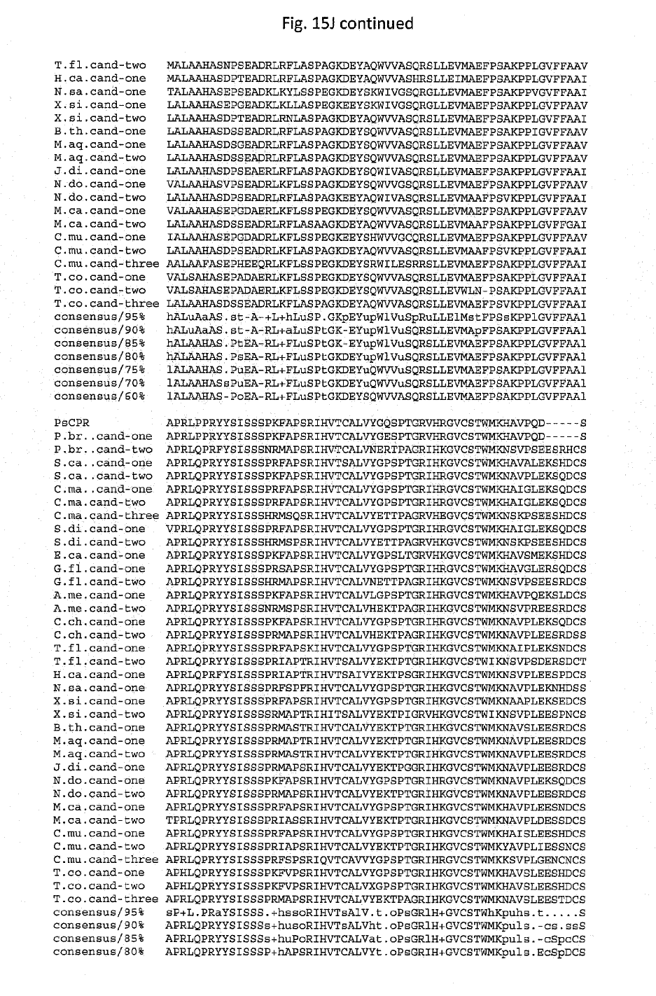

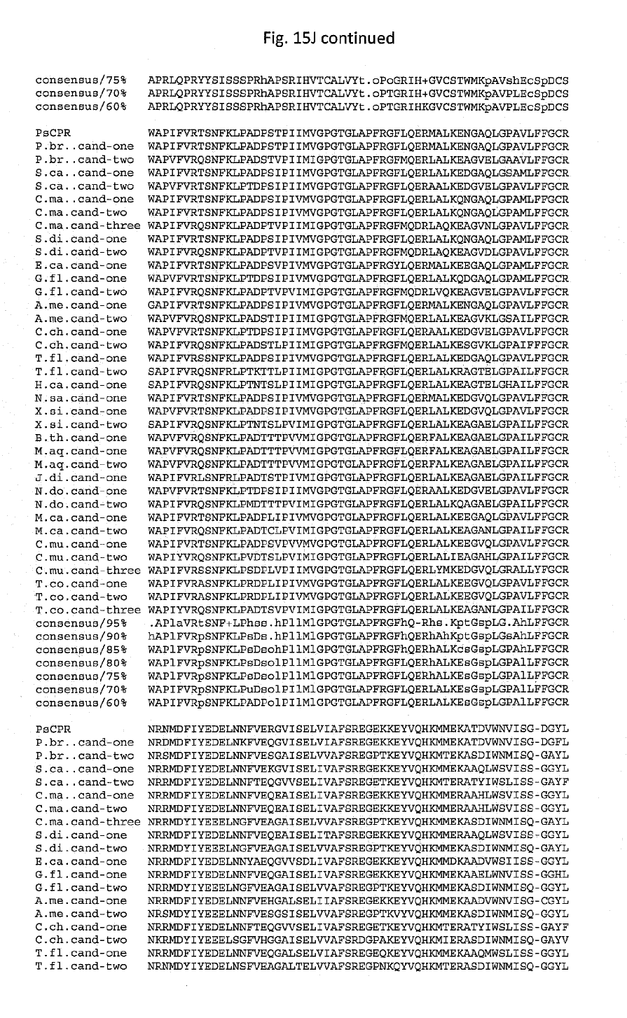

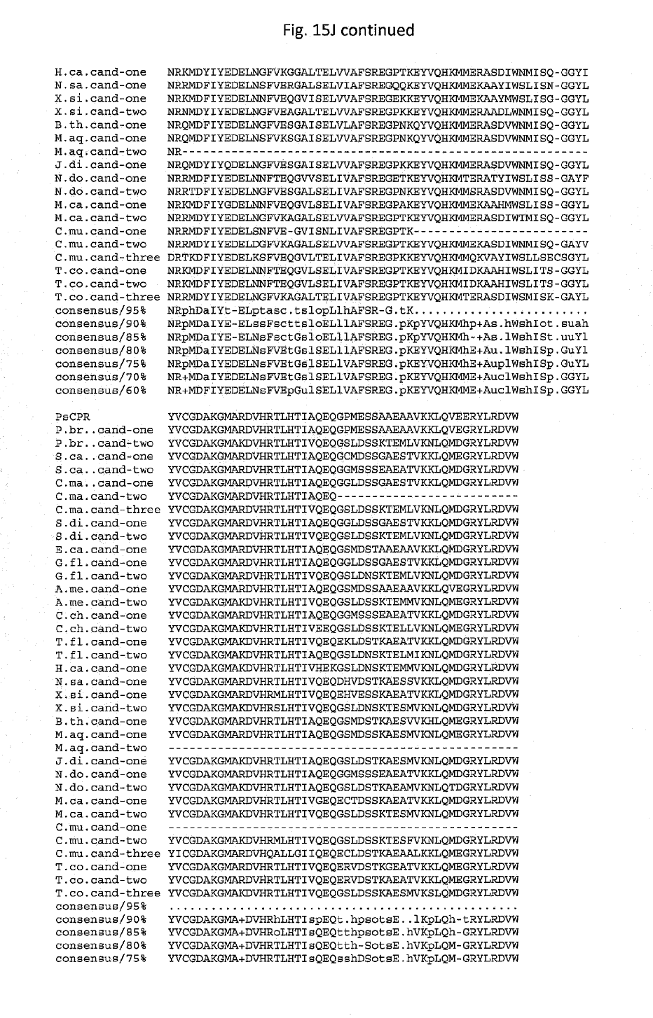

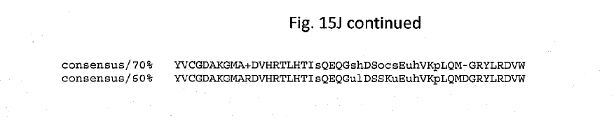

In other specific embodiments of the enzymes as used in the present invention (e.g., consensuses in FIG. 15), the small “o” denotes alcohol and refers to S or T; small “I” denotes aliphatic and refers to I, L or V; period “.” denotes any amino acid; small “a” denotes aromatic and refers to F, H, W or Y; small “c” denotes charged and refers to D, E, H, K or R; small “h” denotes hydrophobic and refers to A, C, F, G, H, I, K, L, M, R, T, V, W or Y; minus sign “−” denotes negative and refers to D or E; small “p” denotes polar and refers to C, D, E, H, K, N, Q, R, S or T; plus sign “+” denotes positive and refers to H, K or R; small “s” denotes small and refers to A, C, D, G, N, P, S, T or V; small “u” denotes tiny and refers to A, G or S; small “t” denotes turnlike and refers to A, C, D, E, G, H, K, N, Q, R, S and T.

Hence enzymes in accordance with the present invention include enzymes having the specific nucleotide or amino acid sequences described in FIGS. 13 to 17, or an amino acid sequence that satisfies any of the consensuses as defined above (e.g., FIGS. 14 and 17). In particular, it includes enzyme sequences satisfying the consensus sequences described in FIGS. 14A to K and 17 (full and truncated (e.g. devoid of shaded domain)) wherein the one or more Xs are defined as above. It also refers to consensus sequences described in FIGS. 15A to J. It also refers to consensus sequences of catalytic domains of these enzymes. Enzyme sequences in accordance with the present invention include the specific sequences described in FIGS. 13 to 17 with up to 10 amino acids (9, 8, 7, 6, 5, 4, 3, 2 or 1) truncated at the N- and/or C-terminal thereof.

In a more specific embodiment, the enzymes are from Papaver somniferum, Eschscholzia californica, Argemone mexicana, Aquilegia formosa, Corydalis cheilanthifolia, Coptis chinensis, Coptis japonica, Chelidonium majus, Cissampelos mucronata, Glaucium flavum, Hydrastis canadensis, Mahonia aquifolium, Menispermum canadense, Nandina domestica, Nelumbo nucifera, Papaver bracteatum, Podophyllum peltatum, Sanguinaria canadensis, Stylophorum diphyllum, Sinopodophyllum hexandrum, Thalictrum flavum or Xanthorhiza simplicissima. In a more specific embodiment, the P6H, when present is from Eschscholzia californica; and/or 6OMT, CNMT, 4′OMT2, BBE, CFS, SPS, TNMT, MSH, P6H, CPR and/or cytochrome b5, when present are from Papaver somniferum and/or the protoberberine Ring A closer (e.g., able to convert scoulerine into nandinine and/or cheilanthifoline into stylopine) are from Eschscholzia californica, Argemone mexicana, Aquilegia formosa, Corydalis cheilanthifolia, Coptis japonica, Chelidonium majus, Glaucium flavum, Mahonia aquifolium, Nandina domestica, Sanguinaria canadensis, Stylophorum diphyllum, Thalictrum flavum or Xanthorhiza simplicissima and/or the protoberberine Ring B closer (e.g. able to convert scoulerine into cheilanthifoline and/or nandinine into stylopine) are from Papaver somniferum, Eschscholzia californica, Argemone mexicana, Corydalis cheilanthifolia, Chelidonium majus, Glaucium flavum, Nandina domestica, Sanguinaria canadensis, or Stylophorum diphyllum.

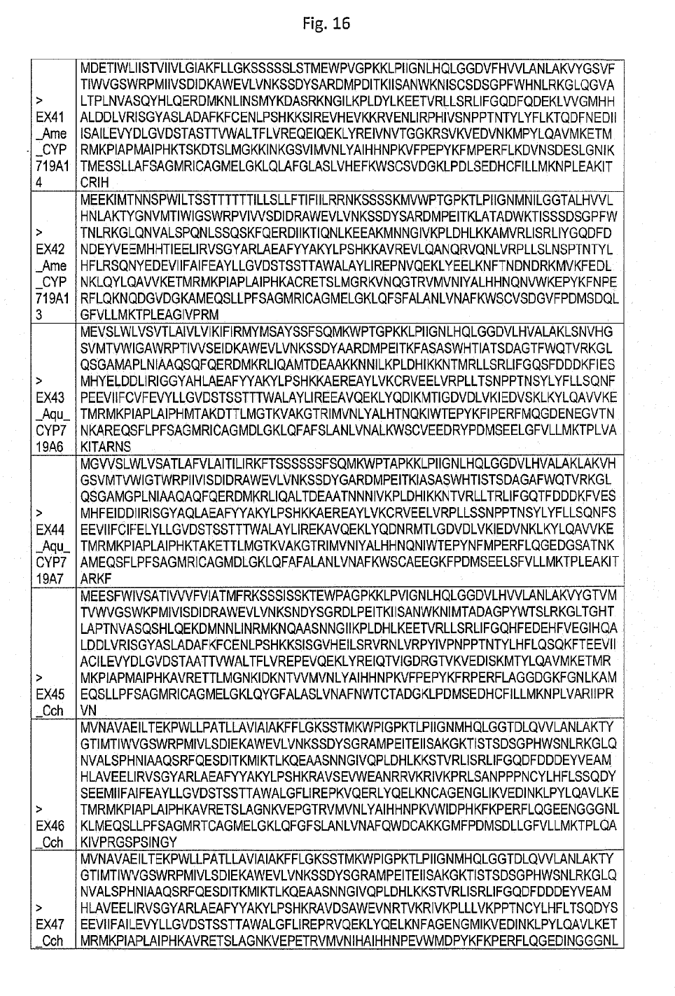

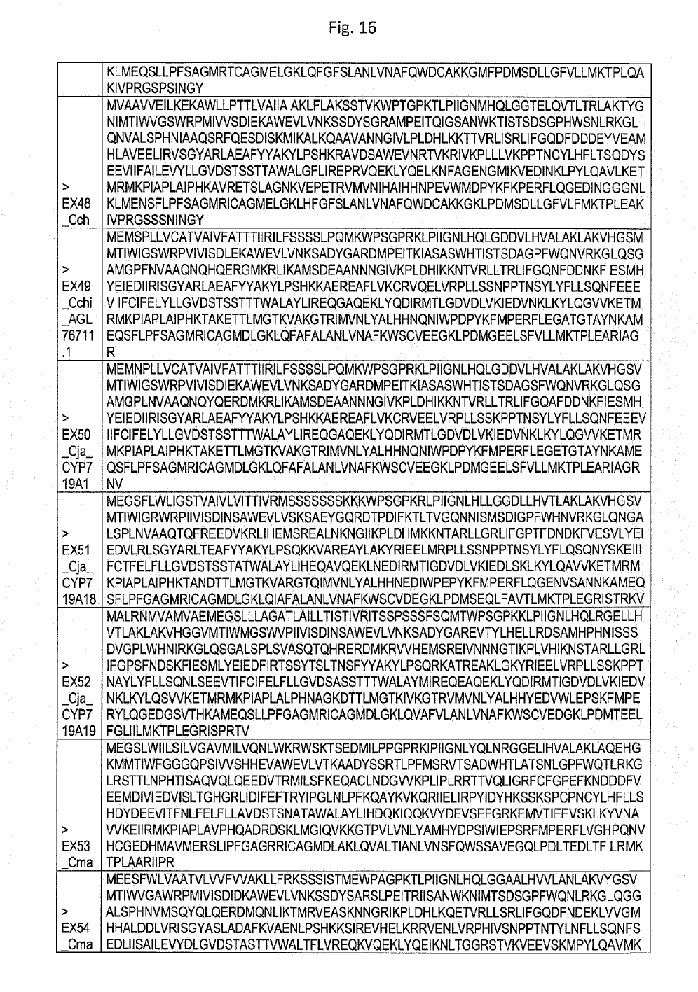

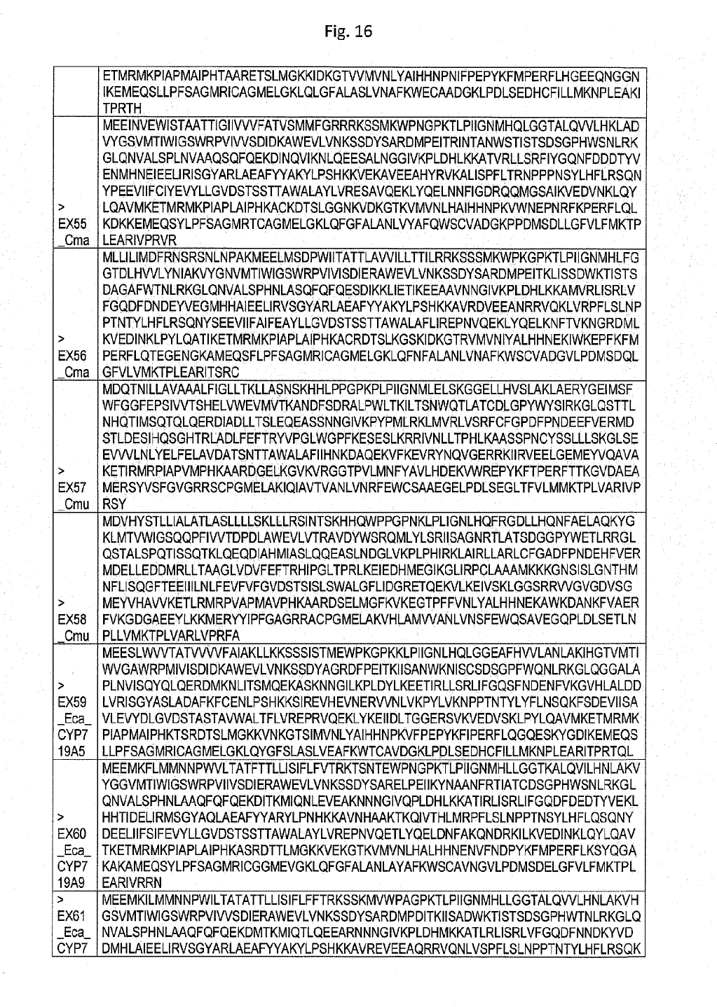

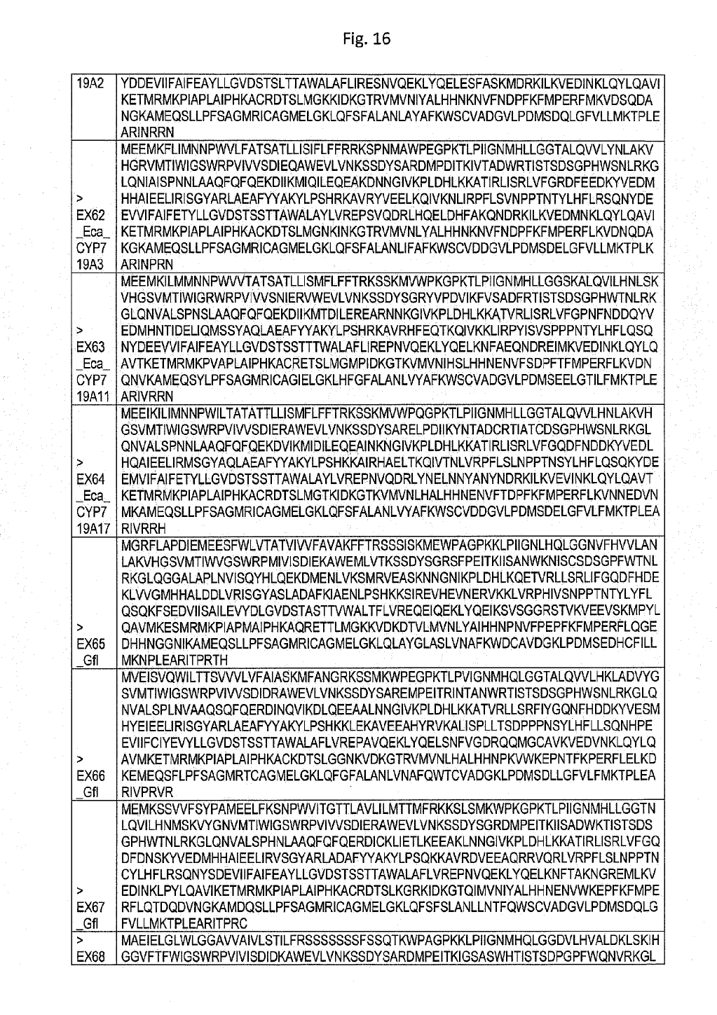

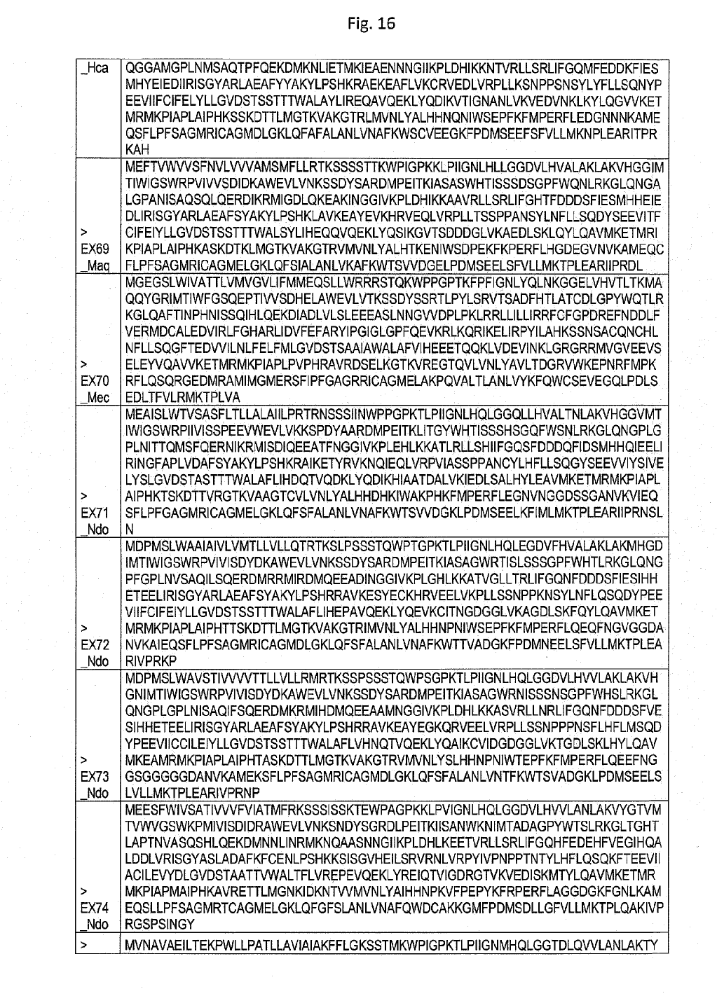

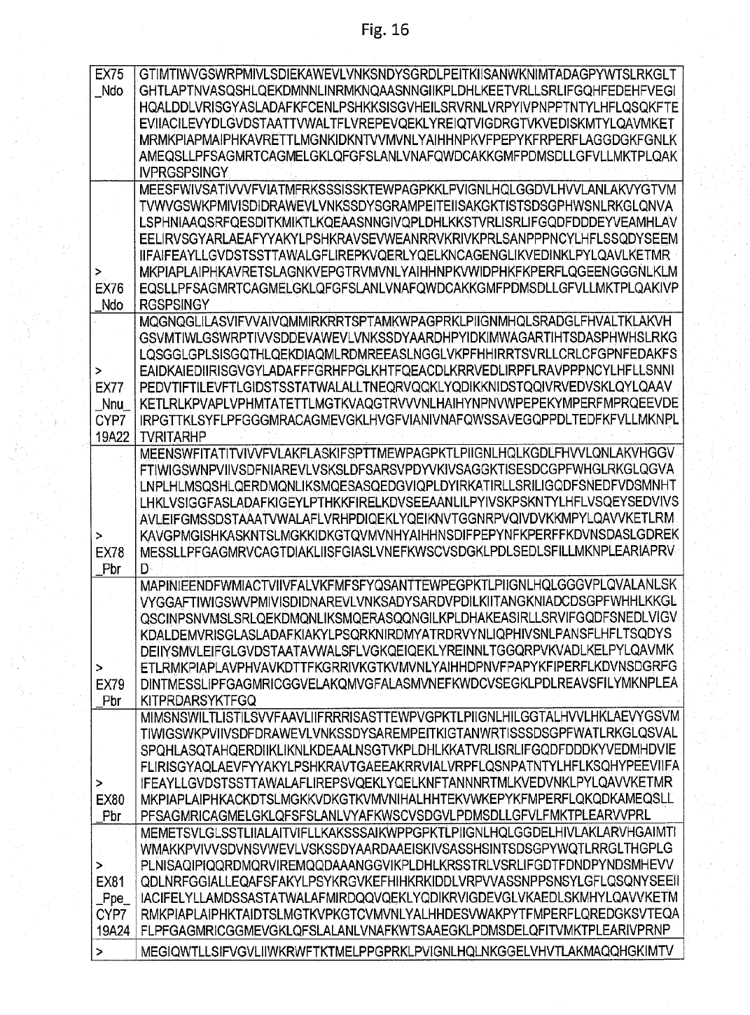

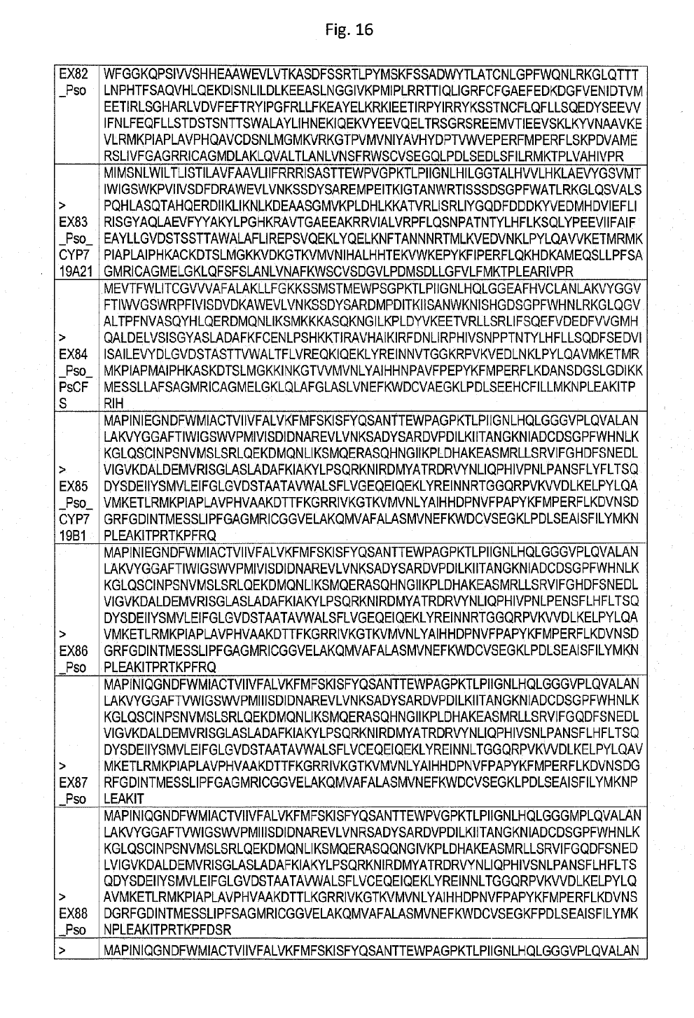

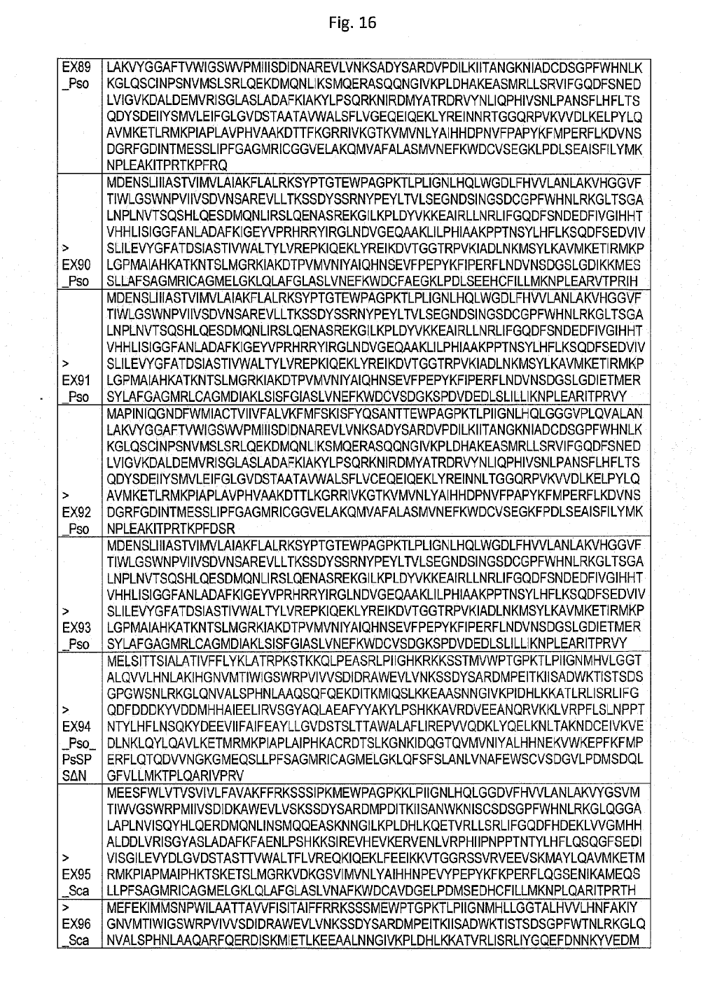

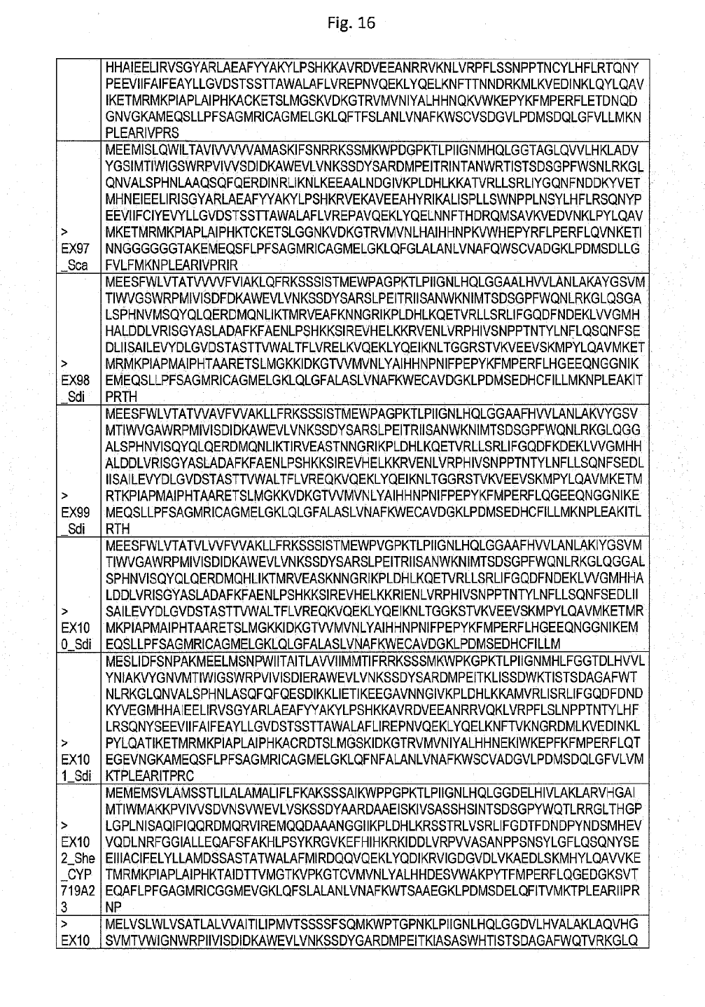

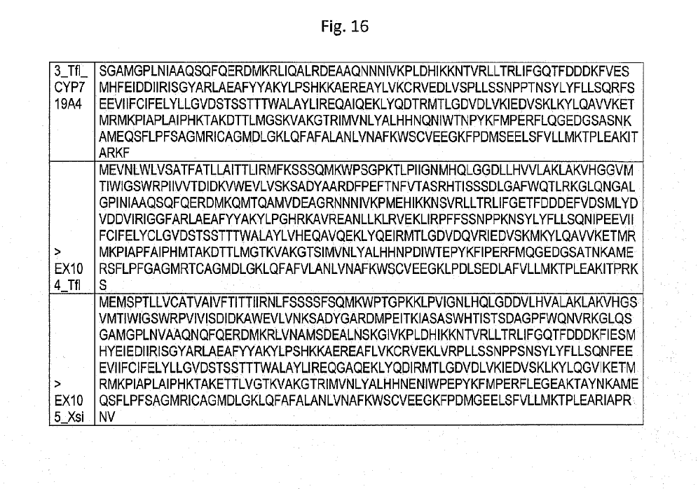

For example, the enzymes may be as described in FIG. 13. Hence 6-OMT as depicted in FIG. 13, SEQ ID NO: 34 (Papaver somniferum derived—codon-optimized by DNA2.0 for optimal expression in yeast) and encoded by SEQ ID NO: 35; or as depicted in in FIG. 13, SEQ ID NO: 36 (Papaver somniferum native) and encoded by SEQ ID NO: 37; CNMT as depicted in FIG. 13, SEQ ID NO: 38 (Papaver somniferum derived—codon-optimized by DNA2.0 for optimal expression in yeast) and encoded by SEQ ID NO: 39; or as depicted in in FIG. 13, SEQ ID NO: 40 (Papaver somniferum native) and encoded by SEQ ID NO: 41; 4′OMT2 as depicted in FIG. 13, SEQ ID NO: 42 (Papaver somniferum derived—codon-optimized by DNA2.0 for optimal expression in yeast) and encoded by SEQ ID NO: 43; or as depicted in in FIG. 13, SEQ ID NO: 44 (Papaver somniferum native) and encoded by SEQ ID NO: 45; BBE as depicted in FIG. 13, SEQ ID NO: 46 (Papaver somniferum truncated (PsBBEΔN)) and encoded by SEQ ID NO: 47; or as depicted in in FIG. 13, SEQ ID NO: 48 (Papaver somniferum native (PsBBE)) and encoded by SEQ ID NO: 49; CFS as depicted in FIG. 13, SEQ ID NO: 50 (ADB89213 Papaver somniferum CFS) and encoded by SEQ ID NO: 51; or SEQ ID NO: 52 (psCFS cloned into pGC994) and encoded by SEQ ID NO: 53; SPS as depicted in FIG. 13, SEQ ID NO: 54 (truncated Papaver somniferum with N-terminus LsGAO spanning domain (SPSΔN)) and encoded by SEQ ID NO: 55; TNMT as depicted in FIG. 13, SEQ ID NO: 58 (native Papaver somniferum TNMT) and encoded by SEQ ID NO: 59; MSH as depicted in FIG. 13, SEQ ID NO: 60 (native Papaver somniferum MSH) and encoded by SEQ ID NO: 61; P6H as depicted in FIG. 13, SEQ ID NO: 62 (native Eschscholzia californica P6H) and encoded by SEQ ID NO: 63; CPR as depicted in FIG. 13, SEQ ID NO: 64 (Papaver somniferum derived—codon-optimized by DNA2.0 for optimal expression in yeast) and encoded by SEQ ID NO: 65; or as depicted in FIG. 13, SEQ ID NO: 66 (Papaver somniferum native) and encoded by SEQ ID NO: 67; cytochrome b5 as depicted in FIG. 13, SEQ ID NO: 68 (Papaver somniferum b5) and encoded by SEQ ID NO: 69; or as depicted in FIG. 13, SEQ ID NO: 70 (Artemisia annua derived b5) and encoded by SEQ ID NO: 71; Protoberine Ring B closer as depicted in FIG. 16, FIG. 17A, FIG. 17B or FIG. 17C (e.g., EX45 (SEQ ID NO: 324); EX74 (SEQ ID NO: 353); EX41 (SEQ ID NO: 320); EX84 (SEQ ID NO: 363); EX59 (SEQ ID NO: 338); EX99 (SEQ ID NO: 378); EX54 (SEQ ID NO: 333); EX98 (SEQ ID NO: 377); EX65 (SEQ ID NO: 344); and EX95 (SEQ ID NO: 374)); and Protoberine Ring A closer as depicted in FIG. 16, 17D or 17E (e.g., EX76 (SEQ ID NO: 355); EX48 (SEQ ID NO: 327); EX46 (SEQ ID NO: 325); EX47 (SEQ ID NO: 326); EX66 (SEQ ID NO: 345); EX60 (SEQ ID NO: 339); EX42 (SEQ ID NO: 321); EX61 (SEQ ID NO: 340); EX96 (SEQ ID NO: 375); EX67 (SEQ ID NO: 346); EX56 (SEQ ID NO: 335); EX101 (SEQ ID NO: 380); EX44 (SEQ ID NO: 323); EX103 (SEQ ID NO: 382); EX50 (SEQ ID NO: 329); EX105 (SEQ ID NO: 384); EX69 (SEQ ID NO: 348); and EX72 (SEQ ID NO: 351)) and an alignment derived therefrom (SEQ ID NO: 486).

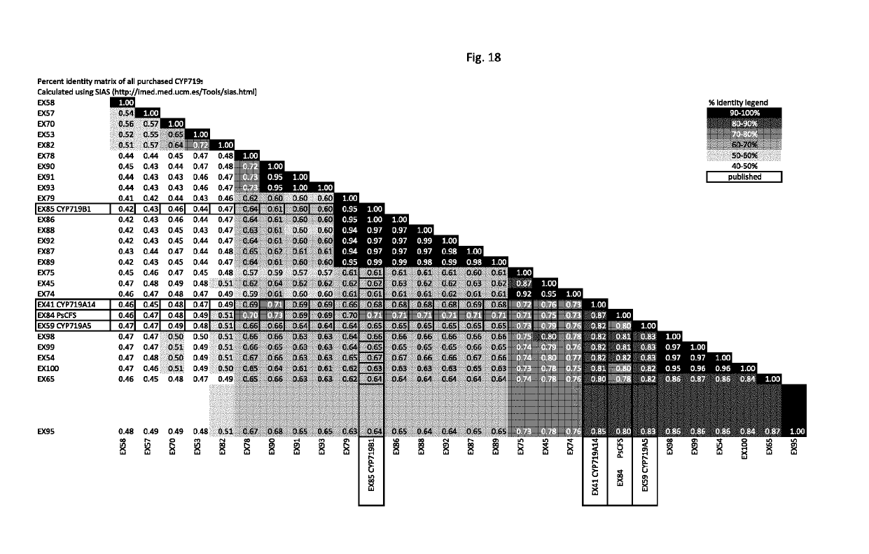

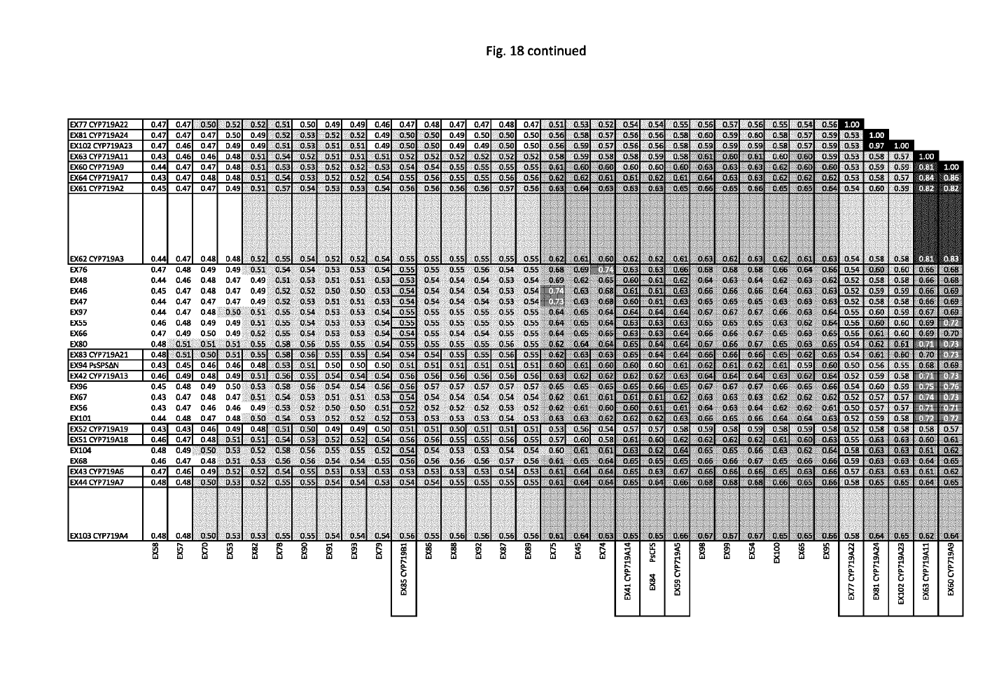

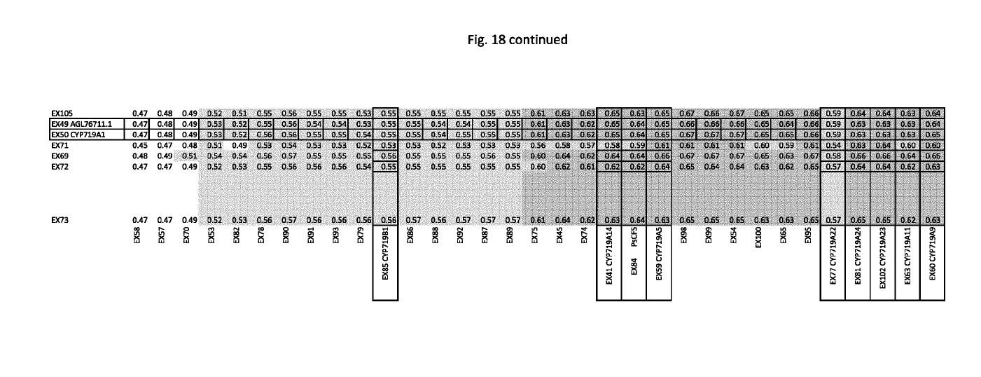

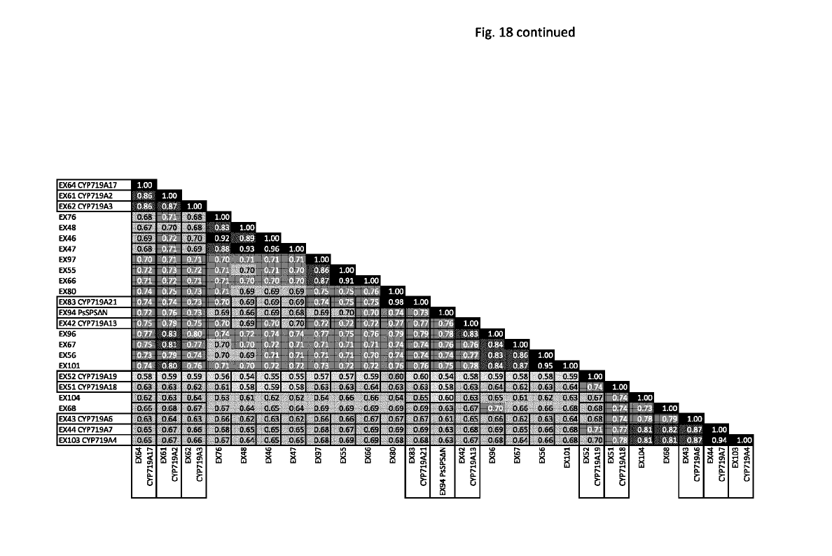

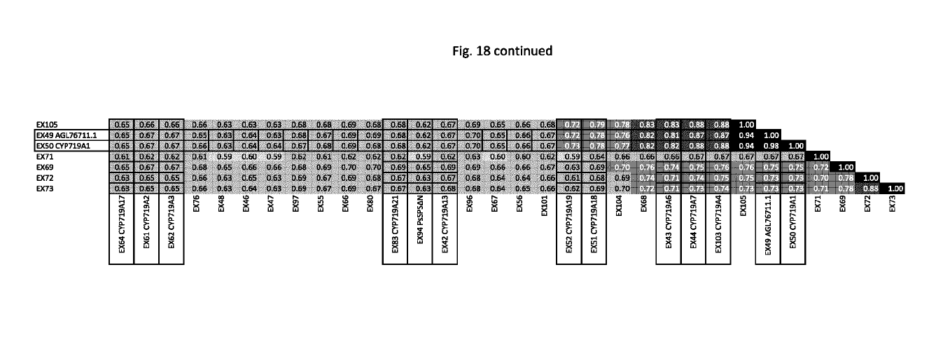

Percent identities between amino acid sequences of certain enzymes of the present invention are also presented (see e.g., FIG. 18 showing percent identities of pairs protoberberine Ring A or Ring B closers of the present invention). Hence enzyme sequences in accordance with the present invention include enzymes with amino acid sequences having high percent identities (e.g., at least 50%, 55%, 60%, 65%, 70%, 75%, 80%, 85%, 90%, 95%, 96%, 97%, 97%, 98% and 99% identity) with enzymes specifically disclosed in the present invention and in particular with those shown to display useful activity (see e.g., Figs. of present invention).

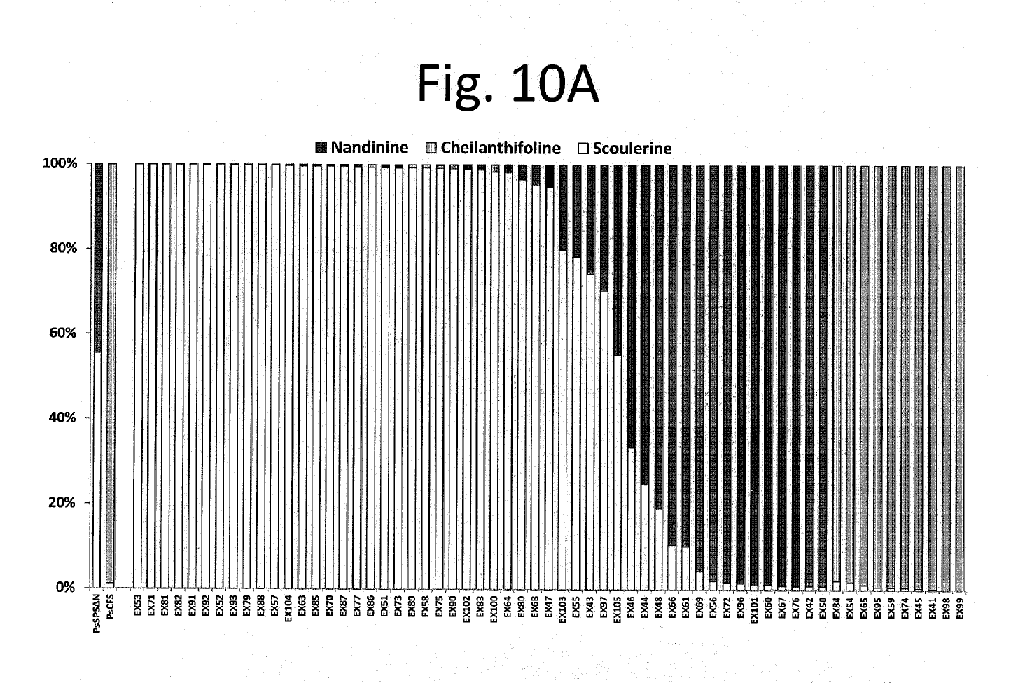

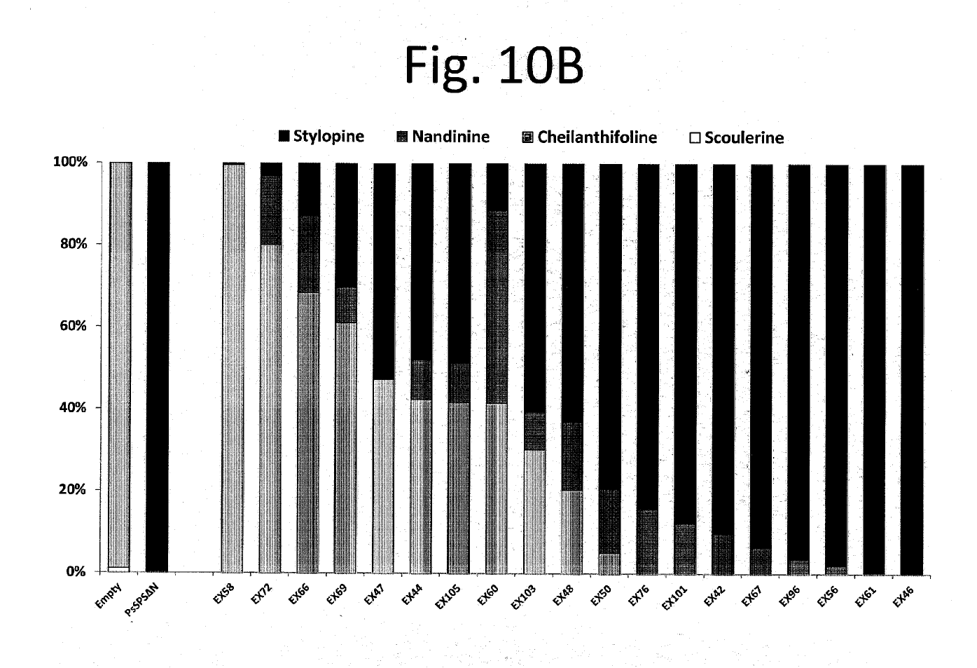

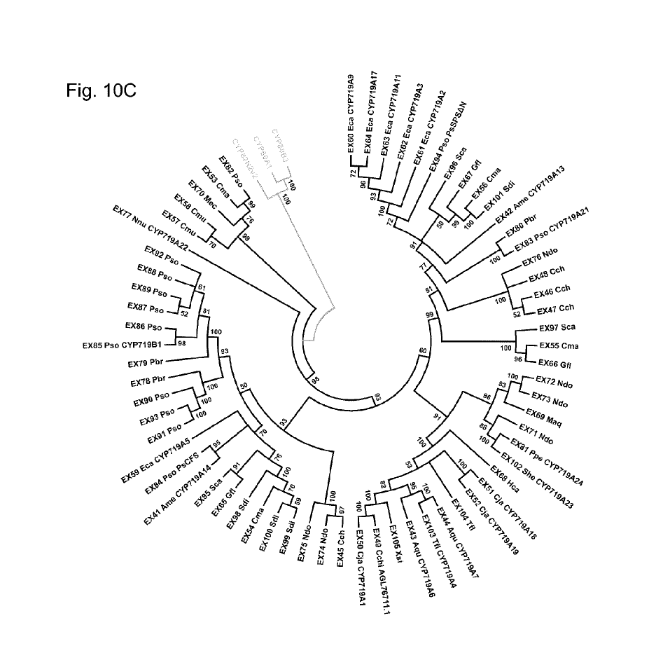

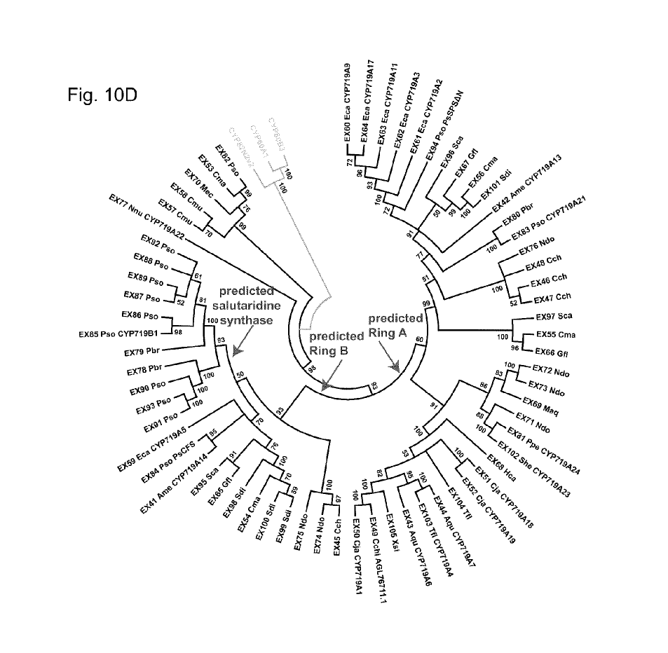

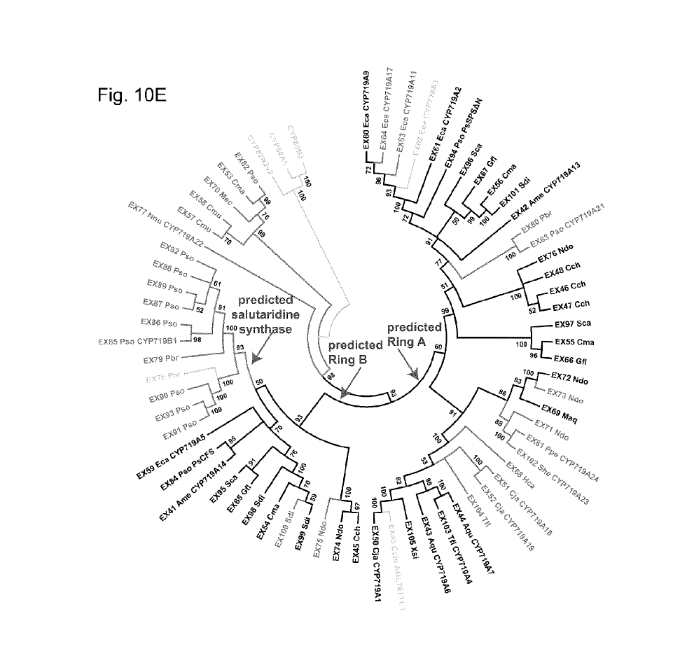

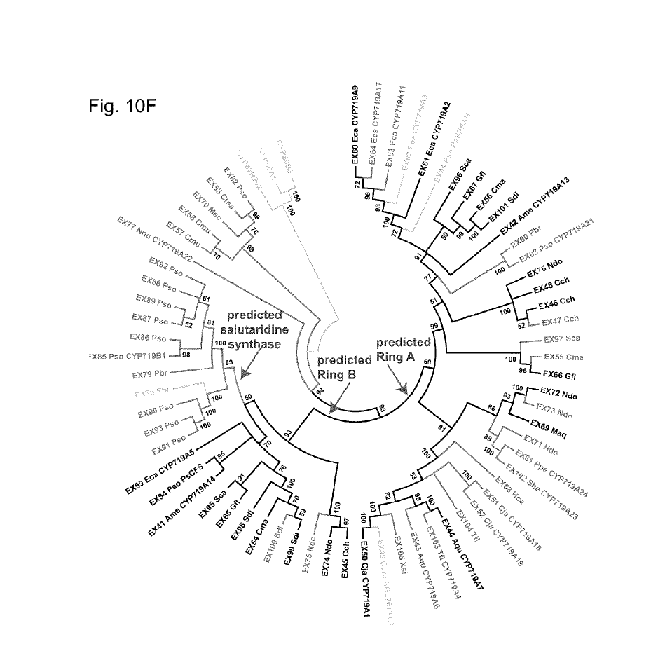

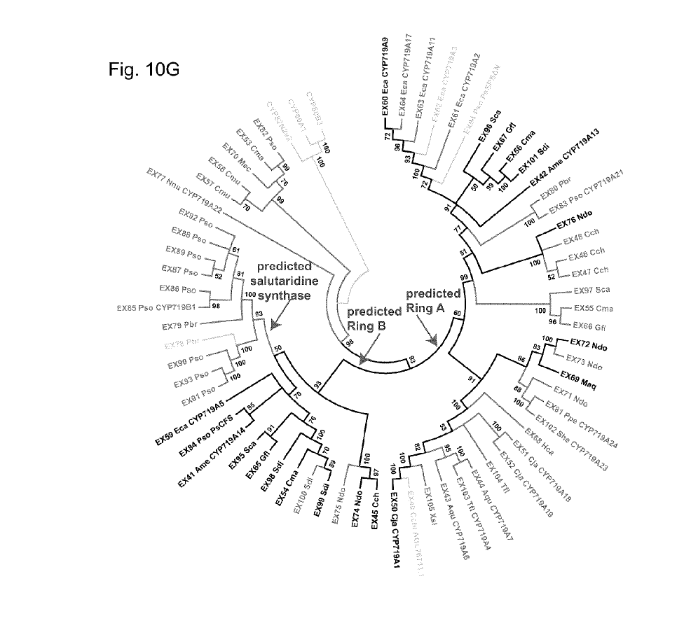

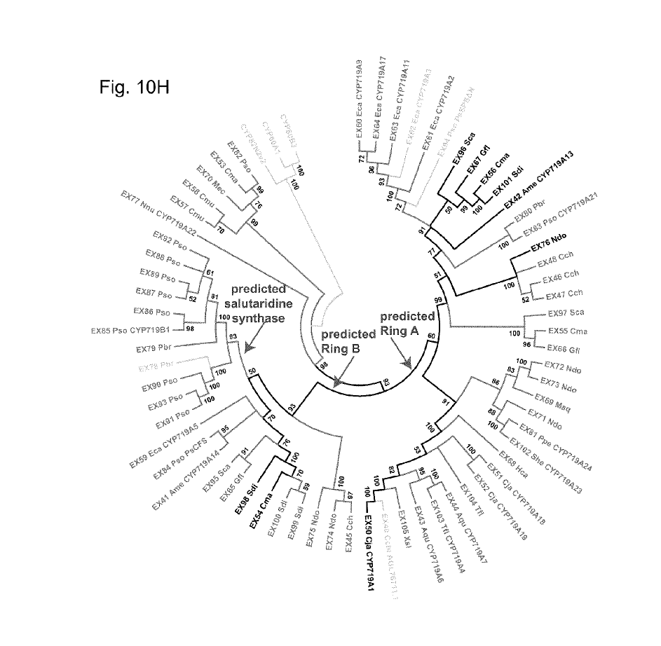

Relatedness of enzymes of the present invention are also presented by way of phylogenetic trees (see e.g., FIG. 10C-H for protoberberine Ring A or Ring B closers of the present invention). Hence enzyme sequences in accordance with the present invention include enzymes shown to be related with enzymes specifically disclosed in the present invention and in particular with those shown to display useful activity for a purpose of the present invention (see e.g., Figs. of present invention) through phylogenetic trees.

The enzymes could also be modified for better expression/stability/yield in the host cell (e.g., replacing the native N-terminal membrane-spanning domain by the N-terminal membrane-spanning domain from another plant or yeast gene (e.g., Lactuca sativa (lettuce) germacrene A oxidase) or from a yeast ER bound protein (e.g., erg1 or erg8); codon optimization for expression in the heterologous host; use of different combinations of promoter/terminators for optimal coexpression of multiple enzymes; spatial colocalization of sequential enzymes using a linker system or organelle-specific membrane domain. In a more specific embodiment, useful enzymes are as shown in FIGS. 13-17. Transmembrane domains can be predicted using, for example, the software TMpred™ (ExPASy) http://www.ch.embnet.org/software/TMPRED_form.html. Tmpred predicted alpha-helix transmembrane domains for: CFS: AA 3 to 24; MSH: AA 18 to 36; and P6H: AA 8 to 27. These domains could be replaced by different transmembrane domains and/or simply truncated (e.g., BBEΔN) and lead to proper folded, stable and functional transmembrane proteins.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g., size, charge, or polarity).

In a non-limiting example, a conservative mutation may be an amino acid substitution. Such a conservative amino acid substitution may be a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (He or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2, BLAST-P, BLAST-N, COBALT or FASTA-N, or any other appropriate software/tool that is known in the art (Johnson M, et al. (2008) Nucleic Acids Res. 36:W5-W9; Papadopoulos J S and Agarwala R (2007) Bioinformatics 23:1073-79).

The substantially identical sequences of the present invention may be at least 75% identical; in another example, the substantially identical sequences may be at least 80, 85, 90, 95, 96, 97, 98 or 99% identical at the amino acid level to sequences described herein. The substantially identical sequences retain substantially the activity and specificity of the reference sequence.

### Nucleic Acids, Host Cells

The present invention also relates to nucleic acids comprising nucleotide sequences encoding the above-mentioned enzymes. The nucleic acid may be codon-optimized. The nucleic acid can be a DNA or an RNA. The nucleic acid sequence can be deduced by the skilled artisan on the basis of the disclosed amino acid sequences. In a specific embodiment, the nucleic acid encodes one of the amino acid sequences as presented in any one of FIGS. 13 to 17 (orthologues and/or consensuses). In another specific embodiment, the nucleic acid for one or more enzymes is as shown in FIG. 13.

The present invention also encompasses vectors (plasmids) comprising the above-mentioned nucleic acids. The vectors can be of any type suitable, e.g., for expression of said polypeptides or propagation of genes encoding said polypeptides in a particular organism. The organism may be of eukaryotic or prokaryotic origin (e.g., yeast). The specific choice of vector depends on the host organism and is known to a person skilled in the art. In an embodiment, the vector comprises transcriptional regulatory sequences or a promoter operably-linked to a nucleic acid comprising a sequence encoding an enzyme involved in the BIA pathway of the invention. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequence. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory sequences” or “transcriptional regulatory elements” are generic terms that refer to DNA sequences, such as initiation and termination signals (terminators), enhancers, and promoters, splicing signals, polyadenylation signals, etc., which induce or control transcription of protein coding sequences with which they are operably-linked.

Plasmids useful to express the enzymes of the present invention include the modified centromeric plasmids pGREG503 (FIG. 13, SEQ ID NO: 1), pGREG504 (FIG. 13, SEQ ID NO: 2), pGREG505 (FIG. 13, SEQ ID NO: 3) and pGREG506 (FIG. 13, SEQ ID NO: 4) from the pGREG series⁵⁵, the 2μ plasmids pYES2 (Invitrogen) (FIG. 13, SEQ ID NO: 5), pESC-leu2 derivative pESC-leu2d (Erhart E. and Hollenberg C. P., J. Bacteriol 1983, p 625) (FIG. 13, SEQ ID NO: 6), pGC550 (SEQ ID NO: 556), pGC552 (SEQ ID NO: 557), pGC1322 (SEQ ID NO: 558), pBOT-TRP (SEQ ID NO: 561), pBOT-URA (SEQ ID NO: 562), pBOT-HIS (SEQ ID NO: 563) and pBOT-LEU (SEQ ID NO: 564). Yeast Artificial Chromosome (YACs) able to clone fragments of 100-1000 kpb could also be used to express multiple enzymes (e.g., 10). Many other useful yeast expression vectors, either autonomously replicating low copy-number vectors (YCp or centromeric) or autonomously replicating high copy-number vectors (YEp or 2μ) are commercially available, e.g., from Invitrogen (www.lifetechnologies.com), the American Type Culture Collection (ATCC; www.atcc.org) or the Euroscarf collection (http://web.uni-frankfurt.deffb15/mikro/euroscarf/).

Plasmids including enzymes in accordance with specific embodiments of the present invention include pGC1189 (CPR); pGC1190 (CPRb-CFS); pGC1191(CPRb-SPSΔNb); pGC1062 (Block 1) (FIG. 13, SEQ ID NO: 7); pGC994 (Block 2) (FIG. 13, SEQ ID NO: 8); pGC997 (Block 3) (FIG. 13, SEQ ID NO: 9); pGC557 (CPR) (FIG. 13, SEQ ID NO: 10); pGC655 (BBEΔN-2μ) (FIG. 13, SEQ ID NO: 11); pGC717 (CPR-TNMT); and pBOT-TRP-(EX41-105), etc. as shown in Table 1. Plasmids in accordance with the present invention may also include nucleic acid molecule(s) encoding one or more of the polypeptides as shown in FIGS. 13 to 17 (orthologues or consensuses).

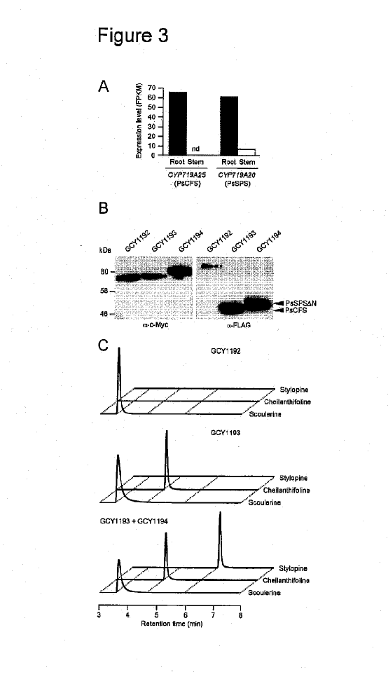

Promoters useful to express the enzymes of the present invention include the constitutive promoters from the following S. cerevisiae CEN.PK2-1 D genes: glyceraldehyde-3-phosphate dehydrogenase 3 (P_{TDH3}) (FIG. 13, SEQ ID NO: 12), fructose 1,6-bisphosphate aldolase (P_{FBA1}) (FIG. 13, SEQ ID NO: 13), pyruvate decarboxylase 1 (P_{PDC1}) (FIG. 13, SEQ ID NO: 14) and plasma membrane H⁺-ATPase 1 (P_{PMA1}) (FIG. 13, SEQ ID NO: 15). The inducible promoters from galactokinase (P_{GAL1}) (FIG. 13, SEQ ID NO: 16), UDP-glucose-4-epimerase (P_{GAL10}) (FIG. 13, SEQ ID NO: 17) from pESC-leu2d are also useful for the present invention. For example, they were used for the first characterization of PsCFS and PsSPS. The present invention also encompasses using other available promoters (e.g., yeast promoters), with different strengths and different expression profiles. Examples are the P_{TEF1} (FIG. 13, SEQ ID NO: 18) and P_{TEF2} (FIG. 13, SEQ ID NO: 19) promoters from the translational elongation factor EF-1 alpha paralogs TEF1 and TEF2; promoters of gene coding for enzymes involved in glycolysis such as 3-phosphoglycerate kinase (P_{PGK1}) (FIG. 13, SEQ ID NO: 20), pyruvate kinase (P_{PYK1}) (FIG. 13, SEQ ID NO: 21), triose-phosphate isomerase (P_{TPI1}) (FIG. 3, SEQ ID NO: 22), glyceraldehyde-3-phosphate dehydrogenase (P_{TDH2}) (FIG. 13, SEQ ID NO: 23), enolase II (P_{ENO2}) (FIG. 13, SEQ ID NO: 24) or hexose transporter 9 (P_{HXT9}) (FIG. 13, SEQ ID NO: 25). Other useful promoters in accordance with the present invention encompass those found through the promoter database of S. cerevisiae (http://rulai.cshl.edu/cgi-bin/SCPD/getgenelist).

Terminators useful for the present invention include terminators from the following S. cerevisiae CEN.PK2_1 D genes: cytochrome C1 (T_{CYC1}) (FIG. 13, SEQ ID NO: 26), alcohol dehydrogenase 1 (T_{ADH1}) (FIG. 13, SEQ ID NO: 27), phosphoglucoisomerase 1 glucose-6-phosphate isomerase (T_{PGI1}) (FIG. 13, SEQ ID NO: 28). The present invention also encompasses using other suitable yeast terminators, e.g., terminators from genes encoding for enzymes involved in glycolysis and gluconeogenesis such as alcohol dehydrogenase 1 (T_{ADH2}) (FIG. 13, SEQ ID NO: 29), enolase II (T_{EN02}) (FIG. 13, SEQ ID NO: 30), fructose 1,6-bisphosphate aldolase (T_{FBA1}) (FIG. 13, SEQ ID NO: 31), glyceraldehyde-3-phosphate dehydrogenase (T_{TDH2}) (FIG. 13, SEQ ID NO: 32) and triose-phosphate isomerase (T_{TPI1}) (FIG. 13, SEQ ID NO: 33). Other useful terminators in accordance with the present invention encompass those found from genes indicated in the promoter database of S. cerevisiae (http://rulai.cshl.edu/cgi-bin/SCPD/getgenelist).

The term “heterologous coding sequence” refers herein to a nucleic acid molecule that is not normally produced by the host cell in nature.

The terms “benzylisoquinoline alkaloid metabolite” or “BIA metabolite” as used herein refer to any BIA metabolite produced by the host cells of the present invention when fed the relevant substrate. Such BIA metabolites include plant native (e.g., reticuline) and non-native metabolites (e.g., N-methylscoulerine and N-methylcheilanthifoline). Without being so limited, it includes (R,S)-6-O-methyl-norlaudanosoline, (R,S)-3′-hydroxy-N-methylcoclaurine, (R,S)-reticuline, (R)-reticuline, (S)-reticuline, (S)-scoulerine, (S)-cheilanthifoline, (S)-stylopine, (S)—N-cis-methylstylopine, protopine, 6-hydroxyprotopine, dihydrosanguinarine, sanguinarine, N-methylscoulerine, N-methylcheilanthifoline, racemic mixtures of any of these compounds and stereoisomers of any of these compounds.

A recombinant expression vector (plasmid) comprising a nucleic acid sequence of the present invention may be introduced into a cell, e.g., a host cell, which may include a living cell capable of expressing the protein coding region from the defined recombinant expression vector. Accordingly, the present invention also relates to cells (host cells) comprising the nucleic acid and/or vector as described above. The suitable host cell may be any cell of eukaryotic (e.g., yeast) or prokaryotic (bacterial) origin that is suitable, e.g., for expression of the enzymes or propagation of genes/nucleic acids encoding said enzyme. The eukaryotic cell line may be of mammalian, of yeast, or invertebrate origin. The specific choice of cell line is known to a person skilled in the art. The terms “host cell” and “recombinant host cell” are used interchangeably herein. Such terms refer not only to the particular subject cell, but also to the progeny or potential progeny of such a cell. Because certain modifications may occur in succeeding generations due to either mutation or environmental influences, such progeny(ies) may not, in fact, be identical to the parent cell, but are still included within the scope of the term as used herein. Vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), Sambrook and Russell (supra) and other laboratory manuals. Methods for introducing nucleic acids into mammalian cells in vivo are also known, and may be used to deliver the vector DNA of the invention to a subject for gene therapy.

In a specific embodiment, the host cells can be a yeast or a bacteria (E. coli). In a more specific embodiment, it can be a Saccharomycetaceae such as a Saccharomyces, Pichia or Zygosaccharomyces. In a more specific embodiment, it can be a Saccharomyces. In a more specific embodiment, it can be a Saccharomyces cerevisiae (S. cerevisiae). Yeast is advantageous in that cytochrome P450 proteins, involved in certain steps in the BIA pathways, are able to fold properly into the endoplasmic reticulum membrane so that activity is maintained, as opposed to bacterial cells which lack such intracellular compartments. The present invention encompasses the use of yeast strains that are aploid, and contain auxotropies for selection that facilitate the manipulation with plasmid. Yeast strains that can be used in the invention include, but are not limited to, CEN.PK, S288C, W303, A363A and YPH499, strains derived from S288C (FY4, DBY12020, DBY12021, XJ24-249) and strains isogenic to S288C (FY1679, AB972, DC5). In specific examples, the yeast strain is any of CEN.PK2-1D (MATalpha ura3-52; trp1-289; leu2-3,112; his3Δ 1; MAL2-8^{C}; SUC2) or CEN.PK2-1C (MATa ura3-52; trp1-289; leu2-3,112; his3Δ 1; MAL2-8c; SUC2) or any of their single, double or triple auxotrophs derivatives. In a more specific embodiment, the yeast strain is any of the yeast strains listed in Table 1 or Table 2. In another specific embodiment, the particular strain of yeast cell is S288C (MATalpha SUC2 mal mel gal2 CUP1 flo1 flo8-1 hap1), which is commercially available. In another specific embodiment, the particular strain of yeast cell is W303.alpha (MAT.alpha.; his3-11,15 trp1-1 leu2-3 ura3-1 ade2-1), which is commercially available. The identity and genotype of additional examples of yeast strains can be found at EUROSCARF, available through the World Wide Web at web.uni-frankfurt.deffb15/mikro/euroscarf/col_index.html or through the Saccharomyces Genome Database (www.yeastgenome.org).

The above-mentioned nucleic acid or vector may be delivered to cells in vivo (to induce the expression of the enzymes and generates BIA metabolites in accordance with the present invention) using methods well known in the art such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo. A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor. Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes.

### Methods of Preparing a Benzylisoquinoline Alkaloid (BIA) Metabolite

The present invention encompasses a method of using a host cell as described above expressing enzymes in accordance with the present invention for generating a significant yield of benzylisoquinoline alkaloid. The applicants have surprisingly discovered that by using first buffering conditions enabling the maintenance of a useful pH of over about 7, and, optionally, second buffering conditions between about 3 and about 6, the host cells of the present invention produced a significantly improved yield of BIA metabolite.

The present invention therefore provide a method of using a host cell as described above expressing enzymes in accordance with the present invention for generating a significant yield of benzylisoquinoline alkaloid using a first useful pH. As used herein, the terms “first useful pH” refer to a pH used for a first fermentation and refer to a pH of over about 7 (over about 7, 7.1, 7.2, 7.3, 7.4, 7.5, 7.6, 7.7, 7.8, 7.9 or 8, etc.), more preferably between about 7 (or about 7, 7.1, 7.2, 7.3, 7.4, 7.5, 7.6, 7.7, 7.8, 7.9 or 8, etc.) and about 10 (or about 9, 9.1, 9.2, 9.3, 9.4, 9.5, 9.6, 9.7, 9.8, 9.9 or 10), more preferably, about 7 (or about 7, 7.1, 7.2, 7.3, 7.4, 7.5, 7.6, 7.7, 7.8 or 7.9, etc.) to about 9 (or about 8, 8.1, 8.2, 8.3, 8.4, 8.5, 8.6, 8.7, 8.8, 8.9 or 9); about 7 to about 9.9; about 7 to about 9.8; about 7 to about 9.7; about 7 to about 9.6; about 7 to about 9.5; about 7 to about 9.4; about 7 to about 9.3; about 7 to about 9.2; about 7 to about 9.1; about 7.1 to about 8.9; about 7.1 to about 8.8; about 7.1 to about 8.7; about 7.1 to about 8.6; about 7.1 to about 8.5; about 7.1 to about 8.4; about 7.1 to about 8.3; about 7.1 to about 8.2; about 7.1 to about 8.1; about 7.2 to about 9.9; about 7.2 to about 9.8; about 7.2 to about 9.7; about 7.2 to about 9.6; about 7.2 to about 9.5; about 7.2 to about 9.4; about 7.2 to about 9.3; about 7.2 to about 9.2; about 7.2 to about 9.1; about 7.2 to about 8.9; about 7.2 to about 8.8; about 7.2 to about 8.7; about 7.2 to about 8.6; about 7.2 to about 8.5; about 7.2 to about 8.4; about 7.2 to about 8.3; about 7.2 to about 8.2; about 7.2 to about 8.1; about 7.2 to about 8.6; about 7.2 to about 8.5; about 7.2 to about 8.4; about 7.2 to about 8.3; about 7.2 to about 8.2; about 7.2 to about 8.1; about 7.2 to about 9.9; about 7.2 to about 9.8; about 7.2 to about 9.7; about 7.2 to about 9.6; about 7.2 to about 9.5; about 7.2 to about 9.4; about 7.2 to about 9.3; about 7.2 to about 9.2; about 7.2 to about 9.1; about 7.2 to about 8.9; about 7.2 to about 8.8; about 7.2 to about 8.7; about 7.2 to about 8.6; about 7.2 to about 8.5; about 7.2 to about 8.4; about 7.2 to about 8.3; about 7.2 to about 8.2; about 7.2 to about 8.1; about 7.3 to about 8.6; about 7.3 to about 8.5; about 7.3 to about 8.4; about 7.3 to about 8.3; about 7.3 to about 8.2; about 7.3 to about 8.1; about 7.3 to about 9.9; about 7.3 to about 9.8; about 7.3 to about 9.7; about 7.3 to about 9.6; about 7.3 to about 9.5; about 7.3 to about 9.4; about 7.3 to about 9.3; about 7.3 to about 9.2; about 7.3 to about 9.1; about 7.3 to about 8.9; about 7.3 to about 8.8; about 7.3 to about 8.7; about 7.3 to about 8.6; about 7.3 to about 8.5; about 7.3 to about 8.4; about 7.3 to about 8.3; about 7.3 to about 8.2; about 7.3 to about 8.1; about 7.4 to about 8.6; about 7.4 to about 8.5; about 7.4 to about 8.4; about 7.4 to about 8.3; about 7.4 to about 8.2; about 7.4 to about 8.1; about 7.4 to about 9.9; about 7.4 to about 9.8; about 7.4 to about 9.7; about 7.4 to about 9.6; about 7.4 to about 9.5; about 7.4 to about 9.4; about 7.4 to about 9.3; about 7.4 to about 9.2; about 7.4 to about 9.1; about 7.4 to about 8.9; about 7.4 to about 8.8; about 7.4 to about 8.7; about 7.4 to about 8.6; about 7.4 to about 8.5; about 7.4 to about 8.4; about 7.4 to about 8.3; about 7.4 to about 8.2; about 7.4 to about 8.1; about 7.5 to about 8.6; about 7.5 to about 8.5; about 7.5 to about 8.4; about 7.5 to about 8.3; about 7.5 to about 8.2; about 7.5 to about 8.1; about 7.5 to about 9.9; about 7.5 to about 9.8; about 7.5 to about 9.7; about 7.5 to about 9.6; about 7.5 to about 9.5; about 7.5 to about 9.4; about 7.5 to about 9.3; about 7.5 to about 9.2; about 7.5 to about 9.1; about 7.5 to about 8.9; about 7.5 to about 8.8; about 7.5 to about 8.7; about 7.5 to about 8.6; about 7.5 to about 8.5; about 7.5 to about 8.4; about 7.5 to about 8.3; about 7.5 to about 8.2; about 7.5 to about 8.1; about 7.6 to about 8.6; about 7.6 to about 8.5; about 7.6 to about 8.4; about 7.6 to about 8.3; about 7.6 to about 8.2; about 7.6 to about 8.1; about 7.6 to about 9.9; about 7.6 to about 9.8; about 7.6 to about 9.7; about 7.6 to about 9.6; about 7.6 to about 9.5; about 7.6 to about 9.4; about 7.6 to about 9.3; about 7.6 to about 9.2; about 7.6 to about 9.1; about 7.6 to about 8.9; about 7.6 to about 8.8; about 7.6 to about 8.7; about 7.6 to about 8.6; about 7.6 to about 8.5; about 7.6 to about 8.4; about 7.6 to about 8.3; about 7.6 to about 8.2; about 7.6 to about 8.1; about 7.7 to about 8.6; about 7.7 to about 8.5; about 7.7 to about 8.4; about 7.7 to about 8.3; about 7.7 to about 8.2; about 7.7 to about 8.1; about 7.7 to about 9.9; about 7.7 to about 9.8; about 7.7 to about 9.7; about 7.7 to about 9.6; about 7.7 to about 9.5; about 7.7 to about 9.4; about 7.7 to about 9.3; about 7.7 to about 9.2; about 7.7 to about 9.1; about 7.7 to about 8.9; about 7.7 to about 8.8; about 7.7 to about 8.7; about 7.7 to about 8.6; about 7.7 to about 8.5; about 7.7 to about 8.4; about 7.7 to about 8.3; about 7.7 to about 8.2; about 7.7 to about 8.1; about 7.8 to about 8.6; about 7.8 to about 8.5; about 7.8 to about 8.4; about 7.8 to about 8.3; about 7.8 to about 8.2; about 7.8 to about 8.1; about 7.8 to about 9.9; about 7.8 to about 9.8; about 7.8 to about 9.7; about 7.8 to about 9.6; about 7.8 to about 9.5; about 7.8 to about 9.4; about 7.8 to about 9.3; about 7.8 to about 9.2; about 7.8 to about 9.1; about 7.8 to about 8.9; about 7.8 to about 8.8; about 7.8 to about 8.7; about 7.8 to about 8.6; about 7.8 to about 8.5; about 7.8 to about 8.4; about 7.8 to about 8.3; about 7.8 to about 8.2; about 7.8 to about 8.1; about 7.9 to about 8.6; about 7.9 to about 8.5; about 7.9 to about 8.4; about 7.9 to about 8.3; about 7.9 to about 8.2; about 7.9 to about 8.1; about 7.9 to about 9.9; about 7.9 to about 9.8; about 7.9 to about 9.7; about 7.9 to about 9.6; about 7.9 to about 9.5; about 7.9 to about 9.4; about 7.9 to about 9.3; about 7.9 to about 9.2; about 7.9 to about 9.1; about 7.9 to about 8.9; about 7.9 to about 8.8; about 7.9 to about 8.7; about 7.9 to about 8.6; about 7.9 to about 8.5; about 7.9 to about 8.4; about 7.9 to about 8.3; about 7.9 to about 8.2; about 7.9 to about 8.1. As used herein, the terms “second useful pH” refer to the pH used for the optional second fermentation and refer to a pH of between about 2.7 and about 6.3 (e.g., YNB conditions), between about 2.8 and about 6.2, between about 2.9 and about 6.1, between about 3 and about 6.0, between about 3 and about 5.9, between about 3 and about 5.8, between about 3 and about 5.7, between about 3 and about 5.6, between about 3 and about 5.5.

Without being so limited, useful buffering conditions capable of maintaining a pH of about 7 to about 10 include: a buffer or mixture of buffers such as Tris; yeast growing medium (e.g., yeast nitrogen broth, synthetic dropout supplement, 2% α-D-glucose and amino acids) (YNB); YNB and a sufficient concentration of Tris; YNB and HEPES; Tris; and Tris and EDTA. Additional examples of such buffers are PBS, PIPES, MOPS, and taurine. A more exhaustive list can be found online at http://www.sigmaaldrich.com/life-science/core-bioreagents/biological-buffers/learning-center/buffer-reference-center.html. In a specific embodiment, such conditions include using about 5 mM to about 150 mM of Tris or Tris and EDTA. In a more specific embodiment, the range is of about 10 to 150 mM; 10 to 140 mM; 10 to 130 mM; 10 to 120 mM; 10 to 110 mM; 10 to 100 mM; 10 to 90 mM; 10 to 80 mM; 10 to 70 mM; 10 to 60 mM; 10 to 55 mM; 10 to 50 mM; 20 to 150 mM; 20 to 140 mM; 20 to 130 mM; 20 to 120 mM; 20 to 110 mM; 20 to 100 mM; 20 to 90 mM; 20 to 80 mM; 20 to 70 mM; 20 to 60 mM; 20 to 55 mM; 20 to 50 mM; 30 to 150 mM; 30 to 140 mM; 30 to 130 mM; 30 to 120 mM; 30 to 110 mM; 30 to 100 mM; 30 to 90 mM; 30 to 80 mM; 30 to 70 mM; 30 to 60 mM; 30 to 55 mM; 30 to 50 mM; 40 to 150 mM; 40 to 140 mM; 40 to 130 mM; 40 to 120 mM; 40 to 110 mM; 40 to 100 mM; 40 to 90 mM; 40 to 80 mM; 40 to 70 mM; 40 to 60 mM; 40 to 55 mM; 40 to 50 mM; 45 to 150 mM; 45 to 140 mM; 45 to 130 mM; 45 to 120 mM; 45 to 110 mM; 45 to 100 mM; 45 to 90 mM; 45 to 80 mM; 45 to 70 mM; 45 to 60 mM; 45 to 55 mM; or 45 to 50 mM.

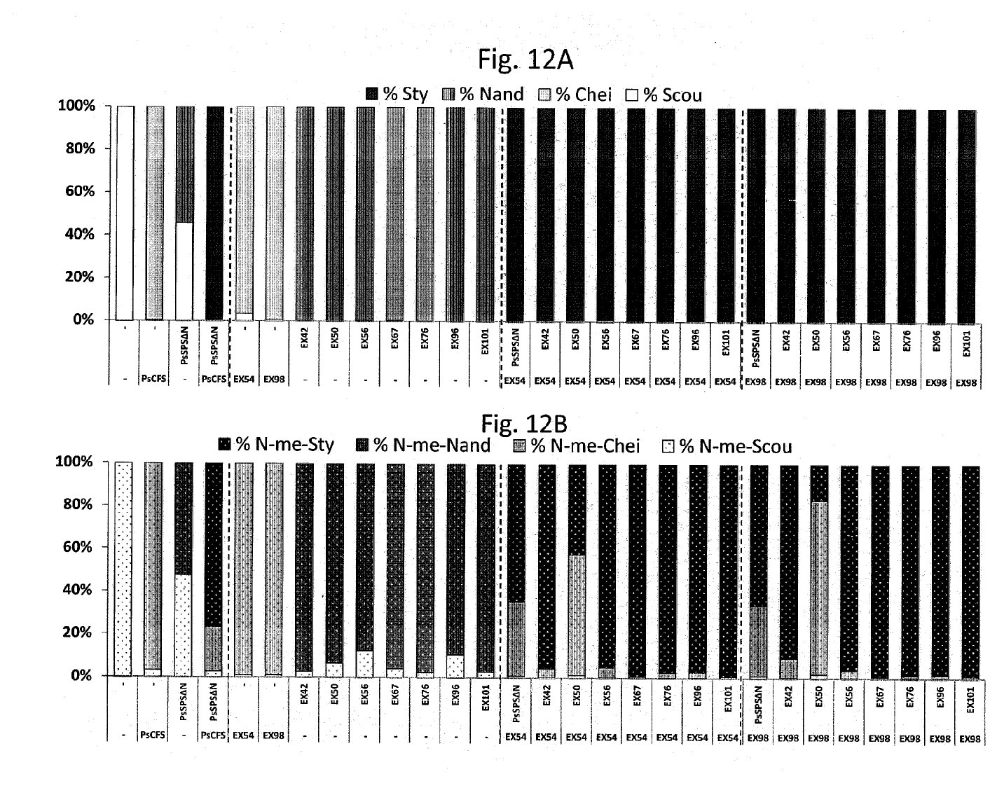

In one embodiment, the method comprising incubating (R,S)-norlaudanosoline (fed substrate) with a host cell expressing 6OMT, CNMT and 4′OMT2 in buffering conditions enabling a useful pH (namely in that case a pH of about 8) yielded about 20% of (S)-reticuline. As used herein the yield may be defined as the ratio of the end product (metabolite) produced to the fed substrate. Hence 20% of the total fed (R,S)-norlaudanosoline was converted to (S)-reticuline in the host cell combined supernatant and cell extract. In another embodiment, the method comprising incubating (S)-scoulerine (fed substrate) with a host cell expressing BBE, CFS, SPS and CPR in buffering conditions enabling a useful pH (namely in that case a pH of about 8) yielded about 19% of (S)-stylopine. In another embodiment, the method comprising incubating (S)-stylopine (fed substrate) with a host cell expressing TNMT, MSH, P6H and CPR in buffering conditions enabling a useful pH (namely in that case a pH of about 8) yielded about 57% of dihydrosanguinarine. In another embodiment, the method comprising incubating (S)-scoulerine (fed substrate) with a host cell expressing BBE, CFS, SPS, TNMT, MSH, P6H and CPR in buffering conditions enabling a useful pH (namely in that case a pH of about 8) yielded about 7.5% of dihydrosanguinarine. In another embodiment, the method comprising incubating (R,S)-norlaudanosoline (fed substrate) with a host cell expressing 6OMT, CNMT, 4′OMT2, BBE, CFS, SPS, TNMT, MSH, P6H and CPR in buffering conditions enabling a useful pH (namely in that case a pH of about 8) yielded about 1.5% of dihydrosanguinarine. As used herein, a significant yield of BIA metabolite includes about 1.5% or more. In another embodiment, the method comprising incubating scoulerine (fed substrate) with a host expressing a Ring A closer and A Ring B closer (see FIGS. 12A-B) generated yields of more than 95% stylopine.

The present invention is illustrated in further details by the following non-limiting examples.

### Example 1

### Methods

### Chemicals and Reagents

(S)-Reticuline was a gift from Johnson & Johnson. (R,S)-Norlaudanosoline was purchased from Enamine Ltd. (Kiev, Ukraine), (S)-scoulerine and (S)-stylopine from ChromaDex (Irvine, Calif., USA), protopine from TRC Inc. (North York, Ontario, Canada) and sanguinarine from Sigma. Dihydrosanguinarine was prepared by NaBH₄ reduction of sanguinarine⁵⁰. Antibiotics, growth media and α-D-glucose were purchased from Sigma-Aldrich. Restriction enzymes, T4 DNA polymerase, and T4 DNA ligase were from New England Biolabs (NEB). Polymerase chain reactions (PCRs) for the assembly of expression cassettes were performed using Phusion™ High-Fidelity DNA polymerase (NEB/Thermo Scientific). Taq polymerase (Fermentas/Thermo Scientific) was used in PCRs confirming DNA assembly or chromosomal integration. PCR-amplified products were gel purified using the QIAquick™ purification kit (Qiagen). Plasmid extractions were done using the GeneJET™ plasmid mini-prep kit (Thermo Scientific). Genomic DNA preparations were done using the DNeasy™ blood and tissue kit (Qiagen). HPLC-grade water was purchased from Fluke. HPLC-grade methanol and acetonitrile were purchased from Fischer Scientific.

### Identification and Characterization of PsCFS and PsSPS

RNA extraction from root or stems of the Papaver somniferum (opium poppy) cultivar Bea's Choice, cDNA library construction, Illumine sequencing, sequence assembly and annotation, and gene expression analysis were performed as described previously⁶.

The pESC-CPR vector encoding opium poppy cytochrome P450 reductase (PsCPR) fused to a c-Myc tag was used for the heterologous expression of plant proteins in S. cerevisiae²⁵. The native PsCPR sequence and that optimized for the host are shown in FIG. 13. PsCFS (CYP719A25; GenBank ADB89213) was amplified from opium poppy cell culture cDNA using the primer listed in Table 3. The amplicon was inserted into NotI and SpeI restriction sites of pESC-CPR in-frame with a FLAG-tag sequence yielding pESC-CPR/CFS. A synthetic SPS (CYP719A20) gene (GenBank KF481962), codon-optimized (SEQ ID NO: 55) (PsSPSΔN) for expression in S. cerevisiae and containing a sequence encoding the N-terminal membrane-spanning domain from the Lactuca sativa (lettuce) germacrene A oxidase (43 amino acids)⁵¹ replacing the corresponding native domain (29 amino acids) to increase protein stability⁵² (recombinant psSPSΔN), was inserted into the NotI and SpeI restriction sites of pESC-CPR²⁵ in-frame with a FLAG-tag sequence yielding pESC-CPR/SPSΔN. Heterologous gene expression in S. cerevisiae strain YPH499, the preparation of microsomes and cytochrome P450 enzyme assays were performed as described previously²⁵. Substrate concentrations in enzyme assays were 125 μM for (S)-scoulerine and 500 μM for NADPH.

Reconstitution of the Sanguinarine Pathway in S. cerevisiae

For liquid cultures, S. cerevisiae was grown in yeast nitrogen broth, synthetic dropout supplement, 2% α-D-glucose and amino acids as appropriate (YNB-DO-GLU) at 30° C. and 200 rpm. For solid media, selection for plasmid transformation was on YNB-DO-GLU/agar, while selection for chromosomal integration was on YPD/agar (yeast extract peptone-dextrose) with the appropriate antibiotic. Lithium acetate transformation was performed according to Gietz and Schiestl⁵³, electroporation was performed according to Shao et al⁹.

Coding sequences for 6OMT, CNMT, 4′OMT2, BBE, CFS, SPS, TNMT, MSH AND CPR are from P. somniferum and that of P6H is from Eschscholzia californica. Coding sequences for protoberberine Ring A and Ring B closers are from various species as listed in Table 6. Synthetic sequences of Ps6OMT (GenBank KF554144), PsCNMT (GenBank KF661326), Ps4′OMT2 (GenBank KF661327), PsSPSΔN and PsP450R(CPR) (GenBank KF661328) were codon-optimized by DNA2.0 for optimal expression in yeast (See FIG. 13, SEQ ID NOs: 34-35, 38-39, 54-55; and 64-65, respectively). The lettuce germacrene A oxidase N-terminus membrane-spanning domain was incorporated into N-truncated P. somniferum stylopine synthase (SPS) as described above for stylopine synthase. Twenty-three amino acids were truncated from N-terminus of the opium poppy BBE, resulting in a truncated version of BBE (PsBBEΔN)^{22,54} (See corresponding sequences in FIG. 13, SEQ ID NOs: 46-47). When not otherwise specified, coding sequences in Table 1 and Table 2 correspond to the plant cDNA sequences. The partial Kozak sequence AAAACA (SEQ ID NO: 482) was introduced upstream of all coding sequences by PCR or as an integral part of gene synthesis.

TABLE 1
List of Saccharomyces cerevisiae strains and plasmids used herein. Full genotypes are available in Table 2

| Strain | Plasmid(s) | Brief description of genotype |
|---|---|---|
| GCY1192 | pGC1189 | MATa leu2-Δ1 |
| GCY1193 | pGC1190 | MATa leu2-Δ1 |
| GCY1194 | pGC1191 | MATa leu2-Δ1 |
| GCY1086 | pGC1062 (Block 1) | MATα leu2-3 |
| GCY1090 | pGC557 (CPR) (SEQ ID NO: 10) | MATα trp1-289 his3 Δ1 |
| | pGC994 (Block 2) (SEQ ID NO: 8) | |
| GCY1094 | pGC557 (CPR) (SEQ ID NO: 10) | MATα ura3-52 his3 Δ1 |
| | pGC997 (Block 3) (SEQ ID NO: 9) | |
| GCY1098 | pGC557 (CPR) (SEQ ID NO: 10) | MATα ura3-52 trp1-289 leu2-3, 112 his3 Δ1 |
| | pGC1062 (Block 1) (SEQ ID NO: 7) | |
| | pGC994 (Block 2) (SEQ ID NO: 8) | |
| | pGC997 (Block 3) (SEQ ID NO: 9) | |
| GCY1101 | pGC557 (CPR) (SEQ ID NO: 10) | Block 2 integrant. MATα ura3-52 trp1-289 leu2- |
| | | 3, 112 his3 Δ1 |
| GCY1104 | pGC557 (CPR) (SEQ ID NO: 10) | Block 2 and 3 integrant. MATα ura3-52 trp1-289 |
| | | leu2-3, 112 his3 Δ1 |
| GCY1108 | pGC557 (CPR) (SEQ ID NO: 10) | Block 2 and 3 integrant. MATα ura3-52 trp1-289 |
| | pGC1062 (Block 1) (SEQ ID NO: 7) | leu2-3, 112 his3 Δ1 |
| GCY1125 | pGC557 (CPR) (SEQ ID NO: 10) | Block 2 and 3 integrant. MATα ura3-52 trp1-289 |
| | pGC1062 (Block 1) (SEQ ID NO: 7) | leu2-3, 112 his3 Δ1 |
| | pGC655 (BBEΔN-2μ) (SEQ ID NO: | |
| | 11) | |
| GCY1127 | pGC717 (CPR-TNMT) | |
| GCY1333 | None | CPR integrant. MATα ura3-52 trp1-289 leu2-3, 112 |
| | | his3 Δ1 |
| GCY1270 | None | CPR and TNMT integrant. MATα ura3-52 trp1-289 |
| | | leu2-3, 112 his3 Δ1 |
| GCY1301 | pGC550 (SOMT + CAS) | CPR and TNMT integrant. MATα ura3-52 trp1-289 |
| | | leu2-3, 112 his3 Δ1 |
| GCY1316 | None | CPR and CFS integrant. MATα ura3-52 trp1-289 |
| | | leu2-3, 112 his3 Δ1 |
| GCY1411 | pGC552 (PsCFS) | CPR integrant. MATα ura3-52 trp1-289 leu2-3, 112 |
| | | his3 Δ1 |
| GCY1412 | pGC1322 (PsSPSΔN) | CPR integrant. MATα ura3-52 trp1-289 leu2-3, 112 |
| | | his3 Δ1 |
| GCY1413 | pGC552 (PsCFS) | CPR integrant. MATα ura3-52 trp1-289 leu2-3, 112 |
| | pGC1322 (PsSPSΔN) | his3 Δ1 |
| GCY1414 | pGC1322 (PsSPSΔN) | CPR and CFS integrant. MATα ura3-52 trp1-289 |
| | | leu2-3, 112 his3 Δ1 |
| GCY1415 | pGC552 (PsCFS) | CPR and TNMT integrant. MATα ura3-52 trp1-289 |
| | | leu2-3, 112 his3 Δ1 |
| GCY1416 | pGC1322 (PsSPSΔN) | CPR and TNMT integrant. MATα ura3-52 trp1-289 |
| | | leu2-3, 112 his3 Δ1 |
| GCY1417 | pGC552 (PsCFS) | CPR and TNMT integrant. MATα ura3-52 trp1-289 |
| | pGC1322 (PsSPSΔN) | leu2-3, 112 his3 Δ1 |
| SF1333-EX₍ₙ₎ | pBOT-TRP EX₍ₙ₎ (41-105) | CPR integrant. MATα ura3-52 trp1-289 leu2-3, 112 |
| | | his3 Δ1 |
| SF1333-LEU-EX₍ₙ₎ | pBOT-LEU-EX₍ₙ₎ (54, 98) | CPR integrant. MATα ura3-52 trp1-289 leu2-3, 112 |
| | | his3 Δ1 |
| SF1316-EX₍ₙ₎ | pBOT-TRP-EX₍ₙ₎ (41-105) | CPR and CFS integrant. MATα ura3-52 trp1-289 |
| | | leu2-3, 112 his3 Δ1 |
| SF1270-EX_{(n1, n2)} | pBOT-TRP-EX₍ₙ₁₎ or none | CPR and TNMT integrant. MATα ura3-52 trp1-289 |
| | pBOT-LEU-EX₍ₙ₂₎ or none | leu2-3, 112 his3 Δ1 |

| Plasmid | Brief description of genotype ^{a} |
|---|---|
| pGC1189 | CPR^{b} |
| pGC1190 | CPR^{b}-CFS |
| pGC1191 | CPR^{b}-SPSΔN^{b} |
| pGC1062 (Block 1) (SEQ ID NO: 7) | 6OM^{b}-4′OMT2^{b}-CNMT^{b} |
| pGC994 (Block 2) (SEQ ID NO: 8) | CFS-BBEΔN-recombinant psSPSΔN^{b} |
| pGC997 (Block 3) (SEQ ID NO: 9) | P6H-MSH-TNMT |
| pGC557 (CPR) (SEQ ID NO: 10) | CPR^{b} |
| pGC655 (BBEΔN-2μ) (SEQ ID NO: 11) | BBEΔN |
| pGC717 | CPR^{b}-TNMT |
| pGC550 | SOMT-CAS |
| pGC552 | CFS |
| pGC1322 | SPSΔN |
| EX₍ₙ₎ | CYP719 (numbers span from 41-105) |
| LEU-EX₍ₙ₎ | CYP719 (either 54 or 98) |

^{a} All coding sequences are from Papaver somniferum except PH6 which is from Eschscholzia californica
^{b}Synthetic gene. Codon-optimized sequence for expression in Saccharomyces cerevisiae.

TABLE 2
List of Saccharomyces cerevisiae strains and plasmids used the examples presented herein

| Strain | Genotype ^{a, c} | Plasmid | Source |
|---|---|---|---|
| YPH499 | MATa ura3-52 lys2-801_amber ade2-101_ochre trp1- | None | [57, 58] |
| | Δ63 his3-Δ200 leu2-Δ1 | | |
| GCY1192 | YPH499 | pGC1189 | This Application |
| GCY1193 | YPH499 | pGC1190 | This Application |
| GCY1194 | YPH499 | pGC1191 | This Application |
| CEN.PK113-16B | MATα leu2-3 MAL2-8C SUC2 | None | [59] |
| CEN.PK113-16C | MATα trp1-289 his3 Δ1 MAL2-8C SUC2 | None | [59] |
| CEN.PK113-3B | MATα ura3-52 his3 Δ1 MAL2-8C SUC2 | None | [59] |
| CEN.PK2-1D | MATα ura3-52 trp1-289 leu2-3, 112 his3 Δ1 MAL2-8C | None | [59] |
| | SUC2 | | |
| GCY1086 | CEN.PK113-16B | pGC1062 | This Application |
| GCY1090 | CEN.PK113-16C | pGC994, pGC557 | This Application |
| GCY1094 | CEN.PK113-3B | pGC997, pGC557 | This Application |
| GCY1098 | CEN.PK2-1D | pGC1062, pGC994, | This Application |
| | | pGC997, pGC557 | |
| GCY1074 | CEN.PK2-1D YORWΔ17(ChrXV)::C1-P_{TDH3}-CFS-T_{CYC1}- | None | This Application |
| | C6-H1-C1-P_{PDC1}-BBEΔN-T_{ADH1}-C6-H2-C1-P_{PMA1}- | | |
| | SPSΔN^{b}-T_{PGI1}-C6 | | |
| GCY1101 | GCY1074 (Block 2 integrant) | pGC557 (CPR) | This Application |
| GCY1082 | YPRCΔ15(ChrXVI)::C1-P_{PDC1}-P6H-T_{CYC1}-C6-H1-C1- | None | This Application |
| | P_{TDH3}-MSH-T_{ADH1}-C6-C1-P_{FBA1}-TNMT-T_{PGI1}-C6 with | | |
| | pGC557 | | |
| GCY1104 | GCY1082 (Block 2 and 3 integrant) | pGC557 (CPR) | This Application |
| GCY1108 | GCY1082 (Block 2 and 3 integrant) | pGC1062 (Block1) | This Application |
| | | pGC557 (CPR) | |
| GCY1125 | GCY1082 (Block 2 and 3 integrant) | pGC1062 (Block1) | This Application |
| | | pGC557 (CPR) | |
| | | pGC655 (BBE-2μ) | |
| GCY1127 | GCY1074 (Block 2 integrant) | pGC717 | This Application |
| | | (TNMT + CPR) | |
| GCY1333 | CEN.PK2-1D YNRCΔ9 (ChrXIV)::C1-P_{TDH3}-CPR-T_{CYC1}-C6 | None | This application |
| GCY1270 | CEN.PK2-1D YORWΔ17(ChrXV)::C1-P_{FBA1}-CPR-T_{CYC1}- | None | This application |
| | C6-H1-C1-P_{TDH3}-TNMT-T_{ADH1}-C6 | | |
| GCY1301 | GCY1300 | pGC550 | This application |
| | | (SOMT + CAS) | |
| GCY1316 | CEN.PK2-1D YNRCΔ9 (ChrXIV)::C1-P_{TDH3}-CPR-T_{CYC1}-C6 | None | This application |
| | YORWΔ17(ChrXV)::C1-P_{TDH3}-CFS-T_{CYC1}-C6 | | |
| GCY1411 | GCY1333 (CPR integrant) | pGC552 | This application |
| GCY1412 | GCY1333 (CPR integrant) | pGC1322 | This application |
| SF1333-EX41 | GCY1333 (CPR integrant) | pBOT-TRP-EX41 | This application |
| SF1333-EX42 | GCY1333 (CPR integrant) | pBOT-TRP-EX42 | This application |
| SF1333-EX43 | GCY1333 (CPR integrant) | pBOT-TRP-EX43 | This application |
| SF1333-EX44 | GCY1333 (CPR integrant) | pBOT-TRP-EX44 | This application |
| SF1333-EX45 | GCY1333 (CPR integrant) | pBOT-TRP-EX45 | This application |
| SF1333-EX46 | GCY1333 (CPR integrant) | pBOT-TRP-EX46 | This application |
| SF1333-EX47 | GCY1333 (CPR integrant) | pBOT-TRP-EX47 | This application |
| SF1333-EX48 | GCY1333 (CPR integrant) | pBOT-TRP-EX48 | This application |
| GCY1411 | GCY1333 (CPR integrant) | pGC552 | This application |
| SF1333-EX50 | GCY1333 (CPR integrant) | pBOT-TRP-EX50 | This application |
| SF1333-EX51 | GCY1333 (CPR integrant) | pBOT-TRP-EX51 | This application |
| SF1333-EX52 | GCY1333 (CPR integrant) | pBOT-TRP-EX52 | This application |
| SF1333-EX53 | GCY1333 (CPR integrant) | pBOT-TRP-EX53 | This application |
| SF1333-EX54 | GCY1333 (CPR integrant) | pBOT-TRP-EX54 | This application |
| SF1333-EX55 | GCY1333 (CPR integrant) | pBOT-TRP-EX55 | This application |
| SF1333-EX56 | GCY1333 (CPR integrant) | pBOT-TRP-EX56 | This application |
| SF1333-EX57 | GCY1333 (CPR integrant) | pBOT-TRP-EX57 | This application |
| SF1333-EX58 | GCY1333 (CPR integrant) | pBOT-TRP-EX58 | This application |
| SF1333-EX59 | GCY1333 (CPR integrant) | pBOT-TRP-EX59 | This application |
| SF1333-EX60 | GCY1333 (CPR integrant) | pBOT-TRP-EX60 | This application |
| SF1333-EX61 | GCY1333 (CPR integrant) | pBOT-TRP-EX61 | This application |
| SF1333-EX63 | GCY1333 (CPR integrant) | pBOT-TRP-EX63 | This application |
| SF1333-EX64 | GCY1333 (CPR integrant) | pBOT-TRP-EX64 | This application |
| SF1333-EX65 | GCY1333 (CPR integrant) | pBOT-TRP-EX65 | This application |
| SF1333-EX66 | GCY1333 (CPR integrant) | pBOT-TRP-EX66 | This application |
| SF1333-EX67 | GCY1333 (CPR integrant) | pBOT-TRP-EX67 | This application |
| SF1333-EX68 | GCY1333 (CPR integrant) | pBOT-TRP-EX68 | This application |
| SF1333-EX69 | GCY1333 (CPR integrant) | pBOT-TRP-EX69 | This application |
| SF1333-EX70 | GCY1333 (CPR integrant) | pBOT-TRP-EX70 | This application |
| SF1333-EX71 | GCY1333 (CPR integrant) | pBOT-TRP-EX71 | This application |
| SF1333-EX72 | GCY1333 (CPR integrant) | pBOT-TRP-EX72 | This application |
| SF1333-EX73 | GCY1333 (CPR integrant) | pBOT-TRP-EX73 | This application |
| SF1333-EX74 | GCY1333 (CPR integrant) | pBOT-TRP-EX74 | This application |
| SF1333-EX75 | GCY1333 (CPR integrant) | pBOT-TRP-EX75 | This application |
| SF1333-EX76 | GCY1333 (CPR integrant) | pBOT-TRP-EX76 | This application |
| SF1333-EX77 | GCY1333 (CPR integrant) | pBOT-TRP-EX77 | This application |
| SF1333-EX79 | GCY1333 (CPR integrant) | pBOT-TRP-EX79 | This application |
| SF1333-EX80 | GCY1333 (CPR integrant) | pBOT-TRP-EX80 | This application |
| SF1333-EX81 | GCY1333 (CPR integrant) | pBOT-TRP-EX81 | This application |
| SF1333-EX82 | GCY1333 (CPR integrant) | pBOT-TRP-EX82 | This application |
| SF1333-EX83 | GCY1333 (CPR integrant) | pBOT-TRP-EX83 | This application |
| SF1333-EX84 | GCY1333 (CPR integrant) | pBOT-TRP-EX84 | This application |
| SF1333-EX85 | GCY1333 (CPR integrant) | pBOT-TRP-EX85 | This application |
| SF1333-EX86 | GCY1333 (CPR integrant) | pBOT-TRP-EX86 | This application |
| SF1333-EX87 | GCY1333 (CPR integrant) | pBOT-TRP-EX87 | This application |
| SF1333-EX88 | GCY1333 (CPR integrant) | pBOT-TRP-EX88 | This application |
| SF1333-EX89 | GCY1333 (CPR integrant) | pBOT-TRP-EX89 | This application |
| SF1333-EX90 | GCY1333 (CPR integrant) | pBOT-TRP-EX90 | This application |
| SF1333-EX91 | GCY1333 (CPR integrant) | pBOT-TRP-EX91 | This application |
| SF1333-EX92 | GCY1333 (CPR integrant) | pBOT-TRP-EX92 | This application |
| SF1333-EX93 | GCY1333 (CPR integrant) | pBOT-TRP-EX93 | This application |
| SF1333-EX95 | GCY1333 (CPR integrant) | pBOT-TRP-EX95 | This application |
| SF1333-EX96 | GCY1333 (CPR integrant) | pBOT-TRP-EX96 | This application |
| SF1333-EX97 | GCY1333 (CPR integrant) | pBOT-TRP-EX97 | This application |
| SF1333-EX98 | GCY1333 (CPR integrant) | pBOT-TRP-EX98 | This application |
| SF1333-EX99 | GCY1333 (CPR integrant) | pBOT-TRP-EX99 | This application |
| SF1333-EX100 | GCY1333 (CPR integrant) | pBOT-TRP-EX100 | This application |
| SF1333-EX101 | GCY1333 (CPR integrant) | pBOT-TRP-EX101 | This application |
| SF1333-EX102 | GCY1333 (CPR integrant) | pBOT-TRP-EX102 | This application |
| SF1333-EX103 | GCY1333 (CPR integrant) | pBOT-TRP-EX103 | This application |
| SF1333-EX104 | GCY1333 (CPR integrant) | pBOT-TRP-EX104 | This application |
| SF1333-EX105 | GCY1333 (CPR integrant) | pBOT-TRP-EX105 | This application |
| GCY1413 | GCY1333 (CPR integrant) | pGC1322 pGC552 | This application |
| SF1333- | GCY1333 (CPR integrant) | pGC1322 | This application |
| pGC1322-EX54 | | pBOT-LEU-EX54 | |
| SF1333-EX(42, 54) | GCY1333 (CPR integrant) | pBOT-TRP-EX42 | This application |
| | | pBOT-LEU-EX54 | |
| SF1333-EX(50, 54) | GCY1333 (CPR integrant) | pBOT-TRP-EX50 | This application |
| | | pBOT-LEU-EX54 | |
| SF1333-EX(56, 54) | GCY1333 (CPR integrant) | pBOT-TRP-EX56 | This application |
| | | pBOT-LEU-EX54 | |
| SF1333-EX(67, 54) | GCY1333 (CPR integrant) | pBOT-TRP-EX67 | This application |
| | | pBOT-LEU-EX54 | |
| SF1333-EX(76, 54) | GCY1333 (CPR integrant) | pBOT-TRP-EX76 | This application |
| | | pBOT-LEU-EX54 | |
| SF1333-EX(96, 54) | GCY1333 (CPR integrant) | pBOT-TRP-EX96 | This application |
| | | pBOT-LEU-EX54 | |
| SF1333- | GCY1333 (CPR integrant) | pBOT-TRP-EX101 | This application |
| EX(101, 54) | | pBOT-LEU-EX54 | |
| SF1333- | GCY1333 (CPR integrant) | pGC1322 | This application |
| pGC1322-EX98 | | pBOT-LEU-EX98 | |
| SF1333-EX(42, 98) | GCY1333 (CPR integrant) | pBOT-TRP-EX42 | This application |
| | | pBOT-LEU-EX98 | |
| SF1333-EX(50, 98) | GCY1333 (CPR integrant) | pBOT-TRP-EX50 | This application |
| | | pBOT-LEU-EX98 | |
| SF1333-EX(56, 98) | GCY1333 (CPR integrant) | pBOT-TRP-EX56 | This application |
| | | pBOT-LEU-EX98 | |
| SF1333-EX(67, 98) | GCY1333 (CPR integrant) | pBOT-TRP-EX67 | This application |
| | | pBOT-LEU-EX98 | |
| SF1333-EX(76, 98) | GCY1333 (CPR integrant) | pBOT-TRP-EX76 | This application |
| | | pBOT-LEU-EX98 | |
| SF1333-EX(96, 98) | GCY1333 (CPR integrant) | pBOT-TRP-EX96 | This application |
| | | pBOT-LEU-EX98 | |
| SF1333- | GCY1333 (CPR integrant) | pBOT-TRP-EX101 | This application |
| EX(101, 98) | | pBOT-LEU-EX98 | |
| GCY1415 | GCY1270 (CPR, TNMT integrant) | pGC552 | This application |
| GCY1416 | GCY1270 (CPR, TNMT integrant) | pGC1322 | This application |
| GCY1417 | GCY1270 (CPR, TNMT integrant) | pGC552 pGC1322 | This application |
| SF1270- | GCY1270 (CPR, TNMT integrant) | pGC1322 | This application |
| pGC1322-EX54 | | pBOT-LEU-EX54 | |
| SF1270-EX(42, 54) | GCY1270 (CPR, TNMT integrant) | pBOT-TRP-EX42 | This application |
| | | pBOT-LEU-EX54 | |
| SF1270-EX(50, 54) | GCY1270 (CPR, TNMT integrant) | pBOT-TRP-EX50 | This application |
| | | pBOT-LEU-EX54 | |
| SF1270-EX(56, 54) | GCY1270 (CPR, TNMT integrant) | pBOT-TRP-EX56 | This application |
| | | pBOT-LEU-EX54 | |
| SF1270-EX(67, 54) | GCY1270 (CPR, TNMT integrant) | pBOT-TRP-EX67 | This application |
| | | pBOT-LEU-EX54 | |
| SF1270-EX(76, 54) | GCY1270 (CPR, TNMT integrant) | pBOT-TRP-EX76 | This application |
| | | pBOT-LEU-EX54 | |
| SF1270-EX(96, 54) | GCY1270 (CPR, TNMT integrant) | pBOT-TRP-EX96 | This application |
| | | pBOT-LEU-EX54 | |
| SF1270- | GCY1270 (CPR, TNMT integrant) | pBOT-TRP-EX101 | This application |
| EX(101, 54) | | pBOT-LEU-EX54 | |
| SF1270- | GCY1270 (CPR, TNMT integrant) | pGC1322 | This application |
| pGC1322-EX98 | | pBOT-LEU-EX98 | |
| SF1270-EX(42, 98) | GCY1270 (CPR, TNMT integrant) | pBOT-TRP-EX42 | This application |
| | | pBOT-LEU-EX98 | |
| SF1270-EX(50, 98) | GCY1270 (CPR, TNMT integrant) | pBOT-TRP-EX50 | This application |
| | | pBOT-LEU-EX98 | |
| SF1270-EX(56, 98) | GCY1270 (CPR, TNMT integrant) | pBOT-TRP-EX56 | This application |
| | | pBOT-LEU-EX98 | |
| SF1270-EX(67, 98) | GCY1270 (CPR, TNMT integrant) | pBOT-TRP-EX67 | This application |
| | | pBOT-LEU-EX98 | |
| SF1270-EX(76, 98) | GCY1270 (CPR, TNMT integrant) | pBOT-TRP-EX76 | This application |
| | | pBOT-LEU-EX98 | |
| SF1270-EX(96, 98) | GCY1270 (CPR, TNMT integrant) | pBOT-TRP-EX96 | This application |
| | | pBOT-LEU-EX98 | |
| SF1270- | GCY1270 (CPR, TNMT integrant) | pBOT-TRP-EX101 | This application |
| EX(101, 98) | | pBOT-LEU-EX98 | |

| Plasmid name | Genotype ^{a, c} | Source |
|---|---|---|
| pESC-leu2d | 2μ^{ori}, pUC^{ori}, leu2d, Amp^{R}, P_{GAL1}-c-Myc tag-T_{CYC1}, P_{GAL10}-FLAG tag-T_{ADH1} | [58] |
| pGC1189 | pESC-leu2d::CPR^{b} | This Application |
| pGC1190 | pESC-leu2d::CPR^{b}-CFS | This Application |
| pGC1191 | pESC-Leu2d::CPR^{b}-SPSΔN^{b} | This Application |
| pGREG503 | CEN6/ARS4^{ori}, pMB1^{ori}, HIS3, Amp^{R}, loxP-Kan^{R}, P_{GAL1}-HISstuffer-T_{CYC1} | [55] |
| pGREG504 | CEN6/ARS4^{ori}, pMB1^{ori}, TRP1, Amp^{R}, loxP-Kan^{R}, P_{GAL1}-HISstuffer-T_{CYC1} | [55] |
| pGREG505 | CEN6/ARS4^{ori}, pMB1^{ori}, LEU2, Amp^{R}, loxP-Kan^{R}, P_{GAL1}-HISstuffer-T_{CYC1} | [55] |
| pGREG506 | CEN6/ARS4^{ori}, pMB1^{ori}, URA3, Amp^{R}, loxP-Kan^{R}, P_{GAL1}-HISstuffer-T_{CYC1} | [55] |
| pGC964 | pGREG503 ΔKpnl⁽³⁵⁵⁵⁻²⁵⁶⁰⁾A(3558)G, ΔKpnl⁽⁴⁵⁰⁹⁻⁴⁵¹⁴⁾ A(4512)G | This Application |
| pGC965 | pGREG504 ΔKpnl⁽³⁵⁵⁵⁻²⁵⁶⁰⁾A(3558)G | This Application |
| pGC966 | pGREG505 ΔKpnl⁽³⁵⁵⁵⁻²⁵⁶⁰⁾A(3558)G, ΔKpnl⁽⁵¹⁷⁶⁻⁵¹⁸¹⁾ A(5179)G | This Application |
| pGC967 | pGREG506 ΔKpnl⁽³⁵⁹³⁻³⁵⁹⁸⁾A(3596)G | This Application |
| pGC1062 | pGC966::C1-P_{TDH3}-6OMT^{b}-T_{CYC1}-C6-H1-C1-P_{FBA1}-4′OMT2^{b}-T_{ADH1}-C6-H2- | This Application |
| (Block 1) | C1- P_{PDC1}-CNMT^{b}-T_{PGI1}-C6, | |
| pGC994 | pGC965::C1-P_{TDH3}-CFS-T_{CYC1}-C6-H1-C1-P_{PDC1}-BBEΔN-T_{ADH1}-C6-H2-C1- | This Application |
| (Block 2) | P_{PMA1}-SPSΔN^{b}-TT_{PGI1}-C6 | |
| pGC997 | pGC967::C1-P_{PDC1}-P6H-T_{CYC1}-C6-H1-C1-P_{TDH3}-MSH-T_{ADH1}-C6-C1-P_{FBA1}- | This Application |
| (Block 3) | TNMT-T_{PGI1}-C6 | |
| pGC557 | pGC964::C1-P_{TDH3}-CPR^{b}-T_{CYC1}-C6 | This Application |
| pYES2 | 2μ^{ori}, pUC^{ori}, URA3, Amp^{R}, P_{GAL1}-T_{CYC1} | This Application |
| pGC655 | pYES2::P_{PMA1}-BBEΔN - T_{PGI1} | This Application |
| pGC717 | pGC964::C1- P_{FBA1}-CPR^{b}-T_{CYC1}-C6-H1-C1-P_{TDH3}-TNMT-T_{ADH1}-C6 | This Application |
| pGC550 | pGC967::C1- P_{TDH3}-SOMT-T_{CYC1}-C6-H2-C1-P_{PMA1}-CAS-T_{PGI1}-C6 | This Application |
| pBOT-TRP | CEN6/ARS4^{ori}, pMB1^{ori,} TRP1, Amp^{R}, loxP-Kan^{R}, P_{TDH3}-stuffer-T_{CYC1} | This Application |
| pBOT-LEU | CEN6/ARS4^{ori}, pMB1^{ori}, LEU2, Amp^{R}, loxP-Kan^{R}, P_{FBA1}-stuffer-T_{ADH1} | This Application |
| pBOT-TRP-EX41 | pBOT-TRP::EX41 | This Application |
| pBOT-TRP-EX42 | pBOT-TRP::EX42 | This Application |
| pBOT-TRP-EX43 | pBOT-TRP::EX43 | This Application |
| pBOT-TRP-EX44 | pBOT-TRP::EX44 | This Application |
| pBOT-TRP-EX45 | pBOT-TRP::EX45 | This Application |
| pBOT-TRP-EX46 | pBOT-TRP::EX46 | This Application |
| pBOT-TRP-EX47 | pBOT-TRP::EX47 | This Application |
| pBOT-TRP-EX48 | pBOT-TRP::EX48 | This Application |
| pBOT-TRP-EX50 | pBOT-TRP::EX49 | This Application |
| pBOT-TRP-EX51 | pBOT-TRP::EX50 | This Application |
| pBOT-TRP-EX52 | pBOT-TRP::EX51 | This Application |
| pBOT-TRP-EX53 | pBOT-TRP::EX52 | This Application |
| pBOT-TRP-EX54 | pBOT-TRP::EX53 | This Application |
| pBOT-TRP-EX55 | pBOT-TRP::EX54 | This Application |
| pBOT-TRP-EX56 | pBOT-TRP::EX55 | This Application |
| pBOT-TRP-EX57 | pBOT-TRP::EX56 | This Application |
| pBOT-TRP-EX58 | pBOT-TRP::EX57 | This Application |
| pBOT-TRP-EX59 | pBOT-TRP::EX58 | This Application |
| pBOT-TRP-EX60 | pBOT-TRP::EX59 | This Application |
| pBOT-TRP-EX61 | pBOT-TRP::EX60 | This Application |
| pBOT-TRP-EX63 | pBOT-TRP::EX61 | This Application |
| pBOT-TRP-EX64 | pBOT-TRP::EX62 | This Application |
| pBOT-TRP-EX65 | pBOT-TRP::EX63 | This Application |
| pBOT-TRP-EX66 | pBOT-TRP::EX64 | This Application |
| pBOT-TRP-EX67 | pBOT-TRP::EX65 | This Application |
| pBOT-TRP-EX68 | pBOT-TRP::EX66 | This Application |
| pBOT-TRP-EX69 | pBOT-TRP::EX67 | This Application |
| pBOT-TRP-EX70 | pBOT-TRP::EX68 | This Application |
| pBOT-TRP-EX71 | pBOT-TRP::EX69 | This Application |
| pBOT-TRP-EX72 | pBOT-TRP::EX70 | This Application |
| pBOT-TRP-EX73 | pBOT-TRP::EX71 | This Application |
| pBOT-TRP-EX74 | pBOT-TRP::EX72 | This Application |
| pBOT-TRP-EX75 | pBOT-TRP::EX73 | This Application |
| pBOT-TRP-EX76 | pBOT-TRP::EX74 | This Application |
| pBOT-TRP-EX77 | pBOT-TRP::EX75 | This Application |
| pBOT-TRP-EX79 | pBOT-TRP::EX76 | This Application |
| pBOT-TRP-EX80 | pBOT-TRP::EX77 | This Application |
| pBOT-TRP-EX81 | pBOT-TRP::EX78 | This Application |
| pBOT-TRP-EX82 | pBOT-TRP::EX79 | This Application |
| pBOT-TRP-EX83 | pBOT-TRP::EX80 | This Application |
| pBOT-TRP-EX84 | pBOT-TRP::EX81 | This Application |
| pBOT-TRP-EX85 | pBOT-TRP::EX82 | This Application |
| pBOT-TRP-EX86 | pBOT-TRP::EX83 | This Application |
| pBOT-TRP-EX87 | pBOT-TRP::EX84 | This Application |
| pBOT-TRP-EX88 | pBOT-TRP::EX85 | This Application |
| pBOT-TRP-EX89 | pBOT-TRP::EX86 | This Application |
| pBOT-TRP-EX90 | pBOT-TRP::EX87 | This Application |
| pBOT-TRP-EX91 | pBOT-TRP::EX88 | This Application |
| pBOT-TRP-EX92 | pBOT-TRP::EX89 | This Application |
| pBOT-TRP-EX93 | pBOT-TRP::EX90 | This Application |
| pBOT-TRP-EX95 | pBOT-TRP::EX91 | This Application |
| pBOT-TRP-EX96 | pBOT-TRP::EX92 | This Application |
| pBOT-TRP-EX97 | pBOT-TRP::EX93 | This Application |
| pBOT-TRP-EX98 | pBOT-TRP::EX94 | This Application |
| pBOT-TRP-EX99 | pBOT-TRP::EX95 | This Application |
| pBOT-TRP-EX100 | pBOT-TRP::EX96 | This Application |
| pBOT-TRP-EX101 | pBOT-TRP::EX97 | This Application |
| pBOT-TRP-EX102 | pBOT-TRP::EX98 | This Application |
| pBOT-TRP-EX103 | pBOT-TRP::EX99 | This Application |
| pBOT-TRP-EX104 | pBOT-TRP::EX100 | This Application |
| pBOT-TRP-EX105 | pBOT-TRP::EX101 | This Application |
| pBOT-LEU-EX54 | CEN6/ARS4^{ori}, pMB1^{ori}, LEU2, Amp^{R}, loxP-Kan^{R}, P_{TDH3}-stuffer- | This Application |
| | T_{CYC1}::EX54 | |
| pBOT-LEU-EX98 | CEN6/ARS4^{ori}, pMB1^{ori}, LEU2, Amp^{R}, loxP-Kan^{R}, P_{TDH3}-stuffer- | This Application |
| | T_{CYC1}::EX54 | |
| pGC552 | pGC967::C1- P_{TDH3}-CFS-T_{CYC1}-C6 | This Application |
| pGC1322 | pGC965::C1- P_{PMA1}- SPSΔN^{b} -T_{PGI1}-C6 | This Application |

^{a} All coding sequences are from Papaver somniferum except PH6 which is from Eschscholzia californica.
^{b}Synthetic gene. Codon-optimized sequence for expression in Saccharomyces cerevisiae.
^{c} Linkers used for cloning purposes are in bold.

### Assembly of Plasmids by Homologous Recombination

Blocks of enzymes were designed to independently express sequential enzymes of the dihydrosanguinarine pathway. Enzyme blocks were cloned into the pGREG series of E. coli-S. cerevisiae shuttle vectors⁵⁵. Vectors pGREG503, 504, 505 and 506, harbouring the HIS3, TRP1, LEU2 and URA3 auxotrophic markers, respectively, were modified by site-directed mutagenesis to contain a unique KpnI site at the 3′ end of a stuffer cassette in the multiple cloning site using the PCR primers reported in Supplemental Table 3. Gene expression cassettes were inserted by homologous recombination into pGREG vectors previously linearized with AscI/KpnI. Empty pGREG control plasmids were created by intra-molecular ligation of the linearized pGREG made blunt with T4 DNA polymerase.

TABLE 3
Oligonucleotides used in site-directed
mutagenesis of the pGREG vector series in
order to eliminate additional KpnI sites

| Primer name | Sequence 5′→3′ |
|---|---|
| Kpn_forward | TAATTAAGGGTGCCCAATTCGCCCTA |
| | TAGTGAGT (SEQ ID NO: 385) |
| Kpn_reverse | TAGGGCGAATTGGGCACCCTTAATTA |
| | AGACAAC (SEQ ID NO: 386) |
| KpnURA_f | CGTTGGTGCCATTGGGCGAGGTGGCT |
| | TCTCT (SEQ ID NO: 387) |
| KpnURA_r | CCTCGCCCAATGGCACCAACGATGTT |
| | C (SEQ ID NO: 388) |
| KpnLEU_f | CTAAATGGGGTGCCGGTATTAGACCT |
| | GAACAAG (SEQ ID NO: 389) |
| KpnLEU_r | CGTCTAACACTACCGGCACCCCATTT |
| | AGGACCAC (SEQ ID NO: 390) |

The DNA assembler technique, which takes advantage of in vivo homologous recombination in yeast⁹, was used for the assembly of the sanguinarine pathway. Promoters, genes, and terminators were assembled by incorporating a ˜50-bp homologous region between the segments. Expression cassettes were joined to each other and to the vector backbone using DNA linkers (C6-H(n)-C1 linkers in Table 4), with the exception of some components of Block 3. DNA linkers were added to promoters and terminators by PCR using the primers listed in Table 4 and CEN.PK genomic DNA as template. In addition, a NotI site was introduced in the 3′ linker primer containing homology to pGREG backbones, allowing the excision of enzyme blocks by AscI/NotI double digest. PsBBEΔN was also independently cloned into the 2μ, high copy vector pYES2. For DNA assembly, the pYES2 backbone was amplified by PCR using primers pYES2 for and pYES2 rev described in Table 4. Transformation of DNA fragments in yeast for homologous recombination was accomplished by electroporation. Assembled plasmids were transferred to E. coli and sequenced-verified. All the plasmids used in examples presented herein are described in Table 1 and Table 2.

TABLE 4
Oligonucleotides used for amplifications
of expression construct parts

| | |
|---|---|
| Name | Sequence 5′→3′ |

pESC-CPR/CFS

| | |
|---|---|
| PsCFS for | GCGGCCGCAAAAATGGAGGTGACATTTTGGTTG |
| | (SEQ ID NO: 391) |
| PsCFS rev | ACTAGTGCATGGATACGAGGAGTAAT |
| | (SEQ ID NO: 392) |

pGC1062

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 393) |
| TDH3p rev | TTGATACTGTTTCCATTGTTTTTCGAAACTAAG |
| | TTCTTGGTGTTTTAAAAC |
| | (SEQ ID NO: 394) |
| 60MT for | AAACACCAAGAACTTAGTTTCGAAAAACAATGG |
| | AAACAGTATCAAAGATCG |
| | (SEQ ID NO: 395) |
| 60MT rev | TAAGCGTGACATAACTAATTACATGATCAATAT |
| | GGATAGGCTTCGATCAC |
| | (SEQ ID NO: 396) |
| CYCt for | GTGATCGAAGCCTATCCATATTGATCATGTAAT |
| | TAGTTATGTCACGCTTAC |
| | (SEQ ID NO: 397) |
| FBA1p rev | CCAAGGAACCCATTGTTTTTATGTATTACTTGG |
| | TTATGGTTATATATGAC |
| | (SEQ ID NO: 398) |
| 4CMT_2 for | ATATAACCATAACCAAGTAATACATAAAAACAA |
| | TGGGTTCCTTGGATGCG |
| | (SEQ ID NO: 399) |
| 4CMT_2 rev | CAAACCTCTGGCGAAGAAGTCCATTATGGAAAA |
| | GCTTCTATAACAGATTGTATTGC |
| | (SEQ ID NO: 400) |
| ADH1t for | CAATCTGTTATAGAAGCTTTTCCATAATGGACT |
| | TCTTCGCCAGAGGTTTG |
| | (SEQ ID NO: 401) |
| PDC1p rev | TTTGCTTTCAGTTGCATTGTTTTTGATTTGACT |
| | GTGTTATTTTGCGTGAG |
| | (SEQ ID NO: 402) |
| CNMT rev | GCAAAATAACACAGTCAAATCAAAAACAATGCA |
| | ACTGAAAGCAAAGGAAG |
| | (SEQ ID NO: 403) |
| CNMT for | GCAAAATAACACAGTCAAATCAAAAACAATGCA |
| | ACTGAAAGCAAAGGAAG |
| | (SEQ ID NO: 404) |
| PGlt for | TCTTTAAGAAAAAGTAAAACAAATCGCTCTTAA |
| | ATATATACCTAAAGAAC |
| | (SEQ ID NO: 405) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 406) |

pGC994

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 407) |
| TDH3p rev | CAACCAAAATGTCACCTCCATTGTTTTTCGAAA |
| | CTAAGTTCTTGGTGTTTTAAAAC |
| | (SEQ ID NO: 408) |
| CheSyn for | TTTTAAAACACCAAGAACTTAGTTTCGAAAAAC |
| | AATGGAGGTGACATTTTGGTTGATAAC |
| | (SEQ ID NO: 409) |
| CheSyn rev | GTAAGCGTGACATAACTAATTACATGATTAATG |
| | GATACGAGGAGTAATTTTGGC |
| | (SEQ ID NO: 410) |
| CYC1t for | GCCAAAATTACTCCTCGTATCCATTAATCATGT |
| | AATTAGTTATGTCACGCTTAC |
| | (SEQ ID NO: 411) |
| C6:pG rev | CAATCTCGCCCACAGCCCCTTTCTTTAATCATT |
| | CCGACCCCCGCCATGAGACAACTCATGGTGATG |
| | TGATTGCC (SEQ ID NO: 412) |
| C1:H1 for | CTCATGGCGGGGGTCGGAATGATTAAAGAAAGG |
| | GGCTGTGGGCGAGATTGGAGACTGCAGCATTAC |
| | TTTGAGAAG (SEQ ID NO: 413) |
| PDC1p rev | GGAGATTATCATTAACATCACCCATTGTTTTTG |
| | ATTTGACTGTGTTATTTTGCGTGAGG |
| | (SEQ ID NO: 414) |
| BBE for | AATAACACAGTCAAATCAAAAACAATGGGTGAT |
| | GTTAATGATAATCTCCTCTCGTCATG |
| | (SEQ ID NO: 415) |
| BBE rev | AAACCTCTGGCGAAGAAGTCCACAATTCCTTCA |
| | ACATGTAAATTTCCTCAAATTTC |
| | (SEQ ID NO: 416) |
| ADH1t for | GAAATTTGAGGAAATTTACATGTTGAAGGAATT |
| | GTGGACTTCTTCGCCAGAGGTTT |
| | (SEQ ID NO: 417) |
| C6:H2 rev | TGGTGACCTCCATTAGGCCACCATCATGTTTGC |
| | CACGGTTTATTAACTGGACAACTCATGGTGATG |
| | TGATTGCC (SEQ ID NO: 418) |
| C1:H2 for | CCAGTTAATAAACCGTGGCAAACATGATGGTGG |
| | CCTAATGGAGGTCACCAGAGACTGCAGCATTAC |
| | TTTGAGAAG (SEQ ID NO: 419) |
| PMA1p rev | AGGTAGTAATCGATAATTCCATTTTGATAATTA |
| | AATCTTTCTTATCTTCTTATTCTTTTC |
| | (SEQ ID NO: 420) |
| StySyn for | TAAGAAGATAAGAAAGATTTAATTATCAAAATG |
| | GAATTATCGATTACTACCTCAATAGC |
| | (SEQ ID NO: 421) |
| StySyn rev | GTTCTTTAGGTATATATTTAAGAGCGATTTGTT |
| | TTAAACTCTTGGGACTATCCTCGC |
| | (SEQ ID NO: 422) |
| PGl1t for | GCGAGGATAGTCCCAAGAGTTTAAAACAAATCG |
| | CTCTTAAATATATACCTAAAGAAC |
| | (SEQ ID NO: 423) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 424) |

pGC997

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 425) |
| PDC1p rev | AGCAAGCATTAAGGAATCCATTGTTTTTGATTT |
| | GACTGTGTTATTTTG (SEQ ID NO: 426) |
| P6H for | GCAAAATAACACAGTCAAATCAAAAACAATGGA |
| | TTCCTTAATGCTTG (SEQ ID NO: 427) |
| P6H rev | TGTAAGCGTGACATAACTAATTACATGACTATT |
| | CGTACAACTTGTAATGT |
| | (SEQ ID NO: 428) |
| CYC1t for | TCGTCTACATTACAAGTTGTACGAATAGTCATG |
| | TAATTAGTTATGTCACG |
| | (SEQ ID NO: 429) |
| C6:H1 rev | CAATCTCGCCCACAGCCCCTTTCTTTAATCATT |
| | CCGACCCCCGCCATGAGACAACTCATGGTGATG |
| | TGATTGCC (SEQ ID NO: 430) |
| C1:H1 for | CTCATGGCGGGGGTCGGAATGATTAAAGAAAGG |
| | GGCTGTGGGCGAGATTGGAGACTGCAGCATTAC |
| | TTTGAGAAG (SEQ ID NO: 431) |
| TDH3p rev | CGGTCTGTTTGTTTTGATTGATTCGGTTCGCAT |
| | TGTTTTTCGAAACTAAGTTCTTGGTG |
| | (SEQ ID NO: 432) |
| MSH for | TTTAAAACACCAAGAACTTAGTTTCGAAAAACA |
| | ATGCGAACCGAATCAAT |
| | (SEQ ID NO: 433) |
| MSH rev | CCTCTGGCGAAGAAGTCCATCATATCTCGAGTC |
| | GAGGTTTGATC (SEQ ID NO: 434) |
| ADH1t for | GATCAAACCTCGACTCGAGATATGATGGACTTC |
| | TTCGCCAGAGG (SEQ ID NO: 435) |
| ADH1t rev | ACAACTCATGGTGATGTGATTGCCGCATGCCGG |
| | TAGAGGTGTGGT (SEQ ID NO: 436) |
| FBA1p for | ACCACACCTCTACCGGCATGCGGCAATCACATC |
| | ACCATGAGTTGT (SEQ ID NO: 437) |
| FBA1p rev | TCATCTATTGAACCCATTGTTTTTATGTATTAC |
| | TTGGTTATGGTTATATATGACAAAAG |
| | (SEQ ID NO: 438) |
| TNMT for | TATATAACCATAACCAAGTAATACATAAAAACA |
| | ATGGGTTCAATAGATGAGGTCAAGAA |
| | (SEQ ID NO: 439) |
| TNMT rev | GTTCTTTAGGTATATATTTAAGAGCGATTTGTT |
| | CTACTTCTTCTTGAAAAGCAGCTG |
| | (SEQ ID NO: 440) |
| PGl1t for | GCAGCTGCTTTTCAAGAAGAAGTAGAACAAATC |
| | GCTCTTAAATATATACCTAAAG |
| | (SEQ ID NO: 441) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 442) |

pGC577

| | |
|---|---|
| pG:C6 rev | TAACCCTCACTAAAGGGAACAAAAGCTGGAGCT |
| | CGTTTAAACGGCGCGCCGAGACTGCAGCATTAC |
| | TTTGAGAAG (SEQ ID NO: 443) |
| TDH3p rev | GTTTGCCAGGTTGTTTGACCCCATTGTTTTTCG |
| | AAACTAAGTTCTTGGTGTTTTAAAAC |
| | (SEQ ID NO: 444) |
| CPR for | GTTTTAAAACACCAAGAACTTAGTTTCGAAAAA |
| | CAATGGGGTCAAACAACCTGGC |
| | (SEQ ID NO: 445) |
| CPR rev | GTAAGCGTGACATAACTAATTACATGATTACCA |
| | TACATCTCTCAAGTATCTCTC |
| | (SEQ ID NO: 446) |
| CYC1t for | GAGAGATACTTGAGAGATGTATGGTAATCATGT |
| | AATTAGTTATGTCACGCTTAC |
| | (SEQ ID NO: 447) |
| pG:C6 rev | CAATCTCGCCCACAGCCCCTTTCTTTAATCATT |
| | CCGACCCCCGCCATGAGACAACTCATGGTGATG |
| | TGATTGCC (SEQ ID NO: 448) |

pGC655

| | |
|---|---|
| pYES2 for | GGCCCTGCATTAATGAATCG |
| | (SEQ ID NO: 449) |
| pYES2 rev | ACTAGTGGATCATCCCCAC |
| | (SEQ ID NO: 450) |
| pY:C1 for | CCGCCGCGCTTAATGGGGCGCTACAGGGCGCGT |
| | GGGGATGATCCACTAGTGAGACTGCAGCATTAC |
| | TTTGAGAAG (SEQ ID NO: 451) |
| PMA1p rev | CATTAACATCACCCATTGTTTTTTTGATAATTA |
| | AATCTTTCTTATCTTCTTATTCTTTTC |
| | (SEQ ID NO: 452) |
| BBE for | GAAGATAAGAAAGATTTAATTATCAAAAAAACA |
| | ATGGGTGATGTTAATGATAATCTCCTC |
| | (SEQ ID NO: 453) |
| BBE rev | CTTTAGGTATATATTTAAGAGCGATTTGTTCTA |
| | CAATTCCTTCAACATGTAAATTTCC |
| | (SEQ ID NO: 454) |
| PGl1t for | GGAAATTTACATGTTGAAGGAATTGTAGAACAA |
| | ATCGCTCTTAAATATATACCTAAAG |
| | (SEQ ID NO: 455) |
| pY:C6 | CAATACGCAAACCGCCTCTCCCCGCGCGTTGGC |
| | CGATTCATTAATGCAGGACAACTCATGGTGATG |
| | TGATTGCC (SEQ ID NO: 456) |

pGC717

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 457) |
| FBA1p rev | GCCAGGTTGTTTGACCCCATTGTTTTTATGTAT |
| | TACTTGGTTATGGTTATATATGAC |
| | (SEQ ID NO: 458) |
| CPR for | GTCATATATAACCATAACCAAGTAATACATAAA |
| | AACAATGGGGTCAAACAACCTGGC |
| | (SEQ ID NO: 459) |
| CPR rev | GTAAGCGTGACATAACTAATTACATGATTACCA |
| | TACATCTCTCAAGTATCTCTC |
| | (SEQ ID NO: 460) |
| CYC1t for | GAGAGATACTTGAGAGATGTATGGTAATCATGT |
| | AATTAGTTATGTCACGCTTAC |
| | (SEQ ID NO: 461) |
| C6:H1 rev | CAATCTCGCCCACAGCCCCTTTCTTTAATCATT |
| | CCGACCCCCGCCATGAGACAACTCATGGTGATG |
| | TGATTGCC (SEQ ID NO: 462) |
| C1:H1 for | CTCATGGCGGGGGTCGGAATGATTAAAGAAAGG |
| | GGCTGTGGGCGAGATTGGAGACTGCAGCATTAC |
| | TTTGAGAAG (SEQ ID NO: 463) |
| TDH3p rev | CTTGACCTCATCTATTGAACCCATTGTTTTTCG |
| | AAACTAAGTTCTTGGTGTTTTAAAAC |
| | (SEQ ID NO: 464) |
| TNMT for | GTTTTAAAACACCAAGAACTTAGTTTCGAAAAA |
| | CAATGGGTTCAATAGATGAGGTCAAG |
| | (SEQ ID NO: 465) |
| TNMT rev | GACTTGACCAAACCTCTGGCGAAGAAGTCCACT |
| | ACTTCTTCTTGAAAAGCAGCTG |
| | (SEQ ID NO: 466) |
| ADH1t for | GGATGGTTGCGCAGCTGCTTTTCAAGAAGAAGT |
| | AGTGGACTTCTTCGCCAGAGGT |
| | (SEQ ID NO: 467) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 468) |

pGC550

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 488) |
| TDH3p rev | GAAAATTTCGCCATTGGTAGCCATTGTTTTTCG |
| | AAACTAAGTTCTTGGTGTTTTAAAAC |
| | (SEQ ID NO: 489) |
| TDH3p for | GTTTTAAAACACCAAGAACTTAGTTTCGAAAAA |
| | CAATGGCTACCAATGGCGAAATTTTC |
| | (SEQ ID NO: 490) |
| SOMT rev | GTAAGCGTGACATAACTAATTACATGATCATTT |
| | GTGAAACTCAATGACATGAAG |
| | (SEQ ID NO: 491) |
| SOMT for | CTTCATGTCATTGAGTTTCACAAATGATCATGT |
| | AATTAGTTATGTCACGCTTAC |
| | (SEQ ID NO: 492) |
| C6:H1 rev | CAATCTCGCCCACAGCCCCTTTCTTTAATCATT |
| | CCGACCCCCGCCATGAGACAACTCATGGTGATG |
| | TGATTGCC (SEQ ID NO: 493) |
| C1:H1 for | CTCATGGCGGGGGTCGGAATGATTAAAGAAAGG |
| | GGCTGTGGGCGAGATTGGAGACTGCAGCATTAC |
| | TTTGAGAAG (SEQ ID NO: 494) |
| PMA1 rev | GTTACTCATGATCATTGTTTTTTTGATAATTAA |
| | ATCTTTCTTATCTTCTTATTCTTTTC |
| | (SEQ ID NO: 495) |
| PMA1p for | GATAAGAAAGATTTAATTATCAAAAAAACAATG |
| | ATCATGAGTAACTTATGGATTCTTAC |
| | (SEQ ID NO: 496) |
| CanSyn rev | GTTCTTTAGGTATATATTTAAGAGCGATTTGTT |
| | CTACAAACGAGGAACTATACGTGC |
| | (SEQ ID NO: 497) |
| CanSyn for | CGAAGCACGTATAGTTCCTCGTTTGTAGAACAA |
| | ATCGCTCTTAAATATATACCTAAAG |
| | (SEQ ID NO: 498) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 499) |

pGC552

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 500) |
| TDH3p rev | CAACCAAAATGTCACCTCCATTGTTTTTCGAAA |
| | CTAAGTTCTTGGTGTTTTAAAAC |
| | (SEQ ID NO: 501) |
| TDH3p for | TTTTAAAACACCAAGAACTTAGTTTCGAAAAAC |
| | AATGGAGGTGACATTTTGGTTGATAAC |
| | (SEQ ID NO: 502) |
| CheSyn rev | GTAAGCGTGACATAACTAATTACATGATTAATG |
| | GATACGAGGAGTAATTTTGGC |
| | (SEQ ID NO: 503) |
| CheSyn for | GCCAAAATTACTCCTCGTATCCATTAATCATGT |
| | AATTAGTTATGTCACGCTTAC |
| | (SEQ ID NO: 504) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 505) |

pGC1322

| | |
|---|---|
| pG:C1 for | TAACCCTCACTAAAGGGAACAAAAGCTGGAGCT |
| | CGTTTAAACGGCGCGCCGAGACTGCAGCATTAC |
| | TTTGAGAAG (SEQ ID NO: 506) |
| PMA1p rev | CGTCAAAATCTTGTCCGAAAATCAATC |
| | (SEQ ID NO: 507) |
| PMA1p for | GAAGATAAGAAAGATTTAATTATCAAAAAAACA |
| | ATGGAATTATCGATTACTACCTC |
| | (SEQ ID NO: 508) |
| SPSΔN rev | GCGATTTGTTCTAAGCGTAATCTGGAACATCGT |
| | ATGGGTAAACTCTTGGGACTATCCTC |
| | (SEQ ID NO: 509) |
| SPSΔN for | TACCCATACGATGTTCCAGATTACGCTTAGAAC |
| | AAATCGCTCTTAAATATATAC |
| | (SEQ ID NO: 510) |
| pG:C6 rev | ATAACTTCGTATAATGTATGCTATACGAAGTTA |
| | TTAGGTACCGCGGCCGCACAACTCATGGTGATG |
| | TGATTGCC (SEQ ID NO: 511) |

Integration of Enzyme Blocks into the Genome

Integration of enzyme blocks into the genome of S. cerevisiae was achieved through targeted homologous recombination to integration sites shown to support relatively high levels of gene expression³⁵. Enzyme blocks were integrated into the genome using upstream and downstream homology regions, selection cassettes, and gene cassettes, as parts for chromosomal DNA assembly⁹. Selection cassettes Hyg^{R} and G418^{R} (for Blocks 2 and 3, respectively), were amplified from pZC3 and pUG6, while genomic homology regions (site 18 on chromosome XV and site 20 on chromosome XVI for Blocks 2 and 3, respectively) were amplified from CEN.PK genomic DNA using primers described in Table 5. Gene cassettes of Blocks 2 and 3 were excised from their plasmids by AscI//NotI-HF/XbaI digestion. Parts for assembly were transformed into S. cerevisiae by the lithium acetate method and integrants were selected on solid media. Successful integration was verified by PCR using genomic DNA as template.

TABLE 5A
Oligonucleotides used for genomic DNA
amplifications for cassette integration

| | |
|---|---|
| Primer name | Sequence 5′→3′ |

Integration site 18

| | |
|---|---|
| 18 Up for | TGTGCACAAAGGCCATAATATTATGTC |
| | (SEQ ID NO: 469) |
| 18 Up rev | TAATTTCGATAAGCCAGGTTAACCTGC |
| | GGCATGAGTTATGGTTGCACAGT |
| | (SEQ ID NO: 470) |
| HygR for | GGTAACTGTGCAACCATAACTCATGCC |
| | GCAGGTTAACCTGGCTTATCGAA |
| | (SEQ ID NO: 471 |
| HygR rev | CTCAAAGTAATGCTGCAGTCTCGGCGC |
| | GCCGGCTACAATTAATACATAACCTTA |
| | TGTATC |
| | (SEQ ID NO: 472) |
| 18 Down for | CTCAAAGTAATGCTGCAGTCTCGGCGC |
| | GCCGGCTACAATTAATACATAACCTTA |
| | TGTATC |
| | (SEQ ID NO: 473) |
| 18 Down rev | AAAGCTGGCTCCCCTTAGACAA |
| | (SEQ ID NO: 474) |

Integration site 20

| | |
|---|---|
| 20 Up for | GCCAGGCGCCTTTATATCAT |
| | (SEQ ID NO: 475) |
| 20 Up rev | TAATTTCGATAAGCCAGGTTAACCTGC |
| | TTTGCGAAACCCTATGCTCT |
| | (SEQ ID NO: 476) |
| G418 for | TTCAAATCCGAACAACAGAGCATAGGG |
| | TTTCGCAAAGCAGGTTAACCTGGCTTA |
| | TCGAA |
| | (SEQ ID NO: 477) |
| G418 rev | CTCAAAGTAATGCTGCAGTCTCGGCGC |
| | GCCGGCTACAATTAATACATAACCTTA |
| | TGTATC |
| | (SEQ ID NO: 478) |
| 20 Down for | GGCAATCACATCACCATGAGTTGTGCG |
| | GCCGCAATGGAAGGTCGGGATGAG |
| | (SEQ ID NO: 479) |
| 20 Down rev | ATAAAGCAGCCGCTACCAAA |
| | (SEQ ID NO: 480) |

Integration site 16

| | |
|---|---|
| 16 Up for | TTCGTGAAACACGTGGGATACC |
| | (SEQ ID NO: 512) |
| 16 Up rev | TCGTATTAATTTCGATAAGCCA |
| | GGTTAACCTGCTCCGTTAATTC |
| | GGGTTTCAATCACTT |
| | (SEQ ID NO: 513) |
| G418 for | TTCAAATCCGAACAACAGAGCA |
| | TAGGGTTTCGCAAAGCAGGTTA |
| | ACCTGGCTTATCGAA |
| | (SEQ ID NO: 514) |
| G418 rev | CTCAAAGTAATGCTGCAGTCTC |
| | GGCGCGCCGGCTACAATTAATA |
| | CATAACCTTATGTATC |
| | (SEQ ID NO: 515) |
| 16 Down for | GGCAATCACATCACCATGAGTT |
| | GTGCGGCCGCTGCCTACGCAAC |
| | ACTTTAGCTG |
| | (SEQ ID NO: 516) |
| 16 Down rev | TTGTTGGGATTCCATTGTGATT |
| | AAGG |
| | (SEQ ID NO: 517) |

TABLE 6
Primers used to generate the pBOT vectors

| | |
|---|---|
| Primer name | Sequence 5′→3′ |

Generation of four promoter-SapI-
stuffer-KasI-GFP-KasI-terminator cassettes

| | |
|---|---|
| GFP_Sap_TAA_CYCt_F | CACATGGCATGGATGAGCTC |
| | TACAAATAAGCTCTTCTTAA |
| | TCATGTAATTAGTTATGTCA |
| | CG |
| | (SEQ ID NO: 518) |
| GFP_Sap_TAA_Adh1t_f | CACATGGCATGGATGAGCTC |
| | TACAAATAAGCTCTTCTTAA |
| | TGGACTTCTTCGCCA |
| | (SEQ ID NO: 519) |
| GFP_Sap_TAA_PGlt_f | CACATGGCATGGATGAGCTC |
| | TACAAATAAGCTCTTCTTAA |
| | AACAAATCGCTCTTAAATAT |
| | ATAC |
| | (SEQ ID NO: 520) |
| GFP_Sap_TAA_Tpit_f | CACATGGCATGGATGAGCTC |
| | TACAAATAAGCTCTTCTTAA |
| | ACATAGTGTTTAAAGATTAC |
| | GG |
| | (SEQ ID NO: 521) |
| GFP_Koz_sap_ACA_Tdh3p_R | GAAAAGTTCTTCTCCTTTAC |
| | TCATTGTTTGCTCTTCTGTT |
| | TCGAAACTAAGTTCTTGGT |
| | (SEQ ID NO: 522) |
| GFP_Koz_sap_ACA_Tef1p_R | GAAAAGTTCTTCTCCTTTAC |
| | TCATTGTTTGCTCTTCTGTT |
| | ATTAAAACTTAGATTAGATT |
| | GCTATG |
| | (SEQ ID NO: 523) |
| GFP_Koz_sap_ACA_Pdc1p_R | GAAAAGTTCTTCTCCTTTAC |
| | TCATTGTTTGCTCTTCTGTT |
| | TGATTTGACTGTGTTATTTT |
| | G |
| | (SEQ ID NO: 524) |
| pfoldgfp_CYCt_F | TGCTGGGATTACACATGGCA |
| | TGGATGAACTATACAAATAA |
| | TCATGTAATTAGTTATGTCA |
| | CG |
| | (SEQ ID NO: 525) |
| pfoldgfp_Adh1t_f | TGCTGGGATTACACATGGCA |
| | TGGATGAACTATACAAATAA |
| | TGGACTTCTTCGCCA |
| | (SEQ ID NO: 526) |
| pfoldgfp_PGlt_f | TGCTGGGATTACACATGGCA |
| | TGGATGAACTATACAAATAA |
| | AACAAATCGCTCTTAAATAT |
| | ATAC |
| | (SEQ ID NO: 527) |
| pfoldgfp_Tpit_f | TGCTGGGATTACACATGGCA |
| | TGGATGAACTATACAAATAA |
| | ACATAGTGTTTAAAGATTAC |
| | GG |
| | (SEQ ID NO: 528) |
| pfoldgfp_pure_R | TTATTTGTATAGTTCATCCA |
| | TGC |
| | (SEQ ID NO: 529) |
| GFP_pfoldgfp_F | TGCTGGGATTACACATGGCA |
| | TGGATGAACTATACAAATAA |
| | GGATCCGCTGGCT |
| | (SEQ ID NO: 530) |
| L1_Koz_sap_AAACA_Tdh3p_R | TTAATTATTTCTCTTCCTTT |
| | TATAATAAATTTTCTAGGCT |
| | CTTCATGTTTTCGAAACTAA |
| | GTTCTTGGT |
| | (SEQ ID NO: 531) |
| L1_Koz_sap_AAACA_Tef1p_R | TTAATTATTTCTCTTCCTTT |
| | TATAATAAATTTTCTAGGCT |
| | CTTCATGTTTATTAAAACTT |
| | AGATTAGATTGCTATG |
| | (SEQ ID NO: 532) |
| L1_Koz_sap_AAACA_Fba1p_R | TTAATTATTTCTCTTCCTTT |
| | TATAATAAATTTTCTAGGCT |
| | CTTCATGTTTTATGTATTAC |
| | TTGGTTATGGTTATATAT |
| | (SEQ ID NO: 533) |
| L1_Koz_sap_AAACA_Pma1p_R | TTAATTATTTCTCTTCCTTT |
| | TATAATAAATTTTCTAGGCT |
| | CTTCATGTTTTTTGATAATT |
| | AAATCTTTCTTATCTT |
| | (SEQ ID NO: 534) |
| L1_sap_Kas_GFP_F | AGCCTAGAAAATTTATTATA |
| | AAAGGAAGAGAAATAATTAA |
| | ACAGCTCTTCTGGCGCCGCT |
| | GGCTCCGCTG |
| | (SEQ ID NO: 535) |
| Stop2_Kas_GFP_R | TTAGGCGCCTTATTTGTATA |
| | GTTCATCCATG |
| | (SEQ ID NO: 536) |
| pfoldgfp_KasI_TAA_CYCt_F | CACATGGCATGGATGAACTA |
| | TACAAATAAGGCGCCTAATC |
| | ATGTAATTAGTTATGTCACG |
| | (SEQ ID NO: 537) |
| pfoldgfp_KasI_TAA_Adh1t_f | CACATGGCATGGATGAACTA |
| | TACAAATAAGGCGCCTAATG |
| | GACTTCTTCGCCA |
| | (SEQ ID NO: 538) |
| pfoldgfp_KasI_TAA_PGlt_f | CACATGGCATGGATGAACTA |
| | TACAAATAAGGCGCCTAAAA |
| | CAAATCGCTCTTAAATATAT |
| | AC |
| | (SEQ ID NO: 539) |
| pfoldgfp_KasI_TAA_Tpit_f | CACATGGCATGGATGAACTA |
| | TACAAATAAGGCGCCTAAGA |
| | TTAATATAATTATATAAAAA |
| | TATTATCTTCTTT |
| | (SEQ ID NO: 540) |

Generation of modular backbones

| | |
|---|---|
| LX_(Kan)_R | TGACCTAGACTGGCTTTGAT |
| | CTTTAATTACACACTTATCC |
| | CAGCTGAAGCTTCGTACGCT |
| | GCAGG |
| | (SEQ ID NO: 541) |
| LX_NdeI_(HISt)_f | GGATAAGTGTGTAATTAAAG |
| | ATCAAAGCCAGTCTAGGTCA |
| | TATGCGTCGAGTTCAAGAGA |
| | AA |
| | (SEQ ID NO: 542) |
| LX_NdeI_(LEUt)_f | GGATAAGTGTGTAATTAAAG |
| | ATCAAAGCCAGTCTAGGTCA |
| | TATGTCGACTACGTCGTAAG |
| | G |
| | (SEQ ID NO: 543) |
| LX_NdeI_(URAt)_f | GGATAAGTGTGTAATTAAAG |
| | ATCAAAGCCAGTCTAGGTCA |
| | TATGGGTAATAACTGATATA |
| | ATTAAATTGAAG |
| | (SEQ ID NO: 544) |
| Lx_KamMX_R | TGACCTAGACTGGCTTTGAT |
| | CTTTAATTACACACTTATCC |
| | TCGACAACCCTTAATATAAC |
| | TT |
| | (SEQ ID NO: 545) |
| LY_(HISp)_r | CTGTTGCCTGACGTGAGTGG |
| | TGCCTTTGATGATGAGATAC |
| | CGTTTTAAGAGCTTGGTG |
| | (SEQ ID NO: 546) |
| LY_(LEUp)_r | CTGTTGCCTGACGTGAGTGG |
| | TGCCTTTGATGATGAGATAC |
| | CGAGGAGAACTTCTAGTATA |
| | TCC |
| | (SEQ ID NO: 547) |
| LY_(CEN6ARS4)_F | GTATCTCATCATCAAAGGCA |
| | CCACTCACGTCAGGCAACAG |
| | GGACGGATCGCTTG |
| | (SEQ ID NO: 548) |
| LY_(2micron)_F | GTATCTCATCATCAAAGGCA |
| | CCACTCACGTCAGGCAACAG |
| | ATACTCCGTCTACTGTACGA |
| | TAC |
| | (SEQ ID NO: 549) |
| LY_(URAp)_r | CTGTTGCCTGACGTGAGTGG |
| | TGCCTTTGATGATGAGATAC |
| | ATTCATCATTTTTTTTTTAT |
| | TCTT |
| | (SEQ ID NO: 550) |
| LZ_(CEN6ARS4)_R | CTGACGTCGGTAAAGTAGGA |
| | GTGTCTGCAATAGGTCTTAA |
| | GGTCCTTTTCATCACGT |
| | (SEQ ID NO: 551) |
| LZ_(2micron)_R | CTGACGTCGGTAAAGTAGGA |
| | GTGTCTGCAATAGGTCTTAA |
| | GTGCTATATCCCTATATAAC |
| | CTACC |
| | (SEQ ID NO: 552) |
| LZ_(Ecoli_unit)_F_ | TTAAGACCTATTGCAGACAC |
| pBluescript | TCCTACTTTACCGACGTCAG |
| | CAGGTGGCACTTTTCG |
| | (SEQ ID NO: 553) |
| LV3_AscI_(pMB1ori)_R_ | GCATTTTTATTATATAAGTT |
| pBluescript | GTTTTATTCAGAGTATTCCT |
| | GGCGCGCCCGCGTTGCTGGC |
| | GTT |
| | (SEQ ID NO: 554) |
| LV5_NotI_kanmx_F | CCTCTTTATATTACATCAAA |
| | ATAAGAAAATAATTATAACA |
| | CAGATCCGCGGCCGC |
| | (SEQ ID NO: 555) |

### Cell Feeding Assays

Whole cell substrate feeding assays were used to test the function of each enzyme block individually and in combinations. To prepare the cells for the feeding assays, a colony of S. cerevisiae was inoculated in YNB-DO-GLU and incubated for 24 hours. Cultures were diluted to an OD₆₀₀ of 0.8 into 6 ml of fresh YNB-DO-GLU and incubated for an additional 7 hours. Cells were harvested by centrifugation at 2000×g for 2 min. Supernatants were decanted and cells were suspended in 2 ml of Tris-EDTA (10 mM Tris-HCl, 1 mM EDTA, pH 8), containing 10 μM of one of the following feeding substrates: (R,S)-norlaudanosoline, (S)-reticuline, (S)-scoulerine or (S)-stylopine. Cells were incubated for 16 hours then harvested by centrifugation at 15000×g for 1 min. For BIA extraction from cells, the cell pellet was suspended in 500 μl methanol with ˜50 μl acid-washed glass beads and vortexed for 30 min. Cell extracts were clarified by centrifugation at 15000×g for 1 min and used directly for LC-MS analysis.

### MRM Analysis of Alkaloids

Analysis of enzyme assays was performed using an Agilent™ 1200 liquid chromatography system equipped with a 6410 triple-quadrupole mass spectrometer (Agilent Technologies; Santa Clara, Calif.). Ten microliters of the reaction mixtures were separated as described previously²⁵ and the eluate was applied to the mass analyser using the following parameters: capillary voltage, 4000 V; fragmentor voltage, 125 V; source temperature, 350° C.; nebulizer pressure, 50 psi; gas flow, 10 L min⁻¹. Scoulerine, cheilanthifoline, and stylopine were detected in multiple reaction monitoring (MRM) mode using a collision energy of 25 eV and monitored transitions of m/z 328→178, 326→178 and 324→176, respectively.

### FT-MS Analysis of Alkaloids

Detection of alkaloids in the sanguinarine biosynthetic pathway was performed by FT-MS using a 7T-LTQ FT ICR instrument (Thermo Scientific, Bremen, Germany). Alkaloids were separated by reverse phase HPLC (Perkin Elmer SERIES 200 Micropump, Norfolk, Conn.) using an Agilent Zorbax™ Rapid Resolution HT C18 2.1*30 mm, 1.8 micron column. Solvent A (0.1% acetic acid) and solvent B (100% acetonitrile) were used in a gradient elution to separate the metabolites of interest as follows: 0-1 min at 100% A, 1-6 min 0 to 95% B (linear gradient), 7-8 min 95% B, 8-8.2 min 100% A, followed by a 1 min equilibration at 100% A. Three microliters of either cell extract or supernatant fraction were loaded on the HPLC column run at a flow rate of 0.25 ml/min. Dilutions in methanol were performed to keep alkaloid concentrations within the range of standard curve values and avoid saturating FT signals. Following LC separation, metabolites were injected into the LTQ-FT-MS (ESI source in positive ion mode) using the following parameters: resolution, 100000; scanning range, 250 to 450 AMU; capillary voltage, 5 kV; source temperature, 350° C.; AGC target setting for full MS were set at 5×10⁵ ions. Identification of alkaloids was done using retention time and exact mass (<2 ppm) of the monoisotopic mass of the protonated molecular ion [M+H]⁺. LC-FT-MS data were processed using the freely available program Maven⁵⁶. When available authentic standards were used to confirm the identity of the BIA intermediates (using HPLC retention times and exact masses) and to quantify sanguinarine alkaloids. When unavailable, we assumed equal ionization efficiency between an intermediate and the closest available quantifiable alkaloid (m/z 302 and m/z=316: reticuline; cheilanthifoline: stylopine).

### Example 2

### Isolation and In Vitro Characterization of Cheilanthifoline and Stylopine Synthases from Opium Poppy

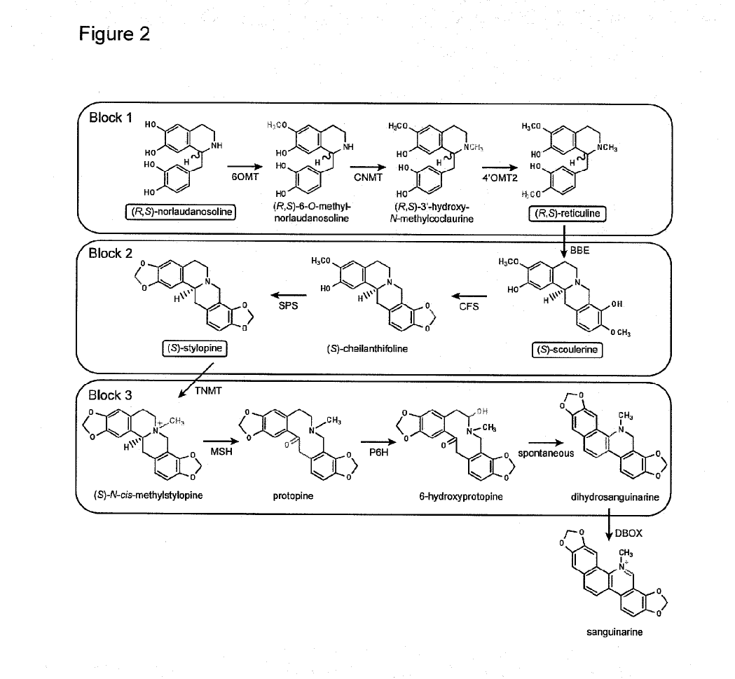

Sanguinarine biosynthesis from (S)-scoulerine proceeds with the formation of two methylenedioxy bridges catalysed by the P450s cheilanthifoline synthase (CFS) and stylopine synthase (SPS), to yield cheilanthifoline and stylopine respectively (FIG. 2). Candidate genes encoding opium poppy (Papaver somniferum) cheilanthifoline synthase (PsCFS) and stylopine synthase (PsSPS) were isolated from assembled opium poppy root and stem transcriptome databases generated using Illumina GA™ sequencing. Query amino acid sequences used to search each database were AmCFS and AmSPS from Argemone mexicana²⁸. The selection of candidate genes was guided by the expectation that PsCFS and PsSPS transcripts would occur exclusively or predominantly in opium poppy roots, owing to the lack of sanguinarine accumulation in stems of the plant²⁹. A PsCFS candidate corresponding to CYP719A25 shared 84% amino acid identity with AmCFS and was expressed exclusively in opium poppy roots (FIG. 3A). A PsSPS candidate corresponding to CYP719A20 shared 79% amino acid identity with AmSPS and was expressed predominantly in roots (FIG. 3A).

Constructs for the heterologous expression of PsCFS and PsSPS in S. cerevisiae were assembled in a vector harbouring PsCPR (Table 1 above). As PsSPS was poorly expressed (data not shown), the native N-terminal membrane-spanning domain was swapped with that of lettuce germacrene A oxidase, generating recombinant PsSPSΔN. Western blot analysis confirmed expression of the recombinant proteins in yeast (FIG. 3B). Microsomes were isolated from all three strains and incubated in vitro with (S)-scoulerine. Scoulerine was converted to cheilanthifoline in the presence of PsCPR and PsCFS, while scoulerine was converted to cheilanthifoline and stylopine in the presence of PsCPR, PsCFS, and recombinant PsSPSΔN (FIG. 3C). No conversion of scoulerine was detected in the negative control. In Examples 3-8, PsCFS and recombinant PsSPSΔN were used for the reconstitution of dihydrosanguinarine synthesis in S. cerevisiae.

### Example 3

### Reconstitution of the Dihydrosanguinarine Pathway

The synthesis of sanguinarine from norlaudanosoline requires ten enzymatic reactions (FIG. 2). In reconstitution of the pathway, dihydrobenzophenanthridine oxidase (DBOX) was omitted herein because of its low activity in S. cerevisiae²⁶ and because dihydrosanguinarine is easily oxidized to sanguinarine ex vivo³⁰. The remaining nine reactions were divided into three “blocks” of three sequential enzymes, as illustrated in FIG. 2. Each block was cloned into a separate plasmid and the cytochrome P450 reductase from P. somniferum (PsCPR) was cloned into a fourth plasmid (Table 1 above).

To confirm functional expression of enzymes, plasmids expressing each of the three blocks were individually transformed into S. cerevisiae and cultures of the strains were supplemented with either (R,S)-norlaudanosoline, (S)-reticuline, (S)-scoulerine or (S)-stylopine. Functional expression was verified by detection of the expected end products. As negative controls, yeast strains lacking enzyme blocks were incubated with each of the pathway intermediate to evaluate substrate recovery and to assess the relative proportion recovered in cellular extracts versus culture supernatants.

### Example 4

### Functional Expression of Individual Enzyme Blocks

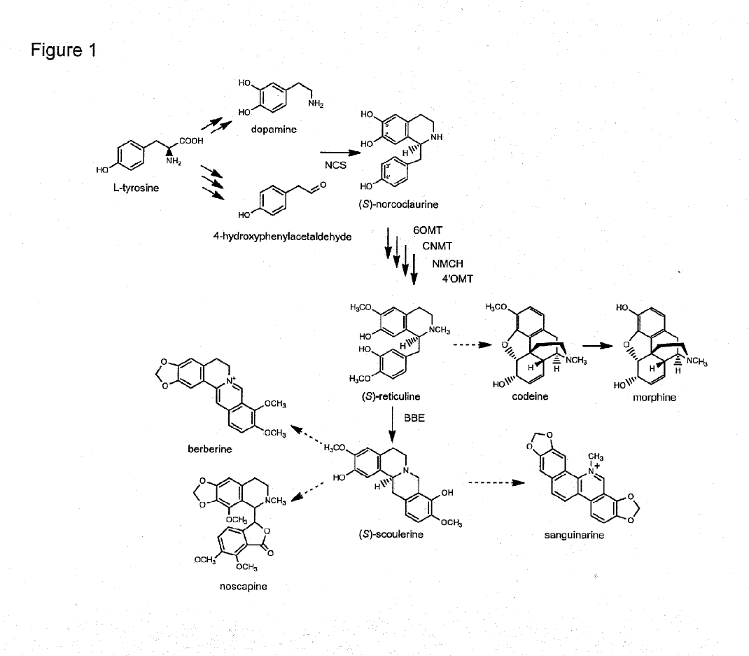

Block 1 contains the P. somniferum enzymes 6-O-methyltransferase (6OMT), coclaurine N-methyltransferase (CNMT), and 4′-O-methyltransferase 2 (4′OMT2), which catalyse three methylation reactions to convert (R,S)-norlaudanosoline to (S)-reticuline. The committed step of BIA synthesis in plants is the condensation of the L-tyrosine derivatives L-dopamine and 4-hydroxyphenylacetaldehyde to produce (S)-norcoclaurine, catalysed by the enzyme (S)-norcoclaurine synthase (NCS) (FIG. 1). The enzymatic synthesis of (S)-norcoclaurine has not yet been achieved in S. cerevisiae, however the 3′-hydroxylated analogue norlaudanosoline can be used to bypass synthesis of (S)-norcoclaurine and the P450 NMCH, N-methylcoclaurine hydroxylase (CYP80B1), during pathway development²². The enzymes 6OMT, CNMT and 4′OMT2 from P. somniferum can be functionally co-expressed in yeast for the synthesis of reticuline from (R,S)-norlaudanosoline²².

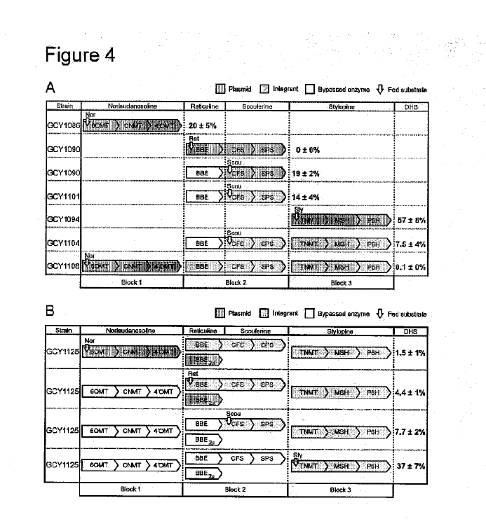

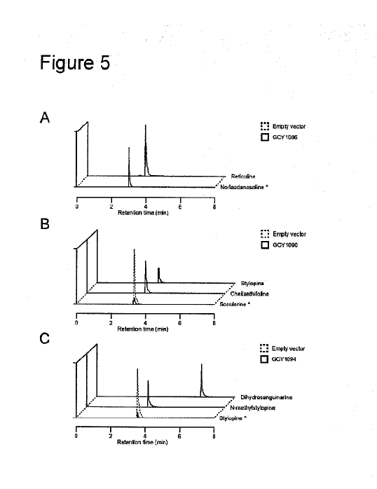

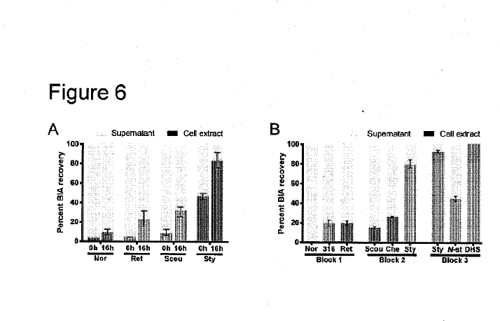

Strain GCY1086, expressing Block 1 enzymes from a plasmid, was incubated with (R,S)-norlaudanosoline. The end product reticuline was produced with a yield of 20% (FIG. 4A and FIG. 5A), twice the previously reported yield²². While Block 1 enzymes are predicted to function in plants in the order depicted in FIG. 1, a multitude of single- and double-methylated products can be formed when these enzymes are expressed in S. cerevisiae and incubated with norlaudanosoline^{22,31,32}. For example, the three methyltransferases expressed in Block 1 can accept norlaudanosoline as a substrate and acceptance of N-methylnorlaudanosoline (laudanosoline) by both 6OMT and 4′OMT2 was also demonstrated²². No single-methylated products (m/z=302) were however detected herein, and the accumulation of double-methylated products (m/z=316) was estimated to be 2%. Low accumulation of intermediates indicated that the limiting reaction of Block 1 is likely the first methylation event. This limitation could be due to the fact that norlaudanosoline is not a natural substrate or that intracellular norlaudanosoline is low due to poor transport. When wild-type cells were incubated with norlaudanosoline, 3% was initially found in the cell extract, which increased to just 10% after 16 hours of incubation (FIG. 6A), suggesting that substrate availability to the intracellular enzymes is low.

### Chiral Analysis of Reticuline

To investigate the possibility that the reticuline produced from (R,S)-norlaudanosoline by the three opium poppy MTs was not racemic, chiral analysis by HPLC was used to reveal the presence or absence of reticuline enantiomers.

Separation of the (R)- and (S)-enantiomers of reticuline was performed using the chiral column CHIRALCEL OD-H (4.6×250 mm, Daicel Chemical Industries) and the solvent system hexane:2-propanol:diethylamine (78:22:0.01) at a flow rate of 0.55 ml min^{-1 60}. Following LC separation, metabolites were injected into an LTQ ion trap mass spectrometer (Thermo Electron, San Jose, Calif.) and detected by selected reaction monitoring (SRM). SRM transitions of m/z 288→164.0 (CID@35) and 330→192 (CID@30) were used to detect reticuline. Retention times for reticuline obtained in samples matched retention times observed with authentic standards.

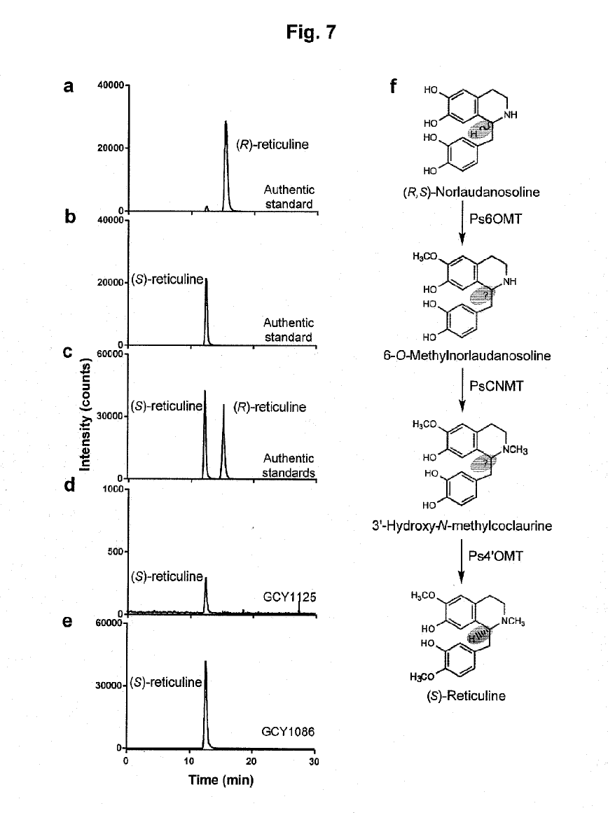

Chiral analysis of enantio-pure standards of (R)- and (S)-reticuline and of racemic (R,S)-reticuline was first performed to confirm the separation of the two enantiomers (FIGS. 7A, 7B and 7C). Analysis of the reticuline produced by the BBE-expressing strain GCY1359, which was assumed to accumulate (R)-reticuline and convert (S)-reticuline into scoulerine, showed instead that only trace (S)-reticuline remained (FIG. 7D). Finally, chiral analysis of the reticuline produced by strain GCY1086 expressing only the three P. somniferum MTs revealed that only (S)-reticuline was produced (FIG. 7E), demonstrating the strict enantioselectivity of one or more of the three MTs on racemic i.e. (R,S)-norlaudanosoline (FIG. 7F).

Block 2 contains the P. somniferum enzymes berberine bridge enzyme (BBE), CFS and SPS. In plants, the flavoprotein oxidase BBE stereoselectively converts (S)-reticuline to (S)-scoulerine³³. A truncated version of BBE (PsBBEΔN) was cloned with CFS and recombinant PsSPSΔN into plasmid pGC994 (Table 1). When cells expressing enzymes of Block 2 and CPR (strain GCY1090) were incubated with (S)-reticuline, no scoulerine, cheilanthifoline, or stylopine were detected (FIG. 4A), suggesting that initial conversion of (S)-reticuline to scoulerine was poor. It was hypothesized BBEΔN activity was limiting the flux of Block 2. To assess the function of CFS and recombinant PsSPSΔN, PsBBEΔN was bypassed by feeding the next pathway intermediate, (S)-scoulerine, to the same yeast strain. Here, the accumulation of cheilanthifoline and 19% conversion of scoulerine to stylopine (FIG. 4A and FIG. 5) were observed, confirming that PsBBEΔN was limiting. 70% conversion to cheilanthifoline was estimated using a stylopine standard curve. Cheilanthifoline was mainly found in the supernatant while stylopine was predominantly found in the cell extract (FIG. 6B). Accumulation of cheilanthifoline during cell feeding assays also indicated that low activity of the stylopine synthase was limiting the flux of scoulerine to stylopine.

The third block encodes the three enzymes catalysing the conversion of stylopine to dihydrosanguinarine. Stylopine is N-methylated to (S)-cis-N-methylstylopine by tetrahydroprotoberberine cis-N-methyltransferase (TNMT)³⁴. The next two biosynthetic steps are catalysed by the P450 hydroxylases (S)-cis-N-methylstylopine 14-hydroxylase (MSH) and protopine 6-hydroxylase (P6H). Both enzymes were recently identified and characterized from P. somniferum and Eschscholzia californica respectively (CYP82N4 and CYP82N2v2)^{25,27}. 6-Hydroxyprotopine spontaneously rearranges to dihydrosanguinarine. When cells expressing Block 3 and CPR (strain GCY1094) were incubated with (S)-stylopine, the majority of the stylopine was consumed, resulting in 57% conversion to dihydrosanguinarine (FIG. 4A and FIG. 5C). N-methylstylopine was the intermediate with the next largest peak area (FIG. 5C) and trace amounts of protopine were detected (not shown). 3% conversion to sanguinarine was also detected indicating spontaneous conversion of dihydrosanguinarine to sanguinarine (not shown).

Approximately 55% of the pathway intermediate N-methylstylopine (N-st) was found in the culture supernatant as opposed to stylopine (Sty) and dihydrosanguinarine (DHS), which were mostly found in the cellular extract fraction (FIG. 6B). This suggests that the charged intermediate N-methylstylopine (N-st) is secreted outside of the cell, thereby reducing the pathway efficiency.

### Example 5

### Integration of Block 2 and Block 3 in the Genome

The vectors harbouring Blocks 1, 2, 3, and CPR are closely related and share several of the same promoters and terminators. A loss of function was occasionally observed from strains harbouring multiple plasmids, which was attributed to recombination. To address this problem, Blocks 2 and 3 were integrated into the genome of S. cerevisiae (Table 1 and Table 2). Block 2 was integrated into YORWΔ17(ChrXV). Block 3 was integrated into YPRCΔ15(ChrXVI). The plasmids were single copy.

Block 2 was integrated into S. cerevisiae and the strain was transformed with CPR (strain GCY1101). When incubated with scoulerine, 14% of the substrate was converted to stylopine, which is comparable to what was obtained by expressing Block 2 from a centromeric plasmid (single copy) (strain GCY1090)(FIG. 4A) Only trace cheilanthifoline and stylopine accumulation was observed in the absence of the heterologously-expressed CPR, indicating the importance of including a cognate CPR for improved cytochrome P450 activity in S. cerevisiae (data not shown).

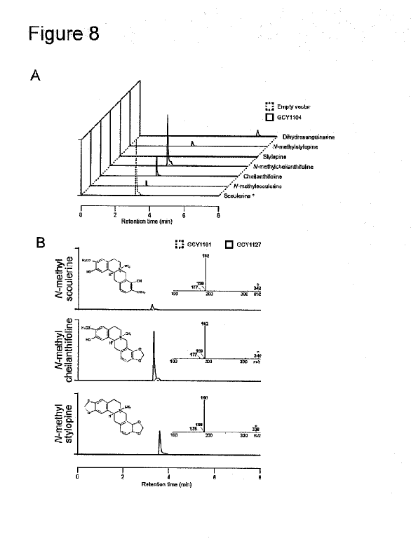

Block 3 was integrated into the Block 2 strain, generating a Block 2-Block 3 double integrant, which was subsequently transformed with CPR (strain GCY1104). Incubation of this strain with (S)-scoulerine resulted in 7.5% conversion to dihydrosanguinarine (FIG. 4A and FIG. 6A), which is an 8-fold decrease in dihydrosanguinarine synthesis compared to cells expressing Block 3 enzymes and incubated with (S)-stylopine (FIG. 4A). Similar to previous feeding assays where cells expressed only Block 3 enzymes, the intermediate N-methylstylopine accumulated (FIG. 8A).

### Example 6

### TNMT N-Methylates Scoulerine and Cheilanthifoline

When cells of the Block 2-Block 3 double integrant were incubated with (S)-scoulerine, compounds with exact masses of 342.1710 m/z and 340.1548 m/z were detected in addition to expected sanguinarine pathway intermediates. These exact masses, and their CIDs, correspond to N-methylscoulerine and N-methylcheilanthifoline, respectively³⁶. The compounds had been previously identified in opium poppy cell culture and were predicted to be a product of TNMT activity, an enzyme that had also been shown to methylate (S)-canadine³⁴. To determine whether TNMT was responsible for the N-methylation of cheilanthifoline and scoulerine, the Block 2 integrant strain harbouring CPR on a plasmid (strain GCY1101) was compared with the Block 2 integrant strain harbouring both CPR and TNMT on a plasmid (strain GCY1127). The resulting chromatograms and CIDs (FIG. 8B) for N-methylscoulerine, N-methylcheilanthifoline, and N-methylstylopine demonstrate that all three compounds are present only in the presence of TNMT. These data provide the first experimental evidence that TNMT accepts both scoulerine and cheilanthifoline as substrates for methylation.

Because the applicants lacked standards to quantify the N-methylated products and since the three compounds are similar in structure, they assumed equal ionization efficiency and estimated their relative proportions by peak area: when strain GCY1127 is fed (S)-scoulerine, the expected product N-methylstylopine is 33%, while N-methylscoulerine is 7% and N-methylcheilanthifoline is 60% (FIG. 8B). Diversion of the intermediates scoulerine and cheilanthifoline from dihydrosanguinarine synthesis affects the efficiency of the pathway and showed that favouring N-methylation of stylopine will be useful to increase yield to the desired downstream compounds as was confirmed in Examples below.

### Example 7

### Production of Dihydrosanguinarine from Norlaudanosoline

The three functional blocks were combined to assemble a complete dihydrosanguinarine pathway in yeast. The Block 2-Block 3 integrant served as a background strain for the transformation of Block 1 and CPR (strain GCY1108). When this strain was incubated with (R,S)-norlaudanosoline, trace levels of dihydrosanguinarine were observed (FIG. 4A), along with 13% conversion to reticuline and no other accumulation of downstream intermediates or N-methylated side products (data not shown). Lack of conversion of endogenously synthesized (S)-reticuline to scoulerine confirmed that psBBEΔN was not highly active in the applicant's system, as previously observed by incubating cells expressing Block 2 enzymes with (S)-reticuline (FIG. 4A).

The applicants co-transformed a high-copy vector expressing psBBEΔN along with Block 1 and CPR plasmids into the double integrant (strain GCY1125). When strain GCY1125 was incubated with (R,S)-norlaudanosoline, conversion to dihydrosanguinarine improved from trace to 1.5% (FIG. 5A) and (S)-reticuline accumulation dropped from 13% to 0.5%, confirming that low psBBEΔN expression was limiting flux. PsBBE was previously proposed to be functionally identical to Eschscholzia californica BBE^{26,37}, which is enantioselective for (S)-reticuline³³.

Finally, the applicants independently incubated GCY1125 cells with (R,S)-norlaudanosoline, (S)-reticuline, (S)-scoulerine, or (S)-stylopine to assess the efficiency of substrate conversion at different points in the complete pathway (FIG. 4B). In all GCY1125 substrate feeding experiments, spontaneous oxidation of dihydrosanguinarine to sanguinarine was detected, corresponding to 6% of the total dihydrosanguinarine. Feeding (R,S)-norlaudanosoline resulted in 1.5% conversion to dihydrosanguinarine, while bypassing Block 1 using (S)-reticuline resulted in 4% conversion to dihydrosanguinarine. Bypassing PsBBEΔN by incubating the same strain with (S)-scoulerine increased conversion to only 8%. In all cases the accumulation of the N-methylated side-products, especially N-methylcheilanthifoline, diverted intermediates from the dihydrosanguinarine pathway. In fact, the biggest increase in efficiency was observed when strain GCY1125 was incubated with stylopine, bypassing production of scoulerine and cheilanthifoline and their N-methylated products: from 8% to 37%. However, 37% yield is a decrease from the 57% yield observed when Block 3 enzymes were expressed on their own, indicating that expression of Block 1 and 2 negatively affects the yield of Block 3.

### Example 8

### Effect of pH on Yield

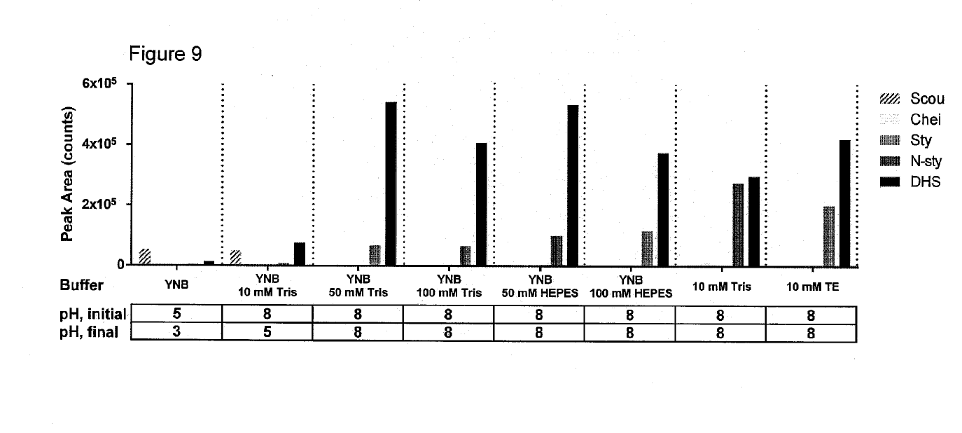

Block 2_Block 3 enzymes were tested in whole cell substrate (GCY1104) feeding assays for the production of dihydrosanguinarine from scoulerine using different buffering conditions (FIG. 9). Yeast growing medium (YNB-DO-GLU) has an initial pH of ˜5, which drops to pH ˜3 during yeast growth.

A colony of S. cerevisiae was inoculated in YNB-DO-GLU and incubated for 24 hours. Cultures were diluted to an OD₆₀₀ of 0.8 into 6 ml of fresh YNB-DO-GLU and incubated for an additional 7 hours. Cells were harvested by centrifugation at 2000×g for 2 min. Supernatants were decanted and cells were suspended in 2 ml of each of the following media containing 10 μM of of (S)-scoulerine:
1. YNB-DO-GLU, pH ˜5 (YNB in the FIG.
2. YNB-DO-GLU+10 mM Tris-HCl pH 8 (YNB+10 mM Tris in the FIG.
3. YNB-DO-GLU+50 mM Tris-HCl pH 8 (YNB+50 mM Tris in the FIG.
4. YNB-DO-GLU+100 mM Tris-HCl pH 8 (YNB+100 mM Tris in the FIG.
5. YNB-DO-GLU+50 mM HEPES pH 8 (YNB+50 mM HEPES in the FIG.
6. YNB-DO-GLU+10 mM HEPES pH 8 (YNB+100 mM HEPES in the FIG.
7. Tris-HCl 10 mM, pH8 (Tris 10 mM in the FIG.
8. 10 mM Tris-HCl, 1 mM EDTA, pH 8 (TE 10 mM in the figure). This is the condition used in Examples 1 to 7 herein.

Cells were incubated for 16 hours and at the end of the feeding the pHs were verified. In sample 1 the pH dropped from 5 to 3 as expected. In sample 2 the pH dropped from 8 to 5, indicating that the buffer strength was not enough to maintain the pH at 8. All other buffers maintained the pH at 8. Extraction of alkaloids and analysis were performed as described above.

Results shown in FIG. 9 indicate that when final pH is 8, dihydrosanguinarine production is 5 to 8 times higher than at pH 3 or 5. Without being bound by such hypothesis, the applicants submit that more alkaline pHs affect substrate solubilities and therefore availability. pH values indicated in FIG. 9 refer to yeast growing medium, not to yeast cells' internal pH.

### Example 10

### Effect of pH on Sanguinarine Pathway BIA Synthesis

Additional pHs of 3, 6, 7, 8, 9 were tested on the individual Blocks 1, 2 and 3 and on the three Blocks 1-2-3 combined in GCY1125 in order to evaluate the effect of pH on sanguinarine pathway BIA synthesis and recovery.

A variety of media was used: the yeast media YNB, which has a starting pH of about 5.5 but decreases to about pH 3 upon fermentation; 10 mM Sørenson's phosphate buffer, pH 6.0; TE buffer pH 7.0 (10 mM Tris, 1 mM EDTA), TE buffer pH 8.0 (10 mM Tris, 1 mM EDTA), and TE buffer pH 9.0 (10 mM Tris, 1 mM EDTA). Yeast expressing no heterologous BIA pathway enzymes were inoculated in YNB with appropriate supplementation overnight, back-diluted 1:10 in 96-well deep-well plates, allowed to grow for 7 h and concentrated 3× in incubation media containing 5 uM of either (R,S)-norlaudanosoline, (S)-scoulerine, or (S)-stylopine. After 16 hours, supernatant was recovered and diluted 1:2 in MeOH before analysis by HPLC-FT-MS. In addition, cell pellets were resuspended in MeOH and vortexed for 30 minutes before analysis by HPLC-FT-MS.

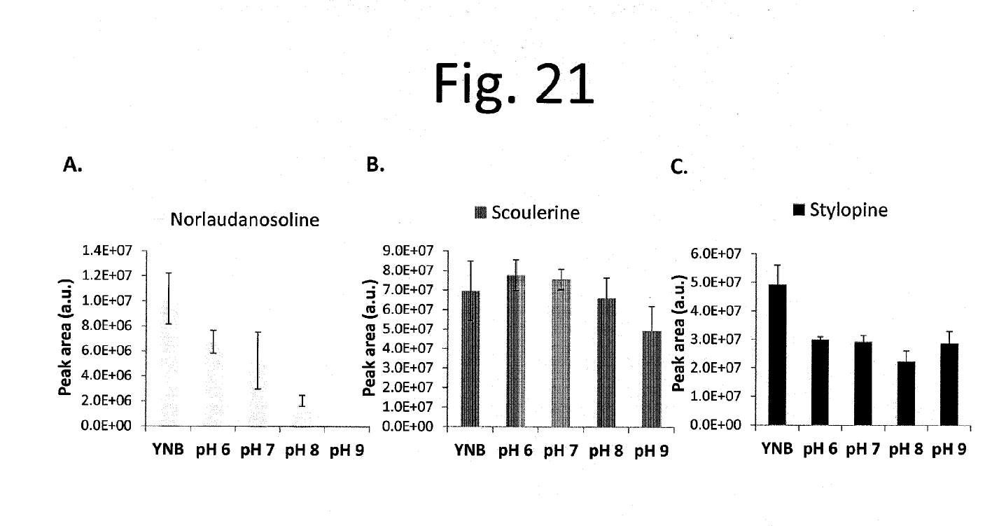

Total recovery of BIAs was not consistent across all pHs, nor was it consistent across all BIAs. (R,S)-norlaudanosoline recovery was highest in YNB, and dropped as pH increased, until at pH 9, no norlaudanosoline was recovered (FIG. 21A). (S)-scoulerine recovery remained consistent across YNB to pH 8, even increasingly slightly at pH 6 and 7; recovery dropped at pH 9 (FIG. 21B). (S)-stylopine recovery was highest in YNB, but remained consistent between pH 6 and 9 (FIG. 21C). All three supplemented BIAs showed high recovery in YNB and lower recovery at higher pHs.

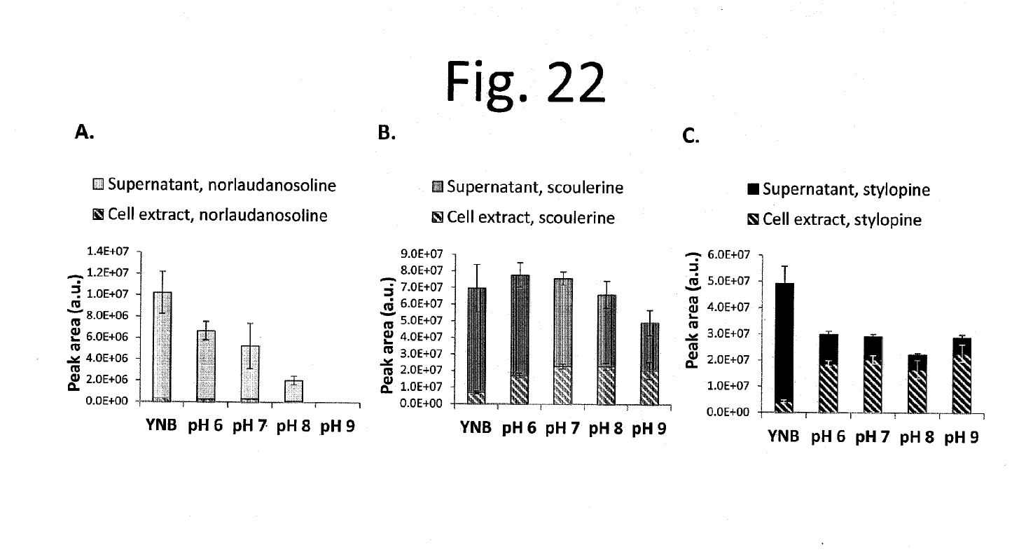

BIAs were not recovered equally from supernatant and cell extract, nor was recovery consistent across the pH range from 3-9. While for (R,S)-norlaudanosoline recovery in cell extract remained within 3-5% across all pHs, (S)-scoulerine and (S)-stylopine recovery in cell extract increased with pH (FIG. 22A, B, C). However, recovery from supernatant tended to decrease as pH increased, and this decrease was not matched by an increase in recovery in cell extract; hence, total recovery tended to drop as pH increased. In summary, incubation of BIAs at pHs above 7 was unfavourable for recovery of supplemented BIAs.

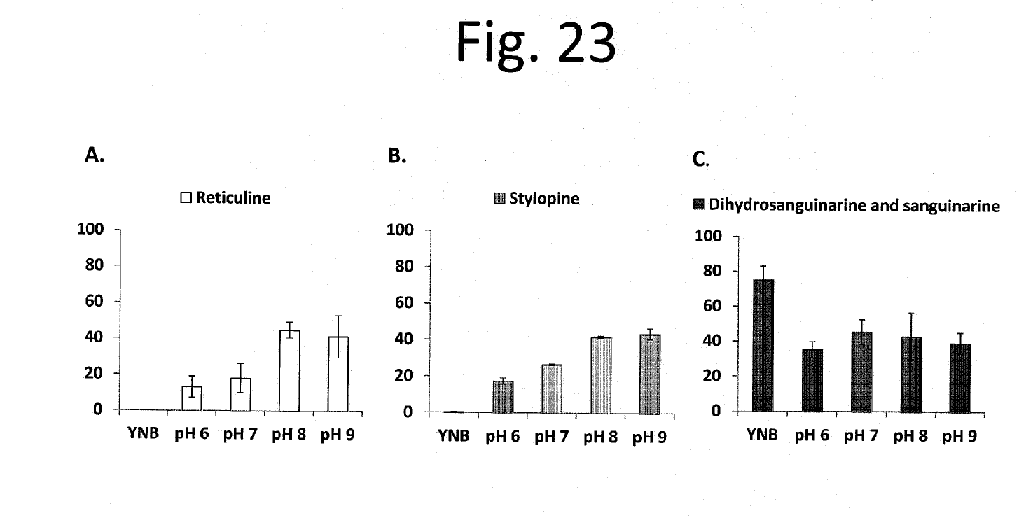

In contrast to recovery studies, activity assays tended to favour higher pHs. Yeast expressing Block 1 (GCY1086), Block 2 and PsCPR (GCY1090), or Block 3 and PsCPR (GCY1094) were inoculated overnight, back-diluted 1:10 in 96-well deep-well plates, allowed to grow for 7 h and concentrated 3× in incubation media containing 5 uM of (R,S)-norlaudanosoline (FIG. 23A), (S)-scoulerine (FIG. 23B), or (S)-stylopine (FIG. 23C), respectively. After 16 hours, supernatant was recovered and diluted 1:2 in MeOH before analysis by HPLC-FT-MS. In addition, cell pellets were resuspended in MeOH and vortexed for 30 minutes before analysis by HPLC-FT-MS. As some intermediates did not have a standard available for quantification, pathway conversion was measured as a molar ratio between product recovered and substrate added. In YNB, there was no detectable conversion of norlaudanosoline to reticuline. Conversion increased from 13% at pH 6 to 41% at pH 9. Similarly, the Block 2 end product stylopine was not observed when cells were incubated in YNB. Increasing the pH to 6 increased yields to 17%, while increasing pH further increased yields to 43% at pH 9. In contrast, the highest conversion of stylopine to dihydrosanguinarine and sanguinarine was 75% in YNB. At pH 6, conversion dropped to 35% and remained consistent as pH rose.

Poor recovery of (R,S)-norlaudanosoline at pHs greater or equal to 6 suggests degradation, perhaps by oxidation as hypothesized by Kim et al 2013⁷¹. They observed higher conversion of fermented (S)-norlaudanosoline to (S)-reticuline at pH 6 than pH 8. While the Applicant observed that norlaudanosoline is highly affected by higher pHs, conversion of norlaudanosoline towards downstream products is also higher at these pHs, thus preventing oxidation. Kim et al.⁷¹ was supplementing dopamine, which must be enzymatically condensed with its derivative 3,4-dHPAA to form norlaudanosoline; it is possible that this enzymatic step provided an extra bottleneck which led to increased oxidation.

The effect of higher pHs on Block 1 and Block 2 conversion was remarkably similar: 0% activity in YNB, 10-25% activity in pHs 6 and 7, and activity levelling off at ˜40% at pHs 8 and 9. In contrast, Block 3 was most active in YNB, where 75% of stylopine was converted into dihydrosanguinarine and sanguinarine. This suggests that a two-step fermentation could be performed, with a higher pH to promote conversion to stylopine, followed by a lower pH to promote conversion to dihydrosanguinarine and sanguinarine.

### Example 10

### Increasing the Activity of SPS and CFS

The present invention encompasses increasing the activity of SPS and CFS, and thus the flux of alkaloids towards sanguinarine. Orthologous genes from different plants can have varying kinetic constants and expression efficiency in yeast.

There are 14 enzyme families and 300 genes in the BIA gene order. The cheilanthifoline and stylopine synthases belong to the CYP719 family.

The Applicant purchased all published CYP719s (including those without published activities). Published CYP719 protein sequences were used as queries for a tblastx™ search of the PhytoMetaSyn™ transcriptome database. The interface for BLAST was on PhytoMetaSyn™'s website. Protein sequences with percent similarity of 55% or greater to published CYP719s were saved for downstream analysis.

In parallel with the BLAST approach, PhytoMetaSyn™'s transcriptome data (RNA) was downloaded and converted into predicted ORFs (protein) using the OrfPredictor algorithm developed in Dr. Tsang's laboratory at Concordia University⁶³. Two motifs were used to search the database of predicted PhytoMetaSyn™ ORFs. The first was the highly conserved heme-binding motif FXXGXRXC (SEQ ID NO: 481). The second was a common N-terminal hydrophobic region downstream of the membrane-anchor sequence, found to be conserved amongst published CYP719s: P(hydrophobic)(hydrophobic)GN⁶⁴. Protein sequences containing both motifs of interest were saved for downstream analysis.

Predicted ORFs identified through the tblastx™ search and/or motif searches described above were sorted into CYP families by percent sequence identity using the program BLAST-CLUST™ (http://toolkit.tuebingen.mpg.de/blastclust). BLAST-CLUST™ requires two inputs: “sequence length to be covered” and “percent identity threshold”. Sequence length was set to 95% to allow for variability in identity and length of membrane-anchor sequences. Percent identity was set to 40% because the CYP nomenclature committee defines CYP families as CYPs with 40% identity or more⁶⁴. All published CYP719s cluster together using these settings. Predicted ORFs that clustered with published CYP719s were selected for further analysis. Additional outliers were discarded using Clustal Omega™'s multiple sequence alignment, and phylogenetic trees were generated using the program MEGA6™⁶⁵ (See FIG. 10C-G).

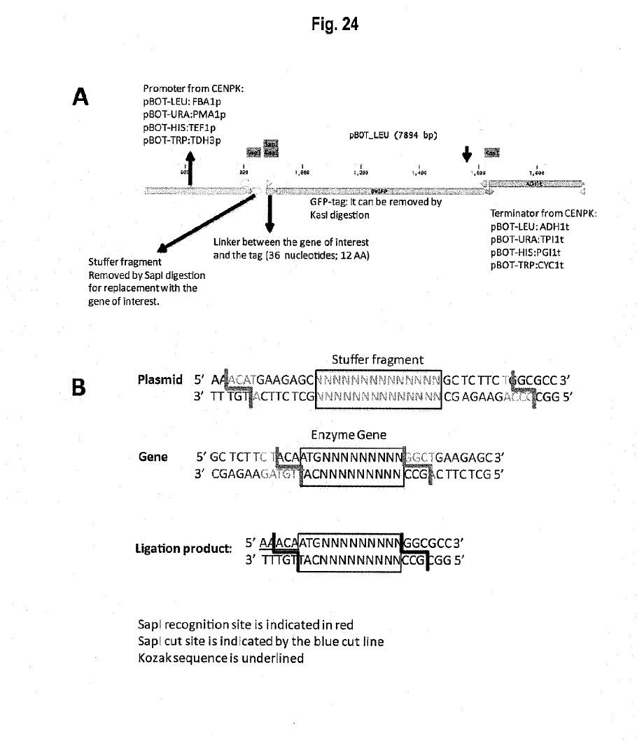

The Applicant ordered 42 CYP719s from the PhytoMetaSyn™ database, along with 19 published CYP719s, from the DNA synthesis company Gen9 (referred to herein as “purchased CYP719”). A phylogenetic tree of the ordered sequences is presented in FIG. 10C. The CYP719s were pre-cloned by Gen9 into the pBOT-TRP vector built by the Martin lab (FIG. 24).

The pBOT vector system is modular and flexible, and can be used to synthesize an unlimited number and type of vector backbones. Each vector feature is amplified individually, flanked by 40 bp linkers such that features can be combined via cloning methods relying on homologous regions of DNA. Any number of features can be used, depending on the nature of linkers used. Features used in the pBOT-TRP vector were: 1) E. coli antibiotic resistance and origin of replication; 2) yeast origin of replication; 3) yeast antibiotic resistance; 4) yeast auxotrophy; and 5) expression cassette.

The four basic pBOT vectors contain unique promoter and terminator combinations, allowing for cassette assembly via cloning methods relying on homologous regions of DNA. Genes were directionally cloned into pBOT expression cassettes as GFP fusion proteins via the type II restriction enzyme SapI. Protein expression can be measured indirectly via GFP fluorescence. GFP can be removed by digestion with KasI followed by dilution and religation, resulting in a functional expression cassette with a two amino acid scar (glycine-alanine) at the C terminus of the gene. The four pBOT versions available contain a different auxotrophy (LEU, URA, HIS or TRP) and different promoter-terminator pairs associated with each auxotrophy. Any gene of interest can be cloned by SapI restriction digestion and ligation. Target genes are PCR amplified using primers that add a SapI site at the 5′ and at the 3′ as follows: 5′-GCTCTTCTACA (SEQ ID NO: 565)-GENE-GGCTGAAGAGC-3′ (SEQ ID NO: 566). Digestion of vector generates 5′ overhangs on vector (TGT and GGC) which complement designed 5′ overhangs on digested gene sequences (ACA and CCG). Ligation of SapI digested plasmid and target gene will reconstitute a functional Kozak sequence at the 5′ of the gene (AAACA (SEQ ID NO: 567) followed by the ATG first codon and no extra UTRs region added as described. A linker of 36 nucleotides (12 amino acids) between the gene and the GFP in present.

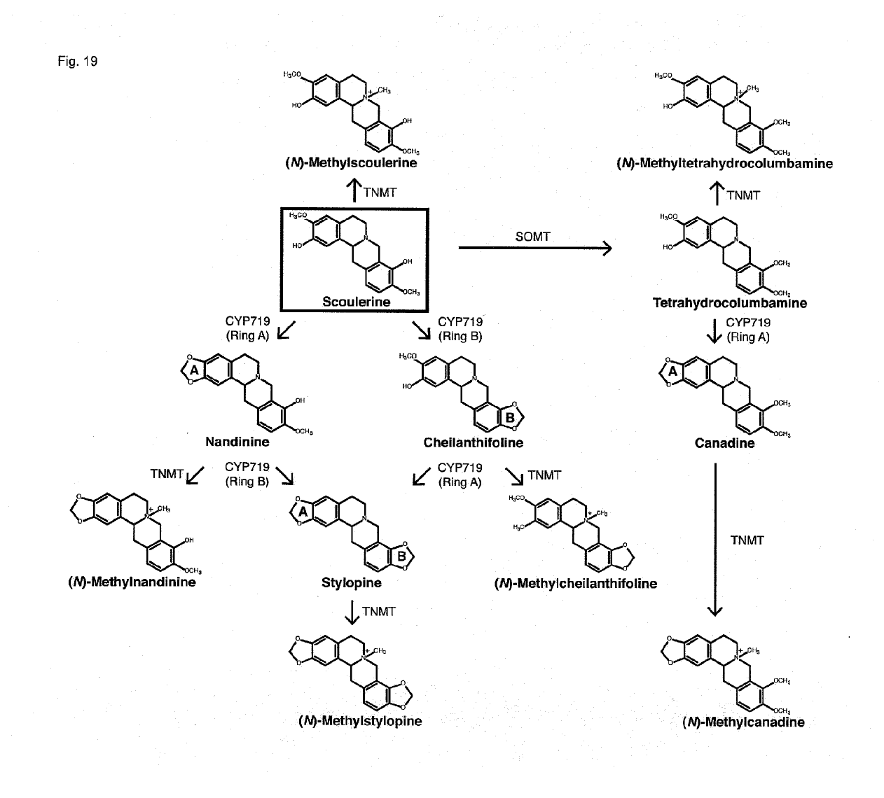

To broadly identify CYP719 activities on BIAs, a substrate affinity test was performed with the protoberberine BIA scoulerine. CYP719s have been described to form methylenedioxy bridges on BIAs from an alcohol group and a methyl group on adjacent carbons. Two different rings can be made on scoulerine, which can be called “Ring A” and “Ring B”, with the BIA products being called “nandinine” and “cheilanthifoline”, respectively (see FIG. 19). CYP719-catalyzed formation of both Ring A and Ring B of scoulerine (“Ring A-closing activity” and “Ring B-closing activity”) has been identified and published. Certain CYP719 enzymes included in the affinity tests have published ring A and Ring B activities. See Table 8 below.

Plasmids harboring CYP719s were transformed into either GC1333 containing an integrated PsCPR (FIG. 10A) or GC1316 containing an integrated PsCPR and an integrated PsCFS (FIG. 10B). Strain GC1333, harboring PsCPR integrated into the chromosome, was transformed with pBOT plasmids, with GFP removed, harboring individual CYP719s (CYP719 EX41-105). (See Tables 7-8 below presenting the nature and activity of these CYP719s).

TABLE 7
List of CYP719s identified herein

| | | Source | | | |
|---|---|---|---|---|---|
| | | | Available online | | |
| Name | Species | PhytoMetaSyn ™ | Genbank | Accession | CYP name |
| EX41 | Argemone mexicana | | Y | B1NF20.1 | CYP719A14 |
| EX42 | Argemone mexicana | | Y | B1NF19.1 | CYP719A13 |
| EX43 | Aquilegia formosa | | N | http://drnelson.uthsc.edu/biblioD.html | CYP719A6 |
| EX44 | Aquilegia formosa | | N | http://drnelson.uthsc.edu/biblioD.html | CYP719A7 |
| EX45 | Corydalis cheilanthifolia | Y | | | |
| EX46 | Corydalis cheilanthifolia | Y | | | |
| EX47 | Corydalis cheilanthifolia | Y | | | |
| EX48 | Corydalis cheilanthifolia | Y | | | |
| EX49 | Coptis chinensis | | Y | AGL76711.1 | n/a |
| EX50 | Coptis japonica | | Y | Q948Y1.1 | CYP719A1 |
| EX51 | Coptis japonica | | Y | BAF98470.1 | CYP719A18 |
| EX52 | Coptis japonica | | Y | BAF98471.1 | CYP719A19 |
| EX53 | Chelidonium majus | Y | | | |
| EX54 | Chelidonium majus | Y | | | |
| EX55 | Chelidonium majus | Y | | | |
| EX56 | Chelidonium majus | Y | | | |
| EX57 | Cissampelos mucronata | Y | | | |
| EX58 | Cissampelos mucronata | Y | | | |
| EX59 | Eschscholzia californica | | Y | B5UAQ8.1 | CYP719A5 |
| EX60 | Eschscholzia californica | | Y | BAG75114.1 | CYP719A9 |
| EX61 | Eschscholzia californica | | Y | Q50LH3.1 | CYP719A2 |
| EX62 | Eschscholzia californica | | Y | Q50LH4.1 | CYP719A3 |
| EX63 | Eschscholzia californica | | Y | BAG75115.1 | CYP719A11 |
| EX64 | Eschscholzia californica | | Y | BAG75116.1 | CYP719A17 |
| EX65 | Glaucium flavum | Y | | | |
| EX66 | Glaucium flavum | Y | | | |
| EX67 | Glaucium flavum | Y | | | |
| EX68 | Hydrastis canadensis | Y | | | |
| EX69 | Mahonia aquifolium | Y | | | |
| EX70 | Menispermum canadense | Y | | | |
| EX71 | Nandina domestica | Y | | | |
| EX72 | Nandina domestica | Y | | | |
| EX73 | Nandina domestica | Y | | | |
| EX74 | Nandina domestica | Y | | | |
| EX75 | Nandina domestica | Y | | | |
| EX76 | Nandina domestica | Y | | | |
| EX77 | Nelumbo nucifera | | | XP_010267084 | CYP719A22 |
| EX78 | Papaver bracteatum | Y | | | |
| EX79 | Papaver bracteatum | Y | | | |
| EX80 | Papaver bracteatum | Y | | | |
| EX81 | Podophyllum peltatum | | Y | AGC29954.1 | CYP719A24 |
| EX82 | Papaver somniferum | Y | | | |
| EX83 | Papaver somniferum | | Y | AFB74615.1 | CYP719A21 |
| EX84** | Papaver somniferum | | Y | ADB89213.1 | PsCFS |
| EX85 | Papaver somniferum | | Y | B1NF18.1 | CYP719B1 |
| EX86 | Papaver somniferum | Y | | | |
| EX87 | Papaver somniferum | Y | | | |
| EX88 | Papaver somniferum | Y | | | |
| EX89 | Papaver somniferum | Y | | | |
| EX90 | Papaver somniferum | Y | | | |
| EX91 | Papaver somniferum | Y | | | |
| EX92 | Papaver somniferum | Y | | | |
| EX93 | Papaver somniferum | Y | | | |
| EX94 | Papaver somniferum | | Y | Derived from AHF65153.1 | PsSPSΔN |
| EX95 | Sanguinaria canadensis | Y | | | |
| EX96 | Sanguinaria canadensis | Y | | | |
| EX97 | Sanguinaria canadensis | Y | | | |
| EX98 | Stylophorum diphyllum | Y | | | |
| EX99 | Stylophorum diphyllum | Y | | | |
| EX100 | Stylophorum diphyllum | Y | | | |
| EX101 | Stylophorum diphyllum | Y | | | |
| EX102 | Sinopodophyllum hexandrum | | Y | AGC29953.1 | CYP719A23 |
| EX103 | Thalictrum flavum | | Y | AAU20771.1 | CYP719A4 |
| EX104 | Thalictrum flavum | Y | | | |
| EX105 | Xanthorhiza simplicissima | Y | | | |

TABLE 8
Activity of CYP719s

| | | | Activity: Ring A | | | | | | |
|---|---|---|---|---|---|---|---|---|---|
| | | | | | Tetrahydro- | Activity: Ring B | | | |
| Published CYP719s | | | Scoulerine | Cheilanthifoline | columbamine | Scoulerine | Nandinine | | |
| | | Published | to | to | to | to | to | | Citations |
| Name | Species | activities | nandinine | stylopine | canadine | cheilanthifoline | stylopine | Activity: Other | (activity) |
| EX41 | Argemone | Y | | | | Y | | | Diaz Chavez et |
| | mexicana | | | | | | | | al 2011²⁸ |
| EX42 | Argemone | Y | Y | Y | Y | | | Coreximine to | Diaz Chavez |
| | mexicana | | | | | | | coreximine | et al 2011²⁸ |
| | | | | | | | | product | |
| EX43 | Aquilegia | N | | | | | | | unpublished |
| | formosa | | | | | | | | |
| EX44 | Aquilegia | N | | | | | | | unpublished |
| | formosa | | | | | | | | |
| EX49 | Coptis chinensis | N | | | | | | | unpublished |
| EX50 | Coptis japonica | Y | | | Y | N | | | Ikezawa 2003⁷³ |
| EX51 | Coptis japonica | N | | | | | | | unpublished |
| EX52 | Coptis japonica | N | | | | | | | unpublished |
| EX59 | Eschscholzia | Y | | | | Y | | | Ikezawa 2009⁶⁶ |
| | californica | | | | | | | | |
| EX60 | Eschscholzia | Y | | | | | | (S)-Reticuline | Ikezawa 2009⁶⁶ |
| | californica | | | | | | | to reticuline | |
| | | | | | | | | | product |
| EX61 | Eschscholzia | Y | Y | Y | N | N | | | Ikezawa 2007³⁹ |
| | californica | | | | | | | | |
| EX62 | Eschscholzia | Y | Y | Y | Y | N | | | Ikezawa 2007³⁹ |
| | californica | | | | | | | | |
| EX63 | Eschscholzia | N | | | | | | | Ikezawa 2009⁶⁶ |
| | californica | | | | | | | | |
| EX64 | Eschscholzia | N | | | | | | | Ikezawa 2009⁶⁶ |
| | californica | | | | | | | | |
| EX77 | Nelumbo nucifera | N | | | | | | | Nelson 2013 |
| EX81 | Podophyllum | Y | | | | | | Matairesinol to | Marques |
| | peltatum | | | | | | | pluviatolide | 2013⁶⁷ |
| EX83 | Papaver | Y | | | Y | | | | Dang 2014⁶⁸ |
| | somniferum | | | | | | | | |
| EX84 | Papaver | Y | | | | Y | | | Fossati 2014⁷⁴ |
| | somniferum | | | | | | | | |
| EX85 | Papaver | Y | | | | | | (R)-Reticuline | Gesell 2009⁶⁹ |
| | somniferum | | | | | | | to salutaridine | |
| EX94 | Papaver | Y | | Y | | | | | Fossati 2014⁷⁴ |
| | somniferum | | | | | | | | |
| EX102 | Sinopodophyllum | Y | | | | | | Matairesinol to | Marques |
| | hexandrum | | | | | | | pluviatolide | 2013⁶⁷ |
| EX103 | Thalictrum | N | | | | | | | Samanani 2005 |
| | flavum | | | | | | | | |

The resulting CYP719-harboring strains (strains SF41-105) were supplemented with scoulerine. After 16 hours, BIAs were extracted and the molar ratio of scoulerine to downstream BIAs was calculated (FIG. 10A).

10 of the 61 assayed CYP719s were observed to convert scoulerine into cheilanthifoline (Ring B closers) (EX41, EX45, EX54, EX59, EX65, EX74, EX84, EX95, EX98 and EX99). All 10 converted over 95% of scoulerine to the Ring B product cheilanthifoline. 23 of the 61 assayed CYP719s converted at least 5% of supplemented scoulerine to the Ring A product nandinine. 10 of 23 converted over 95% of scoulerine to nandinine. This could indicate that while scoulerine was accepted, it was not a preferred substrate for the other 13 Ring A-closing CYP719s.

The 10 CYP719s capable of Ring A closure of >95% of scoulerine (EX42, EX50, EX56, EX60, EX67, EX69, EX72, EX76, EX96 and EX101), along with several other purchased CYP719s with a range of Ring A-closing activity on scoulerine from 0% to 89% (EX44, EX46, EX47, EX48, EX58, EX61, EX66, EX103 and EX105) were introduced to the Block 2 pathway and tested for affinity for cheilanthifoline. Plasmids harboring individual CYP719s were transformed into strain GC1316, which harbored PsCPR and PsCFS integrated into the genome. Strains were supplemented with scoulerine and after 16 hours total BIAs were extracted and the molar ratio was compared (FIG. 10B). The four possible BIAs that could be extracted were scoulerine, the single Ring A product nandinine, the single Ring B product cheilanthifoline, and the double Ring A, Ring B product stylopine. Scoulerine was not observed, because PsCFS alone converted 100% of scoulerine to cheilanthifoline. >98% conversion of scoulerine to stylopine was observed in strains expressing PsCFS and either EX46 or EX61. The rest of the samples displayed a range of ratios of nandinine:cheilanthifoline:stylopine. Residual nandinine in many of the samples, especially that of the strain expressing PsCFS and EX60, indicated that PsCFS had little to no activity on nandinine.

Because nandinine was not a preferred substrate of PsCFS, most stylopine was generated from cheilanthifoline. Therefore, the ratio of nandinine:cheilanthifoline:stylopine was affected by two factors. The first factor was the acceptance of cheilanthifoline by Ring A-closing CYP719s. Of the 10 CYP719s capable of 95% Ring A closure of scoulerine, residual cheilanthifoline was detected in three (EX50, EX60, EX72). Conversely, EX46 was capable of just 66% Ring A closure of scoulerine (see FIG. 10A) and EX61 was capable of 90% ring closure of scoulerine (FIG. 10A), but when combined with PsCFS, both combinations yielded >98% conversion to stylopine. Using scoulerine as an initial screen for Ring A-closing activity had moderate predictive success.

The second factor affecting the ratio of nandinine:cheilanthifoline:stylopine was the relative rate of activity of Ring A and Ring B closure. Scoulerine was a substrate for both Ring A closure and Ring B closure. If Ring A closure occurred at a greater rate than Ring B closure, nandinine was produced, which accumulated. If Ring B closure occurred at a greater rate than Ring A closure, cheilanthifoline was produced, which either accumulated or was converted to stylopine depending on the specificity of the Ring A-closing CYP719. Ring B closure has previously been observed to occur at a higher rate than Ring A closure in the CYP719s of Argemone mexicana (EX41 vs. EX42)²⁸. It is this difference in Ring A and Ring B closure rates that results in cheilanthifoline accumulation in vivo, which is then a substrate for TNMT to generate the undesirable side product N-methylcheilanthifoline.

To optimize the turnover of scoulerine to stylopine, there were two options considered. First, a Ring A-closing CYP719 that did not synthesize nandinine could be identified, avoiding the nandinine side-product. However, most CYP719s predicted to close Ring A of various protoberberines were able to accept scoulerine, limiting the number of Ring A-closers available to compare relative rates of activity in vivo. Alternatively, a Ring B-closing CYP719 could be identified which could accept both scoulerine and nandinine. Consequently, the Ring A product nandinine would no longer be a dead-end but an intermediate. As a result, any CYP719 capable of closing Ring A on scoulerine and/or cheilanthifoline would be a potential candidate for pathway optimization.

The 10 CYP719s with 95% activity on Ring A of scoulerine and the 10 CYP719s with activity on Ring B of scoulerine were tested for activity on cheilanthifoline and nandinine, respectively, in order to generate a branched stylopine synthesis pathway. As the Applicant did not have pure cheilanthifoline or nandinine, it generated these compounds through in vivo conversion of scoulerine. CYP719s PsCFS and EX101, capable of converting >98% of scoulerine to cheilanthifoline and nandinine, respectively, were incubated with scoulerine. After 16 h, cells were pelleted and the supernatant fraction was collected. The supernatant was then applied to fresh yeast strains in order to supplement them with either TE containing nandinine or cheilanthifoline as necessary.

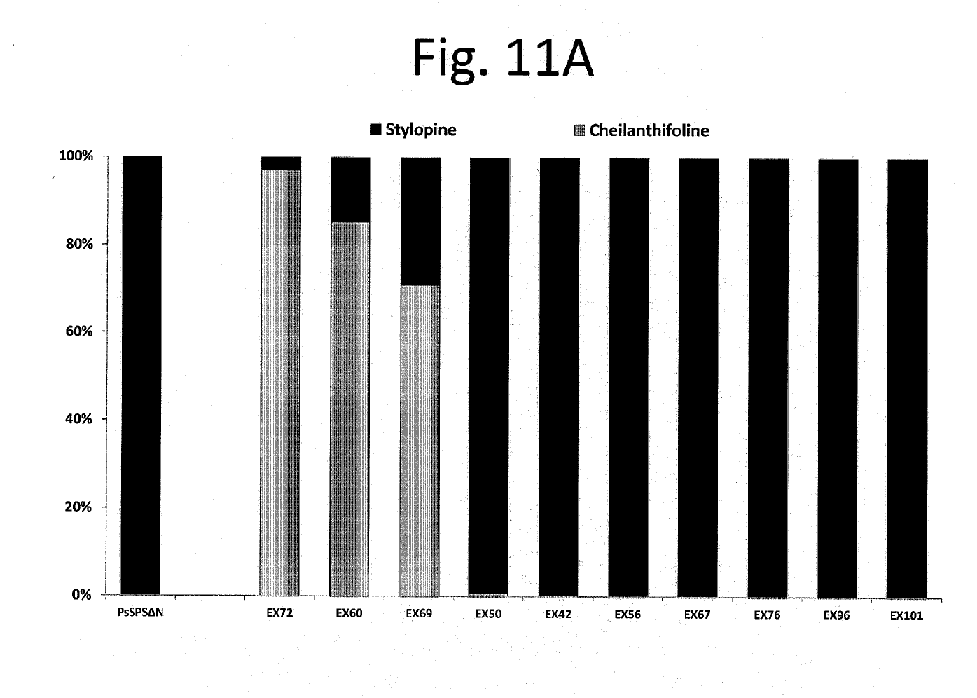

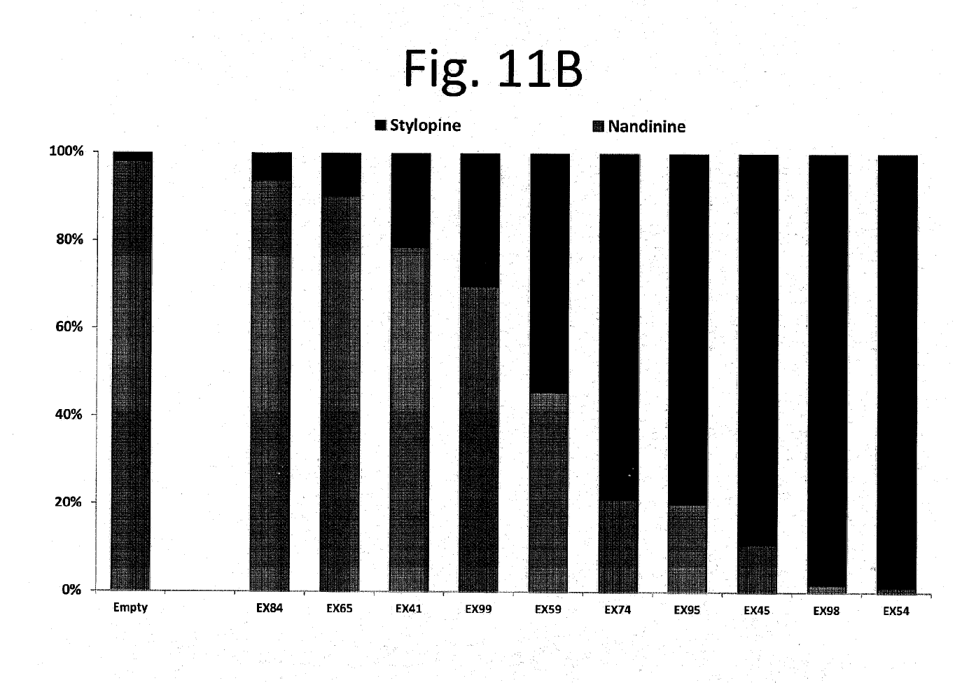

7 of 10 CYP719s with >95% activity on Ring A of scoulerine converted >98% of cheilanthifoline to stylopine: EX42, EX50, EX56, EX67, EX76, EX96, EX101 (FIG. 11A). In addition, PsSPSΔN also converted >98% of cheilanthifoline to stylopine. 2 of 10 CYP719s with >98% activity on Ring B of scoulerine converted Ring B-closers converted >98% of nandinine to stylopine: EX54 and EX98 (FIG. 11B). In comparison, strain GC1316 with PsCFS and CPR integrated into the genome converted 12% of nandinine to stylopine (FIG. 11B). The 7 CYP719s with activity on Ring A, and 2 CYP719s with activity on Ring B were chosen for combinatorial tests in the presence of TNMT to determine relative rates of activity in vivo. Table 9 below show CYP719 Ring A-/Ring B closing activities disclosed in FIGS. 10A-B and 11A and B.

TABLE 9
illustrative purchased CYP719 Ring A-/Ring B closing
activities disclosed in FIGS. 10A-B and 11A and B.

| | | Scoulerine | Cheilanthifoline | Scoulerine to | Nandinine |
|---|---|---|---|---|---|
| Name | Species | to nandinine | to stylopine | cheilanthifoline | to stylopine |
| EX41 | Argemone mexicana | | | X | |
| EX42 | Argemone mexicana | X | X | | |
| EX43 | Aquilegia formosa | X | | | |
| EX44 | Aquilegia formosa | X | | | |
| EX45 | Corydalis cheilanthifolia | | | X | |
| EX46 | Corydalis cheilanthifolia | X | X | | |
| EX47 | Corydalis cheilanthifolia | X | | | |
| EX48 | Corydalis cheilanthifolia | X | X | | |
| EX49 | Coptis chinensis | | | | |
| EX50 | Coptis japonica | X | X | | |
| EX51 | Coptis japonica | | | | |
| EX52 | Coptis japonica | | | | |
| EX53 | Chelidonium majus | | | | |
| EX54 | Chelidonium majus | | | X | X |
| EX55 | Chelidonium majus | X | | | |
| EX56 | Chelidonium majus | X | X | | |
| EX57 | Cissampelos mucronata | | | | |
| EX58 | Cissampelos mucronata | | | | |
| EX59 | Eschscholzia californica | | | X | |
| EX60 | Eschscholzia californica | X | | | |
| EX61 | Eschscholzia californica | X | X | | |
| EX62 | Eschscholzia californica | | | | |
| EX63 | Eschscholzia californica | | | | |
| EX64 | Eschscholzia californica | | | | |
| EX65 | Glaucium flavum | | | X | |
| EX66 | Glaucium flavum | X | | | |
| EX67 | Glaucium flavum | X | X | | |
| EX68 | Hydrastis canadensis | X | | | |
| EX69 | Mahonia aquifolium | X | | | |
| EX70 | Menispermum canadense | | | | |
| EX71 | Nandina domestica | | | | |
| EX72 | Nandina domestica | X | | | |
| EX73 | Nandina domestica | | | | |
| EX74 | Nandina domestica | | | X | |
| EX75 | Nandina domestica | | | | |
| EX76 | Nandina domestica | X | X | | |
| EX77 | Nelumbo nucifera | | | | |
| EX78 | Papaver bracteatum | | | | |
| EX79 | Papaver bracteatum | | | | |
| EX80 | Papaver bracteatum | X | | | |
| EX81 | Podophyllum peltatum | | | | |
| EX82 | Papaver somniferum | | | | |
| EX83 | Papaver somniferum | | | | |
| EX84** | Papaver somniferum | | | X | |
| EX85 | Papaver somniferum | | | | |
| EX86 | Papaver somniferum | | | | |
| EX87 | Papaver somniferum | | | | |
| EX88 | Papaver somniferum | | | | |
| EX89 | Papaver somniferum | | | | |
| EX90 | Papaver somniferum | | | | |
| EX91 | Papaver somniferum | | | | |
| EX92 | Papaver somniferum | | | | |
| EX93 | Papaver somniferum | | | | |
| EX94* | Papaver somniferum | | | | |
| EX95 | Sanguinaria canadensis | | | X | |
| EX96 | Sanguinaria canadensis | X | X | | |
| EX97 | Sanguinaria canadensis | X | | | |
| EX98 | Stylophorum diphyllum | | | X | X |
| EX99 | Stylophorum diphyllum | | | X | |
| EX100 | Stylophorum diphyllum | | | | |
| EX101 | Stylophorum diphyllum | X | X | | |
| EX102 | Sinopodophyllum hexandrum | | | | |
| EX103 | Thalictrum flavum | X | | | |
| EX104 | Thalictrum flavum | | | | |
| EX105 | Xanthorhiza simplicissima | X | | | |

All CYP719s having been ordered pre-cloned into the same expression vector: pBOT-TRP, plasmids harboring CYP719s could not be co-transformed until some enzymes were expressed from a different auxotrophies. The pBOT expression cassettes were designed to be excisable via the restriction enzymes AscI and NotI. Therefore, pBOT-LEU was digested with AscI and NotI, and the expression cassettes of P1E6 and P2A2 were liberated from pBOT-TRP backbones via AscI and NotI digestion. Religation and transformation resulted in P1E6 and P2A2 expressed from pBOT-LEU. As a result, all CYP719s were ready for combinatorial testing.

CYP719s capable of closing Ring A and Ring B were co-transformed into either GC1333, harbouring PsCPR integrated into the genome (FIG. 12A), or GC1270, harbouring PsCPR and TNMT integrated into the genome (FIG. 12B). Conversion of scoulerine to stylopine could be compared for each combination, as well as competition for substrates between CYP719 and TNMT. All strains were incubated for 16 h with scoulerine, and then total BIAs were extracted and ratios were compared.

When expressed individually, all Ring A-closing CYP719s converted >98% of scoulerine to nandinine, and all Ring B-closing CYP719s converted >98% of scoulerine to cheilanthifoline (FIG. 12A). When expressed in combination, all combinations of Ring A- and Ring B-closing CYP719s resulted in >98% stylopine (FIG. 12A).

When individual CYP719s with Ring B-closing activity were expressed in combination with TNMT, >98% of scoulerine was converted to N-methylcheilanthifoline (FIG. 12B). This indicates that the rate of CYP719-catalyzed methylenedioxy bridge formation was quicker than the rate of TNMT-catalyzed methylation of scoulerine (FIG. 12B). When individual CYP719s with Ring A-closing activity were expressed in combination with TNMT, a range of ratios of N-methylnandinine: N-methylscoulerine was observed, from 1% to 12% N-methylnandinine (FIG. 12B). When combinations of CYP719s with both Ring A and Ring B-closing activity were expressed in the presence of TNMT, a range of ratios of N-methylcheilanthifoline: N-methylstylopine were observed (FIG. 12B). Combinations of CYP719s with both Ring A and Ring B-closing activity in the presence of TNMT resulted in a range of ratios of N-methylstylopine: N-methylcheilanthifoline. In the presence of TNMT, 4 CYP719s with Ring A-closing activity, expressed in combination with either CYP719 with Ring B-closing activity, were capable of converting >98% of supplemented scoulerine to N-methylstylopine (FIG. 12B) (namely the combination of either EX54 or EX98 with either of EX67, EX76, EX96 or EX101).

The side-product N-methylcheilanthifoline was not observed (<2% of extracted BIAs) when scoulerine was supplemented to yeast expressing various combinations of CYP719s with Ring A-closing and Ring B-closing activity in the presence of TNMT. Several combinations of CYP719s (e.g., EX54 and EX98 against EX42, EX50, EX67, EX76 and EX101) are expressed in the presence of Block 3 (TNMT, MSH, P6H) and supplemented with scoulerine in order to observe downstream products in the presence of a larger number of heterologous enzymes. Yields are expected to increase when these genes are combined with the rest of the sanguinarine pathway as described herein.

The screens of purchased CYP719s herein have been focused on the synthesis of N-methylstylopine for the purpose of optimization of dihydrosanguinarine yields. In the process, combinations of TNMT and purchased CYP719s were used to efficiently generate a variety of N-methylated and unmethylated protoberberines: cheilanthifoline, nandinine, stylopine, N-methylscoulerine, N-methylnandinine, N-methylcheilanthifoline, and N-methylstylopine.

Other activities of CYP719s on protoberberines have previously been published, such as the Ring A closure of scoulerine-derived tetrahydrocolumbamine to produce canadine. Scoulerine is methylated by scoulerine O-methyl transferase (SOMT) to generate tetrahydrocolumbamin⁷². CYP719-catalyzed Ring A-closing of tetrahydrocolumbamine produces canadine, which can be methylated by TNMT to generate N-methylcanadine, a precursor to noscapine. The presence of both a Ring A-closing CYP719 and TNMT in this pathway will also require CYP719 optimization for efficient yields of noscapine in a microbial host.

### Example 11

### Generation Fo (R)-Reticuline

The major route for the synthesis of (R)-reticuline in P. somniferum is considered to be epimerization from (S)-reticuline, which was proposed to proceed via dehydrogenation of (S)-reticuline to 1,2-dehydroreticuline and subsequent enantioselective reduction to (R)-reticuline. However, the genes encoding these enzymes have never been cloned and those reaction never fully characterized⁶⁰⁻⁶¹. It should however be noted that (R)-reticuline is not the only (R)-BIA intermediate found in Ranunculales⁶². This suggests the possibility of an alternative pathway for the synthesis of (R)-intermediates, possibly the existence of enzymes selective for the (R)-enantiomers from the very beginning of the reticuline synthesis pathway. For example, both (S)- and (R)—N-methylcoclaurine were isolated in Berberis stolonifera. These two enantiomers of N-methylcoclaurine are required by the cytochrome P450 berbamunine synthase for the synthesis of berbamunine in Berberis stolonifera⁶².

While P. somniferum does not make (R,S)-norlaudanosoline, results presented herein indicate that only (S)-reticuline is produced from racemic norlaudanosoline using opium poppy's native methyltransferases. Some evidence for the enantioselectivity of MTs involved in BIA synthesis can be found in the literature. For example, a study reporting on the activity of Coptis japonica MTs for the production of reticuline from racemic norlaudanosoline in engineered E. coli reported a prevalent synthesis of (S)-reticuline over (R)-reticuline¹⁸. This data clearly indicated that some of the C. japonica MTs have a preference for the (S)-enantiomer with limited activity on the (R)-enantiomer. It is therefore possible that MTs strictly enantioselective for the (R)-enantiomer exist and the epimerization to the (R)-enantiomers happens upstream reticuline.

### Example 12

### Increasing the Activity of P450s

Cytochrome b5 has been reported to enhance activity of certain cytochrome P450s⁴⁸. Tuning expression of the four P450s, CPR and cognate cytochrome b5 could increase pathway efficiency. The impact of cytochrome b5 on yield is tested by expressing b5 in a plasmid or integrated in a chromosome in host cells expressing block(s) 1, 1-2, 2-3 or 1-2-3.

### Example 13

### Increasing the Yield of Block 2-Block 3

The high number of P450s expressed the cells may be affecting yields of dihydrosanguinarine. (S)-Scoulerine fed to Block 2-Block 3 integrant strains expressing four P450s yielded the same conversion to dihydrosanguinarine whether or not Block 1 and BBEΔN-2μ were expressed (7.5% vs. 7.7; FIG. 3). In contrast, conversion of the fed substrate (S)-stylopine to dihydrosanguinarine dropped from 57% when Block 3 enzymes were expressed in isolation to 37% when Block 3 enzymes were co-expressed with Block 1, BBEΔN-2μ and integrated Block 2. Because Block 1 and BBEΔN-2μ do not appear to affect yields, the applicants hypothesize that it was the co-expression of Block 2 with Block 3 that was responsible for this decrease in yields. Further, because these two strains were fed stylopine, the promiscuity of TNMT is not responsible for this decrease. This suggests that the co-expression of the four P450s in Blocks 2 and 3 in GCY1125 is not optimal for pathway efficiency.

### Example 14

### Increasing Specificity of TNMT

Synthesis of the side products N-methylcheilanthifoline and N-methylscoulerine by TNMT was shown to be a major limiting factor in the reconstituted pathway. Promiscuity is a common theme among enzymes involved in plant specialized metabolism and is one of the factors contributing to the great chemodiversity of plant secondary metabolites⁴¹. While broad substrate specificity of PsTNMT had been previously described³⁴, the applicants present the first experimental evidence of its acceptance of scoulerine and cheilanthifoline as substrates. The present invention encompasses the use of orthologous plant TNMT enzymes with narrower substrate specificity, enzyme engineering⁴², mutagenesis, substrate channeling and/or spatio-temporal sequestration of the reactions^{43,44}. TNMT orthologues as shown in FIGS. 14G and 15G are tested.

### Example 15

### Generating Quaternary Benzylisoquinoline Alkaloids

While N-methylscoulerine and N-methylcheilanthifoline are undesirable side-products, they may themselves be end products of interest. Both compounds are quaternary benzylisoquinoline alkaloids like sanguinarine and berberine. N-methylscoulerine (cyclanoline) can be extracted from several plants of the genus Stephania and has been described as an acetylcholinesterase inhibitor⁴⁵, but to the best of the applicants' knowledge N-methylcheilanthifoline has never been detected in plants. The promiscuity of TNMT could also be further explored to generate other quaternary benzylisoquinoline alkaloids. Synthesis of N-methylcheilanthifoline, although serendipitous, highlights the potential of combinatorial biology in S. cerevisiae, through which libraries of alkaloids can be generated independent of their abundance in nature.

### Example 16

### Bypassing the Need to Feed Norlaudanosoline

Synthesis of (S)-reticuline from glucose and glycerol has been reported in E. coli^{18,19} but not in S. cerevisiae. Thus, supplemented (R,S)-norlaudanosoline was provided to measure the efficiency of the reconstituted BIA pathway. The applicants observed that just 10% of fed norlaudanosoline was detected in the cell extract after 16 hours of incubation with the negative control yeast cells (FIG. 6A). While the applicants cannot directly compare substrates with different chemical properties and possibly different mechanisms of transport into yeast, they suspect that limited availability of norlaudanosoline to the intracellular enzymes is limiting pathway efficiency. In a previous study, it was shown that reticuline accumulation increased with (R,S)-norlaudanosoline concentration in cell feeding assays²², further supporting the evidence that low intracellular norlaudanosoline concentration is limiting flux. Linking the reconstituted alkaloid pathway to the microbe's central metabolism, thereby bypassing the need to feed norlaudanosoline, will likely boost yields of reticuline and thus the entire dihydrosanguinarine pathway. To bypass the need to feed norlaudanosoline, norcoclaurine (FIG. 1) or norlaudanosoline may be produced in yeast from the precursor tyrosine. Yeast is engineered for an increased tyrosine production and the enzymes for the synthesis of norcoclaurine (or norlaudanosoline) from tyrosine is heterologously expressed (FIG. 1).

### Example 17

### Role of CYP719 and TNMT in the Noscapine Pathway

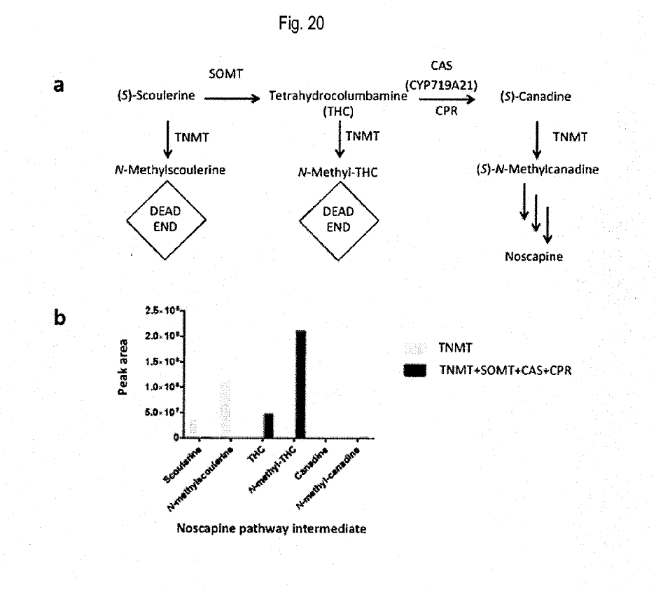

Other activities of CYP719s on protoberberines have previously been published, such as the Ring A closure of scoulerine-derived tetrahydrocolumbamine (THC) to produce canadine. In particular, CYP719s catalyzing the Ring A closure of THC to produce canadine have been described in P. somniferum, C. japonica, A. mexicana, and E. californica⁶⁸. Scoulerine is methylated by scoulerine-O-methyltransferase (SOMT) to generate tetrahydrocolumbamine. CYP719A13 from Argemone mexicana was shown to catalyze the Ring A closure of both cheilanthifoline and tetrahydrocolumbamine²⁸ strongly indicating that the CYP719 library in FIG. 10C also includes tetrahydrocolumbamine Ring A closers. Canadine can be methylated by TNMT to generate N-methylcanadine, a precursor to the cough suppressant and potential anticancer drug noscapine¹⁷. However, TNMT can also N-methylate scoulerine and tetrahydrocolumbamine, generating the undesired side products N-methyl-scoulerine and N-methyltetrahydrocolumbamine (FIG. 20A). For example, when P. somniferum SOMT, CYP719A21 (canadine synthase, CAS; and ref. 68) and TNMT are co-expressed in S. cerevisiae supplemented with scoulerine, the major products accumulating are tetrahydrocolumbamine and N-methyltetrahydrocolumbamine, with only trace accumulation of N-methylcanadine and trace residual scoulerine (FIG. 20B). N-methylscoulerine and canadine were not detected, indicating that PsSOMT converts scoulerine to THC before TNMT can N-methylated it, but then TNMT N-methylates most of THC to the side product N-methyl-THC before PsCAS can convert it to canadine. The presence of both a Ring A-closing CYP719 and TNMT in this pathway will therefore require CYP719 optimization, using candidates from the CYP719 library, for efficient production of N-methylcanadine and ultimately noscapine in a microbial host. The present invention encompasses improving yield of N-methylcanadine comprising using of a THC Ring A closer with a higher affinity to THC than that of TNMT to THC, the Ring A closer having been identified in a method analogous to that used to identify scoulerine Ring a/Ring B closers.

The scope of the claims should not be limited by the preferred embodiments set forth in the examples, but should be given the broadest interpretation consistent with the description as a whole.

### REFERENCES

1 De Luca, V., Salim, V., Atsumi, S. M. & Yu, F. Mining the biodiversity of plants: a revolution in the making. Science 336, 1658-1661 (2012).
Newman, D. J. & Cragg, G. M. Natural products as sources of new drugs over the last 25 years. J. Nat. Prod. 70, 461-477 (2007).
Mishra, B. B. & Tiwari, V. K. Natural products: an evolving role in future drug discovery. Eur. J. Med. Chem. 46, 4769-4807 (2011).
Li, J. W. & Vederas, J. C. Drug discovery and natural products: end of an era or an endless frontier? Science 325, 161-165 (2009).
Giddings, L. A. & Newman, D. J. Microbial natural products: molecular blueprints for antitumor drugs. J. Ind. Microbiol. Biotechnol. doi: 10.1007/s10295-013-1331-1 (2013).
Xiao, M. et al. Transcriptome analysis based on next-generation sequencing of non-model plants producing specialized metabolites of biotechnological interest. J. Biotechnol. 166, 122-134 (2013).
Liu, L., Redden, H. & Alper, H. S. Frontiers of yeast metabolic engineering: diversifying beyond ethanol and Saccharomyces. Curr. Opin. Biotechnol. (2013).
Gibson, D. G. Synthesis of DNA fragments in yeast by one-step assembly of overlapping oligonucleotides. Nucleic Acids Res. 37, 6984-6990 (2009).
Shao, Z. & Zhao, H. DNA assembler, an in vivo genetic method for rapid construction of biochemical pathways. Nucleic Acids Res. 37, e16, doi: 10.1093/nar/gkn991 (2009).
10 Paddon, C. J. et al. High-level semi-synthetic production of the potent antimalarial artemisinin. Nature 496, 528-532 (2013).
11 Chougule, M., Patel, A. R., Sachdeva, P., Jackson, T. & Singh, M. Anticancer activity of noscapine, an opioid alkaloid in combination with cisplatin in human non-small cell lung cancer. Lung Cancer 71, 271-282 (2011).
12 Bessi, I. et al. Spectroscopic, molecular modeling, and NMR-spectroscopic investigation of the binding mode of the natural alkaloids berberine and sanguinarine to human telomeric G-quadruplex DNA. ACS Chem. Biol. 7, 1109-1119 (2012).
13 Rice, K. C. Synthetic opium-alkaloids and derivatives—a short total synthesis of (+/−)-dihydrothebainone, (+/−)-dihydrocodeinone, and (+/−)-nordihydrocodeinone as an approach to a practical synthesis of morphine, codeine, and congeners. J. Org. Chem. 45, 3135-3137 (1980).
14 Pickens, L. B., Tang, Y. & Chooi, Y. H. Metabolic engineering for the production of natural products. Annu. Rev. Chem. Biomol. Eng. 2, 211-236 (2011).
15 Newhouse, T., Baran, P. S. & Hoffmann, R. W. The economies of synthesis. Chem. Soc. Rev. 38, 3010-3021 (2009).
16 Sato, F., Inui, T. & Takemura, T. Metabolic engineering in isoquinoline alkaloid biosynthesis. Curr. Pharm. Biotechnol. 8, 211-218 (2007).
17 Hagel, J. M. & Facchini, P. J. Benzylisoquinoline alkaloid metabolism: a century of discovery and a brave new world. Plant Cell Physiol 54(5), 647-672 (2013).
18 Nakagawa, A. et al. A bacterial platform for fermentative production of plant alkaloids. Nat. Commun. 2, doi: 10.1038/Ncomms1327 (2011).
19 Nakagawa, A. et al. Bench-top fermentative production of plant benzylisoquinoline alkaloids using a bacterial platform. Bioeng. Bugs 3, 49-53 (2012).
20 Minami, H. et al. Microbial production of plant benzylisoquinoline alkaloids. Proc. Natl. Acad. Sci. U.S.A. 105, 7393-7398 (2008).
21 Siddiqui, M. S., Thodey, K., Trenchard, I. & Smolke, C. D. Advancing secondary metabolite biosynthesis in yeast with synthetic biology tools. FEMS Yeast Res. 12, 144-170 (2012).
22 Hawkins, K. M. & Smolke, C. D. Production of benzylisoquinoline alkaloids in Saccharomyces cerevisiae. Nat. Chem. Biol. 4, 564-573 (2008).
23 Godowski, K. C. Antimicrobial action of sanguinarine. J. Clin. Dent. 1, 96-101 (1989).
24 Obiang-Obounou, B. W. et al. The mechanism of action of sanguinarine against methicillin-resistant Staphylococcus aureus. J. Toxicol. Sci. 36, 277-283 (2011).
25 Beaudoin, G. A. & Facchini, P. J. Isolation and characterization of a cDNA encoding (S)-cis-N-methylstylopine 14-hydroxylase from opium poppy, a key enzyme in sanguinarine biosynthesis. Biochem. Biophys. Res. Commun. 431, 597-603 (2013).
26 Hagel, J. M. et al. Characterization of a flavoprotein oxidase from opium poppy catalyzing the final steps in sanguinarine and papaverine biosynthesis. J. Biol. Chem. 287, 42972-42983 (2012).
27 Takemura, T., Ikezawa, N., Iwasa, K. & Sato, F. Molecular cloning and characterization of a cytochrome P450 in sanguinarine biosynthesis from Eschscholzia californica cells. Phytochemistry 91, 100-108 (2013).
28 Diaz Chavez, M. L., Rolf, M., Gesell, A. & Kutchan, T. M. Characterization of two methylenedioxy bridge-forming cytochrome P450-dependent enzymes of alkaloid formation in the Mexican prickly poppy Argemone mexicana. Arch. Biochem. Biophys. 507, 186-193 (2011).
29 Facchini, P. J. & De Luca, V. Phloem-specific expression of tyrosine/dopa decarboxylase genes and the biosynthesis of isoquinoline alkaloids in opium poppy. Plant Cell 7, 1811-1821 (1995).
30 Stipanovic, R. D., Howell, C. R. & Bell, A. A. Sanguinarine: a simplified method of isolation. J. Heterocyclic Chem. 9, 1453-1454 (1972).
31 Ounaroon, A., Decker, G., Schmidt, J., Lottspeich, F. & Kutchan, T. M. (R,S)-Reticuline 7-O-methyltransferase and (R,S)-norcoclaurine 6-O-methyltransferase of Papaver somniferum—cDNA cloning and characterization of methyl transfer enzymes of alkaloid biosynthesis in opium poppy. Plant J 36, 808-819 (2003).
32 Ziegler, J., Diaz-Chavez, M. L., Kramell, R., Ammer, C. & Kutchan, T. M. Comparative macroarray analysis of morphine containing Papaver somniferum and eight morphine free Papaver species identifies an O-methyltransferase involved in benzylisoquinoline biosynthesis. Planta 222, 458-471 (2005).
33 Dittrich, H. & Kutchan, T. M. Molecular cloning, expression, and induction of berberine bridge enzyme, an enzyme essential to the formation of benzophenanthridine alkaloids in the response of plants to pathogenic attack. Proc. Natl. Acad. Sci. U.S.A. 88, 9969-9973 (1991).
34 Liscombe, D. K. & Facchini, P. J. Molecular cloning and characterization of tetrahydroprotoberberine cis-N-methyltransferase, an enzyme involved in alkaloid biosynthesis in opium poppy. J. Biol. Chem. 282, 14741-14751 (2007).
35 Flagfeldt, D. B., Siewers, V., Huang, L. & Nielsen, J. Characterization of chromosomal integration sites for heterologous gene expression in Saccharomyces cerevisiae. Yeast 26, 545-551 (2009).
36 Farrow, S. C., Hagel, J. M. & Facchini, P. J. Transcript and metabolite profiling in cell cultures of 18 plant species that produce benzylisoquinoline alkaloids. Phytochemistry 77, 79-88 (2012).
37 Facchini, P. J., Penzes, C., Johnson, A. G. & Bull, D. Molecular characterization of berberine bridge enzyme genes from opium poppy. Plant Physiol. 112, 1669-1677 (1996).
38 Pompon, D., Louerat, B., Bronine, A. & Urban, P. Yeast expression of animal and plant P450s in optimized redox environments. Methods Enzymol. 272, 51-64 (1996).
39 Ikezawa, N., Iwasa, K. & Sato, F. Molecular cloning and characterization of methylenedioxy bridge-forming enzymes involved in stylopine biosynthesis in Eschscholzia californica. FEBS J. 274, 1019-1035 (2007).
40 Schenkman, J. B. & Jansson, I. The many roles of cytochrome b5. Pharmacol. Ther. 97, 139-152 (2003).
41 Weng, J. K., Philippe, R. N. & Noel, J. P. The rise of chemodiversity in plants. Science 336, 1667-1670 (2012).
42 Yoshikuni, Y., Ferrin, T. E. & Keasling, J. D. Designed divergent evolution of enzyme function. Nature 440, 1078-1082 (2006).
43 Avalos, J. L., Fink, G. R. & Stephanopoulos, G. Compartmentalization of metabolic pathways in yeast mitochondria improves the production of branched-chain alcohols. Nat. Biotechnol. 31, 335-341 (2013).
44 Farhi, M. et al. Harnessing yeast subcellular compartments for the production of plant terpenoids. Metab. Eng. 13, 474-481 (2011).
45 Ingkaninan, K., Phengpa, P., Yuenyongsawad, S. & Khorana, N. Acetylcholinesterase inhibitors from Stephania venosa tuber. J. Pharm. Pharmacol. 58, 695-700 (2006).
46 Facchini, P. J. et al. Synthetic biosystems for the production of high-value plant metabolites. Trends Biotechnol. 30, 127-131 (2012).
47 Engels, B., Dahm, P. & Jennewein, S. Metabolic engineering of taxadiene biosynthesis in yeast as a first step towards Taxol (Paclitaxel) production. Metab. Eng. 10, 201-206 (2008).
48 Mikkelsen, M. D. et al. Microbial production of indolylglucosinolate through engineering of a multi-gene pathway in a versatile yeast expression platform. Metab. Eng. 14, 104-111 (2012).
49 Szczebara, F. M. et al. Total biosynthesis of hydrocortisone from a simple carbon source in yeast. Nat. Biotechnol. 21, 143-149 (2003).
50 Schumacher, H. M. & Zenk, M. H. Partial-purification and characterization of dihydrobenzophenanthridine oxidase from Escholtzia californica cell-suspension cultures. Plant Cell Reports 7, 43-46 (1988).
51 Nguyen, D. T. et al. Biochemical conservation and evolution of germacrene A oxidase in asteraceae. J. Biol. Chem. 285, 16588-16598 (2010).
52 Barriuso, J. et al. Double oxidation of the cyclic nonaketide dihydromonacolin L to monacolin J by a single cytochrome P450 monooxygenase, LovA. J. Am. Chem. Soc. 133, 8078-8081 (2011).
53 Gietz, R. D. & Schiestl, R. H. High-efficiency yeast transformation using the LiAc/SS carrier DNA/PEG method. Nat. Protoc. 2, 31-34 (2007).
54 Bird, D. A. & Facchini, P. J. Berberine bridge enzyme, a key branch-point enzyme in benzylisoquinoline alkaloid biosynthesis, contains a vacuolar sorting determinant. Planta 213, 888-897 (2001).
55 Jansen, G., Wu, C., Schade, B., Thomas, D. Y. & Whiteway, M. Drag & Drop cloning in yeast. Gene 344, 43-51 (2005).
56 Clasquin, M. F., Melamud, E. & Rabinowitz, J. D. LC-MS data processing with MAVEN: a metabolomic analysis and visualization engine. Curr. Protoc. Bioinformatics. Chapter 14, Unit14.11, doi:10.1002/0471250953.bi1411s37 (2012).
57 Sikorski, R. S. & Hieter, P. A system of shuttle vectors and yeast host strains designed for efficient manipulation of DNA in Saccharomyces cerevisiae. Genetics 122, 19-27 (1989).
58 Ro, D. K. et al. Induction of multiple pleiotropic drug resistance genes in yeast engineered to produce an increased level of anti-malarial drug precursor, artemisinic acid. BMC Biotechnol. 8, 83, doi: 10.1186/1472-6750-8-83 (2008).
59 van Dijken, J. P. et al. An interlaboratory comparison of physiological and genetic properties of four Saccharomyces cerevisiae strains. Enzyme Microb. Technol. 26, 706-714 (2000).
60 Hirata K, Poeaknapo C, Schmidt J, Zenk M H (2004) 1,2-Dehydroreticuline synthase, the branch point enzyme opening the morphinan biosynthetic pathway. Phytochemistry 65: 1039-1046. 11, 12.
61 De-Eknamkul W, Zenk M H (1992) Purification and properties of 1,2-dehydroreticuline reductase from Papaver somniferum seedlings. Phytochemistry 31: 813-821.
62 Kraus P F, Kutchan T M (1995) Molecular cloning and heterologous expression of a cDNA encoding berbamunine synthase, a C—O phenol-coupling cytochrome P450 from the higher plant Berberis stolonifera. Proc Natl Acad Sci USA 92: 2071-2075.
63 Min, X. J., Butler, G., Storms, R. & Tsang, A. OrfPredictor: predicting protein-coding regions in EST-derived sequences. Nucleic Acids Res. 33, W677-80 (2005).
64 Nelson, D. R. Cytochrome P450 nomenclature, 2004. Methods Mol. Bio. 320, 1-10 (2006).
65 Tamura, K., Stecher G., Peterson D., Filipski, A. & Kumar, S. MEGA6: Molecular Evolutionary Genetics Analysis version 6.0. Mol. Biol. Evol. 30, 2725-2729 (2013).
66 Ikezawa, N., Iwasa, K., & Sato, F. CYP719A subfamily of cytochrome P450 oxygenases and isoquinoline alkaloid biosynthesis in Eschscholzia californica. Plant Cell Rep. 28, 123-133 (2009).
67 Marques, J. V. et al. Next generation sequencing in predicting gene function in podophyllotoxin biosynthesis. J. Biol. Chem. 288, 466-479 (2013).
68 Dang, T. T. & Facchini, P. J. Cloning and characterization of canadine synthase involved in noscapine biosynthesis in opium poppy. FEBS Lett. 588, 198-204 (2014).
69 Gesell, A. et al. CYP719B1 is salutaridine synthase, the C—C phenol-coupling enzyme of morphine biosynthesis in opium poppy. J. Biol. Chem. 284, 24432-24442 (2009).
70 Dang, T.-T. T., & Facchini, P. J. (2012). Characterization of three O-methyltransferases involved in noscapine biosynthesis in opium poppy. Plant Physiology, 159(2), 618-31. doi:10.1104/pp. 112.194886.
71 Kim, J. S. et al. Improvement of reticuline productivity from dopamine by using engineered Escherichia coli. Biosci. Biotechnol. Biochem. 77, 2166-2168 (2013).
72 Takeshita, N. et al. Molecular cloning and characterization of S-adenosyl-L-methionine:scoulerine-9-O-methyltransferase from cultured cells of Coptis japonica. Plant Cell Physiol. 36, 29-36 (1995).
73 Ikezawa, N. et al. Molecular cloning and characterization of CYP719, a methylenedioxy bridge-forming enzyme that belongs to a novel P450 family, from cultured Coptis japonica cells. J. Biol. Chem. 278, 38557-38565 (2003).
74 Fossati E., et al. Reconstitution of a 10-gene pathway for synthesis of the plant alkaloid dihydrosanguinarine in Saccharomyces cerevisiae. Nature Communications, 5 (3283), (2014)

1. A method of preparing a benzylisoquinoline alkaloid (BIA) metabolite comprising:
(a) culturing a host cell under conditions suitable for protein production, including a first fermentation at a pH of between about 7 and about 10, and, optionally followed by a second fermentation at a pH between about 3 and about 6, said host cell comprising:
a. a first heterologous coding sequence encoding a first enzyme involved in a metabolite pathway that converts (R,S)-norlaudanosoline into the metabolite;
b. a second heterologous coding sequence encoding a second enzyme involved in a metabolite pathway that converts (R,S)-norlaudanosoline into the metabolite; and
c. a third heterologous coding sequence encoding a third enzyme involved in a metabolite pathway that converts (R,S)-norlaudanosoline into the metabolite;
(b) adding a substrate that is (R,S)-norlaudanosoline, (R,S)-reticuline or (S)-stylopine, to the cell culture; and
(c) recovering the metabolite from the cell culture.

2. The method of claim 1, wherein the host cell is a yeast cell, preferably wherein the yeast is Saccharomyces, preferably the Saccharomyces is Saccharomyces cerevisiae.

3. (canceled)

4. (canceled)

5. The method of claim 1, wherein the substrate is (R,S)-norlaudanosoline and the metabolite is (S)-reticuline,
preferably wherein:
a. the first enzyme is 6-O-methyltransferase (6OMT);
b. the second enzyme is coclaurine N-methyltransferase (CNMT); and/or
c. the third enzyme is 4′-O-methyltransferase 2 (4′OMT2),
more preferably wherein:
a. the 6OMT is as set forth in any one of the sequences as depicted in FIG. 14A or 15A;
b. the CNMT is as set forth in any one of the sequences as depicted in FIG. 14B or 15B; and/or
c. the 4′OMT2 is as set forth in any one of the sequences as depicted in FIG. 14C or 15C,
even more preferably wherein:
a. 6OMT is from Papaver somniferum;
b. CNMT is from Papaver somniferum; and/or
c. 4′OMT2 is from Papaver somniferum,
more particularly wherein:
a. Ps6OMT is as set forth in SEQ ID NO: 34 (FIG. 13);
b. PsCNMT is as set forth in SEQ ID NO: 38 (FIG. 13); and/or
c. Ps4′OMT2 is as set forth in SEQ ID NO: 42 (FIG. 13).

6. (canceled)

7. (canceled)

8. (canceled)

9. (canceled)

10. The method of claim 5, wherein the cell further comprises a fourth heterologous coding sequence encoding a fourth enzyme involved in a metabolite pathway that converts (R,S)-norlaudanosoline into the metabolite, preferably wherein the metabolite is (S)-scoulerine.

11. (canceled)

12. The method of claim 10, wherein the fourth enzyme is berberine bridge enzyme (BBE), preferably wherein the BBE is as set forth in any one of the sequences as depicted in FIG. 14D or 15D, more preferably wherein BBE is from Papaver somniferum (Ps).

13. (canceled)

14. (canceled)

15. The method of claim 12, wherein the amino acid N-terminal membrane-spanning domain from PsBBE was truncated (PsBBEΔN), preferably wherein PsBBEΔN is as set forth in SEQ ID NO: 46 (FIG. 13).

16. (canceled)

17. The method of claim 10, wherein the cell further comprises a fifth heterologous coding sequence encoding a fifth enzyme involved in a metabolite pathway that converts (R,S)-norlaudanosoline into the metabolite, preferably wherein the metabolite is nandinine or (S)-cheilanthifoline.

18. (canceled)

19. The method of claim 17, wherein the fifth enzyme is a Ring B closer able to transform scoulerine into cheilanthifoline, preferably wherein the Ring B closer is as set forth in any one of the sequences depicted in FIG. 17A-C, more preferably wherein the Ring B closer is further able to transform nandinine into stylopine, particularly wherein the Ring B closer is as set forth in any one of the sequences depicted in FIG. 17B-C, even more particularly wherein the Ring B closer is as set forth in any one of the sequences depicted in FIG. 17C.

20. (canceled)

21. (canceled)

22. (canceled)

23. (canceled)

24. The method of claim 19, wherein the fifth enzyme is cheilanthifoline synthase (CFS), preferably wherein the CFS is as set forth in any one of the sequences as depicted in FIG. 14E or 15E, more preferably wherein CFS is from Papaver somniferum (Ps), more particularly wherein PsCFS is as set forth in FIG. 13 (SEQ ID NO: 50 or 52).

25. (canceled)

26. (canceled)

27. (canceled)

28. The method of claim 17, wherein the cell further comprises a sixth heterologous coding sequence encoding a sixth enzyme involved in a metabolite pathway that converts (R,S)-norlaudanosoline into the metabolite, preferably wherein the metabolite is (S)-stylopine.

29. (canceled)

30. The method of claim 28, wherein the sixth enzyme is a Ring A closer able to transform cheilanthifoline into (S)-stylopine, preferably wherein the Ring A closer is as set forth in any one of the sequences depicted in FIG. 17D-E, more particularly wherein Ring A closer is further able to transform scoulerine into nandinine, particularly wherein the Ring A closer is as set forth in any one of the sequences depicted in FIG. 17E.

31. (canceled)

32. (canceled)

33. (canceled)

34. The method of claim 30, wherein the Ring B closer is (i) as set forth in SEQ ID NO: 485 and the Ring A closer is as set forth in SEQ ID NO: 487; (or) as set forth in SEQ ID NO: 333; or SEQ ID NO: 377 and the Ring A closer is as set forth in SEQ ID NO: 321, SEQ ID NO: 335, SEQ ID NO: 346, SEQ ID NO: 355, SEQ ID NO: or SEQ ID NO: 380.

35. (canceled)

36. The method of claim 28, wherein the sixth enzyme is stylopine syntase (SPS), preferably wherein the SPS is as set forth in any one of the sequences as depicted in FIG. 14F or 15F, more preferably wherein SPS is from Papaver somniferum (Ps).

37. (canceled)

38. (canceled)

39. The method of claim 28, wherein the method comprises the second fermentation and wherein the cell further comprises a seventh heterologous coding sequence encoding a seventh enzyme involved in a metabolite pathway that converts (R,S)-norlaudanosoline into the metabolite, preferably wherein the metabolite is (S)—N-cis-methylstylopine.

40. (canceled)

41. The method of claim 39, wherein the seventh enzyme is tetrahydroprotoberberine cis-N-methyltransferase (TNMT), preferably wherein the TNMT is as set forth in any one of the sequences as depicted in FIG. 14G or 14G, more preferably wherein TNMT is from Papaver somniferum (Ps), particularly wherein PsTNMT is as set forth in SEQ ID NO: 58 (FIG. 13).

42. (canceled)

43. (canceled)

44. (canceled)

45. The method of claim 39, wherein the cell further comprises a eight heterologous coding sequence encoding a eight enzyme involved in a metabolite pathway that converts (R,S)-norlaudanosoline into the metabolite, preferably wherein the metabolite is protopine.

46. (canceled)

47. The method of claim 45, wherein the eighth enzyme is (S)-cis-N-methylstylopine 14-hydroxylase (MSH), preferably wherein the MSH is as set forth in any one of the sequences as depicted in FIG. 14H or 14H, more preferably wherein MSH is from Papaver somniferum (Ps).

48. (canceled)

49. (canceled)

50. The method of claim 45, wherein the cell further comprises a ninth heterologous coding sequence encoding a ninth enzyme involved in a metabolite pathway that converts (R,S)-norlaudanosoline into the metabolite, preferably wherein the metabolite is 6-hydroxyprotopine.

51. (canceled)

52. The method of claim 50, wherein the ninth enzyme is protopine 6-hydroxylase (P6H), preferably wherein the P6H is as set forth in any one of the sequences as depicted in FIG. 14I or 14I, more preferably wherein P6H is from Eschscholzia californica (Ec), particularly wherein EcP6H is as set forth in SEQ ID NO: 62 (FIG. 13).

53. (canceled)

54. (canceled)

55. (canceled)

56. The method of claim 50, wherein the cell further comprises a tenth heterologous coding sequence encoding a tenth enzyme involved in a metabolite pathway that converts (R,S)-norlaudanosoline into the metabolite.

57. The method of claim 56, wherein the tenth enzyme is cytochrome P450 reductase (CPR), preferably wherein the CPR is as set forth in any one of the sequences as depicted in FIG. 14J or 14J, more preferably wherein CPR is from Papaver somniferum (Ps), particularly wherein PsCPR is as set forth in SEQ ID NO: 66 (FIG. 13).

58. (canceled)

59. (canceled)

60. (canceled)

61. The method of claim 57, wherein (i) 6OMT, CNMT and 4′OMT2 are expressed from a plasmid; and/or (ii) BBE and CPR are expressed from a plasmid and CFS, SPS, TNMT, MSH and P6H are expressed from a chromosome.

62. (canceled)

63. The method of claim 1, wherein the substrate is (R,S)-reticuline and the metabolite is (S)-stylopine, preferably wherein:
a. the first enzyme is berberine bridge enzyme (BBE);
b. the second enzyme is cheilanthifoline synthase (CFS) or a Ring B closer able to transform scoulerine into cheilanthifoline;
c. the third enzyme is stylopine syntase (SPS) or a Ring A closer able to transform cheilanthifoline into (S)-stylopine; and/or
d. the fourth enzyme is cytochrome P450 reductase (CPR),
more preferably wherein:
a. the BBE is as set forth in any one of the sequences as depicted in FIG. 14D or 15D;
b. the CFS is as set forth in any one of the sequences as depicted in FIG. 14E or 15E or the Ring B closer is as set forth in any one of the sequences depiced in 17A-C;
c. the SPS is as set forth in any one of the sequences as depicted in FIG. 14F or 15F or the Ring A closer is as set forth in any one of the sequences depiced in 17D-E; and/or
d. the CPR is as set forth in any one of the sequences as depicted in FIG. 14J or 15J,
particularly wherein:
a. BBE is from Papaver somniferum;
b. CFS is from Papaver somniferum;
c. SPS is from Papaver somniferum; and/or
d. CPR is from Papaver somniferum,
more particularly wherein:
a. PsBBE is as set forth in SEQ ID NO: 48 (FIG. 13);
b. PsCFS is as set forth in SEQ ID NO: 50 or 52 (FIG. 13) or the Ring B closer is as set forth in SEQ ID NO: 485 (FIG. 17);
c. PsSPS is as set forth in SEQ ID NO: 56 (FIG. 13) or the Ring A closer is as set forth in SEQ ID NO: 487 (FIG. 17); and/or
PsCPR is as set forth in SEQ ID NO: 66 (FIG. 13).

64. (canceled)

65. (canceled)

66. (canceled)

67. (canceled)

68. The method of claim 63, wherein the method comprises the second fermentation and wherein the cell further comprises a fifth heterologous coding sequence encoding a fifth enzyme involved in a metabolite pathway that converts (R,S)-norlaudanosoline into the metabolite, preferably wherein the metabolite is (S)—N-cis-methylstylopine.

69. (canceled)

70. The method of claim 68, wherein the fifth enzyme is tetrahydroprotoberberine cis-N-methyltransferase (TNMT), preferably wherein the TNMT is as set forth in any one of the sequences as depicted in FIG. 14G or 15G, more preferably wherein TNMT is from Papaver somniferum (Ps), particularly wherein PsTNMT is as set forth in SEQ ID NO: 58 (FIG. 13).

71. (canceled)

72. (canceled)

73. (canceled)

74. The method of claim 68, wherein the cell further comprises a sixth heterologous coding sequence encoding a sixth enzyme involved in a metabolite pathway that converts (R,S)-norlaudanosoline into the metabolite, preferably wherein the metabolite is protopine.

75. (canceled)

76. The method of claim 74, wherein the sixth enzyme is (S)-cis-N-methylstylopine 14-hydroxylase (MSH), preferably wherein the MSH is as set forth in any one of the sequences as depicted in FIG. 14H or 15H, more preferably wherein MSH is from Papaver somniferum (Ps).

77. (canceled)

78. (canceled)

79. The method of claim 74, wherein the cell further comprises a seventh heterologous coding sequence encoding a seventh enzyme involved in a metabolite pathway that converts (R,S)-norlaudanosoline into the metabolite, preferably wherein the metabolite is 6-hydroxyprotopine.

80. (canceled)

81. The method of claim 79, wherein the seventh enzyme is protopine 6-hydroxylase (P6H), preferably wherein the P6H is as set forth in any one of the sequences as depicted in FIG. 14I or 15I, more preferably wherein P6H is from Eschscholzia californica (Ec), particularly wherein EcP6H is as set forth in SEQ ID NO: 62 (FIG. 13).

82. (canceled)

83. (canceled)

84. (canceled)

85. The method of claim 81, wherein (i) the BBE, CFS, SPS and CPR are expressed from (i) plasmid(s); or (ii) chromosome; and/or (ii) the TNMT, MSH and P6H are are expressed from plasmid(s).

86. (canceled)

87. (canceled)

88. The method of claim 1, wherein the method comprises the second fermentation, the substrate is (S)-stylopine and wherein the metabolite is (S)-dihydrosanguinarine, preferably wherein:
a. the first enzyme is tetrahydroprotoberberine cis-N-methyltransferase (TNMT);
b. the second enzyme is (S)-cis-N-methylstylopine 14-hydroxylase (MSH);
c. the third enzyme is protopine 6-hydroxylase (P6H); and/or
d. the fourth enzyme is cytochrome P450 reductase (CPR),
more preferably wherein:
a. the TNMT is as set forth in any one of the sequences as depicted in FIG. 14G or 15G;
b. the MSH is as set forth in any one of the sequences as depicted in FIG. 14H or 15H;
c. the P6H is as set forth in any one of the sequences as depicted in FIG. 14I or 15I; and/or
d. the CPR is as set forth in any one of the sequences as depicted in FIG. 14J or 15J,
particularly wherein:
a. TNMT is from Papaver somniferum;
b. MSH is from Papaver somniferum;
c. P6H is from Eschscholzia californica; and/or
d. CPR is from Papaver somniferum,
more particularly wherein:
a. PsTNMT is as set forth in SEQ ID NO: 58 (FIG. 13);
b. PsMSH is as set forth in SEQ ID NO: 268 (FIG. 13);
c. EcP6H is as set forth in SEQ ID NO: 62 (FIG. 13); and/or
d. PsCPR is as set forth in SEQ ID NO: 66 (FIG. 13).

89. (canceled)

90. (canceled)

91. (canceled)

92. (canceled)

93. The method of claim 88, wherein the TNMT, MSH and P6H are expressed from a plasmid.

94. The method of claim 5, wherein the host cell further expresses a cytochrome b5 (Cytb5), preferably wherein the Cytb5 is as set forth in any one of the sequences as depicted in FIG. 14K.

95. (canceled)

96. A plasmid comprising nucleic acid encoding: (a) the 6OMT, CNMT and 4′OMT2 enzymes as defined in claim 5; (b) the (i) BBE, (ii) (a) CFS or (b) Ring B closer, and (iii) (a) SPS or (b) Ring A closer enzymes as defined in claim 63; (c) the TNMT, MSH and P6H enzymes as defined in claim 89; (c) the CPR enzyme as defined in claim 57; or (d) the BBE enzyme as defined in claim 63, preferably further comprising a terminator and/or a promoter, more preferably wherein the plasmid is as set forth in:
a. SEQ ID NO: 7 (FIG. 13, pGC1062);
b. SEQ ID NO: 8 (FIG. 13, pGC994); or
c. SEQ ID NO: 9 (FIG. 13, pGC997).

97. (canceled)

98. (canceled)

99. A host cell expressing (a) the 6OMT, CNMT and 4′OMT2 enzymes as defined in claim 5; (b) the (i) BBE, (ii) (a) CFS or (b) Ring B closer, and (iii) (a) SPS or (b) Ring A closer enzymes as defined in claim 63; (c) the TNMT, MSH and P6H enzymes as defined in claim 89, and the CPR enzyme as defined in claim 57; (d) the enzymes of (a) and (b) or (b) and (c); (e) the enzymes of (a), (b) and (c); or (f) one or more of the plasmids as defined in claim 96, preferably further expressing cytochrome b5, more preferably wherein the host cell (i) expresses the enzymes of (a) in a plasmid; (ii) expressing the enzymes of (b) in a plasmid or in a chromosome; (iii) expresses the enzymes of (c) in a plasmid; or (iv) expresses the enzymes of (b) and (c) in a chromosome, and more particularly wherein the host cell expresses in a plasmid the enzymes of (a) and BBE; and in a chromosome, the enzymes of (b) and (c).

100. (canceled)

101. (canceled)

102. (canceled)

103. (canceled)

104. (canceled)

105. (canceled)

106. (canceled)

107. A CYP719 polypeptide that is any one of EX45-48 (SEQ ID NOs: 324-327), EX53-58 (SEQ ID NOs: 332-337), EX65-76 (SEQ ID NOs: 344-355), EX78-80 (SEQ ID NOs: 357-359), EX82 (SEQ ID NO: 361), EX86-93 (SEQ ID NOs: 365-372), EX95-101 (SEQ ID NOs: 374-380) and EX104-105 (SEQ ID NOs: 383-384).

108. A method of preparing a benzylisoquinoline alkaloid (BIA) metabolite comprising contacting (a) a CYP719 polypeptide as defined in claim 107; or (b) A CYP719 polypeptide that is any one of EX43-44 (SEQ ID NOs: 322-323), EX49 (SEQ ID NO:328), EX51-52 (SEQ ID NOs: 330-331), EX63-64 (SEQ ID NOs: 342-343), EX77 (SEQ ID NO: 356) or EX103 (SEQ ID NO: 382), with scoulerine, nandinine and/or cheilanthifoline.

109. A method of producing:
(A) (i) N-methylcheilanthifoline; or (ii) N-methylscoulerine, comprising contacting cheilanthifoline or scoulerine, respectively, with tetrahydroprotoberberine cis-N-methyltransferase (TNMT), whereby (i) N-methylcheilanthifoline; or (ii) N-methylscoulerine are produced; or
(B) nandinine comprising contacting scoulerine with a Ring B closer as set forth in SEQ ID NO: 483, SEQ ID NO: 484, SEQ ID NO: 485, SEQ ID NO: 324, SEQ ID NO: 353, SEQ ID NO: 320, SEQ ID NO: 363, SEQ ID NO: 338, SEQ ID NO: 378, SEQ ID NO: 333, SEQ ID NO: 377, SEQ ID NO: 344, or SEQ ID NO: 374, preferably wherein the Ring B closer as set forth in SEQ ID NO: 484, SEQ ID NO: 485, SEQ ID NO: 324, SEQ ID NO: 333 or SEQ ID NO: 377.

110. (canceled)

111. (canceled)
